(19) United States
(12) Patent Application Publication
(10) Pub. No.: US 2013/0198897 A1
(43) Pub. Date: Aug. 1, 2013

(54) MODIFYING ENZYME ACTIVITY IN PLANTS

(71) Applicants: Karen Keiko Oishi, Neuchatel (CH); Dionisius Elisabeth Antonius Florack, Neuchatel (CH); Prisca Campanoni, Villars-Burquin (CH); Carlo Massimo Pozzi, Milano (IT); Jeremy Catinot, Taipei (TW); Nicolas Joseph Marie Sierro, Neuchatel (CH); Nikolai Valeryevitch Ivanov, Neuchatel (CH)

(72) Inventors: Karen Keiko Oishi, Neuchatel (CH); Dionisius Elisabeth Antonius Florack, Neuchatel (CH); Prisca Campanoni, Villars-Burquin (CH); Carlo Massimo Pozzi, Milano (IT); Jeremy Catinot, Taipei (TW); Nicolas Joseph Marie Sierro, Neuchatel (CH); Nikolai Valeryevitch Ivanov, Neuchatel (CH)

(73) Assignee: PHILIP MORRIS PRODUCTS S.A., Neuchatel, ` (CH)

(21) Appl. No.: 13/636,409

(22) PCT Filed: Mar. 22, 2011
(86) PCT No.: PCT/EP2011/054367

§ 371 Date: Dec. 3, 2012

(30) Foreign Application Priority Data
Mar. 22, 2010 (EP) 10157243.6

(51) Int. Cl. C12N 15/82

(52) U.S. Cl. CPC C12N 15/8257
USPC 800/278; 435/6.12; 435/69.1; 435/69.3; 435/69.4; 435/69.6; 435/468; 435/183; 435/193; 435/196; 435/414; 536/23.2; 800/317.3

(57) ABSTRACT

The present invention is directed to targeting genes and genomes, modifying the activity of enzymes and protein expression in plants. In particular, the present invention relates to methods for reducing the activity of one or more endogenous glycosyltransferases such as N-acetylglucosaminyltransferase, β(1,2)-xylosyltransferase and a(1,3)-fucosyl-transferase in a plant cell and to plants obtained by said method.

The present invention is directed to modifying the activity of specific enzymes in plants. In particular, the present invention relates to methods for reducing, inhibiting or substantially inhibiting the activity of one or more endogenous glycosyltransferases in plants, and to plant cells and plants obtained by said methods.

Many aspects of the N-glycosylation process in plants and mammals are similar and the processes generally involve a number of sequential enzymatic steps. However, critical differences between the mature N-glycan structures of plant glycoproteins and mammalian glycoproteins lie in the specific monosaccharides that are added during the final steps of the process. A mature N-glycan chain of a plant-produced protein typically comprises an alpha-1,3-linked fucose residue (α(1,3) fucose) and a beta-1,2-linked xylose residue (β(1,2)-xylose), both of which are absent in mammalian N-glycans.

Generally, N-glycosylation starts with the addition of a precursor Glc₃-Man₉-GlcNAc₂ oligosaccharide onto an asparagine residue in a glycosylated protein which is then sequentially processed in the endoplasmic reticulum (ER) by a number of enzymes starting with three glucosidases, glucosidase I, glucosidase II and glucosidase III and resulting in a Man₉-GlalAc₂-Asn N-glycan. Subsequently, a mannosidase I enzyme trims the mannose-rich Man₉-GlcNAc₂-Asn N-glycan to a Man₅-GlcNAc₂-Asn N-glycan. This glycosylated protein is then transported from the ER to the cis-Golgi network. Transport is mediated through vesicles and membrane fusion. An ER-derived vesicle buds off from the ER membrane and fuses to the cis-Golgi network. The Man₅-GlcNAc₂-Asn N-glycan in an eukaryote subsequently undergoes maturation in the various compartments of the Golgi apparatus through the action of a number of N-acetylglucosaminyltransferases, mannosidases and glycosyltransferases.

In mammals, including humans, during the final steps of the glycosylation process, a fucose is added in alpha-1,6-linkage (α(1,6)-fucose) onto the proximal N-acetylglucosamine residue at the non-reducing end of the N-glycan. In plants, a fucose in alpha-1,3-linkage (α(1,3)-fucose) and a xylose in beta-1,2 linkage (β(1,2)-xylose) are added to the N-glycan. Fucose residues are added onto an N-glycan chain through the action of fucosyltransferases. More specifically, in plants, an alpha-1,3-linked fucose (α(1,3)-fucose) is added by an alpha-1,3-fucosyltransferase (α(1,3)-fucosyltransferase); a xylose is added in beta-1,2-linkage (β(1,2)-xylose) onto the beta-1,4-linked mannose (β(1,4)-Man) of the tri-mannosyl (Man₃) core structure through the action of a beta-1,2-xylosyltransferase (β(1,2)-xylosyltransferase). The presence of these carbohydrates on a plant-produced protein affects the immunogenic properties of the protein when it is introduced into an animal. The different glycosylation patterns thus present a problem for the therapeutic use of plant-produced proteins in mammals, including humans, and may affect the regulatory approval of the protein.

Recombinant expression of proteins, such as proteins that can be used therapeutically in humans, constitutes an important application of transgenic plants. Tobacco plants have been considered for the production of recombinant proteins. However, tobacco plants have complex genomes. For example, Nicotiana tabacum, is an allotetraploid species that is believed to be an amphidiploid interspecific hybrid between Nicotiana sylvestris and Nicotiana tomentosiformis, and has 48 chromosomes. For each gene, including genes that encode glycosyltransferases, multiple different alleles and variants are expected to exist. Furthermore, Nicotiana tabacum has one of the largest genomes known to date (approximately 4,500 mega basepairs) comprising between 30,000 and 50,000 genes interspersed in more than 70% of “junk” DNA. The size and complexity of the tobacco genome thus present a significant challenge to gene discovery, allele and variant identification, and targeted modification of specific alleles or variants.

Given the potential of producing recombinant proteins in plants, in particular tobacco plants, there is a need for methods to identify the different endogenous glycosyltransferases that are active in glycosylation of proteins, and methods to reduce, inhibit or substantially inhibit the activity of one or more such glycosyltransferases. Particularly, it is desirable to obtain plants and plant cells which are capable of producing proteins which substantially lack alpha-1,3-linked fucose residues, beta-1,2-linked xylose residues, or both, in its N-glycan. Such plant-produced proteins can thus have favourable immunogenic properties for use in humans. It is an object of the present invention to meet these needs.

In various embodiments of the invention, (i) methods for identifying gene sequences encoding glycosyltransferases and fragments thereof, and variants and alleles of such gene sequences, (ii) methods for modifying the gene sequences, and (iii) methods for reducing, inhibiting or substantially inhibiting the enzyme activity of glycosyltransferase encoded by such sequences, are provided. Also provided are polynucleotides encoding glycosyltransferases and their variants and alleles, and fragments and mutants thereof. Also encompassed in the invention are target sites for modifications of the glycosyltransferase gene sequences, and compositions for modifying the glycosyltransferase gene sequences in plant cells, such as but not limited to, proteins comprising zinc finger domains. The invention also provides methods of use of plant cells or plants that comprise modified glycosyltransferase gene sequences for producing one or more heterologous protein, wherein the enzyme activity of one or more glycosyltransferases is reduced, inhibited or substantially inhibited. The invention also provides a plant or plant cell that is characterized by having proteins in which the N-glycans substantially lack xylose in beta-1,2-linkage or fucose in alpha-1,3-linkage, or both. Compositions comprising one or more heterologous proteins that substantially lack alpha-1,3-linked fucose residues, or beta-1,2-linked xylose residues, or both, obtainable from plants or plant cells of the invention, are also encompassed in the invention.

The technical terms and expressions used within the scope of this application are generally to be given the meaning commonly applied to them in the pertinent art of plant biology. All of the following term definitions apply to the complete content of this application. The word “comprising” does not exclude other elements or steps, and the indefinite article “a” or “an” does not exclude a plurality. A single step may fulfil the functions of several features recited in the claims. The terms “essentially”, “about”, “approximately” and the like in connection with an attribute or a value particularly also define exactly the attribute or exactly the value, respectively. The term “about” in the context of a given numerate value or range refers to a value or range that is within 20%, within 10%, or within 5% of the given value or range.

A “plant” as used within the present invention refers to any plant at any stage of its life cycle or development, and its progenies.

A “plant cell” as used within the present invention refers to a structural and physiological unit of a plant. The plant cell may be in form of a protoplast without a cell wall, an isolated single cell or a cultured cell, or as a part of higher organized unit such as but not limited to, plant tissue, a plant organ, or a whole plant.

“Plant cell culture” as used within the present invention encompasses cultures of plant cells such as but not limited to, protoplasts, cell culture cells, cells in cultured plant tissues, cells in explants, and pollen cultures.

“Plant material” as used within the present invention refers to any solid, liquid or gaseous composition, or a combination thereof, obtainable from a plant, including leaves, stems, roots, flowers or flower parts, fruits, pollen, egg cells, zygotes, seeds, cuttings, secretions, extracts, cell or tissue cultures, or any other parts or products of a plant.

“Plant tissue” as used herein means a group of plant cells organized into a structural or functional unit. Any tissue of a plant in planta or in culture is included. This term includes, but is not limited to, whole plants, plant organs, and seeds.

A “plant organ” as used herein relates to a distinct or a differentiated part of a plant such as a root, stem, leaf, flower bud or embryo.

The term “polynucleotide” is used herein to refer to a polymer of nucleotides, which may be unmodified or modified deoxyribonucleic acid (DNA) or ribonucleic acid (RNA). Accordingly, a polynucleotide can be, without limitation, a genomic DNA, complementary DNA (cDNA), mRNA, or antisense RNA. Moreover, a polynucleotide can be single-stranded or double-stranded DNA, DNA that is a mixture of single-stranded and double-stranded regions, a hybrid molecule comprising DNA and RNA, or a hybrid molecule with a mixture of single-stranded and double-stranded regions. In addition, the polynucleotide can be composed of triple-stranded regions comprising DNA, RNA, or both. A polynucleotide can contain one or more modified bases, such as phosphothioates, and can be a peptide nucleic acid (PNA). Generally, polynucleotides provided by this invention can be assembled from isolated or cloned fragments of cDNA, genome DNA, oligonucleotides, or individual nucleotides, or a combination of the foregoing.

The term “nucleotide sequence” refers to the base sequence of a polymer of nucleotides, including but not limited to ribonucleotides and deoxyribonucleotides.

The term “gene sequence” as used herein refers to the nucleotide sequence of a nucleic acid molecule or polynucleotide that encodes a polypeptide or a biologically active RNA, and encompasses the nucleotide sequence of a partial coding sequence that only encodes a fragment of a protein. A gene sequence can also include sequences having a regulatory function on expression of a gene that are located upstream or downstream relative to the coding sequence as well as intron sequences of a gene.

The term “heterologous sequence” as used herein refers to a biological sequence that does not occur naturally in the context of a specific polynucleotide or polypeptide in a cell or an organism of interest.

The term “heterologous protein”, as used herein, refers to a protein that is produced by a cell but does not occur naturally in the cell. For example, the heterologous protein produced in a plant cell can be a mammalian or human protein. A heterologous protein may contain oligosaccharide chains (glycans) covalently attached to the polypeptide in a cotranslational or posttranslational modification. As a non-limiting example, such a protein can comprise an oligosaccharide covalently linked to an asparagine (Asn) on the protein backbone comprising at least a tri-mannosyl (Man₃) core structure with two N-acetylglucosamine (GlcNAc₂) residues at the non-reducing end attached to the protein backbone (Man₃-GlcNAc₂-Asn). In particular, a heterologous protein comprises at least an N-glycan. The abbreviations “GnT” refers to N-acetylglucosaminyltransferase; “Man” refers to mannose; “Glc” refers to glucose; “Xyl” refers to xylose; “Fuc” refers to fucose; and “GlcNAc” refers to N-acetylglucosamine.

The term “N-glycosylation”, as used herein, refers to a process that starts with the transfer of a specific dolichol lipid-linked precursor oligosaccharide, Dol-PP-GlcNAc₂-Man₉-Glc₃, from the dolichol moiety in the endoplasmatic reticulum membrane, onto the free amino group of an asparagine residue (Asn), being part of a Asn-Xaa-Ybb-Xaa sequence motif in the protein backbone, resulting in a Glc₃-Man₉-GlcNAc₂-Asn glycosylated protein, wherein Xaa can be any amino acid but proline, and Ybb can be a serine, threonine or cysteine.

The term “N-glycan” as used herein refers to the carbohydrates that are attached to various asparagine residues that are each a part of a Asn-Xaa-Ybb-Xaa sequence motif in the protein backbone.

The term “non-reducing end of an N-glycan” as used herein refers to the part of the N-glycan that is attached to the asparagine of the protein backbone.

The term “beta-1,2-xylosyltransferase” (β(1,2)-xylosyltransferase) as used within the present invention refers to a xylosyltransferase, designated EC2.4.2.38, that adds a xylose in beta-1,2-linkage (β(1,2)-Xyl) onto the beta-1,4-linked mannose (β(1,4)-Man) of the trimannosyl core structure of a N-glycan of a glycoprotein.

The term “alpha-1,3-fucosyltransferase” (α(1,3)-fucosyltransferase) as used within the present invention refers to a fucosyltransferase, designated EC2.4.1.214, that adds a fucose in alpha-1,3-linkage (α(1,3)-fucose) onto the proximal N-acetylglucosamine residue at the non-reducing end of an N-glycan.

An “N-acetylglucosaminyltransferase I” as used within the present invention refers to an enzyme, designated EC2.4.1.101, that adds an N-acetylglucosamine to a mannose on the 1-3 arm of a Man₅-GlcNAc₂-Asn oligomannosyl receptor.

The term “reduce” or “reduced” as used herein, refers to a reduction of from about 10% to about 99%, or a reduction of at least 10%, at least 20%, at least 25%, at least 30%, at least 40%, at least 50%, at least 60%, at least 70%, at least 75%, at least 80%, at least 90%, at least 95%, at least 98%, or up to 100%, of a quantity or an activity, such as but not limited to enzyme activity, transcriptional activity, and protein expression.

The term “substantially inhibit” or “substantially inhibited” as used herein, refers to a reduction of from about 90% to about 100%, or a reduction of at least 90%, at least 95%, at least 98%, or up to 100%, of a quantity or an activity, such as but not limited to enzyme activity, transcriptional activity, and protein expression.

The term “inhibit” or “inhibited” as used herein, refers to a reduction of from about 98% to about 100%, or a reduction of at least 98%, at least 99%, but particularly of 100%, of a quantity or an activity, such as but not limited to enzyme activity, transcriptional activity, and protein expression.

“Genome editing technology” as used within the present invention refers to any method that results in an alteration of a nucleotide sequence in the genome of an organism, such as but not limited to, zinc finger nuclease-mediated mutagenesis, chemical mutagenesis, radiation mutagenesis, “tilling”, or meganuclease-mediated mutagenesis.

One objective of the invention is to produce in plant a heterologous protein that is suitable for use as a therapeutic, wherein the heterologous protein lacks one or more carbohydrates that would otherwise contribute undesirable immunogenic properties. Without being bound by any theory, the presence of alpha-1,3-linked fucose, beta-1,2-linked xylose, or both, on an N-glycan of a heterologous protein produced in a plant or a plant cell can be reduced or eliminated by (i) reducing, inhibiting or substantially inhibiting the enzyme activity of one or more glycosyltransferases of the invention in a plant or plant cell, or (ii) reducing inhibiting or substantially inhibiting the expression of one or more glycosyltransferases of the invention in a plant or plant cell, or both (i) and (ii).

In a specific embodiment, the glycosyltransferases of the invention are, (i) an N-acetylglucosaminyltransferase, particularly an N-acetylglucosaminyltransferase that catalyses the addition of an N-acetylglucosamine residue to a mannose residue onto the 1-3 arm of a Man₅-GlcNAc₂-Asn at the reducing end of an N-glycan of a glycoprotein; resulting in GlcNAc-Man₅-GlcNAc₂-Asn; (ii) a fucosyltransferase, particularly a fucosyltransferase that catalyzes the addition of a fucose entity in alpha-1,3-linkage to an N-glycan, particularly addition of a fucose in alpha-1,3-linkage (α(1,3)-linkage) onto the proximal N-acetylglucosamine at the non-reducing end of an N-glycan of a glycoprotein, resulting in, for example but not limited to, GlcNAc-Man₃-Fuc-GlcNAc₂-Asn or GlcNAc-Man₃-Fuc-Xyl-GlcNAc₂-Asn glycoproteins; or (iii) a xylosyltransferase, particularly a xylosyltransferase which catalyzes the addition of a xylose entity in beta-1,2-linkage to an N-glycan, particularly addition of a xylose in beta-1,2-linkage (β(1,2)-linkage) onto the beta-1,4-linked mannose (β(1,4)-linked) mannose of the trimannosyl core structure of an N-glycan, resulting in, for example but not limited to, GlcNAc-Man₃-Xyl-GlcNAc₂-Asn or GlcNAc-Man₃-Fuc-Xyl-GlcNAc₂-Asn glycoproteins. In particular, the glycosyltransferases of the invention are tobacco glycosyltransferases. Especially, the glycosyltransferases of the invention are those of Nicotiana tabacum or Nicotiana benthamiana.

In various embodiments, the invention relates to tobacco, sunflower, pea, rapeseed, sugar beet, soybean, lettuce, endive, cabbage, broccoli, cauliflower, alfalfa, duckweed, rice, maize, and carrot. In particular, the invention is directed to modified tobacco plant and modified tobacco cells, modified plants and modified cells of Nicotiana species, and particularly, modified Nicotiana benthamiana and Nicotiana tabacum plants, and Nicotiana tabacum varieties, breeding lines and cultivars, or modified cells of Nicotiana benthamiana and Nicotiana tabacum, Nicotiana tabacum varieties, breeding lines and cultivars.

In another embodiment, the invention provides genetically modified Nicotiana tabacum varieties, breeding lines, or cultivars. Non-limiting examples of Nicotiana tabacum varieties, breeding lines, and cultivars that can be modified by the methods of the invention include N. tabacum accession PM016, PM021, PM92, PM102, PM132, PM204, PM205, PM215, PM216 or PM217 as deposited with NCIMB, Aberdeen, Scotland, or DAC Mata Fina, PO2, BY-64, AS44, RG17, RG8, HB04P, Basma Xanthi BX 2A, Coker 319, Hicks, McNair 944 (MN 944), Burley 21, K149, Yaka JB 125/3, Kasturi Mawar, NC 297, Coker 371 Gold, PO2, Qisliça, Simmaba, Turkish Samsun, AA37-1, B13P, F4 from the cross BU21×Hoja Parado line 97, Samsun NN, Izmir, Xanthi NN, Karabalgar, Denizli and PO1.

In one embodiment, the modified, i.e., the genetically modified, Nicotiana tabacum plant cell, or a Nicotiana tabacum plant, including the progeny thereof, comprising the modified plant cells according to the invention and as described herein further comprises (a) at least a modification of a second coding sequence for a second N-acetyl-glucosaminyltransferase or (b) at least a modification of a third target nucleotide sequence in a genomic region comprising a coding sequence for an N-acetylglucosaminyltransferase or a combination of (a) and (b), such that (i) the activity or the expression of glycosyltransferase in the modified plant cell is reduced, inhibited or substantially inhibited, relative to a unmodified plant cell, and (ii) the alpha-1,3-fucose or beta-1,2-xylose, or both, on an N-glycan of a protein produced in the modified plant cell is reduced relative to a unmodified plant cell. In a specific embodiment, the second coding sequence is an allelic variant of the first target nucleotide sequence, or the third target nucleotide sequence is an allelic variant of the first or second target sequence.

In particular, the present invention relates in one embodiment to a modified, i.e., a genetically modified, Nicotiana tabacum plant cell, or a Nicotiana tabacum plant, including the progeny thereof, comprising the modified plant cells, wherein the modified plant cell comprises at least a modification of a first target nucleotide sequence in a genomic region comprising a coding sequence for a N-acetyl-glucosaminyltransferase such that (i) the activity or the expression of glycosyltransferase in the modified plant cell is reduced, inhibited or substantially inhibited, relative to a unmodified plant cell, and (ii) the alpha-1,3-fucose or beta-1,2-xylose, or both, on an N-glycan of a protein produced in the modified plant cell is reduced relative to a unmodified plant cell.

In one embodiment, the modified, i.e., the genetically modified, Nicotiana tabacum plant cell, or a Nicotiana tabacum plant, including the progeny thereof, comprising the modified plant cells according to the invention and as described herein further comprises (a) at least a modification of a second target nucleotide sequence in a genomic region comprising a coding sequence for β(1,2)-xylosyltransferase or (b) at least a modification of a third target nucleotide sequence in a genomic region comprising a coding sequence for α(1,3)-fucosyltransferase or a combination of (a) and (b). In one embodiment, the modified, i.e., the genetically modified, Nicotiana tabacum plant cell, or a Nicotiana tabacum plant, including the progeny thereof, comprising the modified plant cells according to the invention and as described herein further comprises a modification in an allelic variant of the first target nucleotide sequence, the second target nucleotide sequence, the third target nucleotide sequence, or a combination of any two or more of the foregoing target nucleotide sequences.

In one embodiment, the invention relates to a modified, i.e., a genetically modified, Nicotiana tabacum plant cell, or a Nicotiana tabacum plant, including the progeny thereof, comprising the modified plant cells according to the invention and as described herein, wherein the first target nucleotide sequence is
a. at least 95%, 96%, 97%, 98%, 99% or 100% identical to a nucleotide sequence selected from the group consisting of SEQ ID NOs: 12, 13, 40, 41, 233, 256, 259, 262, 265, 268, 271, 274, 277, 280;
b. at least 95%, 96%, 97%, 98%, 99% or 100% identical to a nucleotide sequence selected from the group consisting of SEQ ID NOs: 20, 21, 212, 213, 219, 220, 223, 227, 229, 234, 257, 260, 263, 266, 269, 272, 275, 278, 281.

In one embodiment, the invention relates to a modified, i.e., a genetically modified, Nicotiana tabacum plant cell, or a Nicotiana tabacum plant, including the progeny thereof, comprising the modified plant cells according to the invention and as described herein, wherein the second target nucleotide sequence is
a. at least 95%, 96%, 97%, 98%, 99% or 100% identical to a nucleotide sequence selected from the group consisting of SEQ ID NOs: 1, 4, 5, and 17;
b. at least 95%, 96%, 97%, 98%, 99% or 100% identical to a nucleotide sequence selected from the group consisting of SEQ ID NOs: 8 and 18.

In one embodiment, the invention relates to a modified, i.e., a genetically modified, Nicotiana tabacum plant cell, or a Nicotiana tabacum plant comprising the modified plant cells according to the invention and as described herein, wherein the third target nucleotide sequence is
a. at least 95%, 96%, 97%, 98%, 99% or 100% identical to a nucleotide sequence selected from the group consisting of SEQ ID NOs 27, 32, 37, and 47;
b. at least 95%, 96%, 97%, 98%, 99% or 100% identical to a nucleotide sequence selected from the group consisting of SEQ ID NOs: 28, 33, 38, and 48.

In one embodiment, the modified, i.e., the genetically modified, Nicotiana tabacum plant cell, or a Nicotiana tabacum plant, including the progeny thereof, comprising the modified plant cells according to the invention and as described herein is Nicotiana tabacum cultivar PM132, the seeds of which were deposited on 6 Jan. 2011 at NCIMB Ltd (an International Depositary Authority under the Budapest Treaty, located at Ferguson Building, Craibstone Estate, Bucksburn, Aberdeen, AB21 9YA, United Kingdom) under accession number NCIMB 41802. In another embodiment, the modified, i.e., the genetically modified, Nicotiana tabacum plant cell, or a Nicotiana tabacum plant, including the progeny thereof, comprising the modified plant cells according to the invention and as described herein is Nicotiana tabacum line PM016, the seeds of which were deposited under accession number NCIMB 41798; Nicotiana tabacum line PM021, the seeds of which were deposited under accession number NCIMB 41799; Nicotiana tabacum line PM092, the seeds of which were deposited under accession number NCIMB 41800; Nicotiana tabacum line PM102, the seeds of which were deposited under accession number NCIMB 41801; Nicotiana tabacum line PM204, the seeds of which were deposited on 6 Jan. 2011 at NCIMB Ltd. under accession number NCIMB 41803; Nicotiana tabacum line PM205, the seeds of which were deposited under accession number NCIMB 41804; Nicotiana tabacum line PM215, the seeds of which were deposited under accession number NCIMB 41805; Nicotiana tabacum line PM216, the seeds of which were deposited under accession number NCIMB 41806; and Nicotiana tabacum line PM217, the seeds of which were deposited under accession number NCIMB 41807.

In still another embodiment of the invention, the Nicotiana tabacum cultivar PM132, deposited under accession NCIMB 41802 comprises a the target nucleotide sequence at least 95%, 96%, 97%, 98%, 99% or 100% identical to a nucleotide sequence selected from the group consisting of SEQ ID NOs: 256, 259, 262, 265, 268, 271, 274, 277 and 280, which sequence is used for designing a mutagenic oligonucleotide capable of recognizing and binding at or adjacent to said target site such that the activity or the expression of the glycosyltransferase, and, optionally, of at least one allelic variant thereof, in the modified plant or plant cell is reduced, inhibited or substantially inhibited relative to an unmodified plant cell and the glycoproteins produced by said modified plant or plant cell lack alpha-1,3-linked fucose residues and beta-1,2-linked xylose residues in their N-glycan.

In a specific embodiment, said target nucleotide sequence is a sequence as shown in SEQ ID No: 256.

In another specific embodiment, said target nucleotide sequence is a sequence as shown in SEQ ID No: 259.

In still another specific embodiment, said target nucleotide sequence is a sequence as shown in SEQ ID No: 262.

In still another embodiment of the invention, the Nicotiana tabacum cultivar PM132, deposited under accession NCIMB 41802 comprises a target nucleotide sequence at least 95%, 96%, 97%, 98%, 99% or 100% identical to a nucleotide sequence selected from the group consisting of SEQ ID NOs: 257, 260, 263, 266, 269, 272, 275, 278, and 281, which sequence is used for designing a mutagenic oligonucleotide capable of recognizing and binding at or adjacent to said target site such that the activity or the expression of the glycosyltransferase, and, optionally, of at least one allelic variant thereof, in the modified plant or plant cell is reduced, inhibited or substantially inhibited relative to an unmodified plant cell and the glycoproteins produced by said modified plant or plant cell lack alpha-1,3-linked fucose residues and beta-1,2-linked xylose residues in their N-glycan.

In a specific embodiment, said target nucleotide sequence is a sequence as shown in SEQ ID No: 257.

In another specific embodiment, said target nucleotide sequence is a sequence as shown in SEQ ID No: 260.

In still another specific embodiment, said target nucleotide sequence is a sequence as shown in SEQ ID No: 263.

In certain embodiments, the invention relates to the progeny of a modified Nicotiana tabacum plant according to the invention and as described herein, wherein said progeny plant comprises at least one of the previously defined modifications, such that the activity or the expression of the glycosyltransferase is reduced, inhibited or substantially inhibited relative to an unmodified plant and (ii) the alpha-1,3-fucose or beta-1,2-xylose, or both, on an N-glycan of a protein produced in the modified plant is reduced relative to an unmodified plant.

In one embodiment, the modified, i.e., the genetically modified, Nicotiana tabacum plant cell, or a Nicotiana tabacum plant, including the progeny thereof, comprising the modified plant cells according to the invention and as described herein can be used in a method for producing a heterologous protein, said method comprising: introducing into a modified Nicotiana tabacum plant cell or plant as defined herein an expression construct comprising a nucleotide sequence that encodes a heterologous protein, particularly a vaccine antigen, a cytokine, a hormone, a coagulation protein, an apolipoprotein, an enzyme for replacement therapy in human, an immunoglobulin or a fragment thereof; and culturing the modified plant cell that comprises the expression construct such that the heterologous protein is produced, and optionally, regenerating a plant from the plant cell, and growing the plant and its progenies.

In one embodiment, the present invention provides methods for reducing, inhibiting or substantially inhibiting the enzyme activity of one or more glycosyltransferases that are involved in the N-glycosylation of proteins in plants. Specifically, the method comprises modifying the coding sequences, particularly the genomic nucleotide sequences, of one or more glycosyltransferases in a plant or a plant cell, and optionally, selecting and/or isolating modified plant cells in which the enzyme activity of one or more of the glycosyltransferases or the total glycosyltransferase activity is reduced, inhibited or substantially inhibited. The method can comprise, optionally, the identification of a glycosyltransferase, a fragment thereof or an allele or variant thereof.

In particular, the invention relates to a method for producing a Nicotiana tabacum plant or plant cell capable of producing humanized glycoproteins, the method comprising:
(i) modifying in the genome of a tobacco plant cell
   a. a first target nucleotide sequence in a genomic region comprising a coding sequence for a N-acetylglucosaminyltransferase; or
   b. the first target nucleotide sequence of a) and a second target nucleotide sequence in a genomic region comprising a coding sequence for a β(1,2)-xylosyltransferase or an α(1,3)-fucosyltransferase; or
   c. the first target nucleotide sequence of a) and the second target nucleotide sequence of b) and a third target nucleotide sequence in a genomic region comprising a coding sequence for a β(1,2)-xylosyltransferase or an α(1,3)-fucosyltransferase; and, optionally,
   d. a target nucleotide in a genomic region comprising an allelic variant of (a), (b) or (c), or of a combination of any two or more of the foregoing target nucleotide sequences.
(ii) identifying and, optionally, selecting a modified plant or plant cell comprising the modification in the target nucleotide sequence,
   wherein the activity or the expression of the glycosyltransferases as defined in a), b), c) and d), and, optionally, of at least one allelic variant thereof in the modified plant or plant cell is reduced, inhibited or substantially inhibited relative to an unmodified plant cell and the glycoproteins produced by said modified plant or plant cell lack alpha-1,3-linked fucose residues and beta-1,2-linked xylose residues in their N-glycan.

In particular, the invention relates to a method for producing a Nicotiana tabacum plant or plant cell capable of producing humanized glycoproteins, the method comprising:
(i) modifying in the genome of a tobacco plant cell
   a. a first target nucleotide sequence in a genomic region comprising a coding sequence for a N-acetylglucosaminyltransferase; or
   b. the first target nucleotide sequence of a) and a second target nucleotide sequence coding sequence for a N-acetylglucosaminyltransferase; or
   c. the first target nucleotide sequence of a) and the second target nucleotide sequence of b) and a third target nucleotide sequence in a genomic region comprising a coding sequence for a N-acetylglucosaminyltransferase; wherein the second or third target nucleotide sequence, or the second and third target nucleotide sequence, comprise an allelic variant of (a).
(ii) identifying and, optionally, selecting a modified plant or plant cell comprising the modification in the target nucleotide sequence,
   wherein the activity or the expression of the glycosyltransferases as defined in a), b) and c) in the modified plant or plant cell is reduced, inhibited or substantially inhibited relative to an unmodified plant cell, and the glycoproteins produced by said modified plant or plant cell lack alpha-1,3-linked fucose residues and beta-1,2-linked xylose residues in their N-glycan.

In particular, in the method for producing a Nicotiana tabacum plant or plant cell capable of producing humanized glycoproteins according to the invention and as described herein, the modification of the genome of the tobacco plant or plant cell comprises
a. identifying in the target nucleotide sequence of a Nicotiana tabacum plant or plant cell and, optionally, in at least one allelic variant thereof, a target site,
b. designing, based on the target nucleotide sequence according to the invention a mutagenic oligonucleotide capable of recognizing and binding at or adjacent to said target site, and
c. binding the mutagenic oligonucleotide to the target nucleotide sequence in the genome of a tobacco plant or plant cell under conditions such that the genome is modified.

In one embodiment, the mutagenic oligonucleotide is used in genome editing technology, particularly in zinc finger nuclease-mediated mutagenesis, tilling, homologous recombination, oligonucleotide-directed mutagenesis, or meganuclease-mediated mutagenesis, or a combination of the foregoing technologies.

In one embodiment, the invention relates to a Nicotiana tabacum plant cell, or a Nicotiana tabacum plant comprising the modified plant cells, produced by the method according to the invention and as described herein.

In another embodiment of the invention, the plant modified to be capable of producing humanized glycoproteins according to the invention and as described herein, is Nicotiana tabacum cultivar PM132, deposited under accession NCIMB 41802.

In still another embodiment of the invention, the target nucleotide sequence identified in Nicotiana tabacum cultivar PM132, deposited under accession NCIMB 41802 and used for designing a mutagenic oligonucleotide capable of recognizing and binding at or adjacent to said target site is a sequence at least 95%, 96%, 97%, 98%, 99% or 100% identical to a nucleotide sequence selected from the group consisting of SEQ ID NOs: 256, 259, 262, 265, 268, 271, 274, 277 and 280.

In a specific embodiment, said target nucleotide sequence is a sequence as shown in SEQ ID No: 256.

In still another embodiment of the invention, the target nucleotide sequence identified in Nicotiana tabacum cultivar PM132, deposited under accession NCIMB 41802 and used for designing a mutagenic oligonucleotide capable of recognizing and binding at or adjacent to said target site is a sequence at least 95%, 96%, 97%, 98%, 99% or 100% identical to a nucleotide sequence selected from the group consisting of SEQ ID NOs: 257, 260, 263, 266, 269, 272, 275, 278, and 281.

In a specific embodiment, said target nucleotide sequence is a sequence as shown in SEQ ID No: 257.

In one embodiment, the modified, i.e., the genetically modified, Nicotiana tabacum plant cell, or a Nicotiana tabacum plant, including the progeny thereof, comprising the modified plant cells according to the invention and as described herein is Nicotiana tabacum cultivar PM132, deposited under accession NCIMB 41802, which further comprises (a) at least a modification of a second target nucleotide sequence in a genomic region comprising a coding sequence for β(1,2)-xylosyltransferase, which sequence is at least 96%, 96%, 97%, 98%, 99% or 100% to a nucleotide sequence selected from the group consisting of SEQ ID Nos: 1, 4, 5, and 17 and SEQ ID NOs: 8 and 18, respectively; or (b) at least a modification of a third target nucleotide sequence in a genomic region comprising a coding sequence for α(1,3)-fucosyltransferase, which sequence is at least 95%, 96%, 97%, 98%, 99% or 100% to a nucleotide sequence selected from the group consisting of SEQ ID Nos: 27, 32, 37, and 47 and SEQ ID NOs: 28, 33, 38, and 48, respectively; or a combination of (a) and (b).

Because of the size and complexity of the tobacco genome and the presence of potentially multiple variants and alleles, a strategy had to be devised to identify gene sequences of the glycosyltransferases. According to the invention, methods for identifying a gene sequence encoding a plant glycosyltransferase are provided. In a specific embodiment, a method of the invention can comprise (i) constructing a plant genomic DNA library, for example, a bacterial artificial chromosome (BAC) genomic DNA library according to methods known in the art, (ii) hybridizing a polynucleotide probe to genomic clones in the genomic DNA library, such as a BAC clone, under conditions that allow the probe to bind to homologous nucleotide sequences, and (iii) identifying a genomic DNA clone that hybridized to the probe. The probe is designed according to nucleotide sequences that encode glycosyltransferases or fragments thereof. The nucleotide sequence of the genomic DNA clone, including fragments or portions of sequence that encodes a glycosyltransferase, can be sequenced according to methods known in the art.

Alternatively, a polynucleotide comprising a sequence that encodes a known glycosyltransferase, such as one that has been identified in a first plant, can be used to screen a collection of exon sequences of a second plant, such as a tobacco plant. An exon sequence with homology to the polynucleotide encoding the known glycosyltransferase can be used to develop probes for screening a genomic DNA library of the second plant, such as a tobacco BAC library, to identify a BAC clone and establish the genomic sequence of a glycosyltransferase of the second plant.

To assist in identifying genomic nucleotide sequences that encode the glycosyltransferases of the invention, the genomic nucleotide sequences are compared in silico to a database of nucleotide sequences of exons that are known to be expressed in a particular plant organ, for example, leaves. Genomic nucleotide sequences that match a desired expression profile, such as genes that are expressed in leaves or genes that are only expressed in leaves, are selected for further characterization. This aspect of the invention focuses the identification process on sequences of relevance and reduces the number of candidate sequences. Pseudogenes, inactive alleles or variants, alleles or variants that are not expressed in a particular organ, such as leaves, are thus excluded.

Accordingly, as a non-limiting example, a genomic DNA sequence encoding a beta-(1,2)-xylosyltransferase of Nicotiana tabacum or a fragment thereof can be identified by screening a Nicotiana tabacum BAC library using a polynucleotide probe. The probe can be designed according to the nucleotide sequence of an exon of a tobacco beta-(1,2)-xylosyltransferase that can be assembled by compiling Nicotiana sequences that show homology to an Arabidopsis thaliana beta-(1,2)-xylosyltransferase. The expression of the exon can be tested by detecting its mRNA in tobacco leaves using a microarray comprising polynucleotides of tobacco exons.

In another non-limiting example, a genomic DNA sequence encoding an alpha (1,3)-fucosyltransferase of Nicotiana tabacum or a fragment thereof can be identified by screening a Nicotiana tabacum BAC library using a polynucleotide probe. The probe can be designed according to the nucleotide sequence of an exon of a tobacco alpha(1,3)-fucosyltransferase that can be compiled by identifying Nicotiana sequences that show homology to an Arabidopsis thaliana alpha(1,3)-fucosyltransferase and tested by detecting its expression in tobacco leaves using a microarray comprising polynucleotides of tobacco exons.

Alternative methods for identifying in a plant cell a genomic DNA sequence encoding glycosyltransferases of the invention may also be used within the method according to the present invention. The polynucleotide sequences of glycosyltransferases disclosed in the present invention can be used to identify additional alleles of these glycosyltransferases and other related glycosyltransferases, according to the methods described above.

In another embodiment of the invention, a genomic DNA sequence comprising a coding sequence for a glycosyltransferase or a fragment thereof can be identified by polymerase chain reaction (PCR) using nucleic acid primers that are designed according to sequences encoding glycosyltransferases. In particular, the following forward primers and reverse primers can be used in combination to identify additional alleles of glycosyltransferases of the invention and other related glycosyltransferases:
a forward primer of SEQ ID NO: 2 and a reverse primer of SEQ ID NO: 3;
a forward primer of SEQ ID NO: 10 and a reverse primer of SEQ ID NO: 11;
a forward primer of SEQ ID NO: 15 and a reverse primer of SEQ ID NO: 16;
a forward primer of SEQ ID NO: 23 and a reverse primer of SEQ ID NO: 24;
a forward primer of SEQ ID NO: 25 and a reverse primer of SEQ ID NO: 26;
a forward primer of SEQ ID NO: 30 and a reverse primer of SEQ ID NO: 31;
a forward primer of SEQ ID NO: 35 and a reverse primer of SEQ ID NO: 36,
a forward primer of SEQ ID NO: 45 and a reverse primer of SEQ ID NO: 46 or
a forward primer of SEQ ID NO: 231 and a reverse primer of SEQ ID NO: 232,
a forward primer of SEQ ID NO: 236 and a reverse primer of SEQ ID NO: 237,
a forward primer of SEQ ID NO: 238 and a reverse primer of SEQ ID NO: 239,
a forward primer of SEQ ID NO: 240 and a reverse primer of SEQ ID NO: 241,
a forward primer of SEQ ID NO: 242 and a reverse primer of SEQ ID NO: 243,
a forward primer of SEQ ID NO: 244 and a reverse primer of SEQ ID NO: 245,
a forward primer of SEQ ID NO: 246 and a reverse primer of SEQ ID NO: 247,
a forward primer of SEQ ID NO: 248 and a reverse primer of SEQ ID NO: 249,
a forward primer of SEQ ID NO: 250 and a reverse primer of SEQ ID NO: 251,
a forward primer of SEQ ID NO: 252 and a reverse primer of SEQ ID NO: 253, or
a forward primer of SEQ ID NO: 254 and a reverse primer of SEQ ID NO: 255.

The present invention provides primers having the sequences shown in SEQ ID NO: 2 and SEQ ID NO: 3 for the amplification of a fragment of contig gDNA_c1736055; SEQ ID NO: 10 and SEQ ID NO: 11 for the amplification of a fragment of GnTI-B of Nicotiana tabacum and Nicotiana benthamiana; SEQ ID NO: 15 and SEQ ID NO: 16 for the amplification of a fragment of contig CHO_OF4335xn13f1; SEQ ID NO: 23 and SEQ ID NO: 24 for the amplification of a fragment of GnTI-A of Nicotiana tabacum and Nicotiana benthamiana; SEQ ID NO: 25 and SEQ ID NO: 26 for the amplification of a fragment of contig CHO_OF3295xj17f1; SEQ ID NO: 30 and SEQ ID NO: 31 for the amplification of a fragment of contig gDNA_c1765694; SEQ ID NO: 35 and SEQ ID NO: 36 for the amplification of a fragment of contig_CHO_OF4881xd22dr1, or SEQ ID NO: 45 and SEQ ID NO: 46 for the amplification of contig CHO_OF4486xe11f1, SEQ ID NO: 231 and SEQ ID NO: 232 for the amplification of a fragment of contig gDNA_c1690982 that contains a Nicotiana tabacum N-acetylglucosaminyltransferase I intron-exon sequence, SEQ ID NO: 236 and SEQ ID NO: 237 for the amplification of FABIJI-homolog of N. tabacum PM132, SEQ ID NO: 238 and SEQ ID NO: 239 for the amplification of CPO GnTI genomic sequence of N. tabacum PM132, SEQ ID NO: 240 and SEQ ID NO: 241 for the amplification of CAC80702.1 homolog of N. tabacum PM132, SEQ ID NO: 242 and SEQ ID NO: 243 for the amplification of GnTI sequence of N. tabacum Hicks Broadleaf, SEQ ID NO: 244 and SEQ ID NO: 245 for the amplification of GnTI sequence of N. tabacum Hicks Broadleaf, SEQ ID NO: 246 and SEQ ID NO: 247 for the amplification of gDNA of N. tabacum PM132 containing 5′ UTR and exons 1 to 7, SEQ ID NO: 248 and SEQ ID NO: 249 for the amplification of gDNA of N. tabacum PM132 containing exons 4 to 13, SEQ ID NO: 250 and SEQ ID NO: 251 for the amplification of gDNA of N. tabacum PM132 containing exons 12 to 19 and 3′ UTR, SEQ ID NO: 252 and SEQ ID NO: 253 for the amplification of gDNA of N. tabacum PM132 containing exons 12 to 19 and 3′ UTR, SEQ ID NO: 254 and SEQ ID NO: 255: for the amplification of gDNA of N. tabacum PM132 containing exons 12 to 19 and 3′ UTR.

The invention also encompasses polynucleotides that comprises the nucleotide sequence of one of the primers set forth in SEQ ID Nos: 2, 3, 10, 11, 15, 16, 23, 24, 25, 26, 30, 31, 35, 36, 45, or 46, 231, 232, 236, 237, 238, 239, 240, 241, 242, 243, 244, 245, 246, 247, 248, 249, 250, 251, 252, 253, 254, or 255 or a subsequence thereof that is greater than or equal to 10 base pairs in length. However, the skilled person is in a position to modify and amend these primers, primer sequences and primer pairs, for example, by elongation or shortening or a combination of elongation and shortening of the sequences or specific nucleotide exchanges.

Based on the methods of the invention as described above, the invention provides nucleotide sequences that encode at least a fragment of a glycosyltransferase of the invention, particularly SEQ ID NOS: 1, 4, 5, 7, 12, 13, 14, 17, 27, 32, 37, 40, 41, and 47, 233. In another embodiment, the invention provides nucleotide sequences that encode at least a fragment of a glycosyltransferase of the invention, particularly SEQ ID NOs: 256, 259, 262, 265, 268, 271, 274, 277 and 280. In another embodiment, the invention provides nucleotide sequences that encode at least a fragment of a glycosyltransferase of the invention, particularly SEQ ID NOs: 18, 20, 21, 22, 28, 33, 38, 48, 212, 213, 219, 220, 223, 225, 227, 229, 234. In another embodiment, the invention provides nucleotide sequences that encode at least a fragment of a glycosyltransferase of the invention, particularly 257, 260, 263, 266, 269, 272, 275, 278, 281.

Also encompassed in the invention are polynucleotides that share at least 90%, at least 95%, at least 96%, at least 97%, at least 98%, or at least 99% sequence identity to the nucleotide sequence of any one of SEQ ID NOS: 1, 4, 5, 7, 12, 13, 14, 17, 27, 32, 37, 40, 41, and 47, 233, to the nucleotide sequence of any one of SEQ ID NOS: 256, 259, 262, 265, 268, 271, 274, 277 and 280, to the nucleotide sequence of any one of SEQ ID NOS: 18, 20, 21, 22, 28, 33, 38, 48, 212, 213, 219, 220, 223, 225, 227, 229, 234, to the nucleotide sequence of any one of SEQ ID NOS: 257, 260, 263, 266, 269, 272, 275, 278, 281. Also encompassed in the invention are polynucleotides which hybridize, particularly under stringent conditions, to a nucleic acid probe that comprises (i) the nucleotide sequence of any one of SEQ ID NOS: 1, 4, 5, 7, 12, 13, 14, 17, 27, 32, 37, 40, 41, and 47, 233; or (ii) the complement of a nucleotide sequence of any one of SEQ ID NOS: 1, 4, 5, 7, 12, 13, 14, 17, 27, 32, 37, 40, 41, and 47, 233.

Also encompassed in the invention are polynucleotides which hybridize, particularly under stringent conditions, to a nucleic acid probe that comprises (1) the nucleotide sequence of any one of SEQ ID NOS: 256, 259, 262, 265, 268, 271, 274, 277 and 280, or (ii) the complement of a nucleotide sequence of any one of SEQ ID NOS: SEQ ID NOS: 256, 259, 262, 265, 268, 271, 274, 277 and 280.

Also encompassed in the invention are polynucleotides which hybridize, particularly under stringent conditions, to a nucleic acid probe that comprises (i) the nucleotide sequence of any one of SEQ ID NOS: 257, 260, 263, 266, 269, 272, 275, 278, 281, or (ii) the complement of a nucleotide sequence of any one of SEQ ID NOS: SEQ ID NOS: 257, 260, 263, 266, 269, 272, 275, 278, 281.

Also encompassed in the invention are polynucleotides which hybridize, particularly under stringent conditions, to a nucleic acid probe that comprises (i) the nucleotide sequence of any one of SEQ ID NOS: 18, 20, 21, 22, 28, 33, 38, 48, 212, 213, 219, 220, 223, 225, 227, 229, 234, or (ii) the complement of a nucleotide sequence of any one of SEQ ID NOS: SEQ ID NOS: 18, 20, 21, 22, 28, 33, 38, 48, 212, 213, 219, 220, 223, 225, 227, 229, 234.

Also encompassed in the invention are fragments of the polynucleotides disclosed above.

Fragments of the polynucleotides of the invention, including but not limited to oligonucleotides or primers, can be at least 16 nucleotides in length. In various embodiments, the fragments can be at least about 20, 30, 40, 50, 60, 70, 80, 90, 100, 200, 300, 400, 500, 1000, 1500, 2000, 2500, 3000, 3500, 4000, 4500, 5000, 6000, 7000, 8000, 9000, or more contiguous nucleotides in length. Alternatively, the fragments can comprise nucleotide sequences that encode about 10, 20, 25, 30, 35, 40, 45, 50, 55, 60, 65, 70, 75, 80, 85, 90, 100, 150, 200, 250, 300, 350, 400, 450, 500, 600, 700, 800, 900, 1000, or more contiguous amino acid residues of a glycosyltransferase of the invention. Fragments of the polynucleotides of the invention can also refer to exons or introns of a glycosyltransferase of the invention, as well as portions of the coding regions of such polynucleotides that encode functional domains such as signal sequences and active site(s) of an enzyme. Many such fragments can be used as nucleic acid probes for the identification of polynculeotifes of the invention.

The present invention further relates to a glucosyltransferase encoded by the above identified polynucleotides of the invention, wherein said glucosyltransferase is
a. an N-acetylglucosaminyltransferase exhibiting an amino acid sequence as shown in SEQ ID NOs: 214, 215, 217, 218, 221, 222, 224, 228, 230, 235, 258, 264, 267, 270, 273, 276, 279 and 262;
b. a β(1,2)-xylosyltransferase exhibiting an amino acid sequence as shown in SEQ ID NOs: 9 and 19;
c. an α(1,3)-fucosyltransferase exhibiting an amino acid sequence as shown in SEQ ID NOs: 29, 34, 39, and 49;
d. an amino acid sequence that is at least 95%, 96%, 97%, 98%, 99% identical to the amino acid sequence of (i), (ii), or (iii).

In one embodiment of the invention, a genomic nucleotide sequence as defined herein is used for identifying a target site in
a. a first target nucleotide sequence in a genomic region comprising a coding sequence for a N-acetylglucosaminyltransferase; or

b. the first target nucleotide sequence of a) and a second target nucleotide sequence in a genomic region comprising a coding sequence for a β(1,2)-xylosyltransferase; or
c. the first target nucleotide sequence of a) and a third target nucleotide sequence in a genomic region comprising a coding sequence for an α(1,3)-fucosyltransferase; or
d. all target nucleotide sequences a), b) and c);

for modification such that (i) the activity or the expression of an N-acetyl-glucosaminyltransferase, or of an N-acetylglucos-aminyltransferase and a β(1,2)-xylosyltransferase, or of an N-acetylglucos-aminyltransferase and an α(1,3)-fucosyl-transferase or of an N-acetylglucos-aminyltransferase, a β(1,2)-xylosyltransferase, and an α(1,3)-fucosyltransferase and, optionally, of at least one allelic variant thereof, in a modified plant cell comprising the modification is reduced relative to a unmodified plant cell, and (ii) the alpha-1,3-fucose or beta-1,2-xylose, or both, on a N-glycan of a protein in a modified plant cell comprising the modification is reduced relative to a unmodified plant cell.

In one embodiment of the invention, a genomic nucleotide sequence as defined herein is used for identifying a target site in
a. a first target nucleotide sequence in a genomic region comprising a coding sequence for a N-acetylglucosaminyltransferase; or
b. the first target nucleotide sequence of a) and a second target nucleotide sequence in a genomic region comprising a coding sequence for a second N-acetyl-glucosaminyltransferase; or
c. the first target nucleotide sequence of a) and a third target nucleotide sequence in a genomic region comprising a coding sequence for a third N-acetyl-glucosaminyltransferase; or
d. all target nucleotide sequences a), b) and c);
   for modification such that (i) the activity or the expression of an N-acetyl-glucosaminyltransferase, or of two or more N-acetylglucosaminyltransferases in a modified plant cell comprising the modification, is reduced relative to a unmodified plant cell, and (ii) the alpha-1,3-fucose or beta-1,2-xylose, or both, on a N-glycan of a protein in a modified plant cell comprising the modification is reduced relative to a unmodified plant cell. The second or third nucleotide sequence, or second and third nucleotide sequence can be allelic variants of the first nucleotide sequence.

In a specific embodiment of the invention, a non-natural zinc finger protein that selectively binds a genome nucleotide sequence or a coding sequence as defined herein is used, for making a zinc finger nuclease that introduces a double-stranded break in at least one of the target nucleotide sequences.

In another embodiment, the present invention is directed toward the regulatory regions that are found upstream and downstream of the coding sequences disclosed herein, which are readily determined and isolated from the genomic sequences provided herein. Included within such regulatory regions are, without limitation, promoter sequences, upstream activator sequences as well as binding sites for regulatory proteins that modulate the expression of the genes identified herein.

RNAi, shRNA (McIntyre and Fanning (2006), BMC Biotechnology 6:1), ribozymes, antisense nucleotide sequences (like antisense DNAs or antisense RNAs), siRNA (Hannon (2003), Rnai: A Guide to Gene Silencing, Cold Spring Harbor Laboratory Press, USA), and PNAs corresponding to genomic DNA sequences of the glycosyltransferase of the invention are also contemplated.

In specific embodiments, the invention provides four gene sequences that encode alpha-1,3-fucosyltransferases, fragments, variants or allelic forms thereof; two gene sequences that encode beta-1,2-xylosyltransferases, fragments, variants or allelic forms thereof; and one gene sequence that encodes N-acetylyglucosaminyltransferase I, fragments, variants or allelic forms thereof. Particularly, the glycosyltransferases of the invention are expressed in leaves.

The term “percent identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. The term “identity” is used herein in the context of a nucleotide sequence or amino acid sequence to describe two sequences that are at least 50%, at least 55%, at least 60%, particularly of at least 70 at least 75% more particularly of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%, identical to one another.

If two sequences which are to be compared with each other differ in length, sequence identity preferably relates to the percentage of the nucleotide residues of the shorter sequence which are identical with the nucleotide residues of the longer sequence. As used herein, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described herein below. For example, sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W.R. Pearson (1990), Methods in Enzymology 183, 63-98, appended examples and http://workbench.sdsc.edu/). For this purpose, the “default” parameter settings may be used.

If the two nucleotide sequences to be compared by sequence comparison, differ in identity refers to the shorter sequence and that part of the longer sequence that matches the shorter sequence. In other words, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence.

Nucleotide or amino acid sequences which have at least 50%, at least 55%, at least 60%, particularly of at least 70%, at least 75% more particularly of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the herein-described nucleotide or amino acid sequences, may represent alleles, derivatives or variants of these sequences which preferably have a similar biological function. They may be either naturally occurring variations, for instance allelic sequences, sequences from other ecotypes, varieties, species, etc., or mutations. The mutations may have formed naturally or may have been produced by deliberate mutagenesis methods, such as those disclosed in the present invention. Furthermore, the variations may be synthetically produced sequences. The allelic variants may be naturally occurring variants or synthetically produced variants or variants produced by recombinant DNA techniques. Deviations from the above-described polynucleotides may have been produced, e.g., by deletion, substitution, addition, insertion or recombination or insertion and recombination. The term “addition” refers to adding at least one nucleic acid residue or amino acid to the end of the given sequence, whereas “insertion” refers to inserting at least one nucleic acid residue or amino acid within a given sequence.

Another indication that two nucleic acid sequences are substantially identical is that the two polynucleotides hybridize to each other under stringent conditions. The phrase: “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a nucleic acid probe and a target nucleic acid and embraces minor mismatches that can be accommodated by reducing the stringency of the hybridization media to achieve the desired detection of the target nucleic acid sequence.

Polynucleotide sequences which are capable of hybridizing with the polynucleotide sequences provided herein can, for instance, be isolated from genomic DNA libraries or cDNA libraries of plants. Particularly, such polynucleotides are from plant origin, particularly preferred from a plant belonging to the genus of Nicotiana, particularly Nicotiana benthamiana or Nicotiana tabacum. Alternatively, such nucleotide sequences can be prepared by genetic engineering or chemical synthesis.

Such polynucleotide sequences being capable of hybridizing may be identified and isolated by using the polynucleotide sequences described herein, or parts or reverse complements thereof, for instance by hybridization according to standard methods (see for instance Sambrook and Russell (2001), Molecular Cloning: A Laboratory Manual, CSH Press, Cold Spring Harbor, N.Y., USA). Nucleotide sequences comprising the same or substantially the same nucleotide sequences as indicated in the listed SEQ ID NOs, or parts or fragments thereof, can, for instance, be used as hybridization probes. The fragments used as hybridization probes can also be synthetic fragments which are prepared by usual synthesis techniques, the sequence of which is substantially identical with that of a nucleotide sequence according to the invention.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, N.Y. Generally, highly stringent hybridization and wash conditions are selected to be about 5° C. lower than the thermal melting point for the specific sequence at a defined ionic strength and pH. Typically, under “stringent conditions” a probe will hybridize to its target subsequence, but to no other sequences.

The thermal melting point is the temperature (under defined ionic strength and pH) at which 50% of the target sequence hybridizes to a perfectly matched probe. Very stringent conditions are selected to be equal to the melting temperature (Tₘ) for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2 times SSC wash at 65° C. for 15 minutes (see Sambrook, infra, for a description of SSC buffer). Often, a high stringency wash is preceded by a low stringency wash to remove background probe signal. An example of medium stringency wash for a duplex of, e.g., more than 100 nucleotides, is 1 times SSC at 45° C. for 15 minutes. An example low stringency wash for a duplex of, e.g., more than 100 nucleotides, is 4-6 times SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2 times (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g. when a copy of a nucleic acid is created using the maximum codon degeneracy permitted by the genetic code.

After a nucleotide sequence encoding at least a fragment of a glycosyltransferase of the invention has been identified, the invention further provides methods for modifying the nucleotide sequence in a plant or a plant cell, resulting in a plant or a plant cell that exhibits a reduction, an inhibition or a substantial inhibition of the enzyme activity of the glycosyltransferase, or a reduced level of expression of the glycosyltransferase. The reduction, an inhibition or a substantial inhibition in enzyme activity or the change in expression level is relative to that in a naturally occurring plant cell, an unmodified plant cell, or a plant cell not modified by a method of the invention, any one of which can be used as a control. A comparison of enzyme activities or expression levels against such a control can be carried out by any methods known in the art.

The term modified plant cell or modified plant is used herein interchangably with the term genetically modified plant cell or gentically modified plant and refers to a plant cell that is artificially modified to contain a mutation or modification in one of the nucleotide sequences comprised within the plant cells genome by applying method known in the art including, but without being limited to, chemical mutagenesis or genome editing technologies such as those described in detail herein below as well as plants comprising such a modified plant cell.

Many methods known in the art can be used to mutate the nucleotide sequence of a glycosyltransferase gene of the invention. Methods that introduce a mutation randomly in a gene sequence can be, without being limited to, chemical mutagenesis, such as but not limited to EMS mutatagenesis and radiation mutagenesis. Methods that introduce targeted mutation into a cell include but are not limited to genome editing technology, particularly zinc finger nuclease-mediated mutagenesis, tilling (targeting induced local lesions in genomes, as described in McCallum et al., Plant Physiol, June 2000, Vol. 123, pp. 439-442 and Henikoff et al., Plant Physiology 135:630-636 (2004)), homologous recombination, oligonucleotide-directed mutagenesis, and meganuclease-mediated mutagenesis. Many methods known in the art for screening mutated gene sequences can be used to identify or confirm a mutation.

The general use of zinc finger nuclease-mediated mutagenesis is known in the art and described in patent publications, such as but not limited to, WO02057293, WO02057294, WO0041566, WO0042219, and WO2005084190, which are incorporated herein by reference in its entirety. The general use of meganuclease-mediated mutagenesis is known in the art and described in patent publications, such as but not limited to, WO96/14408, WO2003025183, WO2003078619, WO2004067736, WO2007047859, and WO2009059195, which are incorporated herein by reference in its entirety.

A method of the invention thus comprises modifying a sequence that encodes a glycosyltransferase of the invention in a plant cell by applying mutagenesis such as chemical mutagenesis or radiation mutagenesis. Another method of the invention comprises modifying a target site in a sequence that encodes a glycosyltransferase of the invention by applying genome editing technology, such as but not limited to zinc finger nuclease-mediated mutagenesis, “tilling” (targeting induced local lesions in genomes), homologous recombination, oligonucleotide-directed mutagenesis and meganuclease-mediated mutagenesis.

Given that multiple glycosyltransferases, variants and alleles, may be active in a plant cell, to achieve a reduction, substantial inhibition or complete inhibition of the enzyme activities, it is contemplated that more than one gene sequences encoding glycosyltransferases are to be modified in the plant cell. In preferred embodiments of the invention, the modifications are produced by applying one or more genome editing technologies that are known in the art. A modified plant cell of the invention can be produced by a number of strategies.

In one embodiment of the invention, a first gene sequence encoding a first glycosyltransferase or a fragment thereof, in a plant cell is modified, followed by identification or isolation of modified plant cells that exhibit a reduced activity of the first glycosyltransferase. The modified plant cells comprising a modified first glycosyltransferase gene are then subject to mutagenesis, wherein a second gene sequence encoding a second glycosyltransferase or a fragment thereof is modified. This is followed by identification or isolation of modified plant cells that exhibit a reduced activity of the second glycosyltransferase, or a further reduction of the glycosyltransferase activity relative to that of cells that carry only the first modification. Modified plant cells can be isolated after identification. The modified plant cell obtained at this stage comprises two modifications in two gene sequences that encode two glycosyltransferases, or two variants or alleles of a glycosyltransferase.

Modified plant cells or modified plants of the invention can be identified by the production of a mutant glycosyltransferase that has a molecular weight which is different from the glycosyltransferase produced in an unmodified plant or plant cell. The mutant glycosyltransferase can be a truncated form or an elongated form of the glycosyltransferase produced in an unmodified plant or plant cell, and can be used as a marker to aid identification of a modified plant or plant cell. The truncation or elongation of the polypeptide typically results from the introduction of a stop codon in the coding sequence or a shift in the reading frame resulting in the use of a stop codon in an alternative reading frame.

The invention further provides that the modified plant cells are subjected to one or more successive rounds of modifications of genes encoding other glycosyltransferases or other variants or alleles of glycosyltransferases, for example, a third, a fourth, a fifth, a sixth, a seventh, or an eighth gene sequence encoding a glycosyltransferase or a variant or allele thereof. It is contemplated that the first gene sequence that is subjected to modification encodes a glycosyltransferase of the invention, such as but not limited to a beta-1,2-xylosyltransferase, an alpha-1,3-fucosyltransferase, or a N-acetylglucosaminyltransferase. The second, third, fourth, fifth, sixth, seventh, or eighth gene sequences encoding a glycosyltransferase or an allele thereof can each be independently, a beta-1,2-xylosyltransferase, an alpha-1,3-fucosyltransferase, or a N-acetylglucosaminyltransferase. The modified plant cells that exhibit a reduced enzyme activity or an inhibition or substantial inhibition of enzyme activity may comprise one, two, three, four, five, six, seven, eight or more modified gene sequences each encoding a glycosyltransferase of the invention, wherein each of the glycosyltransferases can independently be a beta-1,2-xylosyltransferase, an alpha-1,3-fucosyltransferase, or a N-acetylglucosaminyltransferase.

Accordingly, the invention provides modified plant cells comprising two or more modified beta-1,2-xylosyltransferase genomic DNA sequences, two or more alpha-1,3-fucosyltransferase genomic DNA sequences, or two or more modified N-acetylglucosaminyltransferase genomic DNA sequences. Modified plant cells comprising one or more modified beta-1,2-xylosyltransferase genomic DNA sequences and one or more modified N-acetylglucosaminyltransferase genomic DNA sequences are encompassed. Modified plant cells comprising one or more modified alpha-1,3-fucosyltransferase genomic DNA sequences and one or more modified N-acetylglucosaminyltransferase genomic DNA sequences are also provided. Modified plant cells comprising one or more modified alpha-1,3-fucosyltransferase genomic DNA sequences and one or more modified beta-1,2-xylosyltransferase genomic DNA sequences are encompassed.

Another strategy for producing a modified plant or plant cells comprising more than one modified glycosyltransferase gene sequences involves crossing two different plants, wherein each of the two plants comprises one or more different modified glycosyltransferase gene sequences. The modified plants used in a crossing can be produced by methods of the invention as described above.

The modified plants and plant cells that are used in crossings or genome modification as described above can be identified or selected by (i) a reduced or undetectable activity of one or more glycosyltransferases; (ii) a reduced or undetectable expression of one or more glycosyltransferases; (iii) a reduced or undetectable level of alpha-1,3-linked fucose, beta-1,2-linked xylose, or both, on the N-glycan of plant proteins or heterologous protein(s); or (iv) an increase or accumulation of high mannose-type N-glycan, in the modified plant or plant cells.

In an embodiment of the invention, a modified plant or modified plant cell can be produced by zinc finger nuclease-mediated mutagenesis. A zinc finger DNA-binding domain or motif consists of approximately 30 amino acids that fold into a beta-beta-alpha (ββα) structure of which the alpha-helix (α-helix) inserts into the DNA double helix. An “alpha-helix” (α-helix) as used within the present invention refers to a motif in the secondary structure of a protein that is either right- or left-handed coiled in which the hydrogen of each N—H group of an amino acid is bound to the C═O group of an amino acid at position −4 relative to the first amino acid. A “beta-barrel” (β-barrel) as used herein refers to a motif in the secondary structure of a protein comprising two beta-strands (β-strands) in which the first strand is hydrogen bound to a second strand to form a closed structure. A “beta-beta-alpha” (ββα) structure” as used herein refers to a structure in a protein that consists of a β-barrel comprising two anti-parallel β-strands and one α-helix. The term “zinc finger DNA-binding domain” as used within the present invention refers to a protein domain that comprises a zinc ion and is capable of binding to a specific three basepair DNA sequence. The term “non-natural zinc finger DNA-binding domain” as used herein refers to a zinc finger DNA-binding domain that does not occur in the cell or organism comprising the DNA which is to be modified.

The key amino acids within a zinc finger DNA-binding domain or motif that bind the three basepair sequence within the target DNA, are amino acids −1, +1, +2, +3, +4, +5 and +6 relative to the begin of the alpha-helix (α-helix). The amino acids at position −1, +1, +2, +3, +4, +5 and +6 relative to the begin of the α-helix of a zinc finger DNA-binding domain or motif can be modified while maintaining the beta-barrel (β-barrel) backbone to generate new DNA-binding domains or motifs that bind a different three basepair sequence. Such a new DNA-binding domain can be a non-natural zinc finger DNA-binding domain. In addition to the three basepair sequence recognition by the amino acids at position −1, +1, +2, +3, +4, +5 and +6 relative to the start of the α-helix, some of these amino acids can also interact with a basepair outside the three basepair sequence recognition site. By combining two, three, four, five, six or more zinc finger DNA-binding domains or motifs, a zinc finger protein can be generated that specifically binds to a longer DNA sequence. For example, a zinc finger protein comprising two zinc finger DNA-binding domains or motifs can recognize a specific six basepair sequence and a zinc finger protein comprising four zinc finger DNA-binding domains or motifs can recognize a specific twelve basepair sequence. A zinc finger protein can comprise two or more natural zinc finger DNA-binding domains or motifs or two or more non-natural zinc finger DNA-binding domains or motifs derived from a natural or wild-type zinc finger protein by truncation or expansion or a process of site-directed mutagenesis coupled to a selection method such as, but not limited to, phage display selection, bacterial two-hybrid selection or bacterial one-hybrid selection or any combination of natural and non-natural zinc finger DNA-binding domains. “Truncation” as used within this context refers to a zinc finger protein that contains less than the full number of zinc finger DNA-binding domains or motifs found in the natural zinc finger protein “Expansion” as used within this context refers to a zinc finger protein that contains more than the full number of zinc finger DNA-binding domains or motifs found in the natural zinc finger protein. Techniques for selecting a polynucleotide sequence within a genomic sequence for zinc finger protein binding are known in the art and can be used in the present invention. Methods for the construction of non-natural zinc finger proteins binding to such a polynucleotide sequence are also known to those skilled in the art and can be used in the present invention.

In a specific embodiment of the invention, a genomic DNA sequence comprising a part of or all of the coding sequence of a glycosyltransferase of the invention is modified by zinc finger nuclease mediated mutagenesis. The genomic DNA sequence is searched for a unique site for zinc finger protein binding. Alternatively, the genomic DNA sequence is searched for two unique sites for zinc finger protein binding wherein both sites are on opposite strands and close together. The two zinc finger protein target sites can be 0, 1, 2, 3, 4, 5, 6 or more basepairs apart. The zinc finger protein binding site may be in the coding sequence of a glycosyltransferase gene sequence or a regulatory element controlling the expression of a glycosyltransferase, such as but not limited to the promoter region of a glycosyltransferase gene. Particularly, one or both zinc finger proteins are non-natural zinc finger proteins.

Accordingly, the invention provides zinc finger proteins that bind to the glycosyltransferases of the invention, such as but not limited to a beta-1,2-xylosyltransferase or a fragment thereof, an alpha-1,3-fucosyltransferase or a fragment thereof, a N-acetylglucosaminyltransferase, or a fragment thereof. In a preferred embodiment, the zinc finger proteins bind to glycosyltransferases of the invention of Nicotiana tabacum.

It is contemplated that a method for mutating a gene sequence, such as a genomic DNA sequence, that encodes a glycosyltransferase of the invention by zinc finger nuclease-mediated mutagenesis comprises optionally one or more of the following steps: (i) providing at least two zinc finger proteins that selectively bind different target sites in the gene sequence; (ii) constructing two expression constructs each encoding a different zinc finger nuclease that comprises one of the two different non-natural zinc finger proteins of step (i) and a nuclease, operably linked to expression control sequences operable in a plant cell; (iii) introducing the two expression constructs into a plant cell wherein the two different zinc finger nucleases are produced, such that a double stranded break is introduced in the genomic DNA sequence in the genome of the plant cell, at or near to at least one of the target sites. The introduction of the two expression constructs into the plant cell can be accomplished simultaneously or sequentially, optionally including selection of cells that took up the first construct.

A double stranded break (DSB) as used herein, refers to a break in both strands of the DNA or RNA. The double stranded break can occur on the genomic DNA sequence at a site that is not more than between 5 base pairs and 1500 base pairs, particularly not more than between 5 base pairs and 200 base pairs, particularly not more than between 5 base pairs and 20 base pairs removed from one of the target sites. The double stranded break can facilitate non-homologous end joining leading to a mutation in the genomic DNA sequence at or near the target site. “Non homologous end joining (NHEJ)” as used herein refers to a repair mechanism that repairs a double stranded break by direct ligation without the need for a homologous template, and can thus be mutagenic relative to the sequence before the double stranded break occurs.

The method can optionally further comprise the step of (iv) introducing into the plant cell a polynucleotide comprising at least a first region of homology to a nucleotide sequence upstream of the double-stranded break and a second region of homology to a nucleotide sequence downstream of the double-stranded break. The polynucleotide can comprise a nucleotide sequence that corresponds to a glycosyltransferase gene sequence that contains a deletion or an insertion of heterologous nucleotide sequences. The polynucleotide can thus facilitate homologous recombination at or near the target site resulting in the insertion of heterologous sequence into the genome or deletion of genomic DNA sequence from the genome. The resulting genomic DNA sequence in the plant cell can comprise a mutation that disrupts the enzyme activity of an expressed mutant glycosyltransferase, a early translation stop codon, or a sequence motif that interferes with the proper processing of pre-mRNA into an mRNA resulting in reduced expression or inactivation of the gene. Methods to disrupt protein synthesis by mutating a gene sequence coding for a protein are known to those skilled in the art.

A zinc finger nuclease according to the present invention may be constructed by making a fusion of a first polynucleotide coding for a zinc finger protein that binds to a gene sequence of a gene involved in N-glycosylation, such as but not limited to the gylcosyltransferases of the invention, and a second polynucleotide coding for a non-specific endonuclease such as, but not limited to, those of a Type IIS endonuclease. A Type IIS endonuclease is a restriction enzyme having a separate recognition domain and an endonuclease cleavage domain wherein the enzyme cleaves DNA at sites that are removed from the recognition site. Non-limiting examples of Type IIS endonucleases can be, but not limited to, AarI, BaeI, CdiI, DrdlI, EciI, FokI, FauI, GdilI, HgaI, Ksp632I, MbolI, Pfi1108I, Rle108I, RleAI, SapI, TspDTI or UbaPI.

Methods for the design and construction of fusion proteins, methods for the selection and separation of the endonuclease domain from the sequence recognition domain of a Type IIS endonuclease, methods for the design and construction of a zinc finger nuclease comprising a fusion protein of a zinc finger protein and an endonuclease, are known in the art and can be used in the present invention. In a specific embodiment, the nuclease domain in a zinc finger nuclease is that of FokI. A fusion protein between a zinc finger protein and the nuclease of FokI may comprise a spacer consisting of two basepairs or alternatively, the spacer can consist of three, four, five, six or more basepairs. In one aspect, the invention provides a fusion protein with a seven basepair spacer such that the endonuclease of a first zinc finger nuclease can dimerize upon contacting a second zinc finger nuclease, wherein the two zinc finger proteins making up said zinc finger nucleases can bind upstream and downstream of the target DNA sequence. Upon dimerization, a zinc finger nuclease can introduce a double stranded break in a target nucleotide sequence which may be followed by non-homologous end joining or homologous recombination with an exogenous nucleotide sequence having homology to the regions flanking both sides of the double stranded break.

In yet another embodiment, the invention provides a fusion protein comprising a zinc finger protein and an enhancer protein resulting in a zinc finger activator. A zinc finger activator can be used to up-regulate or activate transcription of a target gene in a plant cell such as, but not limited to, one involved in N-glycosylation in a plant cell, comprising the steps of (i) engineering a zinc finger protein that binds a region within a promoter or a sequence operatively linked to a coding sequence of a target gene according to methods of the present invention, (ii) making a fusion protein between said zinc finger protein and a transcription activator, (iii) making an expression construct comprising a polynucleotide sequence coding for said zinc finger activator under control of a promoter active in a plant cell, (iv) introducing said gene construct into a plant cell, and (v) culturing the plant cell and allowing the expression of the zinc finger activator, and (vi) characterizing a plant cell having an increased expression of the target gene. A target gene useful in the invention is a gene that encodes a protein or a nucleic acid that regulates the expression of a glycosyltransferase of the invention.

In yet another embodiment, the invention provides a fusion protein comprising a zinc finger protein and a gene repressor resulting in a zinc finger repressor. A zinc finger repressor can be used to down-regulate or repress the transcription of a gene in a plant such as, but not limited to, those involved in N-glycosylation in a plant cell, comprising the steps of (i) engineering a zinc finger protein that binds to a region within a promoter or a sequence operatively linked to a glycosyltransferase gene according to methods of the present invention, and (ii) making a fusion protein between said zinc finger protein and a transcription repressor, and (iii) developing a gene construct comprising a polynucleotide sequence coding for said zinc finger repressor under control of a promoter active in said plant cell according to methods of the present invention, and (iv) introducing said gene construct into a plant cell according to methods of the present invention, and (v) allowing the expression of the zinc finger repressor, and (vi) characterizing a plant cell having reduced transcription of the target gene. A zinc finger repressor can be used to reduce the level of expression of a glycosyltransferase of the invention in a plant cell.

In yet another embodiment, the invention provides a fusion protein comprising a zinc finger protein and a methylase resulting in a zinc finger methylase. The zinc finger methylase may be used to down-regulate or inhibit the expression of a gene involved in N-glycosylation in a plant cell by methylating a region within the promoter region of said gene involved in N-glycosylation, such as but not limited to the glycosyltransferases of the invention, comprising the steps of (i) engineering a zinc finger protein that can binds to a region within a promoter of the gene involved in N-glycosylation according to methods of the present invention, and (ii) making a fusion protein between said zinc finger protein and a methylase, and (iii) developing a gene construct containing a polynucleotide coding for said zinc finger methylase under control of a promoter active in a plant cell according to methods of the present invention, and (iv) introducing said gene construct into a plant cell according to methods of the present invention, and (v) allowing the expression of the zinc finger methylase, and (vi) characterizing a plant cell having reduced or essentially no expression of a glycosyltransferase of the invention in a plant cell.

In various embodiments of the invention, a zinc finger protein may be selected according to methods of the present invention to bind to a regulatory sequence of a glycosyltransferase of the invention. The glycosyltransferase can be a glycosyltransferase involved in N-glycosylation in plants such as, but not limited to, an N-acetylglucosaminyltransferase, a xylosyltransferase or a fucosyltransferase or more specifically an N-acetylglucosaminyltransferase I, a beta-1,2-xylosyltransferase or an alpha-1,3-fucosyltransferase. More specifically, the regulatory sequence of a gene involved in N-glycosylation in a plant can comprise a transcription initiation site, a start codon, a region of an exon, a boundary of an exon-intron, a terminator, or a stop codon. The zinc finger protein can be fused to a nuclease, an activator, or a repressor protein.

In various embodiments of the invention, a zinc finger nuclease introduces a double stranded break in a regulatory region, a coding region, or a non-coding region of a genomic DNA sequence of a glycosyltransferase of the invention, and leads to a reduction, an inhibition or a substantial inhibition of the level of expression of the glycosyltransferase, or a reduction, an inhibition or a substantial inhibition of the activity of the glycosyltransferase.

The method according to the invention for reducing, inhibiting or substantially inhibiting the activity of an endogenous glycosyltransferase enzyme in a plant cell can comprise the step of selecting a modified cell with a reduced, inhibited or substantially inhibited glycosyltransferase enzyme activity.

In yet another embodiment, the present invention contemplates the use of gene sequences of the invention or a fragment thereof for identifying a target site in said sequence to modify expression of a glycosyltransferase in a plant cell such that (i) the activity of the glycosyltransferase is reduced, inhibited or substantially inhibited; or (ii) the level of alpha-1,3-fucose or beta-1,2-xylose on a N-glycan of one or more proteins in the plant cell is reduced. To identify such target sites on a gene sequence of the invention, a computer program is provided that allows screening an input query sequence for the occurrence of two fixed-length substring DNA motifs separated by a fixed length spacer sequence using a suffix array within a DNA database for the selection of two target sites for zinc finger protein binding that occur a given number of times within the reference DNA database and are separated by a defined number of nucleotides (referred to herein as a spacer sequence). The gene sequences can be genomic DNA or cDNA sequences, such as but not limited to that of an alpha-1,3-fucosyltransferase, a beta-1,2-xylosyltransferase or an N-acetylglucosaminyltransferase. Particularly, the gene sequences are that of Nicotiana species, such as but not limited to Nicotiana tabacum. In a specific embodiment of the invention, the DNA database is a tobacco DNA database.

Particularly, the computer program can be used to search a Nicotiana tabacum gene sequence of the invention for two zinc finger protein binding sites, wherein each of the zinc finger proteins comprises four zinc finger DNA binding domains and the two zinc finger protein binding sites are separated by 0, 1, 2 or 3 basepairs. In other embodiments of the present invention, the computer program can be used to predict target sites for two zinc finger proteins for the design of a pair of zinc finger nucleases. In other embodiments of the present invention, the computer program is used to predict target sites for a meganuclease. Also encompassed in the invention are the target sites present in the gene sequences of the invention, such as those predicted by the computer program described above, and their uses in modifying the gene sequences in a plant or plant cell by genome editing technologies that are described in the invention or known in the art.

In various embodiments of the invention, an expression construct comprising a coding sequence operably linked to expression control sequences that are effective in a plant cell, is introduced into a plant cell to facilitate the expression of a heterologous protein. “Operably linked” refers to a link in which the control sequences and the DNA sequence to be expressed are joined and positioned in such a way as to permit transcription, as well as translation of transcripts. In a specific embodiment, an expression construct is used to produce a non-natural zinc finger protein, zinc finger nuclease, zinc finger repressor, zinc finger activator. In other embodiments of the invention, an expression construct is used to produce a heterologous protein of commercial interest, such as a mammalian or human protein. It is contemplated that plant cells that are being modified either have integrated an expression construct into chromosomal DNA or carry the expression construct extrachromosomally. It is also contemplated that modified plant cells that are used to produce heterologous protein, either have stably integrated a recombinant transcriptional unit comprising a coding sequence of the heterologous protein into chromosomal DNA or carry for a limited time period the recombinant transcriptional unit extrachromosomally.

Expression constructs comprising regulatory elements that are active in plants and plant cells are known and may contain a plant virus promoter and terminator sequence such as, but not limited to, the cauliflower mosaic virus 35S promoter and terminator region, a plastocyanin promoter and terminator region; or a ubiquitin promoter or terminator region. In specific embodiments of the invention, the coding sequence of a first zinc finger nuclease can be cloned under control of one promoter and terminator sequence, and the coding sequence of a second zinc finger nuclease can be cloned under control of a second promoter and terminator sequence, both active in a plant cell. Both zinc finger nuclease expression constructs can also be controlled by the same promoter and terminator sequence and the coding sequences for two zinc finger nucleases can be placed on one vector or separate vectors.

As used herein, the term “transformation” refers to the transfer of a polynucleotide into an organism, such as but not limited to a plant cell. Host organisms containing the transformed polynucleotide are referred to as “transgenic” organisms. Examples of methods of plant transformation include but are not limited to Agrobacterium-mediated transformation (De Blaere et al., Meth. Enzymol. 143:277 (1987)) and particle-accelerated or “gene gun” transformation technology (Klein et al., Nature, London 327:70-73 (1987); U.S. Pat. No. 4,945,050).

Many plant cell transformation protocols and many methods to introduce foreign DNA into a plant cell thereby allowing the expression of a gene comprised within said foreign DNA are known. A vector to introduce an expression construct into a plant cell can be a binary vector and can be introduced into a plant cell via Agrobacterium tumefaciens transformation. Agrobacterium tumefaciens transformation systems are known to those skilled in the art. Agrobacterium tumefaciens strains for infection and transfection of plant cells are known. An Agrobacterium tumefaciens strain that may be suitably used for the purpose of the present invention is GV3101 or AgI0, AgI1, LBA4404, or any other Achy or C58 derived Agrobacterium tumefaciens strain capable of infecting a plant cell and transferring a T-DNA into the plant cell nucleus.

In a non-limiting example, Agrobacterium-mediated transformation can be carried out as follows: A plant expression vector such as for example a binary vector comprising the expression cassettes for the expression of two zinc finger nucleases making up a pair that can target a tobacco glycosyltransferase genomic gene sequence, can be introduced in Agrobacterium tumefaciens strain using standard methods described in the art. The recombinant Agrobacterium tumefaciens strain can be grown overnight in liquid broth containing appropriate antibiotics and cells can be collected by centrifugation, decanted and resuspended in fresh medium according to Murashige & Skoog (1962, Physiol Plant 15(3): 473-497). Leaf explants of aseptically grown tobacco plants can be transformed according to standard methods (see Horsch et al., 1985) and co-cultivated for two days on medium according to Murashige & Skoog (1962) in a petri dish under appropriate conditions as described in the art. After two days of co-cultivation, explants can be placed on selective medium containing an appropriate amount of kanamycin for selection supplemented with vancomycin and cefotaxim antibiotics, and naphthaleneacetic acid and benzaminopurine hormones. The binary vector can be introduced in the Agrobacterium tumefaciens strain. Alternatively, the binary vector can be introduced into other Agrobacterium tumefaciens strains or derived therefrom suitable for the transformation of plant leaf explants, particularly tobacco leaf explants. Alternatively, explants can be seedlings, hypocotyls or stem tissue or any other tissue amenable to transformation. The introduction of the binary vector comprising the expression cassette is carried out via transfection with an Agrobacterium tumefaciens strain.

Alternatively, the introduction can be carried out using particle bombardment or any alternative plant transformation method known to those skilled in the art and commonly used in plant transformation. For example, using a particle gun or biolistic particle delivery system, foreign DNA can be loaded onto a tungsten particle or onto a gold particle and introduced into a plant cell using a Helios PDS 1000/He Biolistic Particle Delivery System.

As a non-limiting example, the regeneration and selection of plants after transfection of plant cells can be carried out within the scope of the present invention as follows: Transgenic plant cells obtained after transfection as described herein above can be regenerated into shoots and plantlets according to standard methods described in the art (see for example, Horsch et al., 1985, Science 227:1229). Genomic DNA can be isolated from shoots or plantlets for example by using the PowerPlant DNA isolation kit (Mo Bio Laboratories Inc., Carlsbad, Calif., USA). DNA fragments comprising the targeted region can be amplified according to standard methods described in the art using the gene sequence. To those skilled in the art it is clear that, for example, the pair of primers as defined in the listed SEQ ID NOs can be used to amplify the fragment comprising the targeted region. PCR products are then sequenced in their entirety using standard sequencing protocols and mutations or modifications at or around a target site, such as a zinc finger nuclease target site, can be identified by comparison with the original sequence.

A modification of a genomic nucleotide sequence according to the invention can be characterized as follows: after the coding region of a glycosyltransferase is targeted for modification in plant cells, cDNA synthesized from mRNA obtained from the modified cells can be cloned and sequenced to confirm the presence of the modification. To those skilled in the art it is clear that any deletion that can result in the disruption of the open reading frame of the respective sequence, and can have a deleterious effect on the biosynthesis of a functional enzyme.

The activity of each of the glycosyltransferases of the invention can be measured using an enzyme assay. The activity of a glycosyltransferase of the invention can be but is not limited to the addition of an N-acetylglucosamine to a mannose on the 1-3 arm of a Man5-GlcNAc2-Asn oligomannosyl receptor; the addition of a fucose entity in alpha-1,3-linkage to an N-glycan, particularly addition of a fucose in alpha-1,3-linkage onto the proximal N-acetylglucosamine at the non-reducing end of an N-glycan of a glycoprotein; or the addition of a xylose entity in beta-1,2-linkage to an N-glycan, particularly addition of a xylose in β(1,2)-linkage onto the β(1,4)-linked mannose of the trimannosyl core structure of an N-glycan. Glycosyltransferases may be isolated from a plant, for example, by isolating microsomes from a plant cell which are enriched for glycosyltransferases. Enzyme activity can be measured using an enzyme assay and a specific substrate and donor molecule such as for example UDP-[¹⁴C]-xylose as donor and GlcNAcβ-1-2-Man-α1-3-[Man-α1-6]Man-β-O—(CH₂)₈—COOH₃ or GlcNAcβ-1-2-Man-α1-3-(GlcNAc-β1-2-Man-α1-6)Man-β1-4GlcNAc-β1-4(Fuc-α1-6)GlcNAc-IgG glycopeptide as an acceptor for measuring beta-1,2-xylosyltransferase activity.

In particular, microsomes can be isolated from fresh plant leaves of mature, full-grown plants, particularly tobacco plants, at the stage of early flowering as follows: remove the midvein, cut leaves into small pieces and homogenize in a precooled stainless-steel Waring blender in microsome isolation buffer for example comprising of 250 mM sorbitol, 5 mM Tris, 2 mM DTT and 7.5 mM EDTA; set at pH 7.8 by using a 1 M solution of Mes (2-(N-morpholino)ethanesulfonic acid. Add a protease inhibitor mixture or cocktail such as for example Complete Mini (Roche Diagnostics). Use ice-cold microsome isolation buffer of fresh-weight tobacco leaves. Filter through nylon cloth and remove debris and leaf material by centrifugation for 10 min at 12,000 g at 4° C. using a Sorvall SS34 rotor. Transfer supernatant containing microsomes to new centrifugation tube and centrifuge in a fixed-angle Centrikon TFT 55.38 rotor for 60 min at 100,000 g at 4° C. in a Centricon T-2070 ultracentrifuge. Resuspend the pellet containing the microsomes in microsome isolation buffer without EDTA and to which glycerol (4% final concentration) has been added. This can be used to measure beta-1,2-xylosyltransferase (β(1,2)-xylosyltransferase) activity.

As a non-limiting example, a gene coding for a beta-1,2-xylosyltransferase (β(1,2)-xylosyltransferase enzyme), activity can be established as follows: a cDNA sequence can be cloned in a mammalian expression vector and electroporated into mammalian cells that normally do not have beta-1,2-xylose (β(1,2)-xylose) on the N-glycans of endogenous glycoproteins. Complementation can be visualized through staining of cells with an antibody that recognizes a beta-1,2-xylose (β(1,2)-xylose) on an N-glycan such as a rabbit anti-horseradish peroxidase antibody, for example Art. No. AS07 267 of Agrisera AB (Vännäs, Sweden), that specifically cross-reacts with xylose residues bound to protein N-glycans. Alternatively, a xylosyltransferase enzyme assay can be performed with the recombinant protein obtained upon expressing a beta-1,2-xylosyltransferase (β(1,2)-xylosyltransferase) cDNA in a suitable host system lacking xylosyltransferase activity. A xylosyltransferase assay can be performed in a reaction mixture comprising 10 mM cacodylate buffer (pH 7.2), 4 mM ATP, 20 mM MnCl₂, 0.4% Triton X-100, 0.1 mM UDP-[¹⁴C]-xylose and 1 mM GlcNAcβ-1-2-Man-α1-3-[Man-α1-6]Man-β-O—(CH₂)₈—COOH₃ using GlcNAcβ-1-2-Man-α1-3-(GlcNAc-β1-2-Man-α1-6)Man-β1-4GlcNAc-β1-4(Fuc-α1-6)GlcNAc-IgG glycopeptide as an acceptor.

To facilitate isolation of a modified glycosyltransferase of the invention or a heterologous protein of interest from a plant or plant cell, many techniques and purification schemes known in the art can be used. As a non-limiting example, His tags, GST, and maltose-binding protein represent peptides that have readily available affinity columns to which they can be bound and eluted. Thus, where the peptide is an N-terminal His tag such as hexahistidine (His.sub.6 tag), the heterologous protein can be purified using a matrix comprising a metal-chelating resin, for example, nickel nitrilotriacetic acid (Ni-NTA), nickel iminodiacetic acid (Ni-IDA), and cobalt-containing resin (Co-resin). See, for example, Steinert et al. (1997) QIAGEN News 4:11-15. Where the peptide is GST, the heterologous protein can be purified using a matrix comprising glutathione-agarose beads (Sigma or Pharmacia Biotech); where the protein fragment is a maltose-binding protein (MBP), the modified glycosyltransferase or heterologous protein can be purified using a matrix comprising an agarose resin derivatized with amylose.

Other non-limiting examples of molecules that can bind to a modified glycosyltransferase of the invention or a heterologous protein of interest may be selected from aptamers (Klussmann (2006), The Aptamer Handbook: Functional Oligonucleotides and their applications, Wiley-VCH, USA), antibodies (Howard and Bethell (2000) Basic Methods in Antibody Production and Characterization, Crc. Pr. Inc), (Hansson, Immunotechnology 4 (1999), 237-252; Henning, Hum Gene Ther. 13 (2000), 1427-1439), affibodies, lectins, trinectins (Phylos Inc., Lexington, Massachusetts, USA; Xu, Chem. Biol. 9 (2002), 933), anticalins (EPB1 1 017 814) and the like.

In various embodiments of the invention, the invention provides modified plants, modified plant tissues, plant materials from modified plants, modified plant cells, or modified plant tissues, or plant compositions from modified plants, that comprises a heterologous protein that has a reduced level or an undetectable level of alpha-1,3-linked fucose, beta-1-2-linked xylose, or both, on the N-glycan. In other embodiments, the invention provides modified plants, modified plant tissues, plant materials from modified plants, modified plant cells, or modified plant tissues, or plant compositions from modified plants, that show reduced or substantially no glycosyltransferase activity. A modified plant of the invention can comprise modified cells and unmodified cells. It is not required that every cell in a modified plant of the invention comprises a modification.

The heterologous protein can be enriched, isolated, or purified by techniques known in the art. Accordingly, the invention provides plant compositions that are enriched for the heterologous protein, or plant compositions that comprise a higher concentration of the heterologous protein relative to the concentration at which the heterologous protein occurs in the plant or plant cell. Also provided are pharmaceutical or cosmetic compositions comprising a heterologous protein obtained from a plant cell, particularly a Nicotiana cell, that comprises a reduced or undetectable level of alpha-1,3-linked fucose and/or beta-1,2-linked xylose on an N-glycan attached to the heterologous protein, and a carrier, such as a pharmaceutically acceptable carrier.

The heterologous protein that can be expressed in a modified plant cell can be an antigen for use in a vaccine, including but not limited to a protein of a pathogen, a viral protein, a bacterial protein, a protozoal protein, a nematode protein; an enzyme, including but not limited to an enzyme used in treatment of a human disease, an enzyme for industrial uses; a cytokine; a fragment of a cytokine receptor; a blood protein; a hormone; a fragment of a hormone receptor, a lipoprotein; an antibody or a fragment of an antibody.

The terms “antibody” and “antibodies” refer to monoclonal antibodies, multispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

In specific embodiments of the invention, the invention provides a method for producing a heterologous protein comprising N-glycans that comprise a reduced or undetectable level of alpha-1,3-fucose or beta-1,2-xylose, or both. The method comprises expressing a polynucleotide comprising a coding sequence for a heterologous protein in a modified plant cell of the invention to produce the heterologous protein. The method can comprise the steps of (i) introducing into a modified plant cell of the invention, a polynucleotide comprising a coding sequence for a heterologous protein, (ii) allowing expression of said polynucleotide to produce the heterologous protein in the modified plant cell, and optionally (iii) isolating the heterologous protein from said modified plant cell. The method can further comprise culturing modified plant cells that comprise the polynucleotide comprising a coding sequence for the heterologous protein. The method can optionally comprise the step of developing the modified plant cell comprising the polynucleotide comprising a coding sequence for the heterologous protein into plant tissue, plant organ, or a plant, and culturing or growing the plant tissue, plant organ, or the plant. The plant cell can be a cell grown in cell culture under aseptic conditions in an aqueous medium or a cell of a monocot such as but not limited to sorghum, maize, wheat, rice, millet, barley or duckweed, or a dicot such as sunflower, pea, rapeseed, sugar beet, soybean, lettuce, endive, cabbage, broccoli, cauliflower, alfalfa, carrot or tobacco. The tobacco cells according to the present invention can be Nicotiana plant cells, particularly Nicotiana plant cells selected from a group consisting of Nicotiana benthamiana or Nicotiana tabacum, Nicotiana tabacum varieties, breeding lines and cultivars, or modified cells of Nicotiana benthamiana and Nicotiana tabacum. Nicotiana tabacum varieties, breeding lines and cultivars.

In another embodiment, the invention provides genetically modified cells of Nicotiana tabacum varieties, breeding lines, or cultivars. Non-limiting examples of Nicotiana tabacum varieties, breeding lines, and cultivars that can be modified by the methods of the invention include N. tabacum accession PM016, PM021, PM92, PM102, PM132, PM204, PM205, PM215, PM216 or PM217 as deposited with NCIMB, Aberdeen, Scotland, or DAC Mata Fina, PO2, BY-64, AS44, RG17, RG8, HB04P, Basma Xanthi BX 2A, Coker 319, Hicks, McNair 944 (MN 944), Burley 21, K149, Yaka JB 125/3, Kasturi Mawar, NC 297, Coker 371 Gold, PO2, Wisliça, Simmaba, Turkish Samsun, AA37-1, B13P, F4 from the cross BU21×Hoja Parado line 97, Samsun NN, Izmir, Xanthi NN, Karabalgar, Denizli and PO1.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion. The carrier can be a parenteral carrier, more particularly a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

In preferred embodiments of the invention, a method for reducing the glycosyltransferase activity of a plant cell is provided, comprising modifying a genomic nucleotide sequence in the genome of a plant cell, wherein the genomic nucleotide sequence comprises a coding sequence for an N-acetylglucosaminyltransferase, particularly an N-acetylglucosaminyltransferase I; a fucosyltransferase, particularly an alpha-1,3-fucosyltransferase; or a xylosyltransferase, particularly a beta-1,2-xylosyltransferase; or a fragment of the foregoing proteins. In specific embodiments, the invention provides a method for reducing the glycosyltransferase activity of a plant cell, comprising modifying a genomic nucleotide sequence in the genome of a plant cell, wherein the genomic nucleotide sequence comprises (i) a nucleotide sequence that consists of the nucleotide sequence as shown in SEQ ID NOS: 1, 4, 5, 7, 12, 13, 14, 17, 27, 32, 37, 40, 41, or 47; (ii) a nucleotide sequence that is at least 95%, particularly at least 98%, particularly at least 99%, identical to a nucleotide sequence as shown in the SEQ ID NOS: 1, 4, 5, 7, 12, 13, 14, 17, 27, 32, 37, 40, 41, or 47; (iii) a nucleotide sequence that allows a polynucleotide probe consisting of the nucleotide sequence of (i) or (ii), or a complement thereof, to hybridize, particularly under stringent conditions. The methods of the invention further comprise identifying and, optionally, selecting a modified plant cell, wherein the activity of the glycosyltransferase of which the genomic nucleotide sequence had been modified in the modified plant cell, or the total glycosyltransferase activity in the modified plant cell is reduced relative to a unmodified plant cell. This method for reducing the glycosyltransferase activity of a plant cell is applicable to cells of sunflower, pea, rapeseed, sugar beet, soybean, lettuce, endive, cabbage, broccoli, cauliflower, alfalfa, duckweed, rice, maize, carrot, or tobacco. Particularly, the plant cells in which the glycosyltransferase activity is reduced is a cell of a Nicotiana species, particularly Nicotiana benthamiana or Nicotiana tabacum, or a cultivar thereof.

The following embodiments of the invention are non-limiting and are included to illustrate aspects of the invention. In specific embodiments, the invention further provides that the methods also comprise the steps of (a) identifying in the genome of a plant cell a genomic nucleotide sequence comprising a coding sequence for a glycosyltransferase or a fragment thereof; particularly the genomic nucleotide sequence can be identified by using polymerase chain reaction with at least one pair of oligonucleotides selected from the group consisting of a forward primer of SEQ ID NO: 2 and a reverse primer of SEQ ID NO: 3; a forward primer of SEQ ID NO: 10 and a reverse primer of SEQ ID NO: 11; a forward primer of SEQ ID NO: 15 and a reverse primer of SEQ ID NO: 16; a forward primer of SEQ ID NO: 23 and a reverse primer of SEQ ID NO: 24; a forward primer of SEQ ID NO: 25 and a reverse primer of SEQ ID NO: 26; a forward primer of SEQ ID NO: 30 and a reverse primer of SEQ ID NO: 31; a forward primer of SEQ ID NO: 35 and a reverse primer of SEQ ID NO: 36, a forward primer of SEQ ID NO: 45 and a reverse primer of SEQ ID NO: 46, or a forward primer of SEQ ID NO: 231 and a reverse primer of SEQ ID NO: 232; and (b) identifying a target site in the genomic nucleotide sequence for modification such that the activity or expression of the glycosyltransferase is reduced in the plant cell, relative to an unmodified plant cell.

In another embodiment, the invention provides an isolated polynucleotide comprising a nucleotide sequence that consists of the nucleotide sequence as shown in SEQ ID NOS: 1, 4, 5, 7, 12, 13, 14, 17, 27, 32, 37, 40, 41, or 47; a nucleotide sequence that is at least 95%, particularly at least 98%, particularly at least 99%, identical to a nucleotide sequence as shown in the SEQ ID NOS: 1, 4, 5, 7, 12, 13, 14, 17, 27, 32, 37, 40, 41, or 47; or a nucleotide sequence that allows a polynucleotide probe consisting of the nucleotide sequence of (i) or (ii), or a complement thereof, to hybridize to the isolated polynucleotide, particularly under stringent conditions. Also provided are the use of a genomic nucleotide sequence of the invention for identifying a target site in the genomic nucleotide sequence for modification such that (i) the activity or the expression of a glycosyltransferase in a modified plant cell comprising the modification is reduced relative to a unmodified plant cell, or (ii) the alpha-1,3-fucose or beta-1,2-xylose, or both, on a N-glycan of a protein in a modified plant cell comprising the modification is reduced relative to a unmodified plant cell. The invention also provides a method for reducing the glycosyltransferase activity of a plant cell comprising identifying a target site in a genomic nucleotide sequence for modification using a genomic nucleotide sequence of the invention such that (i) the activity or the expression of a glycosyltransferase in a modified plant cell comprising the modification is reduced relative to a unmodified plant cell, or (ii) the alpha-1,3-fucose or beta-1,2-xylose, or both, on a N-glycan of a protein in a modified plant cell comprising the modification is reduced relative to a unmodified plant cell.

The invention also provides a method for modifying a plant cell wherein the genome of the plant cell is modified by zinc finger nuclease-mediated mutagenesis, comprising (a) identifying and making at least two non-natural zinc finger proteins that selectively bind different target sites for modification in the genomic nucleotide sequence; (b) expressing at least two fusion proteins each comprising a nuclease and one of the at least two non-natural zinc finger proteins in the plant cell, such that a double stranded break is introduced in the genomic nucleotide sequence in the plant genome, particularly at or close to a target site in the genomic nucleotide sequence; and, optionally (c) introducing into the plant cell a polynucleotide comprising a nucleotide sequence that comprises a first region of homology to a sequence upstream of the double-stranded break and a second region of homology to a region downstream of the double-stranded break, such that the polynucleotide recombines with DNA in the genome. Also included in the invention are plant cells comprising one or more expression constructs that comprise nucleotide sequences that encode one or more of the fusion proteins.

The invention also provides a modified plant cell, or a plant comprising the modified plant cells, wherein the modified plant cell comprises at least one modification in a genomic nucleotide sequence that encodes a glycosyltransferase or a fragment thereof, particularly any one of the genomic nucleotide sequence shown in SEQ ID NOS: 1, 4, 5, 7, 12, 13, 14, 17, 27, 32, 37, 40, 41, 47, 233, or in SEQ ID NOS: 256, 259, 262, 265, 268, 271, 274, 277, 280, or in SEQ ID NOS: 257, 260, 263, 266, 269, 272, 275, 278, 281, or in any combination of the above sequences and wherein (i) the total glycosyltransferase activity of the modified plant cell, or the activity of or the expression of the glycosyltransferase of which the genomic nucleotide sequence had been modified, is reduced relative to a unmodified plant cell, or (ii) the alpha-1,3-fucose or beta-1,2-xylose, or both, on a N-glycan of a protein produced in the modified plant cell is reduced relative to a unmodified plant cell.

The invention also provides a method for producing a heterologous protein, said method comprising introducing into a modified plant cell that comprises a modification in a genomic nucleotide sequence as shown in SEQ ID NOS: 1, 4, 5, 7, 12, 13, 14, 17, 27, 32, 37, 40, 41, or 47, 233, or in SEQ ID NOs: 18, 20, 21, 22, 28, 33, 38, 48, 212, 213, 219, 220, 223, 225, 227, 229, 234; or in SEQ ID NOS: 256, 259, 262, 265, 268, 271, 274, 277 and 280, or in SEQ ID NOS: 257, 260, 263, 266, 269, 272, 275, 278, 281, or in any combination of the above sequences, an expression construct comprising a nucleotide sequence that encodes a heterologous protein, particularly a vaccine antigen, a cytokine, a hormone, a coagulation protein, an immunoglobulin or a fragment thereof; and culturing the modified plant cell that comprises the expression construct such that the heterologous protein is produced, and optionally, regenerating a plant from the plant cell, and growing the plant and its progenies. The invention also provides a method for producing a heterologous protein, said method comprising culturing a modified plant cell that comprises (i) a modification in at least one of the genomic nucleotide sequence set forth in SEQ ID NOS: 1, 4, 5, 7, 12, 13, 14, 17, 27, 32, 37, 40, 41, or 47, 233 or in SEQ ID NOs: 18, 20, 21, 22, 28, 33, 38, 48, 212, 213, 219, 220, 223, 225, 227, 229, 234; or in SEQ ID NOS: 256, 259, 262, 265, 268, 271, 274, 277 and 280, or in SEQ ID NOS: 257, 260, 263, 266, 269, 272, 275, 278, 281, or in any combination of the above sequences, and (ii) an expression construct comprising a nucleotide sequence that encodes a heterologous protein, particularly a vaccine antigen, a cytokine, a hormone, a coagulation protein, an immunoglobulin or a fragment thereof; under conditions that results in the production of the heterologous protein. Also included in the method of invention are steps for enriching or isolating the heterologous protein from the modified plant cells, or modified plants comprising modified plant cells. The invention also contemplates a plant composition comprising a heterologous protein, obtainable from a plant comprising modified plant cells that comprises a modification in a genomic nucleotide sequence as shown in SEQ ID NOS: 1, 4, 5, 7, 12, 13, 14, 17, 27, 32, 37, 40, 41, or 47, 233 or in SEQ ID NOs: 18, 20, 21, 22, 28, 33, 38, 48, 212, 213, 219, 220, 223, 225, 227, 229, 234; or in SEQ ID NOS: 256, 259, 262, 265, 268, 271, 274, 277 and 280, or in SEQ ID NOS: 257, 260, 263, 266, 269, 272, 275, 278, 281, or in any combination of the above sequences, wherein the alpha-1,3-fucose or beta-1,2-xylose, or both, on the N-glycan of the heterologous protein is reduced relative to that produced in a unmodified plant cell.

In the description and examples, reference is made to the following sequences that are represented in the sequence listing:

SEQ ID NO: 1: nucleotide sequence of contig gDNA_c1736055

SEQ ID NO: 2: nucleotide sequence of NGSG10043 forward primer suitable for amplifying a fragment of contig gDNA_c1736055 that contains a Nicotiana beta-1,2-xylosyltransferase (β(1,2)-xylosyltransferase) intron-exon sequence

SEQ ID NO: 3: nucleotide sequence of NGSG10043 reverse primer suitable for amplifying a fragment of contig gDNA_c1736055 that contains a Nicotiana beta-1,2-xylosyltransferase (β(1,2)-xylosyltransferase) intron-exon sequence

SEQ ID NO: 4: basepairs 1-6,000 of the nucleotide sequence of NtPMI-BAC-TAKOMI_{—}6 that contains Nicotiana tabacum beta-1,2-xylosyltransferase (β(1,2)-xylosyltransferase) gene variant 1

SEQ ID NO: 5: genomic nucleotide sequence of the coding fragment of the beta-1,2-xylosyltransferase (β(1,2)-xylosyltransferase) variant 1 of NtPMI-BAC-TAKOMI_{—}6

SEQ ID NO: 6: nucleotide sequence of the promoter region of NtPMI-BAC-TAKOMI_{—}6 upstream of the beta-1,2-xylosyltransferase (β(1,2)-xylosyltransferase) gene variant 1

SEQ ID NO: 7: nucleotide sequence of fragment of NtPMI-BAC-TAKOMI_{—}6 that was amplified by primer set NGSG10043 and used as probe to identify NtPMI-BAC-TAKOMI_{—}6

SEQ ID NO: 8: cDNA sequence of Nicotiana tabacum beta-1,2-xylosyltransferase (β(1,2)-xylosyltransferase) gene variant 1

SEQ ID NO: 9: amino acid sequence of Nicotiana tabacum beta-1,2-xylosyltransferase (β(1,2)-xylosyltransferase) protein variant 1

SEQ ID NO: 10: primer sequence Big3FN for the amplification of fragment. GnTI-B of Nicotiana tabacum and Nicotiana benthamiana

SEQ ID NO: 11: primer sequence Big3RN for the amplification of fragment GnTI-B of Nicotiana tabacum and Nicotiana benthamiana

SEQ ID NO: 12: nucleotide sequence of 3504 bp genomic fragment of Nicotiana tabacum fragment GnTI-B

SEQ ID NO: 13: nucleotide sequence of 2283 bp genomic fragment of Nicotiana tabacum fragment GnTI-B

SEQ ID NO: 14: nucleotide sequence of 3765 bp genomic fragment of Nicotiana benthamiana fragment GnTI-B

SEQ ID NO: 15: nucleotide sequence of NGSG10046 forward primer suitable for amplifying a fragment of contig CHO_OF4335xn13f1 that contains a Nicotiana beta-1,2-xylosyltransferase (β(1,2)-xylosyltransferase) intron-exon sequence

SEQ ID NO: 16: nucleotide sequence of NGSG10046 reverse primer suitable for amplifying a fragment of contig CHO_OF4335xn13f1 that contains a Nicotiana beta-1,2-xylosyltransferase (β(1,2)-xylosyltransferase) intron-exon sequence

SEQ ID NO: 17: basepairs 15,921-23,200 of the nucleotide sequence of NtPMI-BAC-SANIKI_{—}1 that contains Nicotiana tabacum beta-1,2-xylosyltransferase (β(1,2)-xylosyltransferase) gene variant 2

SEQ ID NO: 18: cDNA sequence of Nicotiana tabacum beta-1,2-xylosyltransferase (β(1,2)-xylosyltransferase gene) variant 2

SEQ ID NO: 19: amino acid sequence of Nicotiana tabacum beta-1,2-xylosyltransferase (β(1,2)-xylosyltransferase) protein variant 2

SEQ ID NO: 20: partial cDNA sequence variant 1 of Nicotiana tabacum fragment GnTI-B

SEQ ID NO: 21: partial cDNA sequence variant 1 of Nicotiana tabacum fragment GnTI-B

SEQ ID NO: 22: partial cDNA sequence variant 1 of Nicotiana benthamiana fragment GnTI-B

SEQ ID NO: 23: primer sequence Big1FN for the amplification of fragment GnTI-A of Nicotiana tabacum and Nicotiana benthamiana

SEQ ID NO: 24: primer sequence Big1 RN for the amplification of fragment GnTI-A of Nicotiana tabacum and Nicotiana benthamiana

SEQ ID NO: 25: nucleotide sequence of NGSG10041 forward primer suitable for amplifying a fragment of contig CHO_OF3295xj17f1 that contains a Nicotiana alpha-1,3-fucosyltransferase (α(1,3)-fucosyltransferase) intron-exon sequence

SEQ ID NO: 26: nucleotide sequence of NGSG10041 reverse primer suitable for amplifying a fragment of contig CHO_OF3295xj17f1 that contains a Nicotiana alpha-1,3-fucosyltransferase (α(1,3)-fucosyltransferase) intron-exon sequence

SEQ ID NO: 27: basepairs 2,961-10,160 of the nucleotide sequence of NtPMI-BAC-FETILA_{—}9 that contains Nicotiana tabacum alpha-1,3-fucosyltransferase (α(1,3)-fucosyltransferase) gene variant 1

SEQ ID NO: 28: cDNA sequence of Nicotiana tabacum alpha-1,3-fucosyltransferase (α(1,3)-fucosyltransferase) gene variant 1

SEQ ID NO: 29: amino acid sequence of Nicotiana tabacum alpha-1,3-fucosyltransferase (α(1,3)-fucosyltransferase) protein variant 1

SEQ ID NO: 30: nucleotide sequence of NGSG10032 forward primer suitable for amplifying a fragment of contig gDNA_c1765694 that contains a Nicotiana alpha-1,3-fucosyltransferase (α(1,3)-fucosyltransferase) intron-exon sequence

SEQ ID NO: 31: nucleotide sequence of NGSG10032 reverse primer suitable for amplifying a fragment of contig gDNA_{—}1765694 that contains a Nicotiana alpha-1,3-fucosyltransferase (α(1,3)-fucosyltransferase) intron-exon sequence

SEQ ID NO: 32: basepairs 1,041-7,738 of the nucleotide sequence of NtPMI-BAC-JUMAKE_{—}4 that contains Nicotiana tabacum alpha-1,3-fucosyltransferase (α(1,3)-fucosyltransferase) gene variant 2

SEQ ID NO: 33: partial cDNA sequence of Nicotiana tabacum alpha-1,3-fucosyltransferase (α(1,3)-fucosyltransferase) gene variant 2

SEQ ID NO: 34: partial amino acid sequence of Nicotiana tabacum alpha-1,3-fucosyltransferase (α(1,3)-fucosyltransferase) protein variant 2

SEQ ID NO: 35: nucleotide sequence of NGSG10034 forward primer suitable for amplifying a fragment of contig CHO_OF4881xd22r1 that contains a Nicotiana alpha-1,3-fucosyltransferase (α(1,3)-fucosyltransferase) intron-exon sequence

SEQ ID NO: 36: nucleotide sequence of NGSG10034 reverse primer suitable for amplifying a fragment of contig CHO_OF4881xd22r1 that contains a Nicotiana alpha-1,3-fucosyltransferase (α(1,3)-fucosyltransferase) intron-exon sequence

SEQ ID NO: 37: basepairs 19,001-23,871 of the nucleotide sequence of NtPMI-BAC-JEJOLO_{—}22 that contains partial Nicotiana tabacum alpha-1,3-fucosyltransferase (α(1,3)-fucosyltransferase) gene variant 3

SEQ ID NO: 38: partial cDNA sequence of Nicotiana tabacum alpha-1,3-fucosyltransferase (α(1,3)-fucosyltransferase) gene variant 3

SEQ ID NO: 39: partial amino acid sequence of Nicotiana tabacum alpha-1,3-fucosyltransferase (α(1,3)-fucosyltransferase) protein variant 3

SEQ ID NO: 40: nucleotide sequence of 3152 bp genomic fragment of Nicotiana tabacum fragment GnTI-A

SEQ ID NO: 41: nucleotide sequence of 3140 bp genomic fragment of Nicotiana tabacum fragment GnTI-A

SEQ ID NO: 42: Unique 22 bp targeting sequence in exon 2 of SEQ ID NO: 5 for meganuclease-mediated mutagenesis

SEQ ID NO: 43: first derivative target representing left halve of SEQ ID NO: 42 in palindromic form

SEQ ID NO: 44: second derivative target representing right halve of SEQ ID NO: 42 in palindromic form

SEQ ID NO: 45: nucleotide sequence of NGSG10035 forward primer suitable for amplifying a fragment of contig CHO_OF4486xe11f1 that contains a Nicotiana alpha-1,3-fucosyltransferase (α(1,3)-fucosyltransferase) intron-exon sequence

SEQ ID NO: 46: nucleotide sequence of NGSG10035 reverse primer suitable for amplifying a fragment of contig CHO_OF4486xe11f1 that contains a Nicotiana alpha-1,3-fucosyltransferase (α(1,3)-fucosyltransferase) intron-exon sequence

SEQ ID NO: 47: basepairs 1-11,000 of the nucleotide sequence of NtPMI-BAC-JUDOSU_{—}1 that contains Nicotiana tabacum alpha-1,3-fucosyltransferase (α(1,3)-fucosyltransferase) gene variant 4

SEQ ID NO: 48: partial cDNA sequence of Nicotiana tabacum alpha-1,3-fucosyltransferase (α(1,3)-fucosyltransferase) gene variant 4

SEQ ID NO: 49: partial amino acid sequence of Nicotiana tabacum alpha-1,3-fucosyltransferase (α(1,3)-fucosyltransferase) protein variant 4

SEQ ID NO: 50: 15 basepair output nucleotide sequence of SEQ ID NO: 5 with 4 hits in tobacco genome database of example 1

SEQ ID NO: 51: 15 basepair output nucleotide sequence of SEQ ID NO: 5 with 5 hits in tobacco genome database of example 1

SEQ ID NO: 52: 15 basepair output nucleotide sequence of SEQ ID NO: 5 with 5 hits in tobacco genome database of example 1

SEQ ID NO: 53: 15 basepair output nucleotide sequence of SEQ ID NO: 5 with 5 hits in tobacco genome database of example 1

SEQ ID NO: 54: 15 basepair output nucleotide sequence of SEQ ID NO: 5 with 5 hits in tobacco genome database of example 1

SEQ ID NO: 55: 15 basepair output nucleotide sequence of SEQ ID NO: 5 with 5 hits in tobacco genome database of example 1

SEQ ID NO: 56: 15 basepair output nucleotide sequence of SEQ ID NO: 5 with 4 hits in tobacco genome database of example 1

SEQ ID NO: 57: 15 basepair output nucleotide sequence of SEQ ID NO: 5 with 3 hits in tobacco genome database of example 1

SEQ ID NO: 58: 15 basepair output nucleotide sequence of SEQ ID NO: 5 with 4 hits in tobacco genome database of example 1

SEQ ID NO: 59: 15 basepair output nucleotide sequence of SEQ ID NO: 5 with 3 hits in tobacco genome database of example 1

SEQ ID NO: 60: 15 basepair output nucleotide sequence of SEQ ID NO: 5 with 4 hits in tobacco genome database of example 1

SEQ ID NO: 61: 15 basepair output nucleotide sequence of SEQ ID NO: 5 with 4 hits in tobacco genome database of example 1

SEQ ID NO: 62: 15 basepair output nucleotide sequence of SEQ ID NO: 5 with 5 hits in tobacco genome database of example 1

SEQ ID NO: 63: 24 basepair sequence with 0 hit threshold run for SEQ ID NO: 5 and the tobacco genome sequence assembly of Example 1.

SEQ ID NO: 64: 24 basepair sequence with 0 hit threshold run for SEQ ID NO: 5 and the tobacco genome sequence assembly of Example 1.

SEQ ID NO: 65: 24 basepair sequence with 0 hit threshold run for SEQ ID NO: 5 and the tobacco genome sequence assembly of Example 1.

SEQ ID NO: 66: 24 basepair sequence with 0 hit threshold run for SEQ ID NO: 5 and the tobacco genome sequence assembly of Example 1.

SEQ ID NO: 67: 24 basepair sequence with 0 hit threshold run for SEQ ID NO: 5 and the tobacco genome sequence assembly of Example 1.

SEQ ID NO: 68: 24 basepair sequence with 0 hit threshold run for SEQ ID NO: 5 and the tobacco genome sequence assembly of Example 1.

SEQ ID NO: 69: 24 basepair sequence with 0 hit threshold run for SEQ ID NO: 5 and the tobacco genome sequence assembly of Example 1.

SEQ ID NO: 70: 24 basepair sequence with 0 hit threshold run for SEQ ID NO: 5 and the tobacco genome sequence assembly of Example 1.

SEQ ID NO: 71: 24 basepair sequence with 0 hit threshold run for SEQ ID NO: 5 and the tobacco genome sequence assembly of Example 1.

SEQ ID NO: 72: 24 basepair sequence with 0 hit threshold run for SEQ ID NO: 5 and the tobacco genome sequence assembly of Example 1.

SEQ ID NO: 73: 24 basepair sequence with 0 hit threshold run for SEQ ID NO: 5 and the tobacco genome sequence assembly of Example 1.

SEQ ID NO: 74: 24 basepair sequence with 0 hit threshold run for SEQ ID NO: 5 and the tobacco genome sequence assembly of Example 1.

SEQ ID NO: 75: 24 basepair sequence with 0 hit threshold run for SEQ ID NO: 5 and the tobacco genome sequence assembly of Example 1.

SEQ ID NO: 76: 24 basepair sequence with 0 hit threshold run for SEQ ID NO: 5 and the tobacco genome sequence assembly of Example 1.

SEQ ID NO: 77: 24 basepair sequence with 0 hit threshold run for SEQ ID NO: 5 and the tobacco genome sequence assembly of Example 1.

SEQ ID NO: 78: 24 basepair sequence with 0 hit threshold run for SEQ ID NO: 5 and the tobacco genome sequence assembly of Example 1.

SEQ ID NO: 79: 24 basepair sequence with 0 hit threshold run for SEQ ID NO: 5 and the tobacco genome sequence assembly of Example 1.

SEQ ID NO: 80: 24 basepair sequence with 0 hit threshold run for SEQ ID NO: 5 and the tobacco genome sequence assembly of Example 1.

SEQ ID NO: 81: 24 basepair sequence with 0 hit threshold run for SEQ ID NO: 5 and the tobacco genome sequence assembly of Example 1.

SEQ ID NO: 82: 24 basepair sequence with 0 hit threshold run for SEQ ID NO: 5 and the tobacco genome sequence assembly of Example 1.

SEQ ID NO: 83: 24 basepair sequence with 0 hit threshold run for SEQ ID NO: 5 and the tobacco genome sequence assembly of Example 1.

SEQ ID NO: 84: 24 basepair sequence with 0 hit threshold run for SEQ ID NO: 5 and the tobacco genome sequence assembly of Example 1.

SEQ ID NO: 85: 24 basepair sequence with 0 hit threshold run for SEQ ID NO: 5 and the tobacco genome sequence assembly of Example 1.

SEQ ID NO: 86: 24 basepair sequence with 0 hit threshold run for SEQ ID NO: 5 and the tobacco genome sequence assembly of Example 1.

SEQ ID NO: 87: 24 basepair sequence with 0 hit threshold run for SEQ ID NO: 5 and the tobacco genome sequence assembly of Example 1.

SEQ ID NO: 88: 24 basepair sequence with 0 hit threshold run for SEQ ID NO: 5 and the tobacco genome sequence assembly of Example 1.

SEQ ID NO: 89: 24 basepair sequence with 0 hit threshold run for SEQ ID NO: 5 and the tobacco genome sequence assembly of Example 1.

SEQ ID NO: 90: 24 basepair sequence with 0 hit threshold run for SEQ ID NO: 5 and the tobacco genome sequence assembly of Example 1.

SEQ ID NO: 91: 24 basepair sequence with 0 hit threshold run for SEQ ID NO: 5 and the tobacco genome sequence assembly of Example 1.

SEQ ID NO: 92: 24 basepair sequence with 0 hit threshold run for SEQ ID NO: 5 and the tobacco genome sequence assembly of Example 1.

SEQ ID NO: 93: 24 basepair sequence with 0 hit threshold run for SEQ ID NO: 5 and the tobacco genome sequence assembly of Example 1.

SEQ ID NO: 94: 24 basepair sequence with 0 hit threshold run for SEQ ID NO: 5 and the tobacco genome sequence assembly of Example 1.

SEQ ID NO: 95: 24 basepair sequence with 0 hit threshold run for SEQ ID NO: 5 and the tobacco genome sequence assembly of Example 1.

SEQ ID NO: 96: 24 basepair sequence with 0 hit threshold run for SEQ ID NO: 5 and the tobacco genome sequence assembly of Example 1.

SEQ ID NO: 97: 24 basepair sequence with 0 hit threshold run for SEQ ID NO: 5 and the tobacco genome sequence assembly of Example 1.

SEQ ID NO: 98: 24 basepair sequence with 0 hit threshold run for SEQ ID NO: 5 and the tobacco genome sequence assembly of Example 1.

SEQ ID NO: 99: 24 basepair sequence with 0 hit threshold run for SEQ ID NO: 5 and the tobacco genome sequence assembly of Example 1.

SEQ ID NO: 100: 24 basepair sequence with 0 hit threshold run for SEQ ID NO: 5 and the tobacco genome sequence assembly of Example 1.

SEQ ID NO: 101: 24 basepair sequence with 0 hit threshold run for SEQ ID NO: 5 and the tobacco genome sequence assembly of Example 1.

SEQ ID NO: 102: 24 basepair sequence with 0 hit threshold run for SEQ ID NO: 5 and the tobacco genome sequence assembly of Example 1.

SEQ ID NO: 103: 24 basepair sequence with 0 hit threshold run for SEQ ID NO: 5 and the tobacco genome sequence assembly of Example 1.

SEQ ID NO: 104: 24 basepair sequence with 0 hit threshold run for SEQ ID NO: 5 and the tobacco genome sequence assembly of Example 1.

SEQ ID NO: 105: 24 basepair sequence with 0 hit threshold run for SEQ ID NO: 5 and the tobacco genome sequence assembly of Example 1.

SEQ ID NO: 106: 24 basepair sequence with 0 hit threshold run for SEQ ID NO: 5 and the tobacco genome sequence assembly of Example 1.

SEQ ID NO: 107: 24 basepair sequence with 0 hit threshold run for SEQ ID NO: 5 and the tobacco genome sequence assembly of Example 1.

SEQ ID NO: 108: 24 basepair sequence with 0 hit threshold run for SEQ ID NO: 5 and the tobacco genome sequence assembly of Example 1.

SEQ ID NO: 109: 24 basepair sequence with 0 hit threshold run for SEQ ID NO: 5 and the tobacco genome sequence assembly of Example 1.

SEQ ID NO: 110: 24 basepair sequence with 0 hit threshold run for SEQ ID NO: 5 and the tobacco genome sequence assembly of Example 1.

SEQ ID NO: 111: 24 basepair sequence with 0 hit threshold run for SEQ ID NO: 5 and the tobacco genome sequence assembly of Example 1.

SEQ ID NO: 112: 24 basepair sequence with 0 hit threshold run for SEQ ID NO: 5 and the tobacco genome sequence assembly of Example 1.

SEQ ID NO: 113: 24 basepair sequence with 0 hit threshold run for SEQ ID NO: 5 and the tobacco genome sequence assembly of Example 1.

SEQ ID NO: 114: 24 basepair sequence with 0 hit threshold run for SEQ ID NO: 5 and the tobacco genome sequence assembly of Example 1.

SEQ ID NO: 115: 24 basepair sequence with 0 hit threshold run for SEQ ID NO: 5 and the tobacco genome sequence assembly of Example 1.

SEQ ID NO: 116: 24 basepair sequence with 0 hit threshold run for SEQ ID NO: 5 and the tobacco genome sequence assembly of Example 1.

SEQ ID NO: 117: 24 basepair sequence with 0 hit threshold run for SEQ ID NO: 5 and the tobacco genome sequence assembly of Example 1.

SEQ ID NO: 118: 24 basepair sequence with 0 hit threshold run for SEQ ID NO: 5 and the tobacco genome sequence assembly of Example 1.

SEQ ID NO: 119: 24 basepair sequence with 0 hit threshold run for SEQ ID NO: 5 and the tobacco genome sequence assembly of Example 1.

SEQ ID NO: 120: 24 basepair sequence with 0 hit threshold run for SEQ ID NO: 5 and the tobacco genome sequence assembly of Example 1.

SEQ ID NO: 121: 24 basepair sequence with 0 hit threshold run for SEQ ID NO: 5 and the tobacco genome sequence assembly of Example 1.

SEQ ID NO: 122: 24 basepair sequence with 0 hit threshold run for SEQ ID NO: 5 and the tobacco genome sequence assembly of Example 1.

SEQ ID NO: 123: 24 basepair sequence with 0 hit threshold run for SEQ ID NO: 5 and the tobacco genome sequence assembly of Example 1.

SEQ ID NO: 124: 24 basepair sequence with 0 hit threshold run for SEQ ID NO: 5 and the tobacco genome sequence assembly of Example 1.

SEQ ID NO: 125: 24 basepair sequence with 0 hit threshold run for SEQ ID NO: 5 and the tobacco genome sequence assembly of Example 1.

SEQ ID NO: 126: 24 basepair sequence with 0 hit threshold run for SEQ ID NO: 5 and the tobacco genome sequence assembly of Example 1.

SEQ ID NO: 127: 24 basepair sequence with 0 hit threshold run for SEQ ID NO: 5 and the tobacco genome sequence assembly of Example 1.

SEQ ID NO: 128: 24 basepair sequence with 0 hit threshold run for SEQ ID NO: 5 and the tobacco genome sequence assembly of Example 1.

SEQ ID NO: 129: 24 basepair sequence with 0 hit threshold run for SEQ ID NO: 5 and the tobacco genome sequence assembly of Example 1.

SEQ ID NO: 130: 24 basepair sequence with 0 hit threshold run for SEQ ID NO: 5 and the tobacco genome sequence assembly of Example 1.

SEQ ID NO: 131: 24 basepair sequence with 0 hit threshold run for SEQ ID NO: 5 and the tobacco genome sequence assembly of Example 1.

SEQ ID NO: 132: 24 basepair sequence with 0 hit threshold run for SEQ ID NO: 5 and the tobacco genome sequence assembly of Example 1.

SEQ ID NO: 133: 24 basepair sequence with 0 hit threshold run for SEQ ID NO: 5 and the tobacco genome sequence assembly of Example 1.

SEQ ID NO: 134: 24 basepair sequence with 0 hit threshold run for SEQ ID NO: 5 and the tobacco genome sequence assembly of Example 1.

SEQ ID NO: 135: 24 basepair sequence with 0 hit threshold run for SEQ ID NO: 5 and the tobacco genome sequence assembly of Example 1.

SEQ ID NO: 136: 24 basepair sequence with 0 hit threshold run for SEQ ID NO: 5 and the tobacco genome sequence assembly of Example 1.

SEQ ID NO: 137: 24 basepair sequence with 0 hit threshold run for SEQ ID NO: 5 and the tobacco genome sequence assembly of Example 1.

SEQ ID NO: 138: 24 basepair sequence with 0 hit threshold run for SEQ ID NO: 5 and the tobacco genome sequence assembly of Example 1.

SEQ ID NO: 139: 24 basepair sequence with 0 hit threshold run for SEQ ID NO: 5 and the tobacco genome sequence assembly of Example 1.

SEQ ID NO: 140: 24 basepair sequence with 0 hit threshold run for SEQ ID NO: 5 and the tobacco genome sequence assembly of Example 1.

SEQ ID NO: 141: 24 basepair sequence with 0 hit threshold run for SEQ ID NO: 5 and the tobacco genome sequence assembly of Example 1.

SEQ ID NO: 142: 24 basepair sequence with 0 hit threshold run for SEQ ID NO: 5 and the tobacco genome sequence assembly of Example 1.

SEQ ID NO: 143: 24 basepair sequence with 0 hit threshold run for SEQ ID NO: 5 and the tobacco genome sequence assembly of Example 1.

SEQ ID NO: 144: 24 basepair sequence with 0 hit threshold run for SEQ ID NO: 5 and the tobacco genome sequence assembly of Example 1.

SEQ ID NO: 145: 24 basepair sequence with 0 hit threshold run for SEQ ID NO: 5 and the tobacco genome sequence assembly of Example 1.

SEQ ID NO: 146: 24 basepair sequence with 0 hit threshold run for SEQ ID NO: 5 and the tobacco genome sequence assembly of Example 1.

SEQ ID NO: 147: 24 basepair sequence with 0 hit threshold run for SEQ ID NO: 5 and the tobacco genome sequence assembly of Example 1.

SEQ ID NO: 148: 24 basepair sequence with 0 hit threshold run for SEQ ID NO: 5 and the tobacco genome sequence assembly of Example 1.

SEQ ID NO: 149: 24 basepair sequence with 0 hit threshold run for SEQ ID NO: 5 and the tobacco genome sequence assembly of Example 1.

SEQ ID NO: 150: 24 basepair sequence with 0 hit threshold run for SEQ ID NO: 5 and the tobacco genome sequence assembly of Example 1.

SEQ ID NO: 151: 24 basepair sequence with 0 hit threshold run for SEQ ID NO: 5 and the tobacco genome sequence assembly of Example 1.

SEQ ID NO: 152: 24 basepair sequence with 0 hit threshold run for SEQ ID NO: 5 and the tobacco genome sequence assembly of Example 1.

SEQ ID NO: 153: 24 basepair sequence with 0 hit threshold run for SEQ ID NO: 5 and the tobacco genome sequence assembly of Example 1.

SEQ ID NO: 154: 24 basepair sequence with 0 hit threshold run for SEQ ID NO: 5 and the tobacco genome sequence assembly of Example 1.

SEQ ID NO: 155: 24 basepair sequence with 0 hit threshold run for SEQ ID NO: 5 and the tobacco genome sequence assembly of Example 1.

SEQ ID NO: 156: 24 basepair sequence with 0 hit threshold run for SEQ ID NO: 5 and the tobacco genome sequence assembly of Example 1.

SEQ ID NO: 157: 24 basepair sequence with 0 hit threshold run for SEQ ID NO: 5 and the tobacco genome sequence assembly of Example 1.

SEQ ID NO: 158: 24 basepair sequence with 0 hit threshold run for SEQ ID NO: 5 and the tobacco genome sequence assembly of Example 1.

SEQ ID NO: 159: 24 basepair sequence with 0 hit threshold run for SEQ ID NO: 5 and the tobacco genome sequence assembly of Example 1.

SEQ ID NO: 160: 24 basepair sequence with 0 hit threshold run for SEQ ID NO: 5 and the tobacco genome sequence assembly of Example 1.

SEQ ID NO: 161: 24 basepair sequence with 0 hit threshold run for SEQ ID NO: 5 and the tobacco genome sequence assembly of Example 1.

SEQ ID NO: 162: 24 basepair sequence with 0 hit threshold run for SEQ ID NO: 5 and the tobacco genome sequence assembly of Example 1.

SEQ ID NO: 163: 24 basepair sequence with 0 hit threshold run for SEQ ID NO: 5 and the tobacco genome sequence assembly of Example 1.

SEQ ID NO: 164: 24 basepair sequence with 0 hit threshold run for SEQ ID NO: 5 and the tobacco genome sequence assembly of Example 1.

SEQ ID NO: 165: 24 basepair sequence with 0 hit threshold run for SEQ ID NO: 5 and the tobacco genome sequence assembly of Example 1.

SEQ ID NO: 166: 24 basepair sequence with 0 hit threshold run for SEQ ID NO: 5 and the tobacco genome sequence assembly of Example 1.

SEQ ID NO: 167: 24 basepair sequence with 0 hit threshold run for SEQ ID NO: 5 and the tobacco genome sequence assembly of Example 1.

SEQ ID NO: 168: 24 basepair sequence with 0 hit threshold run for SEQ ID NO: 5 and the tobacco genome sequence assembly of Example 1.

SEQ ID NO: 169: 24 basepair sequence with 0 hit threshold run for SEQ ID NO: 5 and the tobacco genome sequence assembly of Example 1.

SEQ ID NO: 170: 24 basepair sequence with 0 hit threshold run for SEQ ID NO: 5 and the tobacco genome sequence assembly of Example 1.

SEQ ID NO: 171: 24 basepair sequence with 0 hit threshold run for SEQ ID NO: 5 and the tobacco genome sequence assembly of Example 1.

SEQ ID NO: 172: 24 basepair sequence with 0 hit threshold run for SEQ ID NO: 5 and the tobacco genome sequence assembly of Example 1.

SEQ ID NO: 173: 24 basepair sequence with 0 hit threshold run for SEQ ID NO: 5 and the tobacco genome sequence assembly of Example 1.

SEQ ID NO: 174: 24 basepair sequence with 0 hit threshold run for SEQ ID NO: 5 and the tobacco genome sequence assembly of Example 1.

SEQ ID NO: 175: 24 basepair sequence with 0 hit threshold run for SEQ ID NO: 5 and the tobacco genome sequence assembly of Example 1.

SEQ ID NO: 176: 24 basepair sequence with 0 hit threshold run for SEQ ID NO: 5 and the tobacco genome sequence assembly of Example 1.

SEQ ID NO: 177: 24 basepair sequence with 0 hit threshold run for SEQ ID NO: 5 and the tobacco genome sequence assembly of Example 1.

SEQ ID NO: 178: 24 basepair sequence with 0 hit threshold run for SEQ ID NO: 5 and the tobacco genome sequence assembly of Example 1.

SEQ ID NO: 179: 24 basepair sequence with 0 hit threshold run for SEQ ID NO: 5 and the tobacco genome sequence assembly of Example 1.

SEQ ID NO: 180: 24 basepair sequence with 0 hit threshold run for SEQ ID NO: 5 and the tobacco genome sequence assembly of Example 1.

SEQ ID NO: 181: 24 basepair sequence with 0 hit threshold run for SEQ ID NO: 5 and the tobacco genome sequence assembly of Example 1.

SEQ ID NO: 182: 24 basepair sequence with 0 hit threshold run for SEQ ID NO: 5 and the tobacco genome sequence assembly of Example 1.

SEQ ID NO: 183: 24 basepair sequence with 0 hit threshold run for SEQ ID NO: 5 and the tobacco genome sequence assembly of Example 1.

SEQ ID NO: 184: 24 basepair sequence with 0 hit threshold run for SEQ ID NO: 5 and the tobacco genome sequence assembly of Example 1.

SEQ ID NO: 185: 24 basepair sequence with 0 hit threshold run for SEQ ID NO: 5 and the tobacco genome sequence assembly of Example 1.

SEQ ID NO: 186: 24 basepair sequence with 0 hit threshold run for SEQ ID NO: 5 and the tobacco genome sequence assembly of Example 1.

SEQ ID NO: 187: 24 basepair sequence with 0 hit threshold run for SEQ ID NO: 5 and the tobacco genome sequence assembly of Example 1.

SEQ ID NO: 188: 24 basepair sequence with 0 hit threshold run for SEQ ID NO: 5 and the tobacco genome sequence assembly of Example 1.

SEQ ID NO: 189: 24 basepair sequence with 0 hit threshold run for SEQ ID NO: 5 and the tobacco genome sequence assembly of Example 1.

SEQ ID NO: 190: 24 basepair sequence with 0 hit threshold run for SEQ ID NO: 5 and the tobacco genome sequence assembly of Example 1.

SEQ ID NO: 191: 24 basepair sequence with 0 hit threshold run for SEQ ID NO: 5 and the tobacco genome sequence assembly of Example 1.

SEQ ID NO: 192: 24 basepair sequence with 0 hit threshold run for SEQ ID NO: 5 and the tobacco genome sequence assembly of Example 1.

SEQ ID NO: 193: 24 basepair sequence with 0 hit threshold run for SEQ ID NO: 5 and the tobacco genome sequence assembly of Example 1.

SEQ ID NO: 194: 24 basepair sequence with 0 hit threshold run for SEQ ID NO: 5 and the tobacco genome sequence assembly of Example 1.

SEQ ID NO: 195: 24 basepair sequence with 0 hit threshold run for SEQ ID NO: 5 and the tobacco genome sequence assembly of Example 1.

SEQ ID NO: 196: 24 basepair sequence with 0 hit threshold run for SEQ ID NO: 5 and the tobacco genome sequence assembly of Example 1.

SEQ ID NO: 197: 24 basepair sequence with 0 hit threshold run for SEQ ID NO: 5 and the tobacco genome sequence assembly of Example 1.

SEQ ID NO: 198: 24 basepair sequence with 0 hit threshold run for SEQ ID NO: 5 and the tobacco genome sequence assembly of Example 1.

SEQ ID NO: 199: 24 basepair sequence with 0 hit threshold run for SEQ ID NO: 5 and the tobacco genome sequence assembly of Example 1.

SEQ ID NO: 200: 24 basepair sequence with 0 hit threshold run for SEQ ID NO: 5 and the tobacco genome sequence assembly of Example 1.

SEQ ID NO: 201: 24 basepair sequence with 0 hit threshold run for SEQ ID NO: 5 and the tobacco genome sequence assembly of Example 1.

SEQ ID NO: 202: 24 basepair sequence with 0 hit threshold run for SEQ ID NO: 5 and the tobacco genome sequence assembly of Example 1.

SEQ ID NO: 203: 24 basepair sequence with 0 hit threshold run for SEQ ID NO: 5 and the tobacco genome sequence assembly of Example 1.

SEQ ID NO: 204: 24 basepair sequence with 0 hit threshold run for SEQ ID NO: 5 and the tobacco genome sequence assembly of Example 1.

SEQ ID NO: 205: 24 basepair sequence with 0 hit threshold run for SEQ ID NO: 5 and the tobacco genome sequence assembly of Example 1.

SEQ ID NO: 206: 24 basepair sequence with 0 hit threshold run for SEQ ID NO: 5 and the tobacco genome sequence assembly of Example 1.

SEQ ID NO: 207: 24 basepair sequence with 0 hit threshold run for SEQ ID NO: 5 and the tobacco genome sequence assembly of Example 1.

SEQ ID NO: 208: 24 basepair sequence with 0 hit threshold run for SEQ ID NO: 5 and the tobacco genome sequence assembly of Example 1.

SEQ ID NO: 209: 24 basepair sequence with 0 hit threshold run for SEQ ID NO: 5 and the tobacco genome sequence assembly of Example 1.

SEQ ID NO: 210: 24 basepair sequence with 0 hit threshold run for SEQ ID NO: 5 and the tobacco genome sequence assembly of Example 1.

SEQ ID NO: 211: 24 basepair sequence with 0 hit threshold run for SEQ ID NO: 5 and the tobacco genome sequence assembly of Example 1.

SEQ ID NO: 212: partial cDNA sequence of Nicotiana tabacum fragment GnTI-A variant 1

SEQ ID NO: 213: partial cDNA sequence of Nicotiana tabacum fragment GnTI-A variant 1

SEQ ID NO: 214: partial amino acid sequence of Nicotiana tabacum fragment GnTI-B cDNA variant 1

SEQ ID NO: 215: partial amino acid sequence of Nicotiana tabacum fragment GnTI-B cDNA variant 1

SEQ ID NO: 216: partial amino acid sequence of Nicotiana benthamiana fragment GnTI-B cDNA variant 1

SEQ ID NO: 217: partial amino acid sequence of Nicotiana tabacum fragment GnTI-A cDNA variant 1

SEQ ID NO: 218: partial amino acid sequence of Nicotiana tabacum fragment GnTI-A cDNA variant 1

SEQ ID NO: 219: partial cDNA sequence variant 2 of Nicotiana tabacum fragment GnTI-B

SEQ ID NO: 220: partial cDNA sequence variant 3 of Nicotiana tabacum fragment GnTI-B

SEQ ID NO: 221: partial amino acid sequence of Nicotiana tabacum fragment GnTI-B cDNA variant 2

SEQ ID NO: 222: partial amino acid sequence of Nicotiana tabacum fragment GnTI-B cDNA variant 3

SEQ ID NO: 223: partial cDNA sequence variant 2 of Nicotiana tabacum fragment GnTI-B

SEQ ID NO: 224: partial amino acid sequence of Nicotiana tabacum fragment GnTI-B cDNA variant 2

SEQ ID NO: 225: partial cDNA sequence variant 2 of Nicotiana benthamiana fragment GnTI-B

SEQ ID NO: 226: partial amino acid sequence of Nicotiana benthamiana fragment GnTI-B cDNA variant 2

SEQ ID NO: 227: partial cDNA sequence of Nicotiana tabacum fragment GnTI-A variant 2

SEQ ID NO: 228: partial amino acid sequence of Nicotiana tabacum fragment GnTI-A cDNA variant 2

SEQ ID NO: 229: partial cDNA sequence of Nicotiana tabacum GnTI-A variant 2

SEQ ID NO: 230: partial amino acid sequence of Nicotiana tabacum fragment GnTI-A cDNA variant 2

SEQ ID NO: 231: nucleotide sequence of NGSG12045 forward primer suitable for amplifying a fragment of contig gDNA_c1690982 that contains a Nicotiana tabacum N-acetylglucosaminyltransferase I intron-exon sequence

SEQ ID NO: 232: nucleotide sequence of NGSG12045 reverse primer suitable for amplifying a fragment of contig gDNA_cl 690982 that contains a Nicotiana tabacum N-acetylglucosaminyltransferase I intron-exon sequence

SEQ ID NO: 233: basepairs 1-15,000 of the nucleotide sequence of NtPMI-BAC-FABIJI_{—}1 that contains Nicotiana tabacum N-acetylglucosaminyltransferase I gene variant 2

SEQ ID NO: 234: predicted cDNA sequence of Nicotiana tabacum N-acetylglucosaminyltransferase I gene variant 2

SEQ ID NO: 235: amino acid sequence of Nicotiana tabacum N-acetylglucosaminyltransferase I gene variant 2

SEQ ID NO: 236: primer sequence FABIJI-forward for amplification of FABIJI-homolog of N. tabacum PM132

SEQ ID NO: 237: primer sequence FABIJI-reverse for amplification of FABIJI-homolog of N. tabacum PM132

SEQ ID NO: 238: primer sequence CPO-forward for amplification of CPO GnTI genomic sequence of N. tabacum PM132

SEQ ID NO: 239: primer sequence CPO-reverse for amplification of CPO GnTI genomic sequence of N. tabacum PM132

SEQ ID NO: 240: primer sequence CAC80702.1-forward for amplification of CAC80702.1 homolog of N. tabacum PM132

SEQ ID NO: 241: primer sequence CAC80702.1-reverse for amplification of CAC80702.1 homolog of N. tabacum PM132

SEQ ID NO: 242: primer sequence FABIJI-1 homolog-forward for amplification of GnTI sequence of N. tabacum Hicks Broadleaf

SEQ ID NO: 243: primer sequence FABIJI-1 homolog-reverse for amplification of GnTI sequence of N. tabacum Hicks Broadleaf

SEQ ID NO: 244: primer sequence FABIJI-1 homolog-forward for amplification of GnTI sequence of N. tabacum Hicks Broadleaf

SEQ ID NO: 245: primer sequence FABIJI-1 homolog-reverse for amplification of GnTI sequence of N. tabacum Hicks Broadleaf

SEQ ID NO: 246: primer sequence PC181F for amplification of gDNA of N. tabacum PM132 containing 5′ UTR and exons 1 to 7

SEQ ID NO: 247: primer sequence PC190R for amplification of gDNA of N. tabacum PM132 containing 5′ UTR and exons 1 to 7

SEQ ID NO: 248: primer sequence PC191F for amplification of gDNA of N. tabacum PM132 containing exons 4 to 13

SEQ ID NO: 249: primer sequence PC192R for amplification of gDNA of N. tabacum PM132 containing exons 4 to 13

SEQ ID NO: 250: primer sequence PC193F for amplification of gDNA of N. tabacum PM132 containing exons 12 to 19 and 3′ UTR

SEQ ID NO: 251: primer sequence PC187R for amplification of gDNA of N. tabacum PM132 containing exons 12 to 19 and 3′ UTR

SEQ ID NO: 252: primer sequence PC193F for amplification of gDNA of N. tabacum PM132 containing exons 12 to 19 and 3′ UTR

SEQ ID NO: 253: primer sequence PC188R for amplification of gDNA of N. tabacum PM132 containing exons 12 to 19 and 3′ UTR

SEQ ID NO: 254: primer sequence PC193F for amplification of gDNA of N. tabacum PM132 containing exons 12 to 19 and 3′ UTR

SEQ ID NO: 255: primer sequence PC189R for amplification of gDNA of N. tabacum PM132 containing exons 12 to 19 and 3′ UTR

SEQ ID NO: 256: nucleotide sequence of genomic FABIJI-homolog of N. tabacum PM132

SEQ ID NO: 257: nucleotide sequence of coding sequence of FABIJI-homolog N. tabacum PM132

SEQ ID NO: 258: amino acid sequence of FABIJI-homolog N. tabacum PM 132

SEQ ID NO: 259: nucleotide sequence of genomic CPO-gDNA of N. tabacum PM132

SEQ ID NO: 260: nucleotide sequence of predicted coding region of N. tabacum PM132 CPO gene

SEQ ID NO: 261: predicted amino acid sequence of coding region of N. tabacum PM132 CPO gene

SEQ ID NO: 262: nucleotide sequence of N. tabacum PM132 CAC80702.1 homolog

SEQ ID NO: 263: nucleotide sequence of coding region of N. tabacum PM132 CAC80702.1 homolog

SEQ ID NO: 264: predicted amino acid sequence of N. tabacum PM132 CAC80702.1 homolog

SEQ ID NO: 265: nucleotide acid sequence of GnTI contig 1#5 of N. tabacum PM132

SEQ ID NO: 266: nucleotide acid sequence of predicted GnTI coding region contig 1#5

SEQ ID NO: 267: predicted amino acid sequence of GnTI contig 1#5 of N. tabacum PM132

SEQ ID NO: 268: nucleotide acid sequence of GnTI contig 1#8 of N. tabacum PM132

SEQ ID NO: 269: nucleotide acid sequence of predicted GnTI coding region contig 1#8

SEQ ID NO: 270: predicted amino acid sequence of GnTI contig 1#8 of N. tabacum PM132

SEQ ID NO: 271: nucleotide acid sequence of GnTI contig 1#9 of N. tabacum PM132

SEQ ID NO: 272: nucleotide acid sequence of predicted GnTI coding region contig 1#9

SEQ ID NO: 273: predicted amino acid sequence of GnTI contig 1# of N. tabacum PM1329

SEQ ID NO: 274: nucleotide acid sequence of GnTI T10 702 of N. tabacum PM132

SEQ ID NO: 275: nucleotide acid sequence of predicted GnTI coding region T10 702

SEQ ID NO: 276: predicted amino acid sequence of GnTI T10 702 of N. tabacum PM132

SEQ ID NO: 277: nucleotide acid sequence of GnTI contig 1#6 of N. tabacum PM132

SEQ ID NO: 278: nucleotide acid sequence of predicted GnTI coding region contig 1#6

SEQ ID NO: 279: predicted amino acid sequence of GnTI contig 1#6 of N. tabacum PM132

SEQ ID NO: 280: nucleotide acid sequence of GnTI contig 1#2 of N. tabacum PM132

SEQ ID NO: 281: nucleotide acid sequence of predicted GnTI coding region contig 1#2

SEQ ID NO: 282: predicted amino acid sequence of GnTI contig 1#2 of N. tabacum PM132

### EXAMPLES

The following examples are provided as an illustration and not as a limitation. Unless otherwise indicated, the present invention employs conventional techniques and methods of molecular biology, plant biology, bioinformatics, and plant breeding.

### Example 1

### Identification of a Nicotiana tabacum β(1,2)-Xylosyltransferase Variant 1 Genome Sequence

This example illustrates how a genomic nucleotide sequence of a beta-1,2-xylosyltransferase (β(1,2)-xylosyltransferase) of Nicotiana tabacum can be identified. Tobacco BAC library. A Bacterial Artificial Chromosome (BAC) library is prepared as follows: nuclei are isolated from leaves of greenhouse grown plants of the Nicotiana tabacum variety Hicks Broad Leaf. High-molecular weight DNA is isolated from the nuclei according to standard protocols and partially digested with BamHI and HindIII and cloned in the BamHI or HindIII sites of the BAC vector pINDIGO5. More than 320,000 clones are obtained with an average insert length of 135 Megabasepairs covering approximately 9.7 times the tobacco genome.

### Tobacco Genome Sequence Assembly.

A large number of randomly-picked BAC clones are submitted to sequencing using the Sanger method generating more than 1,780,000 raw sequences of an average length of 550 basepairs. Methyl filtering is applied by using a Mcr+ strain of Escherichia coli for transformation and isolating only hypomethylated DNA. All sequences are assembled using the CELERA genome assembler yielding more than 800,000 sequences comprising more than 200,000 contigs and 596,970 single sequences. Contig sizes are between 120 and 15,300 basepairs with an average length of 1,100 basepairs.

### Development and Analysis of Tobacco ExonArray.

272,342 exons are identified by combining and comparing public tobacco EST data and the methyl-filtered sequences obtained from the BAC sequencing. For each of these exons, four 25-mer oligonucleotides are designed and used to construct a tobacco ExonArray. The ExonArray is made by Affymetrix (Santa Clara, USA) using standard protocols. Of the 272,432 exons, eleven (11) are identified having homology to beta-1,2-xylosyltransferase (β(1,2)-xylosyltransferase) gene sequences annotated in public databases. The 11 exons belong to 6 contigs. Using standard hybridization protocols and analytical tools, it appears that ten (10) out of these 11 exons are active in tobacco leaf tissue. One contig showing highest expression values, gDNA_c1736055 is chosen for primer design to identify a BAC clone to obtain the full genomic DNA sequence. SEQ ID NO: 1 represents the full sequence of contig gDNA_c1736055.

### Primer Design.

A primer pair NGSG10043 is designed for contig gDNA_c1736055 using Primer3 (Rozen and Skaletsky, 2000) in a way that both primers making up a pair surrounded an exon-non-coding sequence boundary with a calculated product length between 250 and 500 basepairs. NGSG10043 is designed as follows: primer SEQ ID NO: 2 maps to the untranslated part of gDNA_c1736055 preceeding a putative startcodon on the plus strand and primer SEQ ID NO:3 to a predicted exon part of said sequence to improve specificity. Primer pair NGSG10043 comprising primers SEQ ID NO: 2 and SEQ ID NO: 3 is used for screening the BAC library. This strategy can be useful in distinguishing the different multiple variants and alleles that are present in the genome.

### Screening of BAC Library.

DNA is isolated from BAC clones that are pooled in a three dimensional way to facilitate the identification of individual clones with homology to a certain sequence. Primer pair NGSG10043 is used to screen the full BAC library using PCR and standard BAC screening procedures and single clones are identified that gave the expected fragment size. One of those BAC clones, NtPMI-BAC-TAKOMI_{—}6, is chosen for further analysis and purified DNA of NtPMI-BAC-TAKOMI_{—}6 is sequenced using 454 sequencing on a Genome Sequencer FLX System (Roche Diagnostics Corporation). Assembly of all raw NtPMI-BAC-TAKOMI_{—}6 sequences using Newbler assembler (454 Life Sciences, Branford, USA) and annotation with TAIR and Uniprot entries identifies one contig of 28,936 basepairs, 257B4-contig00006, that contains sequences with homology to an Arabidopsis thaliana beta-1,2-xylosyltransferase (AT5G55500.1; TAIR accession gene 2173891). SEQ ID NO: 4 discloses a 6,000 basepair fragment of the NtPMI-BAC-TAKOMI_{—}6 comprising a fragment of approximately 3,465 basepairs on the minus strand showing homology to Arabidopsis thaliana gene AT5G55500.1 (SEQ ID NO: 5) as well as a fragment of 1,430 basepair following the putative stopcodon and 1,140 basepairs preceeding the putative startcodon of the predicted gene (SEQ ID NO: 6). The 358 basepair fragment of NtPMI-BAC-TAKOMI_{—}6 that is amplified using primer set NGSG10043 is represented by SEQ ID NO: 7.

### Identification of β(1,2)-Xylosyltransferase Gene Sequence.

The 6,000 basepair genomic sequence of NtPMI-BAC-TAKOMI_{—}6 showing homology to an Arabidopsis thaliana beta-1,2-xylosyltransferase (β(1,2)-xylosyltransferase) gene sequence is further annotated with the gene finding programs Augustus (University of Göttingen, Göttingen, Germany) and FgeneSH (Softberry Inc., Mount Kisco, USA) that predicts genes in eukarytic genomic sequences. Both gene finding programs are first trained on known tobacco genes. The predicted FgeneSH and Augustus genes that overlap with the 3,430 basepair fragment showing homology to A. thaliana AT5G55500.1 are further manually annotated by comparison with known β(1,2)-Xylosyltransferase cDNA and amino acid sequences. SEQ ID NO: 8 discloses the cDNA sequence relating to SEQ ID NO: 5. SEQ ID NO: 8 comprises 1,572 basepairs including the stopcodon and codes for a 523 amino acid polypeptide (SEQ ID NO: 9).

### Tobacco Beta-1,2-Xylosyltransferase (β(1,2)Xylosyltransferase) Gene Structure.

By comparing the genomic DNA sequence SEQ ID NO: 5 and the beta-1,2-xylosyltransferase (β(1,2)-xylosyltransferase) cDNA sequence SEQ ID NO: 8 it is concluded that the genomic gene coding sequence comprises three exons on the minus strand, spanning from 4,894 to approximately 4,196 (startcodon-exon1), approximately 2,899 to 2,750 (exon 2) and approximately 2,152 to 1,430 (exon 3-stopcodon) on the minus strand of SEQ ID NO: 4 and two intervening introns.

### Example 2

### Identification of Nicotiana tabacum Beta-1,2-Xylosyltransferase (β(1,2)-Xylosyltransferase) Variant 2

Beta-1,2-xylosyltransferase (β(1,2)-xylosyltransferase) gene variant 2 of Nicotiana tabacum is identified as described in Example 1 but using primer pairs NGSG10046 (SEQ ID NO: 15 and 16) based on contig CHO_OF4335xn13f1, respectively. SEQ ID NO: 12 represents basepairs 60,001-65,698 of the nucleotide sequence of NtPMI-BAC-GEJUJO_{—}2 that contains Nicotiana tabacum beta-1,2-xylosyltransferase (β(1,2)-xylosyltransferase) gene variant 2. SEQ ID NO: 13 represents the cDNA sequence of Nicotiana tabacum beta-1,2-xylosyltransferase (β(1,2)-xylosyltransferase) gene variant 2. SEQ ID NO: 17 represents basepairs 15,921-23,200 of the nucleotide sequence of NtPMI-BAC-SANIKI_{—}1 that contains Nicotiana tabacum beta-1,2-xylosyltransferase (β(1,2)-xylosyltransferase) gene variant 2. SEQ ID NO: 18 represents the cDNA sequence of Nicotiana tabacum beta-1,2-xylosyltransferase (β(1,2)-xylosyltransferase) gene variant 2 and SEQ ID NO: 19 represents the amino acid sequence of Nicotiana tabacum beta-1,2-xylosyltransferase (3(1,2)-xylosyltransferase) protein variant 2.

### Example 3

### Identification of Nicotiana tabacum Alpha-1,3-Fucosyltransferase (α(1,3)Fucosyltransferase) Variants 1 to 4

Four alpha-1,3-fucosyltransferase (α(1,3)-fucosyltransferase) gene variants of Nicotiana tabacum are identified essentially as described in Example 1 using primer pairs NGSG10032 (SEQ ID SEQ ID NO: 30 and 31), NGSG10034 (SEQ ID NO: 35 and 36), NGSG10035 (SEQ ID NO: 45 and 46) and NGSG10041 (SEQ ID NO: 25 and 26). SEQ ID NO: 27 represents basepairs 2,961-10,160 of the nucleotide sequence of NtPMI-BAC-FETILA_{—}9 that contains Nicotiana tabacum alpha-1,3-fucosyltransferase (α(1,3)-fucosyltransferase) gene variant 1, SEQ ID NO: 28 the cDNA sequence of alpha-1,3-fucosyltransferase (α(1,3)-fucosyltransferase) gene variant 1 and SEQ ID NO: 29 the amino acid sequence of alpha-1,3-fucosyltransferase (α(1,3)-fucosyltransferase) protein variant 1. SEQ ID NO: 32 represents basepairs 1,041-7,738 of the nucleotide sequence of NtPMI-BAC-JUMAKE_{—}4 that contains Nicotiana tabacum alpha-1,3-fucosyltransferase (α(1,3)-fucosyltransferase) gene variant 2, SEQ ID NO: 33 the partial cDNA sequence of alpha-1,3-fucosyltransferase (α(1,3)-fucosyltransferase) gene variant 2 and SEQ ID NO: 34 the partial amino acid sequence of alpha-1,3-fucosyltransferase (α(1,3)-fucosyltransferase) protein variant 2. SEQ ID NO: 37 represents basepairs 19,001-23,871 of the nucleotide sequence of NtPMI-BAC-JEJOLO_{—}22 that contains partial Nicotiana tabacum alpha-1,3-fucosyltransferase (α(1,3)-fucosyltransferase) gene variant 3, SEQ ID NO: 38 the partial cDNA sequence of alpha-1,3-fucosyltransferase (α(1,3)-fucosyltransferase) gene variant 3 and SEQ ID NO: 39 the partial amino acid sequence of α(1,3)-fucosyltransferase protein variant 3.

SEQ ID NO: 47 represents basepairs 1-11,000 of the nucleotide sequence of NtPMI-BAC-JUDOSU_{—}1 that contains Nicotiana tabacum alpha-1,3-fucosyltransferase (α(1,3)-fucosyltransferase) gene variant 4, SEQ ID NO: 48 the partial cDNA sequence of alpha-1,3-fucosyltransferase (α(1,3)-fucosyltransferase) gene variant 4 and SEQ ID NO: 49 the partial amino acid sequence of alpha-1,3-fucosyltransferase (α(1,3)-fucosyltransferase) protein variant 4.

### Example 4

### Search Protocol for the Selection of Zinc Finger Nuclease Target Sites

This example illustrates how to search a genomic nucleotide sequence of a given gene to screen for the occurrence of unique target sites within the given gene sequence compared to a given genome database to develop tools for modifying the expression of the gene. The target sites identified by methods of the invention, including those disclosed below, the sequence motifs, and use of any of the sites or motifs in modifying the corresponding gene sequence in a plant, such as tobacco, are encompassed in the invention.

### Search Algorithm.

A computer program is developed that allows one to screen an input query (target) nucleotide sequence for the occurrence of two fixed-length substring DNA motifs separated by a given spacer size using a suffix array within a DNA database, such as for example the tobacco genome sequence assembly of Example 1. The suffix array construction and the search use the open source libdivsufsort library-2.0.0 (http://code.google.com/p/libdivsufsort/) which converts any input string directly into a Burrows-Wheeler transformed string. The program scans the full input (target) nucleotide sequence and returns all the substring combinations occurring less than a selected number of times in the selected DNA database.

### Selection of Target Site for Zinc Finger Nuclease-Mediated Mutagenesis of a Query Sequence.

A zinc finger DNA binding domain recognizes a three basepair nucleotide sequence. A zinc finger nuclease comprises a zinc finger protein comprising one, two, three, four, five, six or more zinc finger DNA binding domains, and the non-specific nuclease of a Type IIS restriction enzyme. Zinc finger nucleases can be used to introduce a double-stranded break into a target sequence. To introduce a double-stranded break, a pair of zinc finger nucleases, one of which binds to the plus (upper) strand of the target sequence and the other to the minus (lower) strand of the same target sequence separated by 0, 1, 2, 3, 4, 5, 6 or more nucleotides are required. By using plurals of 3 for each of the two fixed-length substring DNA motifs, the program can be used to identify two zinc finger protein target sites separated by a given spacer length

### Program Inputs:

1. The target query DNA sequence
2. The DNA database to be searched
3. The fixed size of the first substring DNA motif
4. The fixed size of the spacer
5. The fixed size of the second substring DNA motif
6. The threshold number of occurrences of the combination of program inputs 3 and 5 separated by program input 4 in the chosen DNA database of program input 2

### Program Output:

1. A list of nucleotide sequences with for each sequence the number of times the sequence occurs in the DNA database with a maximum of the program input 6 threshold.

### Example 5

### Selection of Target Sites within Nicotiana tabacum Beta-1,2-Xylosyltransferase (β(1,2)-Xylosyltransferase) Variant 1 Nucleotide Sequence with a Fixed 6 Basepair First and Second Substring, a Fixed 3 Basepair Spacer and a Maximum Threshold of 5 Hits in the Tobacco Genome Sequence Assembly

### Program Inputs:

1. Nicotiana tabacum beta-1,2-xylosyltransferase (β(1,2)-xylosyltransferase) SEQ ID NO: 5 as target query DNA sequence
2. The tobacco genome sequence assembly of Example 1 as DNA database to be searched
3. A fixed 6 basepair first substring DNA motif
4. A fixed 3 basepair spacer
5. A fixed 6 basepair second substring DNA motif
6. A maximum threshold number of occurrences of the combination of program inputs 3 and 5 separated by program input 4 in the chosen DNA database of program input 2 of 5 hits

### Program Output:

ACCGTA NNN GGCGAC (SEQ ID NO: 50): 4 hits
CCGTAT NNN GCGACG (SEQ ID NO: 51): 5 hits
TATCCG NNN ACGGCG (SEQ ID NO: 52): 5 hits
GCGAGG NNN GTGCTA (SEQ ID NO: 53): 5 hits
TCTCGT NNN GGCGAG (SEQ ID NO: 54): 5 hits
CGGTTA NNN GTAGGA (SEQ ID NO: 55): 5 hits
AGTTAG NNN GCGCCG (SEQ ID NO: 56): 4 hits
CGTGGC NNN CAGGGT (SEQ ID NO: 57): 3 hits
CCTTAC NNN ACGTCT (SEQ ID NO: 58): 4 hits
GGCCAT NNN GGGGGC (SEQ ID NO: 59): 3 hits
GCCATA NNN GGGGCG (SEQ ID NO: 60): 4 hits
GCACGG NNN TCCGAG (SEQ ID NO: 61): 4 hits
GCGAAT NNN GGCGCC (SEQ ID NO: 62): 5 hits

This example illustrates that any pair of zinc finger nucleases of which each zinc finger protein comprised two fixed 6 basepair long DNA binding domains with a 3 basepair fixed intervening spacer sequence, for the given target sequence SEQ ID NO: 5, comprising the full genomic sequence for a β(1,2)-xylosyltransferase from ATG-startcodon to TAA-stopcodon and containing three exons and two introns, will target at least three other sites within the tobacco genome. The example also illustrates that only 13 pairs occur less or equal to 5 times in the tobacco genome and all other pairs more than 5 times.

### Example 6

### Selection of Target Sites for Zinc Finger Nuclease Genome Editing of the Exon 2 Fragment of the Coding Sequence of Nicotiana tabacum Beta-1,2-Xylosyltransferase (β(1,2)-Xylosyltransferase) Variant 1

This example illustrates:
1. How a list of target sites for zinc finger mediated mutagenesis of the Nicotiana tabacum beta-1,2-xylosyltransferase (β(1,2)-xylosyltransferase) variant 1 of SEQ ID NO: 5 for exon 2 was compiled
2. How a pair of target sites for the design of two zinc finger nucleases making up a pair to mutagenize the coding sequence was chosen
3. How the output of the program can be used to develop a pair of zinc finger nucleases

### Program Input:

1. Exon 2 fragment of SEQ ID NO: 5 from basepair 2,750 to 2,899 (minus strand is coding sequence) as target query DNA sequence
2. The tobacco genome sequence assembly of Example 1 as DNA database to be searched
3. A fixed 12 basepair size first substring DNA motif
4. A fixed 0 basepair size spacer
5. A fixed 12 basepair size basepair second substring DNA motif
6. A maximum threshold number of 1 occurrence in the chosen DNA database

### Program Output:

All 24 basepair sequences for a 12-0-12 design for exon 2, wherein the first number represents the fixed length of the first substring, the second number the fixed length of the spacer, and the third number the fixed length of the second substring with the above input settings, that were generated by the program with a threshold of maximum 1 occurrence in the tobacco genome database are:

TTTTCATTTCAG TGGATTGAGGAG (SEQ ID NO: 63): 0 hits
TTTCATTTCAGT GGATTGAGGAGC (SEQ ID NO: 64): 0 hits
TTCATTTCAGTG GATTGAGGAGCC (SEQ ID NO: 65): 0 hits
TCATTTCAGTGG ATTGAGGAGCCG (SEQ ID NO: 66): 0 hits
CATTTCAGTGGA TTGAGGAGCCGT (SEQ ID NO: 67): 0 hits
ATTTCAGTGGAT TGAGGAGCCGTC (SEQ ID NO: 68): 0 hits
TTTCAGTGGATT GAGGAGCCGTCA (SEQ ID NO: 69): 0 hits
TTCAGTGGATTG AGGAGCCGTCAC (SEQ ID NO: 70): 0 hits
TCAGTGGATTGA GGAGCCGTCACT (SEQ ID NO: 71): 0 hits
CAGTGGATTGAG GAGCCGTCACTT (SEQ ID NO: 72): 0 hits
AGTGGATTGAGG AGCCGTCACTTT (SEQ ID NO: 73): 0 hits
GTGGATTGAGGA GCCGTCACTTTT (SEQ ID NO: 74): 0 hits
TGGATTGAGGAG CCGTCACTTTTG (SEQ ID NO: 75):.0 hits
GGATTGAGGAGC CGTCACTTTTGA (SEQ ID NO: 76): 0 hits
GATTGAGGAGCC GTCACTTTTGAT (SEQ ID NO: 77): 0 hits
ATTGAGGAGCCG TCACTTTTGATT (SEQ ID NO: 78): 0 hits
TTGAGGAGCCGT CACTTTTGATTA (SEQ ID NO: 79): 0 hits
TGAGGAGCCGTC ACTTTTGATTAC (SEQ ID NO: 80): 0 hits
GAGGAGCCGTCA CTTTTGATTACA (SEQ ID NO: 81): 0 hits
AGGAGCCGTCAC TTTTGATTACAC (SEQ ID NO: 82): 0 hits
GGAGCCGTCACT TTTGATTACACG (SEQ ID NO: 83): 0 hits
GAGCCGTCACTT TTGATTACACGA (SEQ ID NO: 84): 0 hits
AGCCGTCACTTT TGATTACACGAT (SEQ ID NO: 85): 0 hits
GCCGTCACTTTT GATTACACGATT (SEQ ID NO: 86): 0 hits
CCGTCACTTTTG ATTACACGATTT (SEQ ID NO: 87): 0 hits
CGTCACTTTTGA TTACACGATTTG (SEQ ID NO: 88): 0 hits
GTCACTTTTGAT TACACGATTTGA (SEQ ID NO: 89): 0 hits
TCACTTTTGATT ACACGATTTGAG (SEQ ID NO: 90): 0 hits
CACTTTTGATTA CACGATTTGAGT (SEQ ID NO: 91): 0 hits
ACTTTTGATTAC ACGATTTGAGTA (SEQ ID NO: 92): 0 hits
CTTTTGATTACA CGATTTGAGTAT (SEQ ID NO: 93): 0 hits
TTTTGATTACAC GATTTGAGTATG (SEQ ID NO: 94): 0 hits
TTTGATTACACG ATTTGAGTATGC (SEQ ID NO: 95): 0 hits
TTGATTACACGA TTTGAGTATGCA (SEQ ID NO: 96): 0 hits
TGATTACACGAT TTGAGTATGCAA (SEQ ID NO: 97): 0 hits
GATTACACGATT TGAGTATGCAAA (SEQ ID NO: 98): 0 hits
ATTACACGATTT GAGTATGCAAAC (SEQ ID NO: 99): 0 hits
TTACACGATTTG AGTATGCAAACC (SEQ ID NO: 100): 0 hits
TACACGATTTGA GTATGCAAACCT (SEQ ID NO: 101): 0 hits
ACACGATTTGAG TATGCAAACCTT (SEQ ID NO: 102): 0 hits
CACGATTTGAGT ATGCAAACCTTT (SEQ ID NO: 103): 0 hits
ACGATTTGAGTA TGCAAACCTTTT (SEQ ID NO: 104): 0 hits
CGATTTGAGTAT GCAAACCTTTTC (SEQ ID NO: 105): 0 hits
GATTTGAGTATG CAAACCTTTTCC (SEQ ID NO: 106): 0 hits
ATTTGAGTATGC AAACCTTTTCCA (SEQ ID NO: 107): 0 hits
TTTGAGTATGCA AACCTTTTCCAC (SEQ ID NO: 108): 0 hits
TTGAGTATGCAA ACCTTTTCCACA (SEQ ID NO: 109): 0 hits
TGAGTATGCAAA CCTTTTCCACAC (SEQ ID NO: 110): 0 hits
GAGTATGCAAAC CTTTTCCACACA (SEQ ID NO: 111): 0 hits
AGTATGCAAACC TTTTCCACACAG (SEQ ID NO: 112): 0 hits
GTATGCAAACCT TTTCCACACAGT (SEQ ID NO: 113): 0 hits
TATGCAAACCTT TTCCACACAGTT (SEQ ID NO: 114): 0 hits
ATGCAAACCTTT TCCACACAGTTA (SEQ ID NO: 115): 0 hits
TGCAAACCTTTT CCACACAGTTAC (SEQ ID NO: 116): 0 hits
GCAAACCTTTTC CACACAGTTACC (SEQ ID NO: 117): 0 hits
CAAACCTTTTCC ACACAGTTACCG (SEQ ID NO: 118): 0 hits
AAACCTTTTCCA CACAGTTACCGA (SEQ ID NO: 119): 0 hits
AACCTTTTCCAC ACAGTTACCGAT (SEQ ID NO: 120): 0 hits
ACCTTTTCCACA CAGTTACCGATT (SEQ ID NO: 121): 0 hits
CCTTTTCCACAC AGTTACCGATTG (SEQ ID NO: 122): 0 hits
CTTTTCCACACA GTTACCGATTGG (SEQ ID NO: 123): 0 hits
TTTTCCACACAG TTACCGATTGGT (SEQ ID NO: 124): 0 hits
TTTCCACACAGT TACCGATTGGTA (SEQ ID NO: 125): 0 hits
TTCCACACAGTT ACCGATTGGTAT (SEQ ID NO: 126): 0 hits
TCCACACAGTTA CCGATTGGTATA (SEQ ID NO: 127): 0 hits
CCACACAGTTAC CGATTGGTATAG (SEQ ID NO: 128): 0 hits
CACACAGTTACC GATTGGTATAGT (SEQ ID NO: 129): 0 hits
ACACAGTTACCG ATTGGTATAGTG (SEQ ID NO: 130): 0 hits
CACAGTTACCGA TTGGTATAGTGC (SEQ ID NO: 131): 0 hits
ACAGTTACCGAT TGGTATAGTGCA (SEQ ID NO: 132): 0 hits
CAGTTACCGATT GGTATAGTGCAT (SEQ ID NO: 133): 0 hits
AGTTACCGATTG GTATAGTGCATA (SEQ ID NO: 134): 0 hits
GTTACCGATTGG TATAGTGCATAC (SEQ ID NO: 135): 0 hits
TTACCGATTGGT ATAGTGCATACG (SEQ ID NO: 136): 0 hits
TACCGATTGGTA TAGTGCATACGT (SEQ ID NO: 137): 0 hits
ACCGATTGGTAT AGTGCATACGTG (SEQ ID NO: 138): 0 hits
CCGATTGGTATA GTGCATACGTGG (SEQ ID NO: 139): 0 hits
CGATTGGTATAG TGCATACGTGGC (SEQ ID NO: 140): 0 hits
GATTGGTATAGT GCATACGTGGCA (SEQ ID NO: 141): 0 hits
ATTGGTATAGTG CATACGTGGCAT (SEQ ID NO: 142): 0 hits
TTGGTATAGTGC ATACGTGGCATC (SEQ ID NO: 143): 0 hits
TGGTATAGTGCA TACGTGGCATCC (SEQ ID NO: 144): 0 hits
GGTATAGTGCAT ACGTGGCATCCA (SEQ ID NO: 145): 0 hits
GTATAGTGCATA CGTGGCATCCAG (SEQ ID NO: 146): 0 hits
TATAGTGCATAC GTGGCATCCAGG (SEQ ID NO: 147): 0 hits
ATAGTGCATACG TGGCATCCAGGG (SEQ ID NO: 148): 0 hits
TAGTGCATACGT GGCATCCAGGGT (SEQ ID NO: 149): 0 hits
AGTGCATACGTG GCATCCAGGGTT (SEQ ID NO: 150): 0 hits
GTGCATACGTGG CATCCAGGGTTA (SEQ ID NO: 151): 0 hits
TGCATACGTGGC ATCCAGGGTTAC (SEQ ID NO: 152): 0 hits
GCATACGTGGCA TCCAGGGTTACT (SEQ ID NO: 153): 0 hits
CATACGTGGCAT CCAGGGTTACTG (SEQ ID NO: 154): 0 hits
ATACGTGGCATC CAGGGTTACTGG (SEQ ID NO: 155): 0 hits
TACGTGGCATCC AGGGTTACTGGC (SEQ ID NO: 156): 0 hits
ACGTGGCATCCA GGGTTACTGGCT (SEQ ID NO: 157): 0 hits
CGTGGCATCCAG GGTTACTGGCTT (SEQ ID NO: 158): 0 hits
GTGGCATCCAGG GTTACTGGCTTG (SEQ ID NO: 159): 0 hits
TGGCATCCAGGG TTACTGGCTTGC (SEQ ID NO: 160): 0 hits
GGCATCCAGGGT TACTGGCTTGCC (SEQ ID NO: 161): 0 hits
GCATCCAGGGTT ACTGGCTTGCCC (SEQ ID NO: 162): 0 hits
CATCCAGGGTTA CTGGCTTGCCCA (SEQ ID NO: 163): 0 hits
ATCCAGGGTTAC TGGCTTGCCCAG (SEQ ID NO: 164): 0 hits
TCCAGGGTTACT GGCTTGCCCAGT (SEQ ID NO: 165): 0 hits
CCAGGGTTACTG GCTTGCCCAGTC (SEQ ID NO: 166): 0 hits
CAGGGTTACTGG CTTGCCCAGTCG (SEQ ID NO: 167): 0 hits
AGGGTTACTGGC TTGCCCAGTCGG (SEQ ID NO: 168): 0 hits
GGGTTACTGGCT TGCCCAGTCGGC (SEQ ID NO: 169): 0 hits
GGTTACTGGCTT GCCCAGTCGGCC (SEQ ID NO: 170): 0 hits
GTTACTGGCTTG CCCAGTCGGCCA (SEQ ID NO: 171): 0 hits
TTACTGGCTTGC CCAGTCGGCCAC (SEQ ID NO: 172): 0 hits
TACTGGCTTGCC CAGTCGGCCACA (SEQ ID NO: 173): 0 hits
ACTGGCTTGCCC AGTCGGCCACAT (SEQ ID NO: 174): 0 hits
CTGGCTTGCCCA GTCGGCCACATT (SEQ ID NO: 175): 0 hits
TGGCTTGCCCAG TCGGCCACATTT (SEQ ID NO: 176): 0 hits
GGCTTGCCCAGT CGGCCACATTTG (SEQ ID NO: 177): 0 hits
GCTTGCCCAGTC GGCCACATTTGG (SEQ ID NO: 178): 0 hits
CTTGCCCAGTCG GCCACATTTGGT (SEQ ID NO: 179): 0 hits
TTGCCCAGTCGG CCACATTTGGTT (SEQ ID NO: 180): 0 hits
TGCCCAGTCGGC CACATTTGGTTT (SEQ ID NO: 181): 0 hits
GCCCAGTCGGCC ACATTTGGTTTT (SEQ ID NO: 182): 0 hits
CCCAGTCGGCCA CATTTGGTTTTT (SEQ ID NO: 183): 0 hits
CCAGTCGGCCAC ATTTGGTTTTTG (SEQ ID NO: 184): 0 hits
CAGTCGGCCACA TTTGGTTTTTGT (SEQ ID NO: 185): 0 hits
AGTCGGCCACAT TTGGTTTTTGTA (SEQ ID NO: 186): 0 hits
GTCGGCCACATT TGGTTTTTGTAG (SEQ ID NO: 187): 0 hits
TCGGCCACATTT GGTTTTTGTAGA (SEQ ID NO: 188): 0 hits
CGGCCACATTTG GTTTTTGTAGAT (SEQ ID NO: 189): 0 hits
GGCCACATTTGG TTTTTGTAGATG (SEQ ID NO: 190): 0 hits
GCCACATTTGGT TTTTGTAGATGG (SEQ ID NO: 191): 0 hits
CCACATTTGGTT TTTGTAGATGGC (SEQ ID NO: 192): 0 hits
CACATTTGGTTT TTGTAGATGGCC (SEQ ID NO: 193): 0 hits
ACATTTGGTTTT TGTAGATGGCCA (SEQ ID NO: 194): 0 hits
CATTTGGTTTTT GTAGATGGCCAT (SEQ ID NO: 195): 0 hits
ATTTGGTTTTTG TAGATGGCCATT (SEQ ID NO: 196): 0 hits
TTTGGTTTTTGT AGATGGCCATTG (SEQ ID NO: 197): 0 hits
TTGGTTTTTGTA GATGGCCATTGT (SEQ ID NO: 198): 0 hits
TGGTTTTTGTAG ATGGCCATTGTG (SEQ ID NO: 199): 0 hits
GGTTTTTGTAGA TGGCCATTGTGA (SEQ ID NO: 200): 0 hits
GTTTTTGTAGAT GGCCATTGTGAG (SEQ ID NO: 201): 0 hits
TTTTTGTAGATG GCCATTGTGAGG (SEQ ID NO: 202): 0 hits
TTTTGTAGATGG CCATTGTGAGGT (SEQ ID NO: 203): 0 hits
TTTGTAGATGGC CATTGTGAGGTA (SEQ ID NO: 204): 0 hits
TTGTAGATGGCC ATTGTGAGGTAT (SEQ ID NO: 205): 0 hits
TGTAGATGGCCA TTGTGAGGTATG (SEQ ID NO: 206): 0 hits
GTAGATGGCCAT TGTGAGGTATGT (SEQ ID NO: 207): 0 hits
TAGATGGCCATT GTGAGGTATGTT (SEQ ID NO: 208): 0 hits
AGATGGCCATTG TGAGGTATGTTT (SEQ ID NO: 209): 0 hits
GATGGCCATTGT GAGGTATGTTTG (SEQ ID NO: 210): 0 hits
ATGGCCATTGTG AGGTATGTTTGA (SEQ ID NO: 211): 0 hits

A smallest number of hits=0 means that the sequence does not occur in the tobacco genome database of Example 1. For the design of a unique DNA binding domain the threshold is set at 1 provided that the search sequence is present in the DNA database. If the search sequence is not in the DNA database, the threshold is set at 0. To those skilled in the art it is clear that if there are multiple loci with high sequence identity, setting the threshold at 2, 3 or higher generates outputs suitable for the generation of zinc finger nucleases for the target glycosyltransferase.

Similar scores tables can be constructed for any other combination of fixed length substring DNA motifs, threshold setting and fixed length of spacer.

### Development of a Pair of Zinc Finger DNA Binding Domains.

To those skilled in the art it is clear that mutagenesis of the coding sequence can directly affect the ability of the cell to produce a functional protein. The output sequences can be aligned to the part of the DNA sequence of SEQ ID NO: 5 that codes directly for the beta-1,2-xylosyltransferase (β(1,2)-xylosyltransferase) variant 1 protein of SEQ ID NO: 8. To those skilled in the art it is clear that mutagenesis of an exon-intron boundary can also lead to the inability of the pre-mRNA to correctly process into mRNA potentially disrupting enzyme activity. To this end, the output sequences mapping to both ends of exon 2 are aligned to the non-coding part of SEQ ID NO: 5. Next, the two substrings are separated and one of the two substring DNA sequences are complemented and inversed. For example for the program output TCCACACAGTTA CCGATTGGTATA (SEQ ID NO: 127), one zinc finger protein binds TCCACACAGTTA and the other finally making up a pair of zinc finger nucleases for targeting the respective nucleotide sequence SEQ ID NO: 127 is TATACCAATCGG. Next, these zinc finger protein targeting sequences are divided in subsets of three basepairs, each subset of which is targeted by a zinc finger DNA binding domain. For TCCACACAGTTA this is TCC-ACA-CAG-TTA and for TATACCAATCGG this is TAT-ACC-AAT-CGG. Zinc finger DNA binding domains are known as well as methods for engineering zinc finger nucleases by modular design (see Wright et al., 2006). Zinc finger plasmids comprising a zinc finger DNA binding domain for a given 3 basepair sequence are known, for example see catalog of Addgene Inc. 1 kendall Square, Cambridge, Mass., USA. A zinc finger DNA binding domain for ACA nucleotide sequence can be, for example, PGEKPYKCPECGKSFSSPADLTRHQRTH and a zinc finger DNA binding domain that can recognize and bind a AAT nucleotide sequence can be, for example, PGEKPYKCPECGKSFSTTGNLTVHQRTH.

### Example 7

### Targeted Mutagenesis of a Beta-1,2-Xylosyltransferase (β(1,2)-Xylosyltransferase) Gene in Tobacco Using Zinc Finger Nucleases

Development of zinc finger nuclease expression cassettes. For the mutagenesis of the beta-1,2-xylosyltransferase (β(1,2)-xylosyltransferase) variant 1 gene of SEQ ID NO: 5 in tobacco, a pair of zinc finger DNA binding domains specific for exon 2 and each binding a 12 bp sequence of SEQ ID NO: 5, is selected as described in Example 6. Synthetic gene sequences coding for said pair of zinc finger DNA binding domains fused to the catalytic domain of FokI restriction endonuclease, are constructed such that optimal expression in a tobacco cell can be obtained by matching codon bias. First, the zinc finger nuclease comprising the zinc finger DNA binding domain of the first target sequence of the beta-1,2-xylosyltransferase (β(1,2)-xylosyltransferase) variant 1 gene, and the zinc finger nuclease comprising the zinc finger DNA binding domain of the second target sequence of the beta-1,2-xylosyltransferase (β(1,2)-xylosyltransferase) variant 1 gene are cloned downstream of a cauliflower mosaic virus (CaMV) 35S promoter and upstream of a CaMV35S terminator sequence following standard cloning methods. The gene expression cassettes are then cloned in a pBINPLUS-derived binary vector generating a plant expression cassette. Synthetic gene sequences can be made by PCR using 3′-overlapping synthetic oligonucleotides or by ligating fragments comprising phosphorylated complementary oligonucleotides following standard methods described in the art. In this configuration, the codon bias is optimized for expression in tobacco cells. In other configurations, the codon bias can be non optimized. In this configuration, the zinc finger nuclease genes are cloned under control of a cauliflower 35S promoter and terminator sequence. In other configurations, the genes can be cloned under control of a cowpea mosaic virus promoter, a nopaline synthase promoter, a plastocyanin promoter of alfalfa, or any other promoter active in a tobacco plant cell and a nopaline synthase terminator sequence, a plastocyanin terminator sequence or any other sequence that functions as a transcription terminator in a tobacco plant cell. Both genes can be cloned in one binary vector or separately. In this configuration, the expression cassettes are cloned in a pBINPLUS binary vector. In other configurations, the cassettes can be cloned in a pBIN19 vector or any other binary vector. In yet another configuration, the expression cassettes can be cloned in a vector that is introduced into a tobacco cell by particle bombardment or a plant viral expression vector.

### Transfection of Tobacco Cells.

The vector comprising both zinc finger nuclease expression cassettes is introduced in Agrobacterium tumefaciens strain LBA4404(pAL4404) using standard methods described in the art. The recombinant Agrobacterium tumefaciens strain is grown overnight in liquid broth containing appropriate antibiotics and cells are collected by centrifugation, decanted and resuspended in fresh medium according to Murashige & Skoog (1962) containing 20 g/L sucrose and adjusted to 10D595. Leaf explants of aseptically grown tobacco plants are transformed according to standard methods (see Horsh et al., 1985) and co-cultivated for two days on medium according to Murashige & Skoog (1962) supplemented with 20 g/L sucrose and 7 g/L purified agar in a petri dish under appropriate conditions as described in the art. After two days of co-cultivation, explants are placed on selective medium containing kanamycin for selection and 200 mg/L vancomycin and 200 mg/L cefotaxim, 1 g/L NAA and 0.1 g/L BAP hormones. In this example the binary vector is introduced in LBA4404(pAL4404). In other experiments, the binary vector can be introduced into Agrobacterium tumefaciens strain AgI0, AgI1, GV3101 or any other ACH5 or C58 derived Agrobacterium tumefaciens strain suitable for the transformation of tobacco leaf explants. In this example, leaf explants are transfected. In other experiments, explants can be seedlings, hypocotyls or stem tissue or any other tissue amenable to transformation. In this example, a binary vector is introduced via transfection with an Agrobacterium tumefaciens strain comprising the expression cassette. In other experiments, an expression cassette can be introduced using particle bombardment.

Regeneration of Tobacco Plants after Transfection of Tobacco Cells and Analysis.

Transgenic tobacco cells are regenerated into shoots and plantlets according to standard methods described in the art (see for example Horsch et al., 1985). Genomic DNA is isolated from shoots or plantlets for example by using the PowerPlant DNA isolation kit (Mo Bio Laboratories Inc., Carlsbad, Calif., USA). DNA fragments comprising the targeted region are amplified according to standard methods described in the art using the gene sequence of SEQ ID NO:4. To those skilled in the art it is clear that for example the pair of SEQ ID NO:2 and SEQ ID NO:3 can be used to amplify the fragment comprising the targeted region. PCR products are sequenced in their entirety using standard sequencing protocols and mutations and/or modifications at or around the zinc finger nuclease target site are identified by comparison with the original sequence of SEQ ID NO:4.

### Characterisation of Mutation.

In this instance, the coding region of a beta-1,2-xylosyltransferase (β(1,2)-xylosyltransferase) is targeted and the effect of any observed mutation is done by comparison of the predicted translation product of the mutant sequence with the original cDNA sequence of SEQ ID NO:8 and predicted amino acid sequence thereof of SEQ ID NO:9. To those skilled in the art it is clear that any deletion that results in the disruption of the open reading frame of the respective sequence, can have a deleterious effect on the synthesis of a functional protein. Plants with mutant beta-1,2-xylosyltransferase (β(1,2)-xylosyltransferase) gene sequences resulting in predicted disruption of the open reading frame are submitted to a beta-1,2-xylosyltransferase (β(1,2)-xylosyltransferase) enzyme activity assay and the measured enzyme activity is compared to that of the original plant without mutation.

### Beta-1,2-Xylosyltransferase (β(1,2)-Xylosyltransferase) Activity Assay.

Microsomes are isolated from fresh leaves of mature, full-grown plants at the stage of early flowering as follows: remove the midvein, cut leaves into small pieces and homogenize in a precooled stainless-steel Waring blender in microsome isolation buffer (250 mM sorbitol, 5 mM Tris, 2 mM DTT and 7.5 mM EDTA; set at pH 7.8 by using a 1 M solution of Mes (2-(N-morpholino)ethanesulfonic acid. Add a protease inhibitor cocktail (Complete Mini, Roche Diagnostics) and use 3 ml of ice-cold microsome isolation buffer per g of fresh-weight tobacco leaves. Filter through 88 μm nylon cloth and remove debris and leaf material by centrifugation for 10 min at 12,000 g at 4° C. using a Sorvall SS34 rotor. Transfer supernatant containing microsomes to new centrifugation tube and centrifuge in a fixed-angle Centrikon TFT 55.38 rotor for 60 min at 100,000 g at 4° C. in a Centricon T-2070 ultracentrifuge. Resuspend the pellet containing the microsomes in microsome isolation buffer without EDTA and to which glycerol (4% final concentration) has been added. Xylosyltransferase enzyme activity is measured in a 25 μL reaction mixture containing 10 mM cacodylate buffer (pH 7.2), 4 mM ATP, 20 mM MnCl₂, 0.4% Triton X-100, 0.1 mM UDP-[¹⁴C]-xylose and 1 mM GlcNAcβ-1-2-Man-α1-3-[Man-α1-6]Man-β-O—(CH₂)₈—COOH₃ using GlcNAcβ-1-2-Man-α1-3-(GlcNAc-β1-2-Man-α1-6)Man-β1-4GlcNAc-β1-4(Fuc-α1-6)GlcNAc-IgG glycopeptide as an acceptor.

### Example 8

### Targeted Mutagenesis of a Beta-1,2-Xylosyltransferase (β(1,2)-Xylosyltransferase) Gene in Tobacco Using a Single Chain Meganuclease

Engineering of I-CreI Derivatives Cleaving Exon 2 of Tobacco Beta-1,2-Xylosyltransferase (β(1,2)-xylosyltransferase) Variant 1.

For the mutagenesis of exon 2 of the beta-1,2-xylosyltransferase (β(1,2)-xylosyltransferase) variant 1 gene of SEQ ID NO: 5 in tobacco, first a unique 22 bp targeting sequence within exon 2 is selected. This can be done using the search protocol of Example 4 with a fixed 0 basepair size for the spacer and a total of 22 bp for first and second substring DNA motif. However, in this instance, a unique 22 bp sequence is chosen using the outcome of Example 6 and discarding the last 2 bp of the outcome sequence SEQ ID NO: 64 resulting in the following sequence TTTTCATTTCAGTGGATTGAGG. Two derivative targets are designed representing the left and right halves of SEQ ID NO: 42 in palindromic form. SEQ ID NO: 43 (TTTTCATTTCATGAAATGAAAA) represents the left half and SEQ ID NO: 44 (CCTCAATCCTCGTGGATTGAGG) represents the right half. A combinatorial I-CreI mutant library is screened for mutant endonucleases with new specificity towards these two palindromic derivative target sequences (SEQ ID NO: 43; SEQ ID NO: 44) as described by Smith et al. (2006, Nucleic Acid Res. 34:e149). In this instance a single chain meganuclease is developed for target sequence SEQ ID NO: 42. In other instances, obligate heterodimer meganucleases can be developed by those skilled in the art. In this instance, the I-CreI dimeric meganuclease is used as a scaffold for the development of 22 bp specific mutant endonucleases to target SEQ ID NO: 42. In other instances, other scaffolds can be used to develop mutant endonucleases that target a subsequence in exon 2, such as but not limited to I-HmuI, I-HmuII, I-Bast, 1-TevIII, I-CmoeI, I-PpoI, I-SspI, I-SceI, I-CeuI, I-MsoI, I-DmoI, H-DreI, PI-SceI or PI-PfuI.

### Development of Single Chain Meganuclease Expression Cassette.

Functional mutant endonucleases with specificity for SEQ ID NO: 43 and 44 are used to design a single chain meganuclease with specificity to SEQ ID NO: 42, essentially as described by Grizot et al. (2009). The C-terminal part of the first endonuclease SEQ ID NO: 43 targeting the left part of SEQ ID NO: 42 is connected to the N-terminal part of the second endonuclease SEQ ID NO: 44, targeting the right half of SEQ ID NO: 42 with a series of linkers differing in length and sequence and the activity of the proteins is assessed. Functional proteins are used to design a gene construct for expression in tobacco, transfection of tobacco cells and screening for mutant sequences and tobacco plants with modified beta-1,2-xylosyltransferase (β(1,2)-xylosyltransferase) activity, essentially as described in Example 7.

### Example 9

### Combining Mutant Loci by Crossing of Modified Tobacco Plants

Tobacco plants are grown under greenhouse conditions. Mutant loci present in different modified tobacco plants, are combined by crossing. For crossing, tobacco flowers are emasculated at stage 6-10 of flower development before pollen shed (Koltunow et al., 1990, The Plant Cell 2: 1201-1224). Pistils of emasculated flowers of acceptor plants are pollinated at the stage of development resembling anthesis with donor pollen and pollinated flowers are individually enveloped to prevent from cross pollination. Crossings are made in both directions with parent 1 as donor and acceptor, and parent 2 as acceptor and donor, respectively, to avoid potential fertility problems. Seeds are collected and offspring plants are analysed for mutations by sequencing and enzyme activity, as described in Example 7. Plants with combined mutations are grown to maturity, selfed and offspring plants are analysed by sequencing and for enzyme activity, as before. Plants with combined mutations are selected, selfed and their offspring is analysed for homozygosity. Homozygous plants are selected. To those skilled in the art it is clear that by crossing one can combine mutant loci for beta-1,2-xylosyltransferase (β(1,2)-xylosyltransferase) gene sequences present in different modified tobacco plants, or combine mutant loci for alpha-1,3-fucosyltransferase (α(1,3)-fucosyltransferase) gene sequences present in different plants, or mutant loci for beta-1,2-xylosyltransferase (β(1,2)-xylosyltransferase) gene sequences and alpha-1,3-fucosyltransferase (α(1,3)-fucosyltransferase) gene sequences such that tobacco plants are generated that have no beta-1,2-xylosyltransferase (β(1,2)-xylosyltransferase) enzyme activity, no alpha-1,3-fucosyltransferase (α(1,3)-fucosyltransferase) enzyme activity or no beta-1,2-xylosyltransferase (β(1,2)-xylosyltransferase) and no alpha-1,3-fucosyltransferase (α(1,3)-fucosyltransferase) enzyme activity.

### Example 10

### Identification of Nicotiana tabacum and Nicotiana benthamiana N-Acetylglucosaminyltransferase I Genome Sequences

This example illustrates how genomic nucleotide sequences of a N-acetylglucosaminyltransferase I are identified using PCR.

High-molecular weight DNA is isolated from the nuclei of Nicotiana benthamiana and Nicotiana tabacum according to standard protocols. Primer set are developed to amplify an approximately 3100 bp (GnTI-A) and 3500 bp (GnTI-B) fragment based on known N-acetylglucosaminyltransferase I sequences. Primer set used are SEQ ID NO: 23: primer sequence Big1FN and primer sequence SEQ ID NO: 24: Big1RN for the amplification of fragment GnTI-A and primer set SEQ ID NO: 10: primer sequence Big3FN and SEQ ID NO: 11: primer sequence Big3RN for the amplification of a fragment GnTI-B. PCR is carried out on the high molecular weight genomic DNA using standard protocols. Fragment GnTI-A of Nicotiana tabacum and fragment GnTI-B of Nicotiana tabacum and Nicotiana benthamiana are sequenced according to standard protocols. No nucleotide sequence fragment is amplified corresponding to fragment GnTI-A using high-molecular weight DNA of Nicotiana benthamiana.

SEQ ID NO: 40 discloses a 3152 bp nucleotide sequence corresponding to the genomic fragment of Nicotiana tabacum fragment GnTI-A.

SEQ ID NO: 41 discloses a 3140 bp nucleotide sequence corresponding to the genomic fragment of Nicotiana tabacum fragment GnTI-A.

SEQ ID NO: 212 discloses a partial cDNA sequence variant 1 of Nicotiana tabacum fragment GnTI-A (SEQ ID NO: 40) and SEQ ID NO: 227, a partial cDNA sequence variant 2 as predicted by FgeneSH.

SEQ ID NO: 213 and SEQ ID NO: 229, disclose partial cDNA sequences variant 1 and 2 of Nicotiana tabacum GnTI-A (SEQ ID NO: 41) as predicted by FgeneSH.

SEQ ID NO: 217 and SEQ ID NO: 228, disclose the predicted partial amino acid sequences of Nicotiana tabacum fragment GnTI-A cDNA variant 1 (SEQ ID NO: 213) and variant 2 (SEQ ID NO: 229).

SEQ ID NO: 218 and SEQ ID NO: 230, disclose the predicted partial amino acid sequences of Nicotiana tabacum fragment GnTI-A cDNA variant 1 (SEQ ID NO: 213) and variant 2 (SEQ ID NO: 229).

SEQ ID NO: 12 discloses a 3504 bp nucleotide sequence corresponding to the genomic fragment of Nicotiana tabacum fragment GnTI-B.

SEQ ID NO: 13 discloses a 2283 bp nucleotide sequence corresponding to the genomic fragment of Nicotiana tabacum fragment GnTI-B.

SEQ ID NO: 14 discloses a 3765 bp nucleotide sequence corresponding to the genomic fragment of Nicotiana benthamiana fragment GnTI-B.

SEQ ID NO: 20 discloses a partial cDNA sequence variant 1 of Nicotiana tabacum fragment GnTI-B (SEQ ID NO: 12), and SEQ ID NO: 219, a partial cDNA sequence variant 2, and SEQ ID NO: 220, a partial cDNA sequence variant 3 of Nicotiana tabacum fragment GnTI-B (SEQ ID NO: 12), as predicted by FgeneSH.

SEQ ID NO: 214 and SEQ ID NO: 221 and SEQ ID NO: 222, disclose the predicted partial amino acid sequences of Nicotiana tabacum fragment GnTI-B cDNA variant 1 (SEQ ID NO: 20), variant 2 (SEQ ID NO: 219) and variant 3 (SEQ ID NO: 220), respectively.

SEQ ID NO: 21 discloses a partial cDNA sequence variant 1 of Nicotiana tabacum fragment GnTI-B (SEQ ID NO: 13), and SEQ ID NO: 223, a partial cDNA sequence variant 2 as predicted by FgeneSH.

SEQ ID NO: 215 and SEQ ID NO: 224 disclose the predicted partial amino acid sequences of Nicotiana tabacum fragment GnTI-B cDNA variant 1 (SEQ ID NO: 21) and variant 2 (SEQ ID NO: 223), respectively.

SEQ ID NO: 22 discloses a partial cDNA sequence variant 1 of Nicotiana benthamiana fragment GnTI-B (SEQ ID NO: 14), and SEQ ID NO: 225, a partial cDNA sequence variant 2 as predicted by FgeneSH.

SEQ ID NO: 216 and SEQ ID NO: 226 disclose the predicted partial amino acid sequences of Nicotiana benthamiana fragment GnTI-B cDNA variant 1 (SEQ ID NO: 22) and variant 2 (SEQ ID NO: 225), respectively.

### Example 11

### Identification of Nicotiana tabacum N-Acetylglucosaminyltransferase I (GnTI) Variant 2

Using primer pair NGSG12045 (SEQ ID NO: 231 and 232) based on contig gDNA_c1690982, the genomic nucleotide sequence of N-acetylglucosaminyltransferase I gene variant 2 of Nicotiana tabacum is identified by the method as described in Example 1. SEQ ID NO: 233 represents 15,000 basepairs of the genomic nucleotide sequence of the BAC clone, BAC-FABIJI_{—}1, that contains a Nicotiana tabacum N-acetylglucosaminyltransferase I gene variant 2. The locations of introns and exons in SEQ ID NO: 233 are predicted using FgeneSH and Augustus, and SEQ ID NO: 234 provides a predicted cDNA sequence of the Nicotiana tabacum N-acetylglucosaminyltransferase I gene variant 2. SEQ ID NO: 235 represents the single letter amino acid sequence of the N-acetylglucosaminyltransferase I gene variant 2 of the cDNA sequence as set forth in SEQ ID NO: 234.

### Example 12

### Identification of N-Acetylglucosaminyltransferase I Sequences of Nicotiana tabacum PM132

In Examples 10 and 11, several N-acetylglucosaminyltransferase I gene sequences of N. tabacum are identified. SEQ ID NO:12 discloses the nucleotide sequence of a 3504 bp genomic region comprising a part of a GnTI gene of N. tabacum PM132. SEQ ID NO:40 discloses a nucleotide sequence of a 3152 bp genomic region comprising a part of a GnTI gene of N. tabacum PM132. SEQ ID NO:13 discloses a nucleotide sequence of a 2283 bp genomic region comprising a part of a GnTI gene of N. tabacum PO2. SEQ ID NO:41 discloses a nucleotide sequence of a 3140 bp genomic region comprising a part of a GnTI gene of N. tabacum PO2. SEQ ID NO:233 discloses a 15,000 bp genomic nucleotide sequence comprising the entire coding region of a GnTI (“FABIJI”) of N. tabacum Hicks Broadleaf with 5′ and 3′ UTR's.

As described above, the only GnTI gene sequence encoding an entire GnTI is that obtained from N. tabacum Hicks Broadleaf (SEQ ID NO:233). PM132 is one of a preferred variety of Nicotiana tabacum for use in the methods of the invention. The seeds of PM132 were deposited on 6 Jan. 2011 at NCIMB Ltd. (an International Depositary Authority under the Budapest Treaty, located at Ferguson Building, Craibstone Estate, Bucksburn, Aberdeen, AB21 9YA, United Kingdom) under accession number NCIMB 41802. The following paragraphs describe the cloning of full length GnTI sequences of N. tabacum PM132.

### FABIJI Homolog.

The genomic sequences comprising the entire gene of FABIJI homolog in N. tabacum PM132 are identified using primers SEQ ID NO:236, SEQ ID NO:237, SEQ ID NO:242, SEQ ID NO:243, SEQ ID NO:244 and SEQ ID NO:245. SEQ ID NO:256 discloses the nucleotide sequence of a genomic region in N. tabacum PM132 which comprises the coding sequence of FABIJI homolog. SEQ ID NO:257 discloses the nucleotide sequence of the coding region of the FABIJI homolog of N. tabacum PM132. SEQ ID NO:258 sets forth the predicted amino acid sequence of the FABIJI homolog of N. tabacum PM132.

### CAC80702.1 Homolog.

EMBL-CDS: CAC80702.1, accession number AJ249883.1, discloses a cDNA sequence of a GnTI obtained from N. tabacum Samsun NN. A homolog of CAC80702.1 in N. tabacum PM132 is cloned by using primer sequences SEQ ID NO:240 and SEQ ID NO:241. Additional sequences are cloned as shown herein below using primer sequences SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254 and SEQ ID NO:255.

SEQ ID NO:262 discloses the nucleotide sequence of a genomic region of N. tabacum PM132 that encodes a homolog of CAC80702.1. SEQ ID NO:263 discloses the nucleotide sequence of the coding region of the CAC80702.1 homolog of N. tabacum PM132. SEQ ID NO:264 discloses the predicted amino acid sequence of the CAC80702.1 homolog of N. tabacum PM132.

### GnTI Pseudogene CPO.

Primers having sequences of SEQ ID NO:238 and SEQ ID NO:239. are used in PCR amplification to identify a genomic sequence of N. tabacum PM132 that comprises the fragments GnTI-A and GnTI-B as described in Example 10. SEQ ID NO:259 discloses the nucleotide sequence of a GnTI-like gene in N. tabacum PM132, now referred to as CPO. SEQ ID NO:260 discloses the predicted coding region of the N. tabacum PM132 CPO gene. SEQ ID NO:261 discloses the predicted amino acid sequence of the N. tabacum PM132 CPO gene. A stop codon is identified in the CPO coding sequence (SEQ ID NO: 259) which corresponds to the C-terminal part of a GnTI, suggesting that CPO is a pseudogene. This suggestion is supported by the lack of cDNA clones encoding CPO, that is prepared from N. tabacum PM132 leaf material. Additional N. tabacum PM132 GnTI sequences. SEQ ID NO:265 discloses the nucleotide acid sequence of GnTI contig 1#5 of N. tabacum PM132. SEQ ID NO:266 discloses the nucleotide acid sequence of GnTI coding region contig 1#5. SEQ ID NO:267 amino acid sequence of putative protein encoded by GnTI contig 1#5 of N. tabacum PM132. SEQ ID NO:268 discloses the nucleotide acid sequence of GnTI contig 1#8 of N. tabacum PM132. SEQ ID NO:269 discloses the nucleotide acid sequence of Gnu coding region contig 1#8. SEQ ID NO:270 amino acid sequence of putative protein encoded by Gnu contig 1#8 of N. tabacum PM132. SEQ ID NO:271 discloses the nucleotide acid sequence of GnTI contig 1#9 of N. tabacum PM132. SEQ ID NO:272 discloses the nucleotide acid sequence of GnTI coding region contig 1#9. SEQ ID NO:273 amino acid sequence of putative protein encoded by GnTI contig 1#9 of N. tabacum PM132. SEQ ID NO:274 discloses the nucleotide acid sequence of GnTI T10 702 of N. tabacum PM132. SEQ ID NO:275 discloses the nucleotide acid sequence of GnTI coding region of T10 702. SEQ ID NO:276 amino acid sequence of putative protein encoded by GnTI T10 702 of N. tabacum PM132. SEQ ID NO:277 discloses the nucleotide acid sequence of GnTI contig 1#6 of N. tabacum PM132. SEQ ID NO:278 discloses the nucleotide acid sequence of GnTI coding region contig 1#6. SEQ ID NO:279 amino acid sequence of putative protein encoded by GnTI contig 1#6 of N. tabacum PM132. SEQ ID NO:280 discloses the nucleotide acid sequence of GnTI contig 1#2 of N. tabacum PM132. SEQ ID NO:281 discloses the nucleotide acid sequence of GnTI coding region contig 1#2. SEQ ID NO:282 amino acid sequence of putative protein encoded by GnTI contig 1#2 of N. tabacum PM132.

Many of the above-described sequences are used to down regulate or knock-out N-acetylglucosaminyltransferase I activity in N. tabacum PM132 plant cells or whole plants—either via but not limited to RNAi technology, chemically induced mutagenesis or genome editing technology such as but not limited to zinc finger nuclease-mediated knock-out, meganuclease-mediated knock-out, mutagenic nucleobase-mediated knock-out or other genome editing technology in tobacco.

The regulatory elements that are identified in the genomic sequences disclosed herein can be used to drive the expression of a heterologous protein in a plant such as but not limited to tobacco and its various species and varieties. The GnTI coding sequences can be used to produce N-acetylglucosaminyltransferase I in an organism such as but not limited to a plant cell, bacterial cell, yeast cell, mammalian cell, a fungal cell or insect cell. The CPO sequence of N. tabacum PM132 containing a stop codon can be used to produce a GnTI-like enzyme lacking the C-terminal part of the protein. Also contemplated is the deletion or replacement of the stop codon thereby restoring the reading frame and resulting in a coding sequence that encodes an enzymatically active GnTI enzyme.

### 12.1 Materials and Methods.

12.1.1 Methods to Obtain FASO Homologs of GnTI Genomic and cDNA Sequences

Genomic DNA is extracted from leaf tissues of N. tabacum PM132 using a CTAB-based extraction method. Leaves of N. tabacum PM132 are grinded in liquid nitrogen into powder. RNA is extracted from 200 mg of powder, using RNA extraction kit (Qiagen) following the supplier's instructions. 1 μg of extracted RNA is then treated with DNaseI (NEB). Starting from 500 ng of DNase-treated RNA, cDNA is synthesized using AMV-Reverse Transcriptase (Invitrogen). First strand cDNA samples are then diluted ten times to serve as PCR template. Plant cDNA or gDNA is amplified by PCR using Mastercycler gradient machine (Eppendorf). Reactions are performed in 50 μl including 25 μl of 2× Phusion mastermix (Finnzyme), 20 μl of water, 1 μl of diluted cDNA, and 2 μL of each primers (10 NM) listed in the tables. The thermocycler conditions are set-up as indicated by the supplier and using 58° C. as annealing temperature. After the PCR, the product is 3′ end adenylated. 50 μl of 2× Taq Mastermix (NEB) are added to the PCR reactions, these were incubated at 72° C. for 10 minutes. The PCR products are then purified using the PCR purification kit (Qiagen). The purified products are cloned into the pCR2.1 using TOPO-TA cloning kit (Invitrogen). The TOPO reactions are transformed into TOP10 E. coli. Individual clones are picked into liquid medium, plasmid DNA is prepared from the cultures and used for sequencing with primers M13 and M13R. Sequence data are compiled using Contig Express and AlignX software (Vector NTI, Invitrogen). Assembled contigs are compared to known sequences.

TABLE 1
Primer sequences used within PCR for obtaining GnTI genomic and cDNA sequences

| Candidate | BAC or gene | | |
|---|---|---|---|
| Gene | name | Primer sequences from 5′ to 3′ | |
| GnT1 | FABIJI | Coding | SEQ ID NO: 236: ATCGCACGATGAGAGGGT |
| | | | SEQ ID NO: 237: TTAAGTATCTTCATTTCCGAGTTG |
| | CPO | Coding | SEQ ID NO: 238: ATGAGAGGGTACAAGTTTTGCTG |
| | | | SEQ ID NO: 239: GTTTGGTACCGGAAAACCACT |
| | CAC80702.1 | Coding | SEQ ID NO: 240: CAGGGCTACATTTCCTCTTTATG |
| | | | SEQ ID NO: 241: ATCGCACGATGAGAGGGA |

12.1.2 Methods Relating to Identifying N. tabacum PM132 FABIJI Homologs.

TABLE 2
Primers used to screen for Hicks Broadleaf BAC-
derived genomic FABIJI_1 homolog for GnT1:

| Forward 5′ to 3′ | Reverse 5′ to 3′ |
|---|---|
| SEQ ID NO: 242: | SEQ ID NO: 243: |
| AACTTGTGGGCAGTCAGGAT | GCGGTTCACCTTATCTTTGC |
| SEQ ID NO: 244: | SEQ ID NO: 245: |
| TAATCGACCTGGGATGTTCAC | GCATCCAAGATCTCCTGCTC |

The nucleotide sequences obtained from sequencing RT-PCR fragments of N. tabacum PM132 are aligned to the full genomic FABIJI_{—}1 sequence of N. tabacum Hicks Broadleaf.

SEQ ID NO: 256: genomic DNA sequence of N. tabacum PM132-FABIJI
atgcaatatccttggaccactccactaccttccttttctgaaacaaaagctctgaagcccactctccttgggactcc
aatccttaacggcctcccattgtctggaaatacccatccacgcggtctgattttagttttccctggccatataacct
gatccaaccgttgagttgcacttgacctattagctggtttggcataaagagactccggaggcacaacggatagccca
gagtagttacaccagtatcctatttgccttaaccatcctttgccaactacattgagaatatcaaacgagggacggaa
catggatctatctggtttaaatgcaatgggaccacttacccctgtcatgttggtctttaatatgttactaagcaact
tcttaccaccatcaaaaatgctaagtgcagcaaggttcatcgtctctccagcaaaactgtccaaattagaatcattt
gagtaggagattttgcatccttgatctaaaaactctttaactgcgtaagcaatcatccaaacagtatcataggcgta
tagaccgtaggcattcaaaccaacggagctattgctcaacttgttccaccttgatacaaaagccctcttcttttggg
aatcaggtgtatggggccgaagggtgagagcaccttgtatagagctagccacctttgttgaaactgaagtcgaatca
aggacaccggaaagccaagaagtagcaatccaaacatattcactcgtcatcatgccaagctcctgggcaacctcaaa
aaccttgagacctgttatggatagtgtatgtagaacaataactcgggattcgattgatttaaccttgagcaactcag
ccacgatcaggtcacgactagacatgagttcaggtggaagaattgccttgtaagaaatcttacaacgtctctcaaca
agtttatcacctagagcggcaatactatttcgaccttgatcatcgtctgagaaaattgcaatgacttctctgtattg
aaaataactgatcatatcggctacggcagtcattagaaaaagatcactgggggcagtctgaatgaaataggggtact
gaagaggtgagagtgtggggtccaatgctgtgaaagaaaggagcgggacatggagttcattcgcaaggtgagagagt
acatgggccattacagaactttgagggccaatcacagctactgtatcggtctccatgaattgtaatgctgggaagcc
agaaaagtagaaaagagttaacaagacgatctagtcaagtgatatctaagagcagtgagagataaattgaaaaagtg
tagtatgaaaaggtgagaactatatatatatacctccaatgatcccaaggaatccgctgtagtttgaatcatggagg
gtgagagcaagttttcttccgtcaagaagagtggtatcagaattgacgtcttggacagcagcttccattgcgattct
agcaaccttgccgttggtggtgccaaaagaaaagatggctccaatcttcacctcataagcttgtctctgctcctctg
aagattgtccaataaagcagacgaacagaattagcagaaaacaatttaaattcatgatgacgcctccaattgcaatt
aatgcgttggtaactgtagaaggatcagattaccaacaaaagtaaaataaaacccaatgtgacgaacaactgttaga
aatggaggagagagcagggctaaagggacgggcaggaagaacttttcaagtctgagaacttggaagttaattctgtc
atgatagaaaataaaaggagacaaccgcagagacagagaggaagcgaccttcaaatcttaaagtttataaactccga
gagaggaaacagagaggacaagaaatgtcctttcgaagaggaagtagtgatactagattactaaagtggcaagccaa
ggtctttcatttgttctgggtagggtagtagccatataaagtgaagttttagtcttttttctgaaggatatcacgag
atatagacagttccctcaagtaaaagaaaaggaaattgtggagcacaccaaaatcaaaatggccaaccacccggagt
aataaaaagttagtagaacatagctatgacaaaggcattagggattaaacaaagaaaaaataatccaaaaggatgga
tggacggtggcctgctttgacatatttgagatttattatgatatgagcagaatgagaatacttgagtatacaggaac
tttaggatataagtttaatagctagcttgtcattctaggattactccattatgcaacttgctcggttggacaaccac
tccactttccgcgcataaaacataaaagtaagatatccgttgttgtcattattaataccctccgccacagcgcacag
ggcttggattggaaattcggaaatctatgatgttatgacacatcttggtgcagcgcaaggattggaagataaaatgt
tgcagcatttatatttccctttggagctcaagcggcaaggagggtaggtcaattcttgttttactctgaggcatcca
tattatttccattgttcaaaaactatcagtttcatggatattaatagcataaactttcaacgcgaaattgagtattt
atgtaagtattatcatgacaatttgctgggttataaatgtacgcagaaacactctttggatatacgcttaatcttta
ttttaacgtgggctagtggtggcattcctttagtcctattgtatgatgaaacctactccttactttattatatcttt
gttcgttaataactaatataatgatcattttaacttgtcaatgaagcaacaaaaaaaaaaaacaaaatcatagacaa
tgatagtgtacatactgaggtaatattaatttataggagtaccatttaatgatcataacacatgatgtttgaacgaa
gacacaggagattatacagtaaatattgatcaaatgaagagacccagcacaacatagattagcaaagagtggagtgg
aagaccataacttagacgcattaggtttctcctgcaagaggaaaagggaaaatcaagaccaggattgcaacaagaaa
gagagaaaccactaagcttgattggtggatttgtcactacgtacacgatgacaagagaaaaatacttactggtcgtt
tagtttgtgggatagggataacaatttcagaataaaaatgcaagattcttttaattatgagattaattataccatag
ttatgatatcatttttatacattctcaatacggaataacaatccccgaattactaatctcaaaataacataccaaaa
tgactaagatacctttttccaaagctcttctctcaaagtcctttagaaaatcttaggtgaaaattagaaataaaaaa
ttatctcaacttatctaagtataaaattaaatacatgttttatatcttgtatatattttatttttatctaattagcc
aaatatctactaataaaattatatcgactaaataatcccgccattatacttctggtattatttattcaccaaccaaa
cgaccctccttaattgttggttgcatgtacaagctattacaatatagtgtttggttgcctcttgaattttgtttaaa
attcagcattatatataggatgtttggttgttgtttttattacctgcataaaaaatatataaataaattacgcaaaa
attaataaatatattattttatagctgggatataaggtgtaataagaatatgaaaattagtaatatatgtattaaaa
caactaaaaagattaaataattttcttctaaataagcaaaacacatattttaatccctgcattataattttatgcat
attattcctgtattaaccgttatattattaatctacagaaaattcatcttatttaaaacacggtaatttttttatat
ttaatttgtgttttttccccttgtgaaatttaattgtcttgtcggagtttatttccaagagagaagagagtatgaaa
aggaccaatattgacttgatcctaactgaacaggcaaagtaaatccacggatgaaacactcataactgaacagtgat
agactattcgctttctcctaaagctttcaatcgaaatcgcacgatgagagggtacaagttttgctgtgatttccggt
acctcctcatcttggctgctgtcgccttcatctacatacaggttctcttatacatggcttatatctcagatctatct
ttcttgtacgattaagatcaccagcaatgaaataggttcattaggttaggtttcttttggaccttagccttctctta
aattaccactgtttcatatgaactctacatgaacataattcgcaatctttaatacagaaaattgatgactaagaaat
tagtggaactaattttgaattacgtagaatttagaacaagtttgttattaaatcttaggaaactagagaacaatttt
aacatcaacttgtgggcagtcaggatttatacctaggggattaaaaaaaaatgcaaacttgcagaatagcttaacta
tcaaggggattcaacaattttttttatatatataaaaaataatttttccctatttgtacagtgtaactttcctcgca
agagattaaagtgaacccccttcaatacatttattgatttagctgtgtcactagtggggtgtgccactttaagcagc
tggttccctcttttagtattttggtcgcaaattccccttggcaaagataaggtgaaccgctaggaaagaattgacat
tcacatgcccaaaagaacttctgtaggctatgcatttgaaattttcatggcttgtaggcgaagcaattgaaactttt
ttctgctattgcaaatttgcaatagattctgacgacactgtaccatctgaggtaaataacttttggtactgtactgt
atggtttagttttggtatctctgttatctctttctaatgtattagacaaaagcaaatatcaagatttaacttctagc
cccaaggttctggcgtaacaaatgaacaatttgggcaacaatattctcatctgcctaagcttggtggatagagttac
ttgatatctgtgctagtaggaggtattaagtacccggtggattagtggagatgcatgcaaccgcaattgtaaaaaga
aaagtttatattgcttagggaaagccaagcaatatatgaggttacttggttttgttgacatgggtattatgaaaaga
atttaccttttttttttgatttctttctttttctttctggattagtgtttgcttaatggtgaattaggtatggtttt
aagtggttgcttttgctacattgctcagatgcggctttttgcgacacagtcagaatatgcagatcgccttgctgctg
cagtatgtatctggactcatctagtcatcctccctacaggaaatctaaataccatagacatatttcttttgttctac
agtttaagaatttgtattcatgtcatgtattgtgaatatgatgtttctaaaatcttcatatgctctacgtgaaggca
tccttcaacaattcaaatgtcattccaaaaatcttctcttttcttctcagaaggatattgcataatctttctttgtg
ttgtcttaacagcatacaactgcgcccttcttcaatgatgcaggctaaagaaagaagtaaagaacttttaattgctc
actatgtgtataaatcattgaatgacacagattgaagcagaaaatcactgtacaagtcagaccagattgcttattga
ccagattagccagcagcaaggaagaatagttgctcttgaaggtgcaatgtgtttttcggtgtagtcctttctttctt
cattgtcctcttgataaatggatttatttcctccattctacaaatggatctattggaaatagtctatcttgaaaatt
ttatgtaagttttggtcctatcataagtgagtacactgaaaatatttgatcaagaagatgcaagagagtgtagaaga
tagtaatggttaactccaagtacaaaaatctagatcagagcatgagctaaccaataccaaaactttgcctgctaggc
cagagtaagagagctaatgaaatctaggaggggaataacgtcatttacaggggaaaggttactccaactaaaaagat
tcatcaaacatatagatttcagggagcaattaggagttgaaatgccatcaaaacatctgctatttctttctgtccaa
atacaccaaaaaatacacgctgggatcatctgccaggtctttttgatggttccgtcaacttcccagaagctccaatt
ttctactgcttcctttaggttctgaggtgttgtccagctaataccaaaaactgataggaacatttaccatatgtctg
cagccactgaacaatgcaaaaaaagatgatttactgactctgaactctgatgacacatgtaacatctgttcaccatt
tgaaaaccccttctacaaatcttgagtcaggatagcttcttcaagggctgtccatgtaaagcacatgacttcagtgg
ggagttttttttctagattagtttccaaggccaatgatcaatcacttcatttgatacgcacattttgttgtaccctg
ccttcactgaataaatgcccttgctggtgttgtcccacattaggatgtctgggttttgtgggttcatcgtgaggtct
tcaagtattctgtatagatcaaagagttcgtccagttcccaatccagcatgttccttttgaattgaatgttcagttg
ttcccatccctgttatgtgctattgtgttgatgtagatctggtctcttttgagaaattttctgtttttgtgttgtaa
gtttcgagacatcttcatggatgagcagtgaggataggaccttttcagtttcttcgtgtcctctttcaatgctttgt
tccttatcctttctgtgataatacagatcatgttgaatatttgcttctgttactgctgatttatgatttactagaat
aataagtagtttagtcgtaggaggggtctttgtttaaatgtaaatttagttggataagttagttgagatatttgagg
tttttgaaatttgaatatttattctgcagattatgttttcaagttggctatttaaagccctctggttaataaaatta
aaatgagagacaatttcaaccattcttttaatcttcttgctgctccatctctttaaaaaacctaacagatcccaatt
aataaaatctggtgtttgctgtcagaaactgaaatgctacttatctcttttgtatgaagggaacaggtagttgtatt
ttttggggggaggggaagaaaggtaatgggtaattttactttccttatcttcatcttgctacattttcagaacaaat
gaagcgtcaggaccaggagtgccgacagttaagggctcttgttcaggatcttgaaagtaagttcataaactcctctt
cttctttcagcttttagtccaaaagccactgcttttagtcacagtaatatgaaatgtttgcctgtaataatgaaacc
cattgtacgtggcaaataaagatctgtcagtgtcaatgtgtctgttcatatcattgagttattaatattatgggctc
taatcctagatatacccatgctacaagtatttgtacttatttatatagttgatattgttaatttatttgttacaggt
aagggcataaaaaagttgatcggaaatgtacaggtgtacatacattctcatatcctcagtcatgctttcactatcaa
catctgttgacttcatttctgtcaaatttgtgcatcacctaattactatatttactagatgccagtggctgctgtag
ttgttatggcttgcaatcgggctgactacctggaaaagactattaaatccatcttaaagtatgttttgtatcaaaac
aattttgtctgcttcttattgcatattagatgcctcagctgataagcccggtacttccattgttgtcatcagatacc
aaatatctgttgcgccaaaatatcctcttttcatatcccaggtacccatttattttcgcacataactttctattgta
tgcttgtcttctttttgttgttgaacctacttttcgatctacctccctttggcaggatggatcacatcctgatgtta
ggaagcttgctttgagctatgatcagctgacgtatatgcaggtaatcttctctaccgcgtgagaagggaaaacagga
tgtttggcgtatctctatctttgaaatttaaatcaggtatatgtctttacttggaggggaagtatagacttaagaat
aagaactcattgttgccaggcttgtttttacttgcaatactcaatcatcatcattaccaataaccatattatgtaca
gggaaacaagttagtagaaatattgcccataaggagttttcatctgctaaaagattgaaagggaaaagatacattat
ttatatttaacctgtagatattttccttatcatttcgacccttttattacttcagctttgtatcattgtgtgacaca
atttgtccttttccctataagacagcacaagtggaagaggcatgtattgtttgatttatgcttttatgttgcagctt
ttccccctctcttcatatatatgtgatttctctctctctctctctctctctctctcttatgagtagccacacttctg
ttccatatattcattcatctactgcaataggttcatagttttgtaacctatcgattgctttttctacctaatgtttt
tctctgataaaagctacgcattgcataggatatgaatctgtctgcttcattttatcatttggctgcagttactttag
tctttatctttaaccttttgctgcctagctgataactgttctggcctggcaatgtgaaatgtagttaacaattgctt
ctgcttaagctcggtatcaaactcttcttggcgctttttcttgacagttcttaagaaaagactttttcgattcttta
tcaacagcacttggattttgaacctgtgcatactgaaagaccaggggagctgattgcatactacaaaattgcacgta
aggatgatttggtcctttttttcccatcttttttcgtaactcatttttattccaactagtgctagtcttgccttagc
cattgtcgatcactctttccgtaggtcattacaagtgggcattggatcagctgttttacaagcataattttagccgt
gttatcatactagaaggtactgctgatctatcttaatcactatgttgcatgttctttgctctttttcttctcacaat
atctgtgcctctgacatgcagatgatatgaaaattgcccctgatttttttgacttttttgaggctggagctactctt
cttgacagagacaagtaaggcactcttaaaggatccggatgttgcgttgttttactttcaaagaattattcaattca
tcctagtctcaggaaaattactatttttttactcgtgtccaactcccccctcattttcttaaaagaaccaacataat
tgaatcagattcaacagcatccaagatctcctgctattccaggcttgtgataggagaaaatctgatggcagcgaggg
ggatagattgatttccattttggttatataatattcttagcaaaaggattaaaagcttttccctcgtagactgacgt
ccaaatatgctagatagtgaacgaactagaatgggattagcctaaaacatggggataaaaagcctgttctaaatgtc
ccaagtatgttataagaatttcttaaatacttatggtgaacatcccaggtcgattatggctatttcttcttggaatg
acaatggacaaatgcagtttgtccaagatccttgtaagttttttctttcttccttcttttttgtcctttgtgattgg
tggttatgatttttcttttgaactcttctcctgtttcaattggaaattttactgaccgttattcaatgaagaaaccc
aaacgctgcttagtgcagatggtttctttttctgttctgttgaatggttatacttcattttctttttgattccttgg
aagaaattatatcctaaaacagcgtaaaggatttgcttttgagtactttacttttgatatacctctgcagttttttc
tttattccttttcgatgactggttcttggatttgtctgccacatgtctctctttctgtgactggttcctgaatttct
ctgccattgtctctctttctccttgctcaacccatatcctttttaatcatcaacttgaaattgaatcatattactca
tgctaatacaagcatcagtaagaagactggtagtgttacaatatactagtggtttttctttcattcaatcatcactt
gtttgacagcttaaactaggctccactttagagataggtttttggtcttaattaaaataggtcaagggcgcgtcgga
acagtcggtagctgcttagtactgaattttaacgtctcctcttttcgttttggagaaaccaatgaaaaaggggaaaa
gttgaaaatttgctcgttggagttgtaacaggaagttttatgagaaattggaaaacaaaaacaagaaaagaaaatat
atttttaaaatttttaggacagggaattaccttttcttgaactgataggagccaatcgttttcgcatgtgaatcaag
cagtcgtaagtgacttgttcttttggtacaaacacaaatattttatggctaagattgtcgtaagagaaaattttggg
gcgctacggttctcttttcaaatccatagccctttctaggattggcttcaattgaatattttggactgtccaaaaga
aaaaggagttgcatgtttttaccccattgatttcattgttgggctgagcaaaagtatatcctccatggaggttaatc
ccattgttttcttctcgatgttgcggaatttattgatattatttaggtgtcttttagcaaagtacacagagctctgc
ttttctgatctcactgaaatgctttataatttactctgcagatgctctttaccgctctgatttttttcccggtcttg
gatggatgctttcaaaatctacttgggacgaactatctccaaagtggccaaaggcatatcctttcgaactgatgtgc
ttatttcttgcctaaattgactaccttggaaacttcaaagattttctttgaccttacttttacttactgggacgact
ggctaagactcaaagagaatcacagaggtcgacaatttattcgcccagaagtttgcagatcatataattttggtgag
catgtatgtgctccttgaaatcagtgctagatgactttggctcagtagacatagttgagcttgaattctgatcttca
atggtgtgatattattaatgtttcttactgatcaagaaaaagttaatatgtatctcattgctcttcttactcattta
catgcttatcaagagaaaaaatgtttttgctgttcttaaagatggaaattttattaatttccaccatctaagtcaat
aacattaaatctttccccatatttaccatcatttacagaaacttctccttaagccttgtcaacaatcttacattatt
tgcagggttctagtttggggcagtttttcaagcagtatcttgagccaattaaactaaatgatgtccaggcatgttat
tttattttattgccatcaccccttttcttgcctactcattctttccatttgtatgacatgtattctaccttgaattt
tgttggaaggttgattggaagtcaatggaccttagttaccttttggaggtaatgacttgaagattatttttgtgctg
aaagatttagagaacttgtgaatgctgacaaattattagatggttgattgagaaatttgtcatttaaaccatcttgc
gtaggtaacttgtggtttcgtgttctcaggacaattacgtgaaacactttggtgacttggttaaaaaggctaagccc
atccatggagctgatgctgttttgaaagcatttaacatagatggtgatgtgcgtattcagtacagagatcaactaga
ctttgaagatatcgcacggcaatttggcatttttgaagaatggaaggtaatgcatatgtgacccttctcttcatatt
gaattgattatgacctgagatttgatcatatttgtttgagtgggttctttagatgcagtcattacgtatgtcgagta
tggctacgtatagcatattagccgtctatctacttaactctgaaacagactgttgagcagttcaaaattcatgcctg
attttatccttttaccacttggagatttattgtttcacagccatatgacattttccttcgatatatcatcgatgcaa
acagttgctgatctgataacacaaacgctggtaatagtattgcgacgcaaaaatatgcaggtgctcttagtgttaga
gtaagcaatcagaatccaattgcacaactcattcccttctagaattcaggtgcaaatggaggtgaaatttgaaatac
atgcctgccatcttctctttcatttatgcctatctatgggcttgggcccdagtaactttccatgcaatatgtgcttg
ggctagaggttgcgtctgcacgaacgaaaaatgaggtttgccaatatgggcaaaacttgaaccgtgttaggctagcc
tgcttggtctcatatatttattacaattcatatttttcaaataattgatatagaaagcattcttttggataggttga
tatgttatattttgatatgtattcattcttggttttgtaccacatgtatagaatgagtaaaaatgaaataggagatt
ttttaggttcatatattaaaatttagactgatctatagccattttaaatagaattagtgaaaatgaaataggaggag
atcttttaggtccataggttgcaatttagattgagctatagtcatttacttgtcttatttgtggctttggttacttg
gttacttaattcttaaacaaactgtttctgcaaatttagttactttttggtaaatatagcctagattaatagtcaat
attatagttttcaaatttaaagataaaattttcttaacgcctatttgttgctcaaggccagtactatgggaaagggt
ggggtggagttgaaattagacctatgatagcccgaccgtagtgatgttaattgtggttacattcataagtagcttgg
tccatctttattccatttcatatatgtctgaggatgttaatattgaccattgactggcccatatctgttctttgcct
gaaccgtggacaggtctattcacactagctgtgactggatttgtcctctttcatggttctccttttgctttctgtaa
aacttgcactaactgttcgtttcatcaggatggtgtaccacgggcagcatataaaggaatagtggttttccggtacc
aaacgtccagacgtgtattccttgttggccctgattcgcttcaacaactcggaaatgaagatacttaacaaagatat
gattggtaagtttctgtccataatgagcaaaactattgagtactctatacacaaagctttagtactttgtcttttaa
ttttttgcatggaattttttttattcttcttcatgaaggaaaatactcaaatgagaataatgtaggaatatgtttgg
aaacattgtaaaaccacttactttaactccaggaggctaatgtaaactattttggaacaaaatattgaagaaatagc
atcaaatattttgagacgtaaggtagaaagatcccaacttgctttgggattgaggcgtagtagctcatcttgttgta
aaatagaaagagggtcatataaattgagatggagggtctatgttacggtcccctgttatagatctagttatgggacg
ctgtaagacaaaatcagagtaagtttgggtagaggttttcttttttcgacctatagtgttggttcgttaagagagaa
agagagaacctgcaatctcgtgagttgaagtactcaaagattggaataattttttgcataccttttactgaattcaa
ataatttttgatacaaacactgatggattaaccatacacctaaaaattgggaaataacttcctaacataactggaat
gagaaagtggcctcctactgataactgctactactaataagtaataactgccacaggaaatatatgaacataactaa
cagatgcctaaagttgctgagctcatctacttccgatcttctgaaacttattatgtgtaatttgttggtaggctaaa
ggggtgctaacattactcccctttgtcaaatcgcgcttgtcctcaagcgggaagtatggaaagcgttgttggttggc
aaatcagtgttaggatatgactcttgtgcatcagattcttctcctcttcctttatagattcaatccacatttatggc
ctgggtgaatggttcatcacaattgaaacaaagtcccttaaggcgtctttcctccatctcagatctggtcaatttct
ttacaaatctggtgtgttgttgcgatgagaaatctgttgtctttgatctacggacatctgataattccgaatgaagg
ggttgtcccttgcgttcataaagccgggacatactcattgctgtcgccaaatctggagggttatgcaactccacttc
agttgctatatagtcagcaagaccactgatataaagttcaatttcttgcgactgtgtgagggtaccagcctgcgaaa
ccaattgctcaaactttttctggtaatctgccacagacccgatctggcacaacttagccaattctcccaacttttga
cttcttattggtggcccagaacgaaggtttcattgacgtttgaattcatccaagatggttgaggcatatctgtctct
agtttaaagaaccagagttgtgcattcccctctaaatgaaaagaagcacgtccaacattttcttcttcttcagtttg
cttgtgtcgaaagaagtgttcgcacctattcaaccatcccaaagggtcgtctttcccactaaaatgtgggaaattca
actttgtatatttgggaatgctggaacttcctccagtttctgatcctgaccttccttcaactccagctttacccttc
caacctcgattgtacctggtattttcgattgattcaaaatcggccttcg8atttgctaaatcctttgtggttgcgag
catatatggcttcctgtctggttagacacgttcttcttggaacaagttcaccaactgtgctagttgttgttgcattt
ggtctccaataactctcggctgtgataccaagttgtcacggtccctttttatagatgtagttatgggacgctgtaag
ataaaatcagagttagtttgggaagagattttcttttttcgacctatggtgctggttcgttaagagagaacctgtaa
tctcttgagttgaagtactcaaaggcaggaataattttatgcatacctttcactgaattcaaataaatttaaatata
aacactgatggattaaccacccacctaaaaattgggaaataacccctaacacaactggaatgagaaagtgatctacc
aatgtgtgactgccacaggaaatatatgaacataactaatagatgactggactcatctacttcctgatcttttgaaa
ctttccatgtgtaaattgttggtagactaaaggggtgctaacagtatattattgtgaaaataacatttgacctgttt
ttttaccaataagtaccatatttgctgacactgatgtgtatttcactctctactactccattcaacaggagcccgga
caaagatttagacttattgggtaggatgcatcgagctgacaccaaaccatgagtttactagttacatacaacgtttt
aattgttatatggaggagctcactgttctagtgttgaagggatatcggcttcttaatattggatgaatcatcacaac
ctattttttttaagccaagtgttccgaacataaagaggaaatgtagccctgtaaagacaatacctgggacgatcata
atcacaggtcaatagttttgcttctcagaaggaacattacaattgtgagcactccgcacgccctcttttggaagaat
atgagaacttttctcatttactctagtctattttggaaatgcagattcctcagaatttatattactcttagtgttgt
caaattgacgaacacaactgtgagcacgtaattttttccctacaaaatactcctacaaaaattcacaaaaaatggat
ttttctacttgtttttgattttatagtttttaggaattcctttttaattgtttatttgcattgtagttgcatttctt
gtgcatgttaaatatcttaaaatcatagaaaataccataaaaatgtccaattcttctttgcatagcattttagattt
taattgcattttttaggatttattcacatattaattacataattgataaatgaaaatcacaaaaataccctagtcat
tttacattttttgtttttggttttcagattaataattttcttttattagttcatattgttaaagtaattaattagtt
aattaataaataaagtagtaaaagaattaattttgcaatttgagttctaggtgctatttgggtttaaagtggctaac
attgcaaaaattaaagaagggaaaggaagaggttagtcttcgttgaaaactgggctaagagcacatttgaataggtg
gcccaaattgccaaattcgcctaagcccaatcttcctaaaacccggtccagctcccctttaaacccaaaacgccctc
gtttcagatccttaatcctagtgtcccttgagtttaatccgatggtccggaattgacaacccccatcccatataact
gtctcacccccctcccccccaaacctagagaccaaacctcgtttccccatctcccctatctctcccattccccactc
aaaactctagccgccccaactctttaccccaactctttaccccatgaccctcaaagcctcttattccttaactcatt
tttatattcccctaaagagccctagaactcatcccgtaacagatctcacaataggttaaccccaaatcttttctttc
gatttctaccattcggaggatgaacgcagcgaatttcatttttctctccgacttcagtagtcattagcacgtattca
ctagccgaattctaaaagcacaaggtcagtgattactcgttgatgaccactgttggtcagagaaacccttgaccaag
cgtttgtttgcattttcaaaaggtaacctcgaaatctttgctttgtttttcgttttcgtttaaacctatcttgtggt
gtttttcaatttctgttaaaatcgtcaaaaaaataaataattgcatgttctcgtttaaagtttataatctgtcaggt
ttcgcacctttaacttgcaaatagttatataaattatgttttgatgttttgtttaatagtttgtttctaattttctt
tgttattagattttttttttttttggtttggtttatttttgtttattgtttagacctcaatcttagttaaatgagtt
tagtttttttaatcagagttcaagttaggaataatttagaatcagttggttaaagaagttttgaaagggcatgggta
attataaggaataggaagggtaattttgtatttaaaaattatgaaatattttctgttataaataagagagagaagag
aactgtctctgaaggacataaaataaaaacggtttgggaatctggggtataagtgaacaaaaataaagtttaaaagg
tataactgaattagaaaaatcaggtttggttgccctaaaaatcttgttataaaaggtctcattctcacccattttgg
tgagaaaaaacttagaaaaaagggtcatacggttgctagcaatttaggttccaaatctgagttttaatagctgcaaa
aacactccaaaaatagaaagaaaacaatcaagaaagagggattaaaagctgattctaacctctttggactcttgcat
catttctggattcaaaaagcttggtttgatttgaaagatgttggttttactgttgctgtcactctgttgtttagatg
ggatttggattgttctcatgatttctgctgttgatctgttttgagctcactcaatagctgttttccttggcctttat
ttggcaaaagttcagcttgatttacaagtttaggtacatacctctcactcgtattttgttctgattttgtaaatttt
acctcttaccatttaattgaaggaaatttacgattttaaaaatactaaaatgagtaattaagttaaacttttattgt
tggcttgcgtgacagtggtgttaggcgccatcacgacctttaatggatttttggtcgtgacaccctatgtctaaaat
aaaatcaattaaggggtgaagaccctatgttagaaaagtgactagggagtgaagaccctatgtcagaaaattaaatc
aactagggagtgtagaccctatgtcagaaaataaaatcaactagggagtggagaccctatgttgaaaaagagactag
ggagtagagaccctatgtctaaaataaaatcaactagggagtgaagaccctatgttggaaaataagactagtgagtg
gagaccctatgtctaaaataaatatcaactagggagtgaagaccctatgttggaaaagagactagggagtggagacc
ctatgttggaaaagagactagggagtggagatcctatgttggaaaagagactaggaagtggagaccctatgtctaaa
ataaaatcaactagggagtggagaccctatgttgaaaaacgcagctagggattggaaaccctatactaccatgattt
tgaactttttttttttactaagagaatgagtaaaatgcgggaaagaatttggaaaagacttccctttcagagttgtt
gctgctgcagagctgtttctagcccccgcaatttctttttggttgcacctgcttcttgcaaggttgcttttggattg
cacacgtttcctatttttcaaacaaagaacaattgttagtttgaaacaatggttgattttgtggcattgagtgtttc
ggtcacttgatctcggtccggcttctttgatgatgatttcaaatgcaactggttgtttcctggataccgattgcatt
tctgaccctggagaacctttggcttttttgaaactctaccatgacgattggtcatgtgggacttaaccttttccaac
tttattttgcctttgtaggcctttgacttctttctcccaattttaaattcagagcaacggggaatccttggcttttc
aaaccttgccacgacggttagtcgcgtgggactcaaccttttcaacttcatttttgctcttgtaggcacttcaattt
gatttccttctttcgagagttttcaatttcaaaacatcagctaccatgcccagtcggggtcaacttgatatccctgg
cgaggttgggtacctttttgcatattagcttgtatcaaataa
SEQ ID NO: 257: N. tabacum PM132 coding sequence of FABIJI
atgagagggtacaagttttgctgtgatttccggtacctcctcatcttggctgctgtcgccttcatctacatacagat
gcggctttttgcgacacagtcagaatatgcagatcgccttgctgctgcaattgaagcagaaaatcactgtacaagtc
agaccagattgcttattgaccagattagccagcagcaaggaagaatagttgctcttgaagaacaaatgaagcgtcag
gaccaggagtgccgacagttaagggctcttgttcaggatcttgaaagtaagggcataaaaaagttgatcggaaatgt
acagatgccagtggctgctgtagttgttatggcttgcaatcgggctgactacctggaaaagactattaaatccatct
taaaataccaaatatctgttgcgccaaaatatcctcttttcatatcccaggatggatcacatcctgatgttaggaag
cttgctttgagctatgatcagctgacgtatatgcagcacttggattttgaacctgtgcatactgaaagaccagggga
gctgattgcatactacaaaattgcacgtcattacaagtgggcattggatcagctgttttacaagcataattttagcc
gtgttatcatactagaagatgatatggaaattgccccctgatttttttgacttttttgaggaggagctactcttctt
gacagagacaagtcgattatggctatttcttcttggaatgacaatggacaaatgcagtttgtccaagatccttatgc
tctttaccgctctgatttttttcccggtcttggatggatgctttcaaaatctacttgggacgaactatctccaaagt
ggccaaaggcttactgggacgactggctaagactcaaagagaatcacagaggtcgacaatttattcgcccagaagtt
tgcagatcatataattttggtgagcatggttatagtttggggcagtttttcaagcagtatcttgagccaattaaact
aaatgatgtccaggttgattggaagtcaatggaccttagttaccttttggaggacaattacgtgaaacactttggtg
acttggttaaaaaggctaagcccatccatggagctgatgctgttttgaaagcatttaacatagatggtgatgtgcgt
attcagtacagagatcaactagactttgaagatatcgcacggcaatttggcatttttgaagaatggaaggatggtgt
accacgggcagcatataaaggaatagtggttttccggtaccaaacgtccagacgtgtattccttgttggccctgatt
cgcttcaacaactcggaaatgaagatacttaa
SEQ ID NO: 258: Protein sequence of N. tabacum PM132 of FABIJI
MRGYKFCCDFRYLLILAAVAFIYIQMRLFATQSEYADRLAAAIEAENHCTSQTRLLIDQISQQQ
GRIVALEEQMKRQDQECRQLRALVQDLESKGIKKLIGNVQMPVAAVVVMACNRADYLEKTIKSI
LKYQISVAPKYPLFISQDGSHPDVRKLALSYDQLTYMQHLDFEPVHTERPGELIAYYKIARHYK
WALDQLFYKHNFSRVIILEDDMEIAPDFFDFFEAGATLLDRDKSIMAISSWNDNGQMQFVQDPY
ALYRSDFFPGLGWMLSKSTWDELSPKWPKAYWDDWLRLKENHRGRQFIRPEVCRSYNFGEHGSS
LGQFFKQYLEPIKLNDVQVDWKSMDLSYLLEDNYVKHFGDLVKKAKPIHGADAVLKAFNIDGDV
RIQYRDQLDFEDIARQFGIFEEWKDGVPRAAYKGIVVFRYQTSRRVFLVGPDSLQQLGNEDT

12.1.3 Methods to Obtain GnTI Sequences of N. tabacum PM132 CPO

Genomic DNA is extracted from leaf tissues of N. tabacum PM132 using a CTAB-based extraction method. Leaves of N. tabacum PM132 are grinded in liquid nitrogen into powder. RNA is extracted from 200 mg of powder, using RNA extraction kit (Qiagen) following the supplier's instructions. 1 μg of extracted RNA is then treated with DNaseI (NEB). Starting from 500 ng of DNase-treated RNA, cDNA is synthesized using AMV-Reverse Transcriptase (Invitrogen). First strand cDNA samples are then diluted ten times to serve as PCR template. Plant cDNA or gDNA is amplified by PCR using Mastercycler gradient machine (Eppendorf). Reactions are performed in 50 μl including 25 μl of 2× Phusion mastermix (Finnzyme), 20 μl of water, 1 μl of diluted cDNA, and 2 μL of each primers (10 μM) listed in the tables. The thermocycler conditions are set-up as indicated by the supplier and using 58° C. as annealing temperature. After the PCR, the product is 3′ end adenylated. 50 μl of 2× Taq Mastermix (NEB) are added to the PCR reactions, these were incubated at 72° C. for 10 minutes. The PCR products are then purified using the PCR purification kit (Qiagen). The purified products are cloned into the pCR2.1 using TOPO-TA cloning kit (Invitrogen). The TOPO reactions are transformed into TOP10 E. coli. Individual clones are picked into liquid medium, plasmid DNA is prepared from the cultures and used for sequencing with primers M13 and M13R. Sequence data are compiled using Contig Express and AlignX software (Vector NTI, Invitrogen). Assembled contigs are compared to known sequences.

TABLE 3
Primer sequence used within PCR for obtaining CPO sequences

| Candidate | BAC or gene | |
|---|---|---|
| Gene | name | Primer sequences from 5′ to 3′ |
| GnTI | CPO | SEQ ID NO: 238: ATGAGAGGGTACAAGTTTTGCTG |
| | Coding | SEQ ID NO: 239: GTTTGGTACCGGAAAACCACT |

### 12.1.4 Methods Relating to Identifying CPO Homologs

Sequencing is performed on overlapping PCR fragments obtained by amplification of gDNA from N. tabacum PM132 and N. tabacum PO2 varieties using the following primers:

TABLE 4
Primers used within PCR for obtaining gDNA from N.
tabacum PM132 and N. tabacum PO2 varieties.

| Fragment | Primer | Sequence 5′ to 3′ |
|---|---|---|
| 5′ UTR to Exon 7 | PC181F | SEQ ID NO: 246 |
| | | TCGCTTTCTCCTAAAGCCTTC |
| | PC190R | SEQ ID NO: 247 |
| | | tgggatatgaaaagaggatattttg |
| Exon 4 to Exon 13 | PC191F | SEQ ID NO; 248 |
| | | aaatgaagcgtcaggaccag |
| | PC192R | SEQ ID NO: 249 |
| | | gaaagcatccatccaagacc |
| Exon 12 to 3′ UTR | PC193F | SEQ ID NO: 250 |
| | | ggaatgacaatggacaaatgc |
| | PC187R | SEQ ID NO: 251 |
| | | aacatgcacaagaaatgcaa |
| Exon 12 to 3′ UTR | PC193F | SEQ ID NO: 252 |
| | | ggaatgacaatggacaaatgc |
| | PC188R | SEQ ID NO: 253 |
| | | gctcacagttgtgttcgtcaa |
| Exon 12 to 3′ UTR | PC193F | SEQ ID NO: 254 |
| | | ggaatgacaatggacaaatgc |
| | PC189R | SEQ ID NO: 255 |
| | | cagggctacatttcctctttatg |

Screening of a N. tabacum PM132 cDNA Library.

No cDNA sequences were obtained that matched the genomic CPO sequence suggesting the latter to actually be a pseudogene. cDNA sequences are obtained corresponding to FABIJI or highly identical thereto and to CAC80702.1.

TABLE 5
Summary of GnT1 clones identified in N. tabacum Hicks
Broadleaf BAC library, by PCR on genomic DNA isolated
from N. tabacum PM132 and a cDNA library.

| | Found | | | Coding: |
|---|---|---|---|---|
| | in BAC | PCR on PM132 | Coding | PCR on |
| GnT1gene name | library | genomic DNA | predicted | PM132 cDNA |
| 1 FABIJI | yes | Confirmed and | yes | Confirmed and |
| | | corrected | | corrected |
| 2 CAC80702.1 | no | No | yes | Yes (highly |
| and derivatives | | | | represented) |

The nucleotide sequence is confirmed by sequencing of overlapping PCR fragments obtained by amplification of gDNA from PM132—the seeds of which were deposited under accession number NCIMB 41802—and N. tabacum PO2 varieties using primers:

TABLE 6
Primers used within PCR for obtaining gDNA from N.
tabacum PM132 and N. tabacum PO2 varieties

| Fragment | Primer | Sequence 5′ to 3′ |
|---|---|---|
| 5′ UTR to Exon 7 | PC181F | SEQ ID NO: 246 |
| | | TCGCTTTCTCCTAAAGCCTTC |
| | PC19OR | SEQ ID NO: 247 |
| | | tgggatatgaaaagaggatattttg |
| Exon 4 to Exon 13 | PC191F | SEQ ID NO: 248 |
| | | aaatgaagcgtcaggaccag |
| | PC192R | SEQ ID NO: 249 |
| | | gaaagcatccatccaagacc |
| Exon 12 to 3′ UTR | PC193F | SEQ ID NO: 250 |
| | | ggaatgacaatggacaaatgc |
| | PC187R | SEQ ID NO: 251 |
| | | aacatgcacaagaaatgcaa |
| Exon 12 to 3′ UTR | PC193F | SEQ ID NO: 252 |
| | | ggaatgacaatggacaaatgc |
| | PC188R | SEQ ID NO: 253 |
| | | gctcacagttgtgttcgtcaa |
| Exon 12 to 3′ UTR | PC193F | SEQ ID NO: 254 |
| | | ggaatgacaatggacaaatgc |
| | PC189R | SEQ ID NO: 255 |
| | | cagggctacatttcctctttatg |

SEQ ID NO: 259: gDNA from CPO gene.
agactattcgctttctcctaaagccttcaatcgaaatcgcacgatgagagggtacaagttttgctgtgatttccggt
acctcctcatcttggctgatgtcgccttcatctacatacaggttctcttatacatggcttatatctcagatctatct
ttcttgtacgattaagatcaccagcaatgaaataggttcattaggttaggtttcttttggaccttagccttctctta
aattaccactgtttcatatgaactctacatgaacataatttgcaatctttaatacagaaaattgatgactaagaaat
tagtggaactaattttgaattacgtagaatttagaacaagtttgttattaaatcttaggaaactagagaacaatttt
aacatcaacttgtgggcagtcaggatttatacctaggggattaaaaaaaaatgcaaacttgcagaatagcttaacta
tcaaggggattcaacaattttttttatatatataaaaaataatttttccctatttgtacagtgtaactttcctcgca
agagattaaagtgaacccccttcaatacatttattgatttagctgtgtcactagtggggtgtgccactttaagcagc
tggttccctcttttagtattttggtcgcaaattccccttggcaaagataaggtgaaccgctaggaaagaattgacat
tcacatgcccaaaagaacttctgtaggctatgcatttgaaattttcatggcttgtaggcgaagcaattgaaactttt
ttctgctattgcaaatttgcaatagattctgacgacactgtaccatctgaggtaaataadttttggtactgtactgt
atggtttagttttggtatctctgttatctctttctaatgtattagacaaaagcaaatatcaagatttaacttctagc
cccaaggttctggcgtaacaaatgaacaatttgggcaacaatattctcatctgcctaagcttggtggatagagttac
ttgatatctgtgctagtaggaggtattaagtacccggtggattagtggagatgcatgcaaccgcaattgtaaaaaga
aaagtttatattgcttagggaaagccaagcaatatatgaggttacttggttttgttgacatgggtattatgaaaaga
atttaccttttttttttgatttctttctttttctttctggattagtgtttgcttaatggtgaattaggtatggtttt
aagtggttgcttttgctacattgctcagatgcggctttttgcgacacagtcagaatatgcagatcgccttgctgctg
cagtatgtatctggactcatctagtcatcctccctacaggaaatctaaataccatagacatatttcttttgttctac
agtttaagaatttgtattcatgtcatgtattgtgaatatgatgtttctaaaatcttcatatgctctacgtgaaggca
tccttcaacaattcaaatgtcattccaaaaatcttctattttcttctcagaaggatattgcataatctttctttgtg
ttgtcttaacagcatacaactgcgcccttcttcaatgatgcaggctaaagaaagaagtaaagaacttttaattgctc
actatgtgtataaatcattgaatgacacagattgaagcagaaaatcactgtacaagtcagaccagattgcttattga
ccagattagccagcagcaaggaagaatagttgctcttgaaggtgcaatgtgtttttcggtgtagtcctttctttctt
cattgtcctcttgataaatggatttatttcctccattctacaaatggatctattggaaatagtctatcttgaaaatt
ttatgtaagttttggtcctatcataagtgagtacactgaaaatatttgatcaagaagatgcaagagagtgtagaaga
tagtaatggttaactccaagtacaaaaatctagatcagagcatgagctaaccaataccaaaactttgcctgctaggc
cagagtaagagagctaatgaaatctaggaggggaataacgtcatttacaggggaaaggttactccaactaaaaagat
tcatcaaacatatagatttcagggagcaattaggagttgaaatgccatcaaaacatctgctatttctttctgtccaa
atacaccaaaaaatacacgctgggatcatctgccaggtctttttgatggttccgtcaacttOccagaagctccaatt
ttctactgcttcctttaggttctgaggtgttgtccagctaataccaaaaactgataggaacatttaccatatgtctg
cagccactgaacaatgcaaaaaaagatgatttactgactctgaactctgatgacacatgtaacatctgttcaccatt
tgaaaaccccttctacaaatcttgagtcaggatagcttcttcaagggctgtccatgtaaagcacatgacttcagtgg
ggagttttttttctagattagtttccaaggccaatgatcaatcacttcatttgatacgcacattttgttgtaccctg
ccttcactgaataaatgcccttgctggtgttgtcccacattaggatgtctgggttttgtgggttcatcgtgaggtct
tcaagtattctgtatagatcaaagagttcgtccagttcccaatccagcatgttccttttgaattgaatgttcagttg
ttcccatccctgttatgtgctattgtgttgatgtagatctggtctcttttgagaaattttctgtttttgtgttgtaa
gtttcgagacatcttcatggatgagcagtgaggataggaccttttcagtttcttcgtgtcctctttcaatgctttgt
tacttatcctttctgtgataatacagatcatgttgaatatttgcttctgttactgctgatttatgatttactagaat
aataagtagtttagtagtaggaggggtctttgtttaaatgtaaatttagttggataagttagttgagatatttgagg
tttttgaaatttgaatatttattctgcagattatgttttcaagttggctatttaaagccctctggttaataaaatta
aaatgagagacaatttcaaccattcttttaatcttcttgctgctccatctctttaaaaaacctaacagatcccaatt
aataaaatctggtgtttgctgtcagaaactgaaatgctacttatctcttttgtatgaagggaacaggtagttgtatt
ttttggggggaggggaagaaaggtaatgggtaattttactttccttatcttcatcttgctacattttcagaacaaat
gaagcgtcaggaccaggagtgccgacagttaagggctcttgttcaggatcttgaaagtaagttcataaactcctctt
cttctttcagcttttagtccaaaagccactgcttttagtcacagtaatatgaaatgtttgcctgtaataatgaaacc
cattgtacgtggcaaataaagatctgtcagtgtcaatgtgtctgttcatatcattgagttattaatattatgggctc
taatcctagatatacccatgctacaagtatttgtacttatttatatagttgatattgttaatttatttgttacaggt
aagggcataaaaaagttgatcggaaatgtacaggtgtacatacattctcatatcctcagtcatgctttcactatcaa
catctgttgacttcatttctgtcaaatttgtgcatcacctaattactatatttactagatgccagtggctgctgtag
ttgttatggcttgcaatcgggctgactacctggaaaagactattaaatccatcttaaagtatgttttgtatcaaaac
aattttgtctgcttcttattgcatattagatgcctcagctgataagcccggtacttccattgttgtcatcagatacc
aaatatctgttgcgccaaaatatcctcttttcatatcccaggtacccatttattttcgcacataactttctattgta
tgcttgtcttctttttgttgttgaacctacttttcgatctacctccctttggcaggatggatcacatcctgatgtta
ggaagcttgctttgagctatgatcagctgacgtatatgcaggtaatcttctctaccgcgtgagaagggaaaacagga
tgtttggcgtatctctatctttgaaatttaaatcaggtatatgtctttacttggaggggaagtatagacttaagaat
aagaactcattgttgccaggcttgtttttacttgcaatactcaatcatcatcattaccaataaccatattatgtaca
gggaaacaagttagtagaaatattgcccataaggagttttcatctgctaaaagattgaaagggaaaagatacattat
ttatatttaacctgtagatattttccttatcatttcgacccttttattacttcagctttgtatcattgtgtgacaca
atttgtccttttccctataagacagcacaagtggaagaggcatgtattgtttgatttatgcttttatgttgcagctt
ttccccctctcttcatatatatgtgatttctctctctctctctctctctctctctcttatgagtagccacacttctg
ttccatatattcattcatctactgcaataggttcatagttttgtaacctatcgattgctttttctacctaatgtttt
tctctgataaaagctacgcattgcataggatatgaatctgtctgcttcattttatcatttggctgcagttactttag
tctttatctttaaccttttgctgcctagctgataactgttctggcctggcaatgtgaaatgtagttaacaattgctt
ctgcttaagctcggtatcaaactcttcttggcgctttttcttgacagttcttaagaaaagactttttcgattcttta
tcaacagcacttggattttgaacctgtgcatactgaaagaccaggggagctgattgcatactacaaaattgcacgta
aggatgatttggtccttttttttcccatcttttttcgtaactcatttttattccaactagtgctagtcttgccttag
ccattgtcgatcactctttccgtaggtcattacaagtgggcattggatcagctgttttacaagcataattttagccg
tgttatcatactagaaggtactgctgatctatcttaatcactatgttgcatgttctttgctctttttcttctcacaa
tatctgtgcctctgacatgcagatgatatggaaattgcccctgatttttttgacttttttgaggctggagctactct
tcttgacagagacaagtaaggcactcttaaaggatccggatgttgcgttgttttactttcaaagaattattcaattc
atcctagtctcaggaaaattactatttttttactcgtgtccaactcccccctcattttcttaaaagaaccaacataa
ttgaatcagattcaacagcatccaagatctcctgctcttccaggcttgtgataggagaaaatctgatggcagcgagg
gggatagattgatttccattttggttatataatattcttagcaaaaggattaaaagcttttccctcgtagactgacg
tccaaatatgctagatagtgaacgaactagaatgggattagcctaaaacatggggataaaaagcctgttctaaatgt
cccaagtatgttataagaatttcttaaatacttatggtgaacatcccaggtcgattatggctatttcttcttggaat
gacaatggacaaatgcagtttgtccaagatccttgtaagttttttctttcttccttcttttttgtcctttgtgattg
gtggttatgatttttcttttgaactcttctcctgtttcaattggaaattttactgaccgttattcaatgaagaaacc
caaacgctgcttagtgcagatggtttctttttctgttctgttgaatggttatacttcattttctttttgattccttg
gaagaaattatatcctaaaacagcgtaaaggatttgcttttgagtactttacttttgatatacctctgcagtttttt
ctttattccttttcgatgactggttcttggatttgtctgccacatgtctctctttctgtgactggttcctgaatttc
tctgccattgtctctctttctccttgctcaacccatatcctttttaatcatcaacttgaaattgaatcatattactc
atgctaatacaagcatcagtaagaagactggtagtgttacaatatactagtggtttttctttcattcaatcatcact
tgtttgacagcttaaactaggctccactttagagataggtttttggtcttaattaaaataggtcaagggcgcgtcgg
aacagtcggtagctgcttagtactgaattttaacgtctcctcttttcgttttggagaaaccaatgaaaaaggggaaa
agttgaaaatttgctcgttggagttgtaacaggaagttttatgagaaattggaaaacaaaaacaagaaaagaaaata
tatttttaaaatttttaggacagggaattaccttttcttgaactgataggagccaatcgttttcgcatgtgaatcaa
gcagtcgtaagtgacttgttcttttggtacaaacacaaatattttatggctaagattgtcgtaagagaaaattttgg
ggcgctacggttctcttttcaaatccatagccctttctaggattggcttcaattgaatattttggactgtccaaaag
aaaaaggagttgcatgtttttaccccattgatttcattgttgggctgagcaaaagtatatcctccatggaggttaat
cccattgttttcttctcgatgttgcggaatttattgatattatttaggtgtcttttagcaaagtacaaacagagttc
cgcttttctgatctcactgaaatgctttataatttacactgcagatgctctttaccgctcagatttttttcccggtc
ttggatggatgctttcaaaatctacttgagacgaattatctccaaagtggccaaaggcatatcctttcgaactgatg
tgcttatttcttgcctaaattgactaccttggaaccttcaaagatgttctttgaccttacttttacttactgggacg
actggctaagactcaaagagaatcacagaggtcgacaatttattcgcccagaagtttgctgaacatataattttggt
gagcatgtatgtgctccttgaaatcagtgctagatgatattggctcagtagacatagttgagcttgaattttgatct
tcaatggtgtgatattcttagtgtttcttactgatcaagaatttaatatgtatctcattgctcttcttactcattta
gatgcttatcaagaggaaaaatgtttcttgttcttaaagatggaaattttatcaatttccaccatctaagtcaataa
aattaaatctttccccatttttaccatcgtttacagaaacttctccttaaaccttgtcaacaatcttacgttaattg
cagggttttagtttggggcagtttttcaagcagtatcttgagccaattaaactaaatgatgtccaggcatgttattt
tattttattgccatcaccccttttcttgcctactcattctttccacttgtatgacatgtattctaccttgaattttg
taaggttgattgggagtcaatggaccttagttaccttttggaggtaatgacttgaagattatttttgtgctgaaaga
tttagacaacttatgaatgctggcaaattattacatggttgattgagaaatttgtcatttagaccatcttgcgtagg
taacttgtggtttcgtgttctcaggacaattacgtgaaacactttggtgacttggttaaaaaggctaagcccatcca
tggagctgatgctgttttgaaagcatttaacatagatggtgatgtgcgtattcagtacagagatcaacttgactttg
aagatacttaactctttcgatatatcatcgacgcaaacagttgttgatctgatatcacaaacgctggtaatagtatt
gcgacgcaaaagtatgcaggtgctcttagtgttagagtaagcaatcagaatccaattgcataactcattcccttcta
taattcaggtgcaaatggaggtgaaatttgaaatacatgcttgccatattctctttcacttatgcctatctatgggc
ttgggccccagtaactttccatgcaatatgtgcttgggctagaggctgcgtctgcaggaacaaaaaatggggtttgc
caatatgggcaagacttggaccgtgttaggccagcctgtttggcctcatatatttattataattcatttttcatata
attgatatagaaagcattcttttggataggttgatgtagtatattttgatatgtattcattctgggttttataccac
atgtatagaatgagtacaaatgaaataggagatttcttaggttcatatattaaaatttagactgatctatagccatt
ttgaatagaattagtgaaaatgaaataggaggagatcttttagttccataggttacaatttagattgagcttcagtc
atttacttgttttatttgtggctttggttacttggttaattgattacttaattcttaaacaaactgtttctgcaaat
ttagttactttttggtaaataaagcctagattaatattcaatattatagtttttaaatttaaagataaaattttctt
aacgcctatttgttgctcaaggccagtcctatgggaaagggtggggtggagttgaaattagacctatgatagcccga
ccgtagtgatgttaattqtggttacattcataagtagcttggtccatctttattccatttcatatatgtctgaggat
gttaatattgaggatattcaaggcccatatctgttctttgcctgtactgtggacaggtctattcacactagctgtga
ctggatttgtcctctttcatggttctccttttgctttccgtaaaacttgcactaactgttcatttcatcaggatggt
gtaccacgggcagcatataaaggaatagtggttttccggtaccaaacgtccagacgtgtattccttgttggccctga
ttcgcttcaacaactcggaaatgaagatacttaacaaagatatgatt
SEQ ID NO: 260: Predicted coding region from CPO gene
atgagagggtacaagttttgctgtgatttccggtacctcctcatcttggctgctgtcgccttcatctacatacagat
gcggctttttgcgacacagtcagaatatgcagatcgccttgctgctgcaattgaagcagaaaatcactgtacaagtc
agaccagattgcttattgaccagattagccagcagcaaggaagaatagttgctcttgaagaacaaatgaagcgtcag
gaccaggagtgccgacagttaagggctcttgttcaggatcttgaaagtaagggcataaaaaagttgatcggaaatgt
acagatgccagtggctgctgtagttgttatggcttgcaatcgggctgactacctggaaaagactattaaatccatct
taaaataccaaatatctgttgcgccaaaatatcctcttttcatatcccaggatggatcacatcctgatgttaggaag
cttgctttgagctatgatcagctgacgtatatgcagcacttggattttgaacctgtgcatactgaaagaccagggga
gctgattgcatactacaaaattgcacgtcattacaagtgggcattggatcagctgttttacaagcataattttagcc
gtgttatcatactagaagatgatatggaaattgcccctgatttttttgacttttttgaggctggagctactcttctt
gacagagacaagtcgattatggctatttcttcttggaatgacaatggacaaatgcagtttgtacaagatcctgatgc
tctttaccgctcagatttttttcccggtcttggatggatgctttcaaaatctacttgggacgaattatctccaaagt
ggccaaaggcatattgggacgactggctaagactcaaagagaatcacagaggtcgacaatttattcgcccagaagtt
tgctga
SEQ ID NO: 261: putative protein coded by CPO gene
MRGYKFCCDFRYLLILAAVAFIYIQMRLFATQSEYADRLAAAIEAENHCTSQTRLLIDQISQQQGRIVALEEQMKRQ
DQECRQLRALVQDLESKGIKKLIGNVQMPVAAVVVMACNRADYLEKTIKSILKYQISVAPKYPLFISQDGSHPDVRK
LALSYDQLTYMQHLDFEPVHTERPGELIAYYKIARHYKWALDQLFYKHNFSRVIILEDDMEIAPDFFDFFEAGATLL
DRDKSIMAISSWNDNGQMQFVQDPDALYRSDFFPGLGWMLSKSTWDELSPKWPKAYWDDWLRLKENHRGRQFIRPEV
C*

12.1.5 Methods Relating to Identifying CAC80702.1 Homologs in N. tabacum PM132 and Other GnTI Sequences

The N. tabacum Hicks Broadleaf BAC library as described in Example 1 is screened for clones having sequences homologous to CAC80702. No BAC clone is identified. Additional nucleotide sequences of N. tabacum PM132 having homology to GnTI sequences are identified and disclosed hereinbelow.

Individual identified GnTI sequence variants of N. tabacum PM132 are as follows:

SEQ ID NO: 262: N. tabacum PM132 CAC80702.1 homolog
Cattgacttgatcctaactgaacaggcaaagtaaatccagcgatgaaacacteataactgaacactgagagactatt
cgctttctcctaaagccttcaatcgaattcgcacgatgagagggaacaagttttgctgtgatttccggtacctcctc
atcttggctgctgtcgccttcatctacacacagatgcggctttttgcgacacagtcagaatatgcagatcgccttgc
tgctgcaattgaagcagaaaatcattgtacaagccagaccagattgcttattgaccagattagcctgcagcaaggaa
gaatagttgctottgaagaacaaatgaagcgtcaggaccaggagtgccgacaattaagggctcttgttcaggatctt
gaaagtaagggcataaaaaagttgatcggaaatgtacagatgccagtggctgctgtagttgttatggcttgcaatcg
ggctgattacctggaaaagactattaaatccatcttaaaataccaaatatctgttgcgtcaaaatatcctcttttca
tatcccaggatggatcacatcctgatgtcaggaagcttgctttgagctatgatcagctgacgtatatgcagcacttg
gattttgaacctgtgcatactgaaagaccaggggagctgattgcatactacaaaattgcacgtcattacaagtgggc
attggatcagctgttttacaagcataattttagccgtgttatcatactagaagatgatatggaaattgcccctgatt
tttttgacttttttgaggctggagctactcttcttgacagagacaagtcgattatggctatttcttcttggaatgac
aatggacaaatgcagtttgtccaagatccttatgctctttaccgctcagatttttttcccggtcttggatggatgct
ttcaaaatctacttgggacgaattatctccaaagtggccaaaggcttactgggacgactggctaagactcaaagaga
atcacagaggtcgacaatttattcgcccagaagtttgcagaacatataattttggtgagcatggttctagtttgggg
cagtttttcaagcagtatcttgagccaattaaactaaatgatgtccaggttgattggaagtcaatggaccttagtta
ccttttggaggacaattacgtgaaacactttggtgacttggttaaaaaggctaagcccatccatggagctgatgctg
tcttgaaagcatttaacatagatggtgatgtgcgtattcagtacagagatcaactagactttgaaaatatcgcacgg
caatttggcatttttgaagaatggaaggatggtgtaccacgtgcagcatataaaggaatagtagttttccggtacca
aacgtccagacgtgtattccttgttggccatgattcgcttcaacaactcggaattgaagatacttaacaaagatatg
attgcaggagcccgggcaaaatttttgacttattgggtaggatgcatcgagctgacactaaaccatgattttaccag
ttacatacaacgttttaatgttatacggaggagctcactgttatagtgttgaagggatatcggcttcttagtattgg
atgaatcatcaacacaacctattattttaagtgttcagaacataaagaggaaatgtagccctgtaaagactatacat
gggaccatcataat
SEQ ID NO: 263: coding N. tabacum PM132 CAC80702.1 homolog
atgagagggaacaagttttgctgtgatttccggtacctcctcatcttggctgctgtcgccttcatctacacacagat
gcggctttttgcgacacagtcagaatatgcagatcgccttgctgctgcaattgaagcagaaaatcattgtacaagcc
agaccagattgcttattgaccagattagcctgcagcaaggaagaatagttgctcttgaagaacaaatgaagcgtcag
gaccaggagtgccgacaattaagggctcttgttcaggatcttgaaagtaagggcataaaaaagttgatcggaaatgt
acagatgccagtggctgctgtagttgttatggcttgcaatcgggctgattacctggaaaagactattaaatccatct
taaaataccaaatatctgttgcgtcaaaatatcctcttttcatatcccaggatggatcacatcctgatgtcaggaag
cttgctttgagctatgatcagctgacgtatatgcagcacttggattttgaacctgtgcatactgaaagaccagggga
gctgattgcatactacaaaattgcacgtcattacaagtgggcattggatcagctgttttacaagcataattttagcc
gtgttatcatactagaagatgatatggaaattgcccctgatttttttgacttttttgaggctggagctactcttctt
gacagagacaagtcgattatggctatttcttcttggaatgacaatggacaaatgcagtttgtccaagatccttatgc
tctttaccgctcagatttttttcccggtcttggatggatgctttcaaaatctacttgggacgaattatctccaaagt
ggccaaaggcttactgggacgactggctaagactcaaagagaatcacagaggtcgacaatttattcgcccagaagtt
tgcagaacatataattttggtgagcatggttctagtttggggcagtttttcaagcagtatcttgagccaattaaact
aaatgatgtccaggttgattggaagtcaatggaccttagttaccttttggaggacaattacgtgaaacactttggtg
acttggttaaaaaggctaagcccatccatggagctgatgctgtcttgaaagcatttaacatagatggtgatgtgcgt
attcagtacagagatcaactagactttgaaaatatcgcacggcaatttggcatttttgaagaatggaaggatggtgt
accacgtgcagcatataaaggaatagtagttttccggtaccaaacgtccagacgtgtattccttgttggccatgatt
cgcttcaacaactcggaattgaagatacttaa
SEQ ID NO: 264: Putative protein encoded by N. tabacum PM132 CAC80702.1
homolog
MRGNKFCCDFRYLLILAAVAFIYTQRLFATQSEYADRLAAAIEAENHCTSQTRLLIDQISLQQGRIVALEEQMNKRQ
DQECRQLRALVQDLESKGIKKLIGNVQMPVAAVVVMCNRADYLEKTIKSILKYQISVASKYPLFISQDGSHPDVRKL
ALSYDQLTYMQHLDFEPVHTERPGELIAVYKIARHYRWALDQLFYKHNFSRVIILEDDMETAPDFFDFFEAGATLLD
RDKSIMAISSWNDNGQMQFVQDPYALYRSDFFPGLGWMLSKSTWDELSPKWPKAYWDDWLRLKENHRGRQFIRPEVC
RTYNFGEHGSSLGQFFKQYLEPIKLNDVQVDWKSMDLSYLLEDNYVKHFGDLVKKAKPIHGADAVLKAFNIDGDVRI
QYRDQLDFENIARQFGIFEEWKDGVPRAAYKGIVVFRYQTSRRVFLVGHDSLQQLGIEDT*
SEQ ID NO: 265: Contig 1#5
TTTAGCGGCCGCGAATTCGCCCTTCATTGACTTGATCCTAACTGAACAGGCAAAGTAAATCCAGCGATGAAACACTC
ATAACTGAACACTGAGAGACTATTCGCTTTCTCCTAAAGCCTTCAATCGAATTCGCACGATGAGAGGGAACAAGTTT
TGCTGTGATTTCCGGIACCTCCTCATCTTGGCTGCTGTCGCCTTCATCTACACACAGATGCGGCTTTTTGCGACACA
GTCAGAATATGCAGATCGCCTTGCTGCTGCAATTGAAGCAGAAAATCATTGTACAAGCCAGACCAGATTGCTTATTG
ACCAGATTAGCCTGCAGCAAGGAAGAATAGTTGCTCTTGAAGAACAAATGAAGCGTCAGGACCAGGAGTGCCGACAA
TTAAGGGCTCTTGTTCAGGATCTTGAAAGTAAGGGCATAAAAAAGTTGATCGGAAATGTACAGATGCCAGTGGCTGC
TGTAGTTGTTATGGCTTGCAATCGGGCTGATTACCTGGAAAAGACTATTAAATCCATCTTAAAATACCAAATATCTG
TTGCGTCAAAATATCCTCTTTTCATATCCCAGGATGGATCACATCCTGATGTCAGGAAGCTTGCTTTGAGCTATGAT
CAGCTGAGGTATATGCAGCACTTGGATTTTGAACCTGTGCATACTGAAAGACCAGGGGAGCTGATTGCATACTACAA
AATTGCACGTCATTACAAGTGGGCATTGGATCAGCTGTTTTACAAGCATAATTTTAGCCGTGTTATCATACTAGAAG
ATGATATGGAAATTGCCCCTGATTTTTTTGACTTTTTTGAGGCTGGAGCTACTCTTCTTGACAGAGACAAGTCGATT
ATGGCTATTTCTTCTTGGAATGACAATGGACAAATGCAGTTTGTCCAAGATCCTTATGCTCTTTACCGCTCAGATTT
TTTTCCCGGTCTTGGATGGATGCTTTCAAAATCTACTTGGGACGAATTATCTCCAAAGTGGCCAAAGGCTTACTGGG
ACGACTGGCTAAGACTCAAAGAGAATCACAGAGGTCGACAATTTATTCGCCCAGAAGTTTGCAGAACATATAATTTT
GGTGAGCATGGTTCTAGTTTGGGGCAGTTTTTCAAGCAGTATCTTGAGCCAATTAAACTAAATGATGTCCAGGTTGA
TTGGAAGTCAATGGACCTTAGTTACCTTTTGGAGGACAATTACGTGAAACACTTTGGTGACTTGGTTAAAAAGGCTA
AGCCCATCCATGGAGCTGATOCTGTCTTGAAAGCATTTAACATAGATGGTGATGTGCGTATTCAGTACAGAGATCAA
CTAGACTTTGAAAATATCGCACGGCAATTTGGCATTTTTGAAGAATGGAAGGATGGTGTACCACGTGCAGCATATAA
AGGAATAGTAGTTTTCCGGTACCAAACGTCCAGACGTGTATTCCTTGTTGGCCATGATTCGCTTCAACAACTCGGAA
TTGAAGATACTTAACAAAGATATGATTGCAGGAGCCCGGGCAAAATTTTTGACTTATTGGGTAGGATGCGTCGAGCT
GACACTAAACCATGATTTTACCAGTTACATACAACGTTTTAATGTTATACGGAGGAGCTCACTGTTCTAGTGTTGAA
GGGATATCGGCTTCTTAGTATTGGATGAATCATCAACACAACCTATTATTTTAAGTGTTCAGAACATAAAGAGGAAA
TGTAGCCCTGAAGGGCGAATTCGITTAAACCTGCAGGACTAGTCCCTTTAGTGAGGGTTAATTCTGAGCTTGGCGTA
ATCATGGTCATAGCTGTTTCCTGTGTGAAATTGTTATCCGCTCACAATTCCACACAACATACGAGCCGGAAGCATAA
AGTGTAAAGCCTGGGGTGCCTAATGAGTGAGCTAACTCACATTAATTGCGTTGCGCTCACTGCCCGCTTTCCAGTCG
GGAAACCTGTCGTGCCAGCTGCATTAATGAATCGGCCAACGCGCGGGGAGAGGCGGTTTGCGTATTGGGCGCTCTTC
CGCTTCCTCGCTCACTGACTCGCTGCGCTCGGTCGTTCGGCTGCGGCGAGCGGTATCAGCTCACTCAAAGGCGGTAA
TACGGTTATCCACAGAATCAGGGGATAACGCA
SEQ ID NO: 266: coding Contig 1#5
ATGAGAGGGAACAAGTTTTGCTGTGATTTCCGGTACCTCCTCATCTTGGCTGCTGTCGCCTTCATCTACACACAGAT
GCGGCTTTTTGCGACACAGTCAGAATATGCAGATCGCCTTGCTGCTGCAATTGAAGCAGAAAATCATTGTACAAGCC
AGACCAGATTGCTTATTGACCAGATTAGCCTGCAGCAAGGAAGAATAGTTGCTCTTGAAGAACAAATGAAGCGTCAG
GACCAGGAGTGCCGACAATTAAGGGCTCTTGTTCAGGATCTTGAAAGTAAGGGCATAAAAAAGTTGATCGGAAATGT
ACAGATGCCAGTGGCTGCTGTAGTTGTTATGGCTTGCAATCGGGCTGATTACCTGGAAAAGACTATTAAATCCATCT
TAAAATACCAAATATCTGTTGCGTCAAAATATCCTCTTTTCATATCCCAGGATGGATCACATCCTGATGTCAGGAAG
CTTGCTTTGAGCTATGATCAGCTGACGTATATGCAGCACTTGGATTTTGAACCTGTGCATACTGAAAGACCAGGGGA
GCTGATTGCATACTACAAAATTGCACGTCATTACAAGTGGGCATTGGATCAGCTGTTTTACAAGCATAATTTTAGCC
GTGTTATCATACTAGAAGATGATATGGAAATTGCCCCTGATTTTTTTGACTTTTTTGAGGCTGGAGCTACTCTTCTT
GACAGAGACAAGTCGATTATGGCTATTTCTTCTTGGAATGACAATGGACAAATGCAGTTTGTCCAAGATCCTTATGC
TCTTTACCGCTCAGATTTTTTTCCCGGTCTTGGATGGATGCTTTCAAAATCTACTTGGGACGAATTATCTCCAAAGT
GGCCAAAGGCTTACTGGGACGACTGGCTAAGACTCAAAGAGAATCACAGAGGTCGACAATTTATTCGCCCAGAAGTT
TGCAGAACATATAATTTTGGTGAGCATGGTTCTAGTTTGGGGCAGTTTTTCAAGCAGTATCTTGAGCCAATTAAACT
AAATGATGTCCAGGTTGATTGGAAGTCAATGGACCTTAGTTACCTTTTGGAGGACAATTACGTGAAACACTTTGGTG
ACTTGGTTAAAAAGGCTAAGCCCATCCATGGAGCTGATGCTGTCTTGAAAGCATTTAACATAGATGGTGATGTGCGT
ATTCAGTACAGAGATCAACTAGACTTTGAAAATATCGCACGGCAATTTGGCATTTTTGAAGAATGGAAGGATGGTGT
ACCACGTGCAGCATATAAAGGAATAGTAGTTTTCCGGTACCAAACGTCCAGACGTGTATTCCTTGTTGGCCATGATT
CGCTTCAACAACTCGGAATTGAAGATACTTAA
SEQ ID NO: 267: Putative protein encoded by Contig 1#5
MRGNKFCCDFRYLLILAAVAFIYTQMRLFATQSEYADRLAAAIEAENHCTSQTRLLIDQISLQQGRIVALEEQMKRQ
DQECRQLRALVQDLESKGIKKLIGNVQMPVAAVVVMACNRADYLEKTIKSILKYQISVASKYPLFISQDGSHPDVRK
LALSYDQLTYMQHLDFEPVHTERPGELIAYYKIARHYKWALDQLFYKHNFSRVIILEDDMEIAPDFFDFFEAGATLL
DRDKSIMAISSWNDNGQMQFVQDPYALYRSDFFPGLGWMLSKSTWDELSPKWPKAYWDDWLRLKENHRGRQFIRPEV
CRTYNFGEHGSSLGQFFKQYLEPIKLNDVQVDWKSMDLSYLLEDNYVKHFGDLVKKAKPIHGADAVLKAFNIDGDVR
IQYRDQLDFENIARQFGIFEEWKDGVPRAAYKGIVVFMTSRRVFLVGHDSLQQLGIEDT
SEQ ID NO: 268: Contig 1#8
CATTGACTTGATCCTAACTGAACAGGCAAAGTAAATCCAGCGATGAAACACTCATAACTGAACACTGAGA
GACTATTGAATTTAGCGGCCGCGAATTCGCCCTTATCGCACGATGAGAGGGAACAAGTTTTGCTGTGATT
TCCGGTACCTCCTCATCTTGGCTGCTGTCGCCTTCATCTACACACAGATGCGGCTTTTTGCGACACAGTC
AGAATATGCAGATCGCCTTGCTGCTGCAATTGAAGCAGAAAATCATTGTACAAGCCAGACCAGATTGCTT
ATTGACCAGATTAGCCTGCAGCAAGGAAGAATAGTTGCTCTTGAAGAACAAATGAAGCGTCAGGACCAGG
AGTGCCGACAATTAAGGGCTCTTGTTCAGGATCTTGAAAGTAAGGGCATAAAAAAGTTGATCGGAAATGT
ACAGATGCCAGTGGCTGCTGTAGTTGTTATGGCTTGCAATCGGGCTGATTACCTGGAAAAGACTATTAAA
TCCATCTTAAAATACCAAATATCTGTTGCGTCAAAATATCCTCTTTTCATATCCCAGGATGGATCACATC
CTGATGTCAGGAAGCTTGCTTTGAGCTATGATCAGCTGACGTATATGCAGCACTTGGATTTTGAACCTGT
GCATACTGAAAGACCAGGGGAGCTGATTGCATACTACAAAATTGCACGTCATTACAAGTGGGCATTGGAT
CAGCTGTTTTACAAGCATAATTTTAGCCGTGTTATCATACTAGAAGATGATATGGAAATTGCCCCTGATT
TTTTTGACTTTTTTGAGGCTGGAGCTACTCTTCTTGACAGAGACAAGTTGATTATGGCTATTTCTTCTTG
GAATGACAATGGACAAATGCAGTTTGTCCAAGATCCTTATGCTCTTTACCGCTCAGATTTTTTTCCCGGT
CTTGGATGGATGCTTTCAAAATCTACTTGGGACGAATTATCTCCAAAGTGGCCAAAGGCTTACTGGGACG
ACTGGCTAAGACTCAAAGAGAATCACAGAGGTCGACAATTTATTCGCCCAGAAGTTTGCAGAACATGTAA
TTTTGGTGAGCATGGTTCTAGTTTGGGGCAGTTTTTCAAGCAGTATCTTGAGCCAATTAAACTAAATGAT
GTCCAGGTTGATTGGAAGTCAATGGACCTTAGTTACCTTTTGGAGGACAATTACGTGAAACACTTTGGTG
ACTTGGTTAAAAAGGCTAAGCCCATCCATGGAGCTGATGCTGTCTTGAAAGCATTTAACATAGATGGTGA
TGTGCGTATTCAGTACAGAGATCAACTAGACTTTGAAAATATCGCACGGCAATTTGGCATTTTTGAAGAA
TGGAAGGATGGTGTACCACGTGCAGCATATAAAGGAATAGTAGTTTTCCGGTACCAAACGTCCAGACGTG
TATTCCTTGTTGGCCATGATTCGCTTCAACAACTCGGAATTGAAGATACTTAACAAAGATATGATTGCAG
GAGCCCGGGCAAAATTTTTGACTTATTGGGTAGGATGCATCGAGCTGACACTAAACCATGATTTTACCAG
TTACATACAACGTTTTAATGTTATACGGAGGAGCTCACTGTTCTAGTGTTGAAGGGATATCGGCTTAAGG
GCGAATTCGTTTAAACCTGCAGGACTAGTCCCTTTAGTGAGGGTTAATTCTGAGCTTGGCGTAATCATGG
TCATAGCTGTTTCCTGTGTGAAATTGTTATCCGCTCACAATTCCACACAACATACGAGCCGGAAGCATAA
AGTGTAAAGCCTGGGGTGCCTAATGAGTGAGCTAACTCACATTAATTGCGTTGCGCTCACTGCCCGCTTT
CCAGTCGGGAAACCTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGAAAGAACATGTGAGC
AAAAGGCCAGCAAAAGGCCAGGAACCGTAAAAAGGCCGCGTTGCTGGCGTTTTTCCATAGGCTCCGCCCC
CCTGACGAGCATCACAAAAATCGACGCTCAAGTCAGAGGTGGCGAAACCCGACAGGACTAT
SEQ ID NO: 269: coding Contig 1#8
ATGAGAGGGAACAAGTTTTGCTGTGATTTCCGGTACCTCCTCATCTTGGCTGCTGTCGCCTTCATCTACA
CACAGATGCGGCTTTTTGCGACACAGTCAGAATATGCAGATCGCCTTGCTGCTGCAATTGAAGCAGAAAA
TCATTGTACAAGCCAGACCAGATTGCTTATTGACCAGATTAGCCTGCAGCAAGGAAGAATAGTTGCTCTT
GAAGAACAAATGAAGCGTCAGGACCAGGAGTGCCGACAATTAAGGGCTCTTGTTCAGGATCTTGAAAGTA
AGGGCATAAAAAAGTTGATCGGAAATGTACAGATGCCAGTGGCTGCTGTAGTTGTTATGGCTTGCAATCG
GGCTGATTACCTGGAAAAGACTATTAAATCCATCTTAAAATACCAAATATCTGTTGCGTCAAAATATCCT
CTTTTCATATCCCAGGATGGATCACATCCTGATGTCAGGAAGCTTGCTTTGAGCTATGATCAGCTGACGT
ATATGCAGCACTTGGATTTTGAACCTGTGCATACTGAAAGACCAGGGGAGCTGATTGCATACTACAAAAT
TGCACGTCATTACAAGTGGGCATTGGATCAGCTGTTTTACAAGCATAATTTTAGCCGTGTTATCATACTA
GAAGATGATATGGAAATTGCCCCTGATTTTTTTGACTTTTTTGAGGCTGGAGCTACTCTTCTTGACAGAG
ACAAGTTGATTATGGCTATTTCTTCTTGGAATGACAATGGACAAATGCAGTTTGTCCAAGATCCTTATGC
TCTTTACCGCTCAGATTTTTTTCCCGGTCTTGGATGGATGCTTTCAAAATCTACTTGGGACGAATTATCT
CCAAAGTGGCCAAAGGCTTACTGGGACGACTGGCTAAGACTCAAAGAGAATCACAGAGGTCGACAATTTA
TTCGCCCAGAAGTTTGCAGAACATGTAATTTTGGTGAGCATGGTTCTAGTTTGGGGCAGTTTTTCAAGCA
GTATCTTGAGCCAATTAAACTAAATGATGTCCAGGTTGATTGGAAGTCAATGGACCTTAGTTACCTTTTG
GAGGACAATTACGTGAAACACTTTGGTGACTTGGTTAAAAAGGCTAAGCCCATCCATGGAGCTGATGCTG
TCTTGAAAGCATTTAACATAGATGGTGATGTGCGTATTCAGTACAGAGATCAACTAGACTTTGAAAATAT
CGCACGGCAATTTGGCATTTTTGAAGAATGGAAGGATGGTGTACCACGTGCAGCATATAAAGGAATAGTA
GTTTTCCGGTACCAAACGTCCAGACGTGTATTCCTTGTTGGCCATGATTCGCTTCAACAACTCGGAATTG
AAGATACTTAA
SEQ ID NO: 270: Putative protein encoded by Contig 1#8
MRGNKFCCDFRYLLILAAVAFIYTQMRLFATQSEYADRLAAAIEAENHCTSQTRLLIDQISLQQGRIVAL
EEQMKRQDQECRQLRALVQDLESKGIKKLIGNVQMPVAAVVVMACNRADYLEKTIKSILKYQISVASKYP
LFISQDGSHPDVRKLALSYDQLTYMQHLDFEPVHTERPGELIAYYKIARHYKWALDQLFYKHNFSRVIIL
EDDMEIAPDFFDFFEAGATLLDRDKLIMAISSWNDNGQMQFVQDPYALYRSDFFPGLGWMLSKSTWDELS
PKWPKAYWDDWLRLKENHRGRQFIRPEVCRTCNFGEHGSSLGQFFKQYLEPIKLNDVQVDWKSMDLSYLL
EDNYVKHFGDLVKKAKPIHGADAVLKAFNIDGDVRIQYRDQLDFENIARQFGIFEEWKDGVPRAAYKGIV
VFRYQTSRRVFLVGHDSLQQLGIEDT
SEQ ID NO: 271: Contig1#9
CATTGACTTGATCCTAACTGAACAGGCAAAGTAAATCCAGCGATGAAACACTCATAACTGAACACTGAGA
GACTATTCGCTTTCTCGGCCGCGAATTCGCCCTTATCGCACGATGAGAGGGAACAAGTTTTGCTGTGATT
TCCGGTACCTCCTCATCTTGGCTGCTGTCGCCTTCATCTACACACAGATGCGGCTTTTTGCGACACAGTC
AGAATATGCAGATCGCCTTGCTGCTGCAATTGAAGCAGAAAATCATTGTACAAGCCAGACCAGATTGCTT
ATTGACCAGATTAGCCTGCAGCAAGGAAGAATAGTTGCTCTTGAAGAACAAATGAAGCGTCAGGACCAGG
AGTGCCGACAATTAAGGGCTCTTGTTCAGGATCTTGAAAGTAAGGGCATAAAAAAGTTGATCGGAAATGT
ACAGATGCCAGTGGCTGCTGTAGTTGTTATGGCTTGCAATCGGGCTGATTACCTGGAAAAGACTATTAAA
TCCATCTTAAAATACCAAATATCTGTTGCGTCAAAATATCCTCTTTTCATATCCCAGGATGGATCACATC
CTGATGTCAGGAAGCTTGCTTTGAGCTATGATCAGCTGACGTATATGCAGCACTTGGATTTTGAACCTGT
GCATACTGAAAGACCAGGGGAGCTGATTGCATACTACAAAATTGCACGCCATTACAAGTGGGCATTGGAT
CAGCTGTTTTACAAGCATAATTTTAGCCGTGTTATCATACTAGAAGATGATATGGAAATTGCCCCTGATT
TTTTTGACTTTTTTGAGGCTGGAGCTACTCTTCTTGACAGAGACAAGTCGATTATGGCTATTTCTTCTTG
GAATGACAATGGACAAATGCAGTTTGTCCAAGATCCTTATGCTCTTTACCGCTCAGATTTTTTTCCCGGT
CTTGGATGGATGCTTTCAAAATCTACTTGGGACGAATTATCTCCAAAGTGGCCAAAGGCTTACTGGGACG
ACTGGCTAAGACTCAAAGAGAATCACAGAGGTCGACAATTTATTCGCCCAGAAGTTTGCAGAACATATAA
TTTTGGTGAGCATGGTTCTAGTTTGGGGCAGTTTTTCAAGCAGTATCTTGAGCCAATTAAACTAAATGAT
GTCCAGGTTGATTGGAAGTCAATGGACCTTAGTTACCTTTTGGAGGACAATTACGTGAAACACTTTGGTG
ACTTGGTTAAAAAGGCTAAGCCCATCCATGGAGCTGATGCTGTCTTGAAAGCATTTAACATAGATGGTGA
TGTGCGTATTCAGTACAGAGATCAACTAGACTTTGAAAATATCGCACGGCAATTTGGCATTTTTGAAGAA
TGGAAGGATGGTGTACCACGTGCAGCATATAAAGGAATAGTAGTTTTCCGGTACCAAACGTCCAGACGTG
TATTCCTTGTTGGCCATGATTCGCTTCAACAACTCGGGATTGAAGATACTTAACAAAGATATGATTGCAG
GAGCCCGGGCAAAATTTTTGACTTATTGGGTAGGATGCATCGAGCTGACACTAAACCATGATTTTACCAG
TTACATACAACGTTTTAATGTTATACGGAGGAGCTCACTGTTCTAGTGTTGAAGGGATATCGGCTTAAGG
GCGAATTCGTTTAAACCTGCAGGACTAGTCCCTTTAGTGAGGGTTAATTCTGAGCTTGGCGTAATCATGG
TCATAGCTGTTTCCTGTGTGAAATTGTTATCCGCTCACAATTCCACACAACATACGAGCCGGAAGCATAA
AGTGTAAAGCCTGGGGTGCCTAATGAGTGAGCTAACTCACATTAATTGCGTTGCGCTCACTGCCCGCTTT
CCAGTCGGGAAACCTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGAAAGAACATGTGAGC
AAAAGGCCAGCAAAAGGCCAGGAACCGTAAAAAGGCCGCGTTGCTGGCGTTTTTCCATAGGCTCCGCCCC
CCTGACGAGCATCACAAAAATCGACGCTCAAGTC
SEQ ID NO: 272: coding Contig1#9
ATGAGAGGGAACAAGTTTTGCTGTGATTTCCGGTACCTCCTCATCTTGGCTGCTGTCGCCTTCATCTACA
CACAGATGCGGCTTTTTGCGACACAGTCAGAATATGCAGATCGCCTTGCTGCTGCAATTGAAGCAGAAAA
TCATTGTACAAGCCAGACCAGATTGCTTATTGACCAGATTAGCCTGCAGCAAGGAAGAATAGTTGCTCTT
GAAGAACAAATGAAGCGTCAGGACCAGGAGTGCCGACAATTAAGGGCTCTTGTTCAGGATCTTGAAAGTA
AGGGCATAAAAAAGTTGATCGGAAATGTACAGATGCCAGTGGCTGCTGTAGTTGTTATGGCTTGCAATCG
GGCTGATTACCTGGAAAAGACTATTAAATCCATCTTAAAATACCAAATATCTGTTGCGTCAAAATATCCT
CTTTTCATATCCCAGGATGGATCACATCCTGATGTCAGGAAGCTTGCTTTGAGCTATGATCAGCTGACGT
ATATGCAGCACTTGGATTTTGAACCTGTGCATACTGAAAGACCAGGGGAGCTGATTGCATACTACAAAAT
TGCACGCCATTACAAGTGGGCATTGGATCAGCTGTTTTACAAGCATAATTTTAGCCGTGTTATCATACTA
GAAGATGATATGGAAATTGCCCCTGATTTTTTTGACTTTTTTGAGGCTGGAGCTACTCTTCTTGACAGAG
ACAAGTCGATTATGGCTATTTCTTCTTGGAATGACAATGGACAAATGCAGTTTGTCCAAGATCCTTATGC
TCTTTACCGCTCAGATTTTTTTCCCGGTCTTGGATGGATGCTTTCAAAATCTACTTGGGACGAATTATCT
CCAAAGTGGCCAAAGGCTTACTGGGACGACTGGCTAAGACTCAAAGAGAATCACAGAGGTCGACAATTTA
TTCGCCCAGAAGTTTGCAGAACATATAATTTTGGTGAGCATGGTTCTAGTTTGGGGCAGTTTTTCAAGCA
GTATCTTGAGCCAATTAAACTAAATGATGTCCAGGTTGATTGGAAGTCAATGGACCTTAGTTACCTTTTG
GAGGACAATTACGTGAAACACTTTGGTGACTTGGTTAAAAAGGCTAAGCCCATCCATGGAGCTGATGCTG
TCTTGAAAGCATTTAACATAGATGGTGATGTGCGTATTCAGTACAGAGATCAACTAGACTTTGAAAATAT
CGCACGGCAATTTGGCATTTTTGAAGAATGGAAGGATGGTGTACCACGTGCAGCATATAAAGGAATAGTA
GTTTTCCGGTACCAAACGTCCAGACGTGTATTCCTTGTTGGCCATGATTCGCTTCAACAACTCGGGATTG
AAGATACTTAA
SEQ ID NO: 273: Putative protein encoded by Contig1#9
MRGNKFCCDFRYLLILAAVAFIYTQMRLFATQSEYADRLAAAIEAENHCTSQTRLLIDQISLQQGRIVAL
EEQMKRQDQECRQLRAINQDLESKGIKKLIGNVQMPVAAVVVMACNRADYLEKTIKSILKYQISVASKYP
LFISQDGSHPDVRKLALSYDQLTYMQHLDFEPVHTERPGELIAYYKIARHYKWALDQLFYKHNFSRVIIL
EDDMEIAPDFFDFFEAGATLLDRDKSIMAISSWNDNGQMQFVQDPYALYRSDFFPGLGWMLSKSTWDELS
PKWPKAYWDDWLRLKENHRGRQFIRPEVCRTYNFGEHGSSLGQFFKQYLEPIKLNDVQVDWKSMDLSYLL
EDNYVKHFGDLVKKAKPIHGADAVLKAFNIDGDVRIQYRDQLDFENIARQFGIFEEWKDGVPRAAYKGIV
VFRYQTSRRVFLVGHDSLQQLGIEDT
SEQ ID NO: 274: T10 702
CATTGACTTGATCCTAACTGAACAGGCAAAGTAAATCCAGCGATGAAACACTCATAACTGAACACTGAGA
GACTATTCGCTTTCTCCTAAAGCCTTCAATCGAATTCGCACGATGAGAGGGAACAAGTTTTGCTGTGATT
TCCGGTACCTCCTCATCTTGGCTGCTGTCGCCTTCATCTACACACAGATGCGGCTTTTTGCGACACAGTC
AGAATATGCAGATCGCCTTGCTGCTGCAATTGAAGCAGAAAATCATTGTACAAGCCAGACCAGATTGCTT
ATTGACCAGATTAGCCTGCAGCAAGGAAGAATAGTTGCTCTTGAAGAACAAATGAAGCGTCAGGACCAGG
AGTGCCGACAATTAAGGGCTCTTGTTCAGGATCTTGAAAGTAAGGGCATAAAAAAGTTGATCGGAAATGT
ACAGATGCCAGTGGCTGCTGTAGTTGTTATGGCTTGCAATCGGGCTGATTACCTGGAAAAGACTATTAAA
TCCATCTTAAAATACCAAATATCTGTTGCGTCAAAATATCCTCTTTTCATATCCCAGGATGGATCACATC
CTGATGTCAGGAAGCTTGCTTTGAGCTATGATCAGCTGACGTATATGCAGCACTTGGATTTTGAACCTGT
GCATACTGAAAGACCAGGGGAGCTGATTGCATACTACAAAATTGCACGTCATTACAAGTGGGCATTGGAT
CAGCTGTTTTACAAGCATAATTTTAGCCGTGTTATCATACTAGAAGATGATATGGAAATTGCCCCTGATT
TTTTTGACTTTTTTGAGGCTGGAGCTACTCTTCTTGACAGAGACAAGTCGATTATGGCTATTTCTTCTTG
GAATGACAATGGACAAATGCAGTTTGTCCAAGATCCTTATGCTCTTTACCGCTCAGATTTTTTTCCCGGT
CTTGGATGGATGCTTTCAAAATCTACTTGGGACGAATTATCTCCAAAGTGGCCAAAGGCTTACTGGGACG
ACTGGCTAAGACTCAAAGAGAATCACAGAGGTCGACAATTTATTCGCCCAGAAGTTTGCAGAACATATAA
TTTTGGTGAGCATGGTTCTAGTTTGGGGCAGTTTTTCAAGCAGTATCTTGAGCCAATTAAACTAAATGAT
GTCCAGGTTGATTGGAAGTCAATGGACCTTAGTTACCTTTTGGAGGACAATTACGTGAAACACTTTGGTG
ACTTGGTTAAAAAGGCTAAGCCCATCCATGGAGCTGATGCTGTCTTGAAAGCATTTAACATAGATGGTGA
TGTGCGTATTCAGTACAGAGATCAACTAGACTTTGAAAATATCGCACGGCAATTTGGCATTTTTGAAGAA
TGGAAGGATGGTGTACCACGTGCAGCATATAAAGGAATAGTAGTTTTCCGGTACCAAACGTCCAGACGTG
TATTCCTTGTTGGCCATGATTCGCTTCAACAACTCGGAAATGAAGATACTTAACAAAGATATGATTGCAG
GAGCCCGGGCAAAATTTTTGACTTATTGGGTAGGATGCATCGAGCTGACACTAAACCATGATTTTACCAG
TTACATACAACGTTTTAATGTTATACGGAGGAGCTCACIGTTCTAGTGTTGAAGGGATATCGGCTTCTTA
GTATTGGATGAATCATCAACACAACCTATTATTTTAAGTGTTCAGAACATAAAGAGGAAATGTAGCCCTG
TAAAGACTATACATGGGACCATCATAAT
SEQ ID NO: 275: coding T10 702
ATGAGAGGGAACAAGTTTTGCTGTGATTTCCGGTACCTCCTCATCTTGGCTGCTGTCGCCTTCATCTACA
CACAGATGCGGCTTTTTGCGACACAGTCAGAATATGCAGATCGCCTTGCTGCTGCAATTGAAGCAGAAAA
TCATTGTACAAGCCAGACCAGATTGCTTATTGACCAGATTAGCCTGCAGCAAGGAAGAATAGTTGCTCTT
GAAGAACAAATGAAGCGTCAGGACCAGGAGTGCCGACAATTAAGGGCTCTTGTTCAGGATCTTGAAAGTA
AGGGCATAAAAAAGTTGATCGGAAATGTACAGATGCCAGTGGCTGCTGTAGTTGTTATGGCTTGCAATCG
GGCTGATTACCTGGAAAAGACTATTAAATCCATCTTAAAATACCAAATATCTGTTGCGTCAAAATATCCT
CTTTTCATATCCCAGGATGGATCACATCCTGATGTCAGGAAGCTTGCTTTGAGCTATGATCAGCTGACGT
ATATGCAGCACTTGGATTTTGAACCTGTGCATACTGAAAGACCAGGGGAGCTGATTGCATACTACAAAAT
TGCACGTCATTACAAGTGGGCATTGGATCAGCTGTTTTACAAGCATAATTTTAGCCGTGTTATCATACTA
GAAGATGATATGGAAATTGCCCCTGATTTTTTTGACTTTTTTGAGGCTGGAGCTACTCTTCTTGACAGAG
ACAAGTCGATTATGGCTATTTCTTCTTGGAATGACAATGGACAAATGCAGTTTGTCCAAGATCCTTATGC
TCTTTACCGCTCAGATTTTTTTCCCGGTCTTGGATGGATGCTTTCAAAATCTACTTGGGACGAATTATCT
CCAAAGTGGCCAAAGGCTTACTGGGACGACTGGCTAAGACTCAAAGAGAATCACAGAGGTCGACAATTTA
TTCGCCCAGAAGTTTGCAGAACATATAATTTTGGTGAGCATGGTTCTAGTTTGGGGCAGTTTTTCAAGCA
GTATCTTGAGCCAATTAAACTAAATGATGTCCAGGTTGATTGGAAGTCAATGGACCTTAGTTACCTTTTG
GAGGACAATTACGTGAAACACTTTGGTGACTTGGTTAAAAAGGCTAAGCCCATCCATGGAGCTGATGCTG
TCTTGAAAGCATTTAACATAGATGGTGATGTGCGTATTCAGTACAGAGATCAACTAGACTTTGAAAATAT
CGCACGGCAATTTGGCATTTTTGAAGAATGGAAGGATGGTGTACCACGTGCAGCATATAAAGGAATAGTA
GTTTTCCGGTACCAAACGTCCAGACGTGTATTCCTTGTTGGCCATGATTCGCTTCAACAACTCGGAAATG
AAGATACTTAA
SEQ ID NO: 276: Putative protein encoded by T10 702
MRGNKFCCDFRYLLILAAVAFIYTQMRLFATQSEYADRLAAAIEAENHCTSQTRLLIDQISLQQGRIVAL
EEQMKRQDQECRQLRALVQDLESKGIKKLIGNVQMPVAAVVVMACNRADYLEKTIKSILKYQISVASKYP
LFISQDGSHPDVRKLALSYDQLTYMQHLDFEPVHTERPGELIAYYKIARHYKWALDQLFYKHNFSRVIIL
EDDMEIAPDFFDFFEAGATLLDRDKSIMAISSWNDNGQMQFVQDPYALYRSDFFPGLGWMLSKSTWDELS
PKWPKAYWDDWLRLKENHRGRQFIRPEVCRTYNFGEHGSSLGQFFKQYLEPIKLNDVQVDWKSMDLSYLL
EDNYVKHFGDLVKKAKPIHGADAVLKAFNIDGDVRIQYRDQLDFENIARQFGIFEEWKDGVPRAAYKGIV
VFRYQTSRRVFLVGHDSLQQLGNEDT
SEQ ID NO: 277: Contig 1#6
GATTTAGCGGCCGCGAATTCGCCCTTCATTGACTTGATCCTAACTGAACAGGCAAAGTAAATCCACGGAT
GAAACACTCATAACTGAACAGTGATAGACTATTCGCTTTCTCCTAAAGCCTTCAATCGAAATCGCACGAT
GAGAGGGTACAAGTTTTGCTGTGATTTCCGGTACCTCCTCATCTTGGCTGCTGTCGCCTTCATCTACATA
CAGATGCGGCTITITGCGACACAGTCAGAATATGCAGACCGCCTTGCTGCTGCAATTGAAGCAGAAAATC
ACTGTACAAGTCAGACCAGATTGCTTATTGACCAGATTAGCCAGCAGCAAGGAAGAATAGTTGCTCTTGA
AGAACAAATGAAGCGTCAGGACCAGGAGTGCCGACAGTTAAGGGCTCTTGTTCAGGATCTTGAAAGTAAG
GGCATAAAAAAGTTGATCGGAAATGTACAGATGCCAGTGGCTGCTGTAGTTGTTATGGCTTGCAATCGGG
CTGACTACCTGGAAAAGACTATTAAATCCATCTTAAAATACCAAATATCTGTTGCGCCAAAATATCCTCT
TTTCATATCCCAGGATGGATCACATCCTGATGTTAGGAAGCTTGCTTTGAGCTATGATCAGCTGACGTAT
ATGCAGCACTTGGATTTTGAACCTGTGCATACTGAAAGACCAGGGGAGCTGATTGCATACTACAAAATTG
CACGTCATTACAAGTGGGCATTGGATCAGCTGTTTTACAAGCATAATTTTAGCCGTGTTATCATACTAGA
AGATGATATGGAAATTGCCCCTGACTTTTTTGACTTTTTTGAGGCTGGAGCTACTCTTCTTGACAGAGAC
AAGTCGATTATGGCTATTTCTTCTTGGAATGACAATGGACAAATGCAGTTTGTCCAAGATCCTTATGCTC
TTTACCGCTCTGATTTTTTTCCCGGTCTTGGATGGATGCTTTCAAAATCTACTTGGGACGAACTATCTCC
AAAGTGGCCAAAGGCTTACTGGGACGACTGGCTAAGACTCAAAGAGAATCACAGAGGTCGACAATTTATT
CGCCCAGAAGTTTGCAGATCATATAATTTTGGTGAGCATGGTTCTAGTTTGGGGCAGTTTTTCAAGCAGT
ATCTTGAGCCAATTAAACTAAATGATGTCCAGGTTGATTGGAAGTCAATGGACCTTAGTTACCTTTTGGA
GGACAATTACGTGAAACACTTTGGTGACTTGGTTAAAAAGGCTAAGCCCATCCATGGAGCTGATGCTGTT
TTGAAAGCATTTAACATAGATGGTGATGTGCGTATTCAGTACAGAGATCAACTAGACTTTGAAGATATCG
CACGGCAATTTGGCATTTTTGAAGAATGGAAGGATGGTGTACCACGGGCAGCATATAAAGGAATAGTGGT
TTTCCGGTACCAAACGTCCAGACGTGTATTCCTTGTTGGCCCTGATTCGCTTCAACAACTCGGAAATGAA
GATACTTAACAAAGATATGATTGGAGCCCGGACAAAGATTTAGACTTATTGGGTAGGATGCATCGAGCTG
ACACCAAACCATGAGTTTACCAGTTACATACAACGTTTTAATTGTTATATGGAGGAGCTCACTGTTCTAG
TGTTGAAGGGATATCGGCTTCTTAATATTGGATGAATCATCACAACCTATTTTTTTTAAGCCAAGTGTTC
CGAACATAAAGAGGAAATGTAGCCCAAGGGCGAATTCGTTTAAACCTGCAGGACTAGTCCCTTTAGTGAG
GGTTAATTCTGAGCTTGGCGTAATCATGGTCATAGCTGTTTCCTGTGTGAAATTGTTATCCGCTCACAAT
TCCACACAACATACGAGCCGGAAGCATAAAGTGTAAAGCCTGGGGTGCCTAATGAGTGAGCTAACTCACA
TTAATTGCGTTGCGCTCACTGCCCGCTTTCCAGTCGGGAAACCTGTCGTGCCAGCTGCATTAATGAATCG
GCCAACGCGCGGGGAGAGGCGGTTTGCGTATTGGGCGCTCTTCCGCTTCCTCGCTCACTGACTCGCTGCG
CTCGGTCGTTCGGCTGCGGCGAGCGGTATCAGCTCACTCAAAGGCGGTAATACGGTTATCCACAGAATCA
SEQ ID NO: 278: coding Contig 1#6
ATGAGAGGGTACAAGTTTTGCTGTGATTTCCGGTACCTCCTCATCTTGGCTGCTGTCGCCTTCATCTACA
TACAGATGCGGCTTTTTGCGACACAGTCAGAATATGCAGACCGCCTTGCTGCTGCAATTGAAGCAGAAAA
TCACTGTACAAGTCAGACCAGATTGCTTATTGACCAGATTAGCCAGCAGCAAGGAAGAATAGTTGCTCTT
GAAGAACAAATGAAGCGTCAGGACCAGGAGTGCCGACAGTTAAGGGCTCTTGTTCAGGATCTTGAAAGTA
AGGGCATAAAAAAGTTGATCGGAAATGTACAGATGCCAGTGGCTGCTGTAGTTGTTATGGCTTGCAATCG
GGCTGACTACCTGGAAAAGACTATTAAATCCATCTTAAAATACCAAATATCTGTTGCGCCAAAATATCCT
CTTTTCATATCCCAGGATGGATCACATCCTGATGTTAGGAAGCTTGCTTTGAGCTATGATCAGCTGACGT
ATATGCAGCACTTGGATTTTGAACCTGTGCATACTGAAAGACCAGGGGAGCTGATTGCATACTACAAAAT
TGCACGTCATTACAAGTGGGCATTGGATCAGCTGTTTTACAAGCATAATTTTAGCCGTGTTATCATACTA
GAAGATGATATGGAAATTGCCCCTGACTTTTTTGACTTTTTTGAGGCTGGAGCTACTCTTCTTGACAGAG
ACAAGTCGATTATGGCTATTTCTTCTTGGAATGACAATGGACAAATGCAGTTTGTCCAAGATCCTTATGC
TCTTTACCGCTCTGATTTTTTTCCCGGTCTTGGATGGATGCTTTCAAAATCTACTTGGGACGAACTATCT
CCAAAGTGGCCAAAGGCTTACTGGGACGACTGGCTAAGACTCAAAGAGAATCACAGAGGTCGACAATTTA
TTCGCCCAGAAGTTTGCAGATCATATAATTTTGGTGAGCATGGTTCTAGTTTGGGGCAGTTTTTCAAGCA
GTATCTTGAGCCAATTAAACTAAATGATGTCCAGGTTGATTGGAAGTCAATGGACCTTAGTTACCTTTTG
GAGGACAATTACGTGAAACACTTTGGTGACTTGGTTAAAAAGGCTAAGCCCATCCATGGAGCTGATGCTG
TTTTGAAAGCATTTAACATAGATGGTGATGTGCGTATTCAGTACAGAGATCAACTAGACTTTGAAGATAT
CGCACGGCAATTTGGCATTTTTGAAGAATGGAAGGATGGTGTACCACGGGCAGCATATAAAGGAATAGTG
GTTTTCCGGTACCAAACGTCCAGACGTGTATTCCTTGTTGGCCCTGATTCGCTTCAACAACTCGGAAATG
AAGATACTTAA
SEQ ID NO: 279: Putative protein encoded by Contig 1#6
MRGYKFCCDFRYLLILAAVAFIYIQMRLFATQSEYADRLAAAIEAENHCTSQTRLLIDQISQQQGRIVAL
EEQMKRQDQECRQLRALVQDLESKGIKKLIGNVQMPVAAVVVMACNRADYLEKTIKSILKYQISVAPKYP
LFISQDGSHPDVRKLALSYDQLTYMQHLDFEPVHTERPGELIAYYKIARHYKWALDQLFYKHNFSRVIIL
EDDMEIAPDFFDFFEAGATLLDRDKSIMAISSWNDNGQMQFVQDPYALYRSDFFPGLGWMLSKSTWDELS
PKWPKAYWDDWLRLKENHRGRQFIRPEVCRSYNFGEHGSSLGQFFKQYLEPIKLNDVQVDWKSMDLSYLL
EDNYVKHFGDLVKKAKPIHGADAVLKAFNIDGDVRIQYRDQLDFEDIARQFGIFEEWKDGVPRAAYKGIV
VFRYQTSRRVFLVGPDSLQQLGNEDT
SEQ ID NO: 280: Contig 1#2
TAAAGGGACTAGTCCTGCAGGTTTAAACGAATTCGCCCTTCATTGACTTGATCCTAACTGAACAGGCAAA
GTAAATCCACGGATGAAACACTCATAACTGAACAGTGATAGACTATTCGCTTTCTCCTAAAGCCTTCAAT
CGAAATCGCACGATGAGAGGGTACAAGTTTTGCTGTGATTTCCGGTACCTCCTCATCTTGGCTGCTGTCG
CCTTCATCTACATACAGATGCGGCTTTTTGCGACACAGTCAGAATATGCAGATCGCCTTGCTGCTGCAAT
TGAAGCAGAAAATCACTGTACAAGTCAGACCAGATTGCTTATTGACCAGATTAGCCAGCAGCAAGGAAGA
ATAGTTGCTCTTGAAGAACAAATGAAGCGTCAGGACCAGGAGTGCCGACAGTTAAGGGCTCTTGTTCAGG
ATCTTGAAAGTAAGGGCATAAAAAAGTTGATCGGAAATGTACAGATGCCAGTGGCTGCTGTAGTTGTTAT
GGCTTGCAATCGGGCTGACTACCTGGAAAAGACTATTAAATCCATCTTAAAATACCAAATATCTGTTGCG
CCAAAATATCCTCTTTTCATATCCCAGGATGGATCACATCCTGATGTTAGGAAGCTTGCTTTGAGCTATG
ATCAGCTGACGTATATGCAGCACTTGGATTTTGAACCTGTGCATACTGAAAGACCAGGGGAGCTGATTGC
ATACTACAAAATTGCACGTCATTACAAGTGGGCATTGGATCAGCTGTTTTACAAGCATAATTTTAGCCGT
GTTATCATACTAGAAGATGATATGGAAATTGCCCCTGATTTTTTTGACTTTTTTGAGGCTGGAGCTACTC
TTCTTGACAGAGACAAGTCGATTATGGCTATTTCTTCTTGGAATGACAATGGACAAATGCAGTTTGTCCA
AGATCCTTATGCTCTTTACCGCTCTGATTTTTTTCCCGGTCTTGGATGGATGCTTTCAAAATCTACTTGG
GACGAACTATCTCCAAAGTGGCCAAAGGCTTACTGGGACGACTGGCTAAGACTCAAAGAGAATCACAGAG
GTCGACAATTTATTCGCCCAGAAGTTTGCAGATCATATAATTTTGGTGAGCATGGTTCTAGTTTGGGGCA
GTTTTTCAAGCAGTATCTTGAGCCAATTAAACTAAATGATGTCCAGGTTGATTGGAAGTCAATGGACCTT
AGTTACCTTTTGGAGGACAATTACGTGAAACACTTTGGTGACTTGGTTAAAAAGGCTAAGCCCATCCATG
GAGCTGATGCTGTTTTGAAAGCATTTAACATAGATGGTGATGTGCGTATTCAGTACAGAGATCAACTAAA
CTTTGAAGATATCGCACGGCAATTTGGCATTTTTGAAGAATGGAAGGATGGTGTACCACGGGCAGCATAT
AAAGGAATAGTGGTTTTCCGGTACCAAACGTCCAGACGTGTATTCCTTGTTGGCCCTGATTCGCCTCAAC
AACTCGGAAATGAAGATACTTAACAAAGATATGATTGGAGCCCGGACAAAGATTTAGACTTATTGGGTAG
GATGCATCGAGCTGACACCAAACCATGAGTTTACCAGTTACATACAACGTTTTAATTGTTATATGGAGGA
GCTCACTGTTCTAGCGTTGAAGGGATATCGGCTTCTTAATATTGGATGAATCATCACAACCTATTTTTTT
TAAGCCAAGTGTTCCGAACATAAAGAGGAAATGTAGCCCTGAAGGGCGAATTCGCGGCCGCTAAATTCAA
TTCGCCCTATAGTGAGTCGTATTACAATTCACTGGCCGTCGTTTTACAACGTCGTGACTGGGAAAACCCT
GGCGTTACCCAACTTAATCGCCTTGCAGCACATCCCCCTTTCGCCAGCTGGCGTAATAGCGAAGAGGCCC
GCACCGATCGCCCTTCCCAACAGTTGCGCAGCCTATACGTACGGCAGTTTAAGGTTTACACCTATAAAAG
AGAGAGCCGTTATCGTCTGTTTGTGGATGTACAGAGTGATATTATTGACACGCCGGGGCGACGGATGGTG
ATCCCCCTGGCCAGTGCACGTCTGCTGTCAGATAAAGTCTCCCGTGAACTTTACCCGGTGGTGCATATCG
GGGATGAAAGCTGGCGCATGATGACCACCGATATGGCCAGTGTGCCGGTCTCCGTTATCGGGGAAGAAGT
GGCTGATCTCAGCCACCGCGAAAATGACATCAAAAACGCCATTAACCTGATGTTCTGGGGAATATAAATG
TCAGGCATGAGATTATCAAAAAGGATCTTCACCTAGATCCTTTTCACGTAGAAAGCCAGTCC
SEQ ID NO: 281: coding Contig 1#2
ATGAGAGGGTACAAGTTTTGCTGTGATTTCCGGTACCTCCTCATCTTGGCTGCTGTCGCCTTCATCTACA
TACAGATGCGGCTTTTTGCGACACAGTCAGAATATGCAGATCGCCTTGCTGCTGCAATTGAAGCAGAAAA
TCACTGTACAAGTCAGACCAGATTGCTTATTGACCAGATTAGCCAGCAGCAAGGAAGAATAGTTGCTCTT
GAAGAACAAATGAAGCGTCAGGACCAGGAGTGCCGACAGTTAAGGGCTCTTGTTCAGGATCTTGAAAGTA
AGGGCATAAAAAAGTTGATCGGAAATGTACAGATGCCAGTGGCTGCTGTAGTTGTTATGGCTTGCAATCG
GGCTGACTACCTGGAAAAGACTATTAAATCCATCTTAAAATACCAAATATCTGTTGCGCCAAAATATCCT
CTTTTCATATCCCAGGATGGATCACATCCTGATGTTAGGAAGCTTGCTTTGAGCTATGATCAGCTGACGT
ATATGCAGCACTTGGATTTTGAACCTGTGCATACTGAAAGACCAGGGGAGCTGATTGCATACTACAAAAT
TGCACGTCATTACAAGTGGGCATTGGATCAGCTGTTTTACAAGCATAATTTTAGCCGTGTTATCATACTA
GAAGATGATATGGAAATTGCCCCTGATTTTTTTGACTTTTTTGAGGCTGGAGCTACTCTTCTTGACAGAG
ACAAGTCGATTATGGCTATTTCTTCTTGGAATGACAATGGACAAATGCAGTTTGTCCAAGATCCTTATGC
TCTTTACCGCTCTGATTTTTTTCCCGGTCTTGGATGGATGCTTTCAAAATCTACTTGGGACGAACTATCT
CCAAAGTGGCCAAAGGCTTACTGGGACGACTGGCTAAGACTCAAAGAGAATCACAGAGGTCGACAATTTA
TTCGCCCAGAAGTTTGCAGATCATATAATTTTGGTGAGCATGGTTCTAGTTTGGGGCAGTTTTTCAAGCA
GTATCTTGAGCCAATTAAACTAAATGATGTCCAGGTTGATTGGAAGTCAATGGACCTTAGTTACCTTTTG
GAGGACAATTACGTGAAACACTTTGGTGACTTGGTTAAAAAGGCTAAGCCCATCCATGGAGCTGATGCTG
TTTTGAAAGCATTTAACATAGATGGTGATGTGCGTATTCAGTACAGAGATCAACTAAACTTTGAAGATAT
CGCACGGCAATTTGGCATTTTTGAAGAATGGAAGGATGGTGTACCACGGGCAGCATATAAAGGAATAGTG
GTTTTCCGGTACCAAACGTCCAGACGTGTATTCCTTGTTGGCCCTGATTCGCCTCAACAACTCGGAAATG
AAGATACTTAA
SEQ ID NO: 282: Putative protein encoded by Contig 1#2
MRGYKFCCDFRYLLILAAVAFIYIQMRLFATQSEYADRLAAAIEAENHCTSQTRLLIDQISQQQGRIVAL
EEQMKRQDQECRQLRALVQDLESKGIKKLIGNVQMPVAAVVVMACNRADYLEKTIKSILKYQISVAPKYP
LFISQDGSHPDVRKLALSYDQLTYMQHLDFEPVHTERPGELIAYYKIARHYKWALDQLFYKHNFSRVIIL
EDDMEIAPDFFDFFEAGATLLDRDKSIMAISSWNDNGQMQFVQDPYALYRSDFFPGLGWMLSKSTWDELS
PKWPKAYWDDWLRLKENHRGRQFIRPEVCRSYNFGEHGSSLGQFFKQYLEPIKLNDVQVDWKSMDLSYLL
EDNYVKHFGDLVKKAKPIHGADAVLKAFNIDGDVRIQYRDQLNFEDIARQFGIFEEWKDGVPRAAYKGIV
VFRYQTSRRVFLVGPDSPQQLGNEDT
* Where appropriate, coding sequences are underlined, start and stop codons are
given in bold in the the above SEQ ID NOs..

While the invention has been described in detail and foregoing description, such description are to be considered illustrative or exemplary and not restrictive. It will be understood that changes and modifications may be made by those of ordinary skill within the scope and spirit of the following claims. Various publications and patents are cited throughout the specification. The disclosures of each of these publications and patents are incorporated by reference in its entirety.

### Deposit

The following seed samples were deposited with NCIMB, Ferguson Building, Craibstone Estate, Bucksbum, Aberdeen AB21 9YA, Scotland, UK on Jan. 6, 2011 under the provisions of the Budapest Treaty in the name of Philip Morris Products S.A:

| PM seed line designation | Deposition date | Accession No |
|---|---|---|
| PM016 | 6 Jan. 2011 | NCIMB 41798 |
| PM021 | 6 Jan. 2011 | NCIMB 41799 |
| PM092 | 6 Jan. 2011 | NCIMB 41800 |
| PM102 | 6 Jan. 2011 | NCIMB 41801 |
| PM132 | 6 Jan. 2011 | NCIMB 41802 |
| PM204 | 6 Jan. 2011 | NCIMB 41803 |
| PM205 | 6 Jan. 2011 | NCIMB 41804 |
| PM215 | 6 Jan. 2011 | NCIMB 41805 |
| PM216 | 6 Jan. 2011 | NCIMB 41806 |
| PM217 | 6 Jan. 2011 | NCIMB 41807 |

1. A genetically modified Nicotiana tabacum plant cell, comprising:
at least a modification of a first target nucleotide sequence in a genomic region comprising a coding sequence for a N-acetyl-glucosaminyltransferase,
wherein the activity or the expression of glycosyltransferase in the modified plant cell is reduced relative to a unmodified plant cell, and
wherein alpha-1,3-fucose or beta-1,2-xylose, or both, on an N-glycan of a protein produced in the modified plant cell is reduced relative to an unmodified plant cell.

2. The modified Nicotiana tabacum plant cell of claim 1, further comprising:
(a) at least a modification of a second target nucleotide sequence in a genomic region comprising a coding sequence for β(1,2)-xylosyltransferase;
(b) at least a modification of a third target nucleotide sequence in a genomic region comprising a coding sequence for α(1,3)-fucosyltransferase; or
(c) a combination of (a) and (b).

3. The modified Nicotiana tabacum plant cell of claim 2, further comprising a modification in an allelic variant of the first target nucleotide sequence, the second target nucleotide sequence, the third target nucleotide sequence, or a combination of any two or more of the foregoing target nucleotide sequences.

4. The modified Nicotiana tabacum plant cell of claim 1, wherein the first target nucleotide sequence is
a. at least 70% identical to a nucleotide sequence selected from the group consisting of SEQ ID NOs: 12, 13, 40, 41, 233, 256, 259, 262, 265, 268, 271, 274, 277, 280; or
b. at least 95% identical to a nucleotide sequence selected from the group consisting of SEQ ID NOs: 20, 21, 212, 213, 219, 220, 223, 227, 229, 234, 257, 260, 263, 266, 269, 272, 275, 278, 281.

5. The modified Nicotiana tabacum plant cell of claim 2, wherein the second target nucleotide sequence is
a. at least 70% identical to a nucleotide sequence selected from the group consisting of SEQ ID NOs: 1, 4, 5, and 17; or
b. at least 95% identical to a nucleotide sequence selected from the group consisting of SEQ ID NOs: 8 and 18.

6. The modified Nicotiana tabacum plant cell of claim 2, wherein the third target nucleotide sequence is
a. at least 70% identical to a nucleotide sequence selected from the group consisting of SEQ ID NOs 27, 32, 37, and 47; or
b. at least 95% identical to a nucleotide sequence selected from the group consisting of SEQ ID NOs: 28, 33, 38, and 48.

7. The modified Nicotiana tabacum plant cell of claim 1 plant according to any one of the preceding claims, wherein the plant cell is a cell of Nicotiana tabacum cultivar PM132, deposited under accession NCIMB 41802.

8. A plant that is a progeny of the plant of claim 27, wherein said progeny plant comprises at least one of the modifications as defined in claim 1, wherein the activity or the expression of the glycosyltransferase is reduced relative to an unmodified plant and (ii) the alpha-1,3-fucose or beta-1,2-xylose, or both, on the N-glycan of the protein produced in the modified plant is reduced relative to an unmodified plant.

9. A method for producing a heterologous protein, said method comprising:
introducing into a modified Nicotiana tabacum plant cell as defined in claim 1 an expression construct comprising a nucleotide sequence that encodes a heterologous protein; and
culturing the modified plant cell that comprises the expression construct such that the heterologous protein is produced, and optionally, regenerating a plant from the plant cell, and growing the plant and its progenies.

10. A polynucleotide comprising a nucleotide sequence encoding
a. an N-acetylglucosaminyltransferase or a fragment thereof, which nucleotide sequence
(i) is selected from the group consisting of SEQ ID NOs: 12, 13, 40, 41, 233, 256, 259, 262, 265, 268, 271, 274, 277, and 280;
(ii) is selected from the group consisting of SEQ ID NOs: 20, 21, 212, 213, 219, 220, 223, 227, 229, 234, 257, 260, 263, 266, 269, 272, 275, 278, and 281;
(iii) is at least 95% identical to the nucleotide sequence of (i) or (ii); or
(iv) allows a polynucleotide probe consisting of the nucleotide sequence of (i), (ii), or (iii), or a complement thereof, to hybridize, particularly under stringent conditions;
b. a β(1,2)-xylosyltransferase or a fragment thereof, which nucleotide sequence
(i) is selected from the group consisting of SEQ ID NOs: 1, 4, 5, 7 and 17;
(ii) is selected from the group consisting of SEQ ID NOs: 8 and 18;
(iii) is at least 95% identical to the nucleotide sequence of (i) or (ii); or
(iv) allows a polynucleotide probe consisting of the nucleotide sequence of (i), (ii), or (iii), or a complement thereof, to hybridize, particularly under stringent conditions; or
c. an α(1,3)-fucosyltransferase or a fragment thereof, which nucleotide sequence
(i) is selected from the group consisting of SEQ ID NOs: 27, 32, 37, and 47;
(ii) is selected from the group consisting of SEQ ID NOs: 28, 33, 38, and 48;
(iii) is at least 95% identical to the nucleotide sequence of (i) or (ii); or
(iv) allows a polynucleotide probe consisting of the nucleotide sequence of (i), (ii), or (iii), or a complement thereof, to hybridize, particularly under stringent conditions.

11. A polypeptide encoded by a polynucleotide of claim 10, wherein said polypeptide is
a. an N-acetylglucosaminyltransferase exhibiting an amino acid sequence as shown in SEQ ID NOs: 214, 215, 217, 218, 221, 222, 224, 228, 230, 235, 258, 264, 267, 270, 273, 276, 279 and 282;
b. a β(1,2)-xylosyltransferase exhibiting an amino acid sequence as shown in SEQ ID NOs: 9 and 19;
c. an α(1,3)-fucosyltransferase exhibiting an amino acid sequence as shown in SEQ ID NOs: 29, 34, 39, and 49; or
d. an amino acid sequence that is at least 95% identical to the amino acid sequence of (i), (ii), or (iii).

12. Use of a genomic nucleotide sequence as defined in claim 10 for identifying a target site in
a. a first target nucleotide sequence in a genomic region comprising a coding sequence for a N-acetylglucosaminyltransferase; or
b. the first target nucleotide sequence of a) and a second target nucleotide sequence in a genomic region comprising a coding sequence for a β(1,2)-xylosyltransferase; or
c. the first target nucleotide sequence of a) and a third target nucleotide sequence in a genomic region comprising a coding sequence for an α(1,3)-fucosyltransferase; or
d. all target nucleotide sequences a), b) and c);
for modification such that (i) the activity or the expression of an N-acetylglucosaminyltransferase, or of an N-acetylglucosaminyltransferase and a β(1,2)-xylosyltransferase, or of an N-acetylglucosaminyltransferase and an α(1,3)-fucosyltransferase or of an N-acetylglucos-aminyltransferase, a β(1,2)-xylosyltransferase, and an α(1,3)-fucosyltransferase and, optionally, of at least one allelic variant thereof, in a modified plant cell comprising the modification is reduced relative to an unmodified plant cell, and (ii) the alpha-1,3-fucose or beta-1,2-xylose, or both, on a N-glycan of a protein in a modified plant cell comprising the modification is reduced relative to an unmodified plant cell.

13. Use of a non-natural zinc finger protein that selectively binds a genome nucleotide sequence or a coding sequence as defined in claim 10, for making a zinc finger nuclease that introduces a double-stranded break in at least one of the target nucleotide sequences.

14. A plant composition comprising a heterologous protein obtained from plant cells as defined in claim 1, wherein the alpha-1,3-fucose or beta-1,2-xylose, or both, on the N-glycan of the heterologous protein is reduced relative to that produced in an unmodified plant cell.

15. A method for producing a Nicotiana tabacum plant cell or of a Nicotiana tabacum plant comprising the modified plant cells capable of producing humanized glycoproteins, the method comprising:
(i) modifying in the genome of a tobacco plant cell
a. a first target nucleotide sequence in a genomic region comprising a coding sequence for a N-acetylglucosaminyltransferase; or
b. the first target nucleotide sequence of a) and a second target nucleotide sequence in a genomic region comprising a coding sequence for a β(1,2)-xylosyltransferase or an α(1,3)-fucosyltransferase; or
c. the first target nucleotide sequence of a) and the second target nucleotide sequence of b) and a third target nucleotide sequence in a genomic region comprising a coding sequence for a β(1,2)-xylosyltransferase or an α(1,3)-fucosyltransferase; and, optionally,
d. a target nucleotide in a genomic region comprising an allelic variant of (a), (b) or (c), or of a combination of any two or more of the foregoing target nucleotide sequences.
(ii) identifying and, optionally, selecting a modified plant or plant cell comprising the modification in the target nucleotide sequence,
wherein the activity or the expression of the glycosyltransferases as defined in a), b), c) and d), and, optionally, of at least one allelic variant thereof, in the modified plant or plant cell is reduced relative to an unmodified plant cell and the glycoproteins produced by said modified plant or plant cell lack alpha-1,3-linked fucose residues and beta-1,2-linked xylose residues in their N-glycan.

16. The method of claim 15, wherein the target nucleotide sequence comprises a nucleotide sequence of a polynucleotide as defined in claim 10.

17. The method of claim 15, wherein the plant is Nicotiana tabacum cultivar PM132, deposited under accession NCIMB 41802.

18. The method of claim 15, wherein the modification of the genome of a tobacco plant or plant cell comprises
a. identifying in the target nucleotide sequence of a Nicotiana tabacum plant or plant cell and, optionally, in at least one allelic variant thereof, a target site,
b. designing, based on the nucleotide sequence as defined in claim 10, a mutagenic oligonucleotide capable of recognizing and binding at or adjacent to said target site, and
c. binding the mutagenic oligonucleotide to the target nucleotide sequence in the genome of a tobacco plant or plant cell under conditions such that the genome is modified.

19. The method of claim 18, wherein a mutagenic oligonucleotide is used in genome editing technology, particularly in zinc finger nuclease-mediated mutagenesis, tilling, homologous recombination, oligonucleotide-directed mutagenesis, or meganuclease-mediated mutagenesis, or a combination of the foregoing technologies.

20. The modified Nicotiana tabacum plant cell of claim 1, further comprising a modification in an allelic variant of the first target nucleotide sequence

21. The modified Nicotiana tabacum plant cell of claim 4, wherein the first target nucleotide sequence is
a. at least 80% identical to a nucleotide sequence selected from the group consisting of SEQ ID NOs: 12, 13, 40, 41, 233, 256, 259, 262, 265, 268, 271, 274, 277, 280; or
b. at least 98% identical to a nucleotide sequence selected from the group consisting of SEQ ID NOs: 20, 21, 212, 213, 219, 220, 223, 227, 229, 234, 257, 260, 263, 266, 269, 272, 275, 278, 281.

22. The modified Nicotiana tabacum plant cell of claim 4, wherein the first target nucleotide sequence is
a. at least 90% identical to a nucleotide sequence selected from the group consisting of SEQ ID NOs: 12, 13, 40, 41, 233, 256, 259, 262, 265, 268, 271, 274, 277, 280; or
b. at least 99% identical to a nucleotide sequence selected from the group consisting of SEQ ID NOs: 20, 21, 212, 213, 219, 220, 223, 227, 229, 234, 257, 260, 263, 266, 269, 272, 275, 278, 281.

23. The modified Nicotiana tabacum plant cell of claim 5, wherein the second target nucleotide sequence is
a. at least 80% identical to a nucleotide sequence selected from the group consisting of SEQ ID NOs: 1, 4, 5, and 17; or
b. at least 98% identical to a nucleotide sequence selected from the group consisting of SEQ ID NOs: 8 and 18.

24. The modified Nicotiana tabacum plant cell of claim 5, wherein the second target nucleotide sequence is
a. at least 90% identical to a nucleotide sequence selected from the group consisting of SEQ ID NOs: 1, 4, 5, and 17; or
b. at least 99% identical to a nucleotide sequence selected from the group consisting of SEQ ID NOs: 8 and 18.

25. The modified Nicotiana tabacum plant cell of claim 6, wherein the third target nucleotide sequence is
a. at least 80% identical to a nucleotide sequence selected from the group consisting of SEQ ID NOs 27, 32, 37, and 47; or
b. at least 98% identical to a nucleotide sequence selected from the group consisting of SEQ ID NOs: 28, 33, 38, and 48.

26. The modified Nicotiana tabacum plant cell of claim 6, wherein the third target nucleotide sequence is
a. at least 90% identical to a nucleotide sequence selected from the group consisting of SEQ ID NOs 27, 32, 37, and 47; or
b. at least 99% identical to a nucleotide sequence selected from the group consisting of SEQ ID NOs: 28, 33, 38, and 48.

27. A plant comprising a modified Nicotiana tabacum plant cell according to claim 1.

28. The method of claim 9, wherein the heterologous protein is selected from the group consisting of a vaccine antigen, a cytokine, a hormone, a coagulation protein, an apolipoprotein, an enzyme for replacement therapy in human, and an immunoglobulin or a fragment thereof.

29. The polynucleotide of claim 10,
wherein the nucleotide sequence encoding the N-acetylglucosaminyltransferase or the fragment thereof is at least 98% identical to a nucleotide sequence
(i) selected from the group consisting of SEQ ID NOs: 12, 13, 40, 41, 233, 256, 259, 262, 265, 268, 271, 274, 277, and 280; or
(ii) selected from the group consisting of SEQ ID NOs: 20, 21, 212, 213, 219, 220, 223, 227, 229, 234, 257, 260, 263, 266, 269, 272, 275, 278, and 281;
wherein the nucleotide sequence encoding the β(1,2)-xylosyltransferase or a fragment thereof is at least 98% identical to a nucleotide sequence
(i) selected from the group consisting of SEQ ID NOs: 1, 4, 5, 7 and 17; or
(ii) selected from the group consisting of SEQ ID NOs: 8 and 18; or
wherein the nucleotide sequence encoding the α(1,3)-fucosyltransferase or a fragment thereof is at least 98% identical to a nucleotide sequence
(i) selected from the group consisting of SEQ ID NOs: 27, 32, 37, and 47; or
(ii) selected from the group consisting of SEQ ID NOs: 28, 33, 38, and 48.

30. The polynucleotide of claim 10,
wherein the nucleotide sequence encoding the N-acetylglucosaminyltransferase or the fragment thereof is at least 99% identical to a nucleotide sequence
(i) selected from the group consisting of SEQ ID NOs: 12, 13, 40, 41, 233, 256, 259, 262, 265, 268, 271, 274, 277, and 280; or
(ii) selected from the group consisting of SEQ ID NOs: 20, 21, 212, 213, 219, 220, 223, 227, 229, 234, 257, 260, 263, 266, 269, 272, 275, 278, and 281;
wherein the nucleotide sequence encoding the β(1,2)-xylosyltransferase or a fragment thereof is at least 99% identical to a nucleotide sequence
(iii) selected from the group consisting of SEQ ID NOs: 1, 4, 5, 7 and 17; or
(iv) selected from the group consisting of SEQ ID NOs: 8 and 18; or
wherein the nucleotide sequence encoding the α(1,3)-fucosyltransferase or a fragment thereof is at least 99% identical to a nucleotide sequence
(i) selected from the group consisting of SEQ ID NOs: 27, 32, 37, and 47; or
(ii) selected from the group consisting of SEQ ID NOs: 28, 33, 38, and 48.

31. The polypeptide of claim 11, wherein said polypeptide comprises an amino acid sequence that is at least 98% identical to the amino acid sequence of
(i) an N-acetylglucosaminyltransferase exhibiting an amino acid sequence as shown in SEQ ID NOs: 214, 215, 217, 218, 221, 222, 224, 228, 230, 235, 258, 264, 267, 270, 273, 276, 279 and 282;
(ii) a β(1,2)-xylosyltransferase exhibiting an amino acid sequence as shown in SEQ ID NOs: 9 and 19; or
(iii) an α(1,3)-fucosyltransferase exhibiting an amino acid sequence as shown in SEQ ID NOs: 29, 34, 39, and 49.

32. The polypeptide of claim 11, wherein said polypeptide comprises an amino acid sequence that is at least 99% identical to the amino acid sequence of
(i) an N-acetylglucosaminyltransferase exhibiting an amino acid sequence as shown in SEQ ID NOs: 214, 215, 217, 218, 221, 222, 224, 228, 230, 235, 258, 264, 267, 270, 273, 276, 279 and 282;
(ii) a β(1,2)-xylosyltransferase exhibiting an amino acid sequence as shown in SEQ ID NOs: 9 and 19; or
(iii) an α(1,3)-fucosyltransferase exhibiting an amino acid sequence as shown in SEQ ID NOs: 29, 34, 39, and 49.


SEQUENCE LISTING (282 sequences)

SEQ ID NO: 1 - DNA, 1667 bp, Nicotiana tabacum
gcaaagctaattcaacaaaactgagaaattaatttagattgaaattgatatttagaaaataaatgagatacaaattgcaactttccatctcaaagttcactaaaaatgaagtacaaatttgagtaatcaactattttatttgataatcgacgtaggaataagcatccttttgcatacaagatggaatttattaattagcggttttgtcgagaaattatcaaaaacaacatatcgtataaatagcaaacgtatagttatagaaatggtggagaattattattgaaaatctaaatatttgcggtaactacggattgataacttattggtacataatttaagttatatacaacaacattataaattttttaattgtatcgtgtaatagtttttaaagaatattatacctccttatttagaattaaattaatttatttttaaaattctcatttgattctaatttacagtcatatatttatgccttagtttagaaaaacacaaattttaaatttttttatttatttcttaaactaaatactcaatcaaataaattggtacgaaccgagtgtttgtataaaactcaatttaatcaccttagtaaaattattagtactgcatacaaataaggatataatataacggatcaaataccgtaaagtgtaattacaactcaaataatcacagtttactaacgtcgttaaaacctctgtactaagaaaactacatactcctagtacccacgaaaacaaccagtcagagagagaagaagatgaacaagaaaaagctgaaatttcttgtttctctcttcgctctcaactcaatcactctctatctctacttctcttcccactctgatcacttccgtcacaaatccccccaaaaccactttcctaatacccaaaaccactattccctgtcggaaaaccaccatgataatttccactcttctgtcacttcccaatataccaagccttggccaattttgccctcctacctcccctggtctcagaatcctaatgtttctttgagatcgtgcgagggttacttcggtaatgggtttactctcaaagttgatcttctcaaaacttcgccggagcttcaccagaaattcggcgaaaacaccgtatccggcgacggcggatggtttaggtgttttttcagtgagactttgcagagttcgatttgcgagggaggtgctatacgaatgaatccggacgagattttgatgtctcgtggaggcgagaaattggagtcggttattggtaggagtgaagatgatgagctgcccgtgttcaaaaatggagcttttcagattaaagttactgataaactgaaaattgggaaaaaattagtggatgaaaaaatcttgaataaatacttaccggaaggtgcaatttcaaggcacactatgcgtgaattaattgactctattcagttagttggcgccgatgaatttcactgttctgaggttagattttgaaattttgcttgatctttaaattaaaggtttgaactttgtgaatgttggcagatatggaatacaataatggattttgtttgatctgtttaatgaagattgtctagaacctcattgttataaatatggtttgtttgcttcattaattaaagagcattccttaaaatctcgactagatgccagataacaccag

SEQ ID NO: 2 - DNA, 20 bp, artificial sequence
  /note=“Description of artificial sequence NGSG10043 forward primer”
acccacgaaaacaaccagtc

SEQ ID NO: 3 - DNA, 20 bp, artificial sequence
  /note=“Description of artificial sequence NGSG10043 reverse primer”
cggcgaagttttgagaagat

SEQ ID NO: 4 - DNA, 6046 bp, Nicotiana tabacum
gatcgtgccttgcacttgcaccggcatataatatctagttttaatagaccacaaattaatgaaatctctcggacatctaacaaaagtaatcattattctgacgtaccaagaactggtatacagaaagttacaaactatacaaaacagaagagaaaaagataaaacaaaataaaaacgctgttaaagcgcatgcattgatccaattttttttatgaaagtatggatgctttataaaattggatttaagctatatactgatagaataatttattttacaatatcagtataacattgatggtagaactagcaacttatacaaaagaattcaaaaaagatgtcaaaattaggatttaaacatgtgattaaatatctagctaactagtgttactttgaccagttggtccttatttcgttgttcgtcctcaaagttttatcataaaatcttgaggtacaacctctttatttaacacttgggcgactctattttttttaaaacttaatgtcaccattgcagtgagagattgtattattgttattatcgtattataaatgaagcataacaggatagagttaaagtgaaattcaagcacaagtacataaagaagaagaaaatactatctgtaaaggaatgttgaatgtcctcactaacgtctcaaaaacctgaacactatttagcggcagtgtaagagaataacagagacagagcttaaaatatgacaaaatctctgttcaaagtctcatttcttttgtttggttacaagttacaaccatatcttcactgaagcatcatactgacgttcgtgttaatctgcttcttcaatatgtcaaatgtcttatgcctttaaacttgacatctgatgggatgaatgtgttgaatgcaaaactgtaactgggagtgcaagtgttacaactacaaaacaaactctgttgaaatcatctctacataaggagggaagagggtggccggattgatgggaaaacaacacagaacatggatggttttctcatgttcttatcctatgatcattaatgagaataatgaaaaacaattgacaatgaattgtaagtaagtgtaccaacctaatctttcacatacacgtgttaattgtgaattgtaatcttcaacaccataaagaatatgctcttcaatgagctcaccattccacaatcaggattccatgcttccatttgatatgattgctctggtagtccataaccgagtcagagcgggctaataaattagtagctgctggaaattgccaagcttatcaacaccagattgaggacctagattgcaaccattgactacagaactgcgtgtgaaacatatttatcaaaattcccaggctccaagttccagaaccccgaattcctctatccttccccgtcttttagagaaaagccgaactattcaaactgtcgagcggacttagcacccaagactcctcaaaatgctgctgagcttgtcgatcacaactggaggatccgcataagacccctccaaatatatgggatggtactccaatcctttccattgtgcaatcagagcaaaatgggggcgcctatattcgctgcttataatttctagtattacagcttttggtgctgcagaaactatgtgagttagacctgctccatgagcaccaacaatgacagatgcatcttggattgctcgaacttgctctttcatggacatgtgggcaaacaatccgttaattacatttaatttgcactccgagtggttcaaggcccagctctttatggaatcaaatacttgctcttcattgctaagcctagactgtacctttccaccatgacgtgggtgagctaaataatcctcacgtctaacaaagaggacattagggcctgtgactgtccttgggatgttctgtctatccacaggaaatccaaaggctgccctgatcatctccccaaactcggacaaccgtgcagttctcttatcatcaggattttgccacaaatcatgggcagaagctccattacaatctatagtttctgtcagtcccttaaacagggcagtttcatatcccaaaggcgagagaacggcgtgacggaaacaaactgggccactaaagttcttagcataagtgaggcttgaaaagagtgctttccatgtttcctccaattgtgtctgaattttttcccaaacagaaaacagaaaaaaaattactcaaaagaattacatgaagaaaatgaatttgaagataaatcaaaacacgcccccaatatggccaagtaaaattcatgcagcaagcttacagtagtggagtgaaagaactttcttcacattaaagcatctctccaagaattaaaagtggagtgaaagaactttcttcacattaaagcatctctccaagaattaaagtatgcaataggaagcaattttatgagacgtgatgtaaggtaaaaataaattgtcccagaaatgaaatctttgctagtttttcttcatagcaaaagcacttttcaccacaaagggaccattcagagaaacagtgacacgtaaatagaagtaagaatgagccaaccggaacaactaggtattgggacaattattgtagtcagtcaaacagaaagctgaaacaaatggaaactcaggattaagtctgaccaatttgacacaagcatctcaagagcattgcctgctagaaaataattgctgatttcatttctttcatccataacacagtacaatgcatgccatcgttatcaatactttcaaacatacctcacaatggccatctacaaaaaccaaatgtggccgactgggcaagccagtaaccctggatgccacgtatgcactataccaatcggtaactgtgtggaaaaggtttgcatactcaaatcgtgtaatcaaaagtgacggctcctcaatccactgaaatgaaaattagaaaaatgacagtaacgtaaggcatcgattacggaaagatggcatgaatcaaagaaggagcttccttaaaagctagcatggttgcttgaacactaacatctaggctctagcagggataattttttgataagtacataaagaactggaaaatgttgatccagaaatatcccaaagtcatgagcaatgtaaaaaacaaatctaagaccatttgtcatcatttaagctggttctcaatcattagtaaataaggtaacataacctggcagtgctacatccttgatgtgttcctcttattagtgcacaatactgcttcagaatttgattagactccgaagatctaaaattaactggtcaaactcgaactttgagccagtagctcataaccaagcgacagctccctatctctggttgggatttcttcctagatataagttttcataacttacaagccatgtatcagacactgtcaaatcaagtcgatagtcctcaaaatgcaacaaccagttgtacaactaaagcttcaaaaggcatttactcgaattagttatgcaaattttaaggaataaaattaactagttcagacagatgctaccatattctgtgtgtaaaacagaaataaagagatgaaaggagaaaaaatggcttttcttcacttttctattgctactccgtctaaggttcaatctgagtagcaatcaaacaaaggaattgatagaatttgaaaaccaaaatcaataaagggaaaagtaaagagcataaatcaactaactttctccatgttagacatcaaaagttataggagtaatcatcttagaccacatacatttaccattttccaacttgtggttaatttctgactctgtattacttttcaacccttgaatttaatctgttgctcctccccagatgactggggagtacaagtgcagttacccattcagtactagatccaaatccaaaattccaaatacacttaaagatatacactgaaatcatgacaaattctttagtgaaacatttaagaatgactatcttatctgattaaatgaaaaaaaatccataatccaaaagtcaactaactggtgttatctggcatctagtcgagattttaaggaatgctctttaattaatgaagcaaacaaaccatatttataacaatgaggttctagacaatcttcattaaacagatcaaacaaaatccattattgtattccatatctgccaacattcacaaagttcaaacctttaatttaaagatcaagcaaaatttcaaaatctaacctcagaacagtgaaattcatcggcgccaactaactgaatagagtcaattaattcacgcatagtgtgccttgaaattgcaccttccggtaagtatttattcaagattttttcatccactaattttttcccaattttcagtttatcagtaactttaatctgaaaagctccatttttgaacacgggcagctcatcatcttcactcctaccaataaccgactccaatttctcgcctccacgagacatcaaaatctcgtccggattcattcgtatagcacctccctcgcaaatcgaactctgcaaagtctcactgaaaaaacacctaaaccatccgccgtcgccggatacggtgttttcgccgaatttctggtgaagctccggcgaagttttgagaagatcaactttgagagtaaacccattaccgaagtaaccctcgcacgatctcaaagaaacattaggattctgagaccaggggaggtaggagggcaaaattggccaaggcttggtatattgggaagtgacagaagagtggaaattatcatggtggttttccgacagggaatagtggttttgggtattaggaaagtggttttggggggatttgtgacggaagtgatcagagtgggaagagaagtagagatagagagtgattgagttgagagcgaagagagaaacaagaaatttcagctttttcttgttcatcttcttctctctctgactggttgttttcgtgggtactaggagtatgtagttttcttagtacagaggttttaacgacgttagtaaactgtgattatttgagttgtaattacactttacggtatttgatccgttatattatatccttatttgtatgcagtactaataattttactaaggtgattaaattgagttttatacaaacactcggttcgtaccaatttatttgattgagtatttagtttaagaaataaataaaaaaatttaaaatttgtgtttttctaaactaaggcataaatatatgactgtaaattagaatcaaatgagaattttaaaaataaattaatttaattctaaataaggaggtataatattctttaaaaactattacacgatacaattaaaaaatttataatgttgttgtatataacttaaattatgtaccaataagttatcaatccgtagttaccgcaaatatttagattttcaataataattctccaccatttctataactatacgtttgctatttatacgatatgttgtttttgataatttctcgacaaaaccgctaattaataaattccatcttgtatgcaaaaggatgcttattcctacgtcgattatcaaataaaatagttgattactcaaatttgtacttcatttttagtgaactttgagatggaaagttgcaatttgtatctcatttattttctaaatatcaatttcaatctaaattaatttctcagttttgttgaattagctttgctaaacgaaaatgtagtacactttgatcccaagcagtacaaatatattagacaatactactatcagtattacgccagcctttgagttacgagttcggcgtaacttaataagtttggttcaagctctatatttgtcataagaataaaaactataaatttataattgagtaactttagaatattaaaatatcaaagccataaacttcaaaacgtgactccgcctctattcgtaagttacgtacttttgttccaccaacaatcaaaattgcaaattagcgcaagaaaagaaaaaagttgttaagactaagaatcgagatagagattgaagattccatggttagggttaagagaactctgagagaaattggggctttcttattacttgtta

SEQ ID NO: 5 - DNA, 3465 bp, Nictotiana tabacum
atgaacaagaaaaagctgaaatttcttgtttctctcttcgctctcaactcaatcactctctatctctacttctcttcccactctgatcacttccgtcacaaatccccccaaaaccactttcctaatacccaaaaccactattccctgtcggaaaaccaccatgataatttccactcttctgtcacttcccaatataccaagccttggccaattttgccctcctacctcccctggtctcagaatcctaatgtttctttgagatcgtgcgagggttacttcggtaatgggtttactctcaaagttgatcttctcaaaacttcgccggagcttcaccagaaattcggcgaaaacaccgtatccggcgacggcggatggtttaggtgttttttcagtgagactttgcagagttcgatttgcgagggaggtgctatacgaatgaatccggacgagattttgatgtctcgtggaggcgagaaattggagtcggttattggtaggagtgaagatgatgagctgcccgtgttcaaaaatggagcttttcagattaaagttactgataaactgaaaattgggaaaaaattagtggatgaaaaaatcttgaataaatacttaccggaaggtgcaatttcaaggcacactatgcgtgaattaattgactctattcagttagttggcgccgatgaatttcactgttctgaggttagattttgaaattttgcttgatctttaaattaaaggtttgaactttgtgaatgttggcagatatggaatacaataatggattttgtttgatctgtttaatgaagattgtctagaacctcattgttataaatatggtttgtttgcttcattaattaaagagcattccttaaaatctcgactagatgccagataacaccagttagttgacttttggattatggattttttttcatttaatcagataagatagtcattcttaaatgtttcactaaagaatttgtcatgatttcagtgtatatctttaagtgtatttggaattttggatttggatctagtactgaatgggtaactgcacttgtactccccagtcatctggggaggagcaacagattaaattcaagggttgaaaagtaatacagagtcagaaattaaccacaagttggaaaatggtaaatgtatgtggtctaagatgattactcctataacttttgatgtctaacatggagaaagttagttgatttatgctctttacttttccctttattgattttggttttcaaattctatcaattcctttgtttgattgctactcagattgaaccttagacggagtagcaatagaaaagtgaagaaaagccattttttctcctttcatctctttatttctgttttacacacagaatatggtagcatctgtctgaactagttaattttattccttaaaatttgcataactaattcgagtaaatgccttttgaagctttagttgtacaactggttgttgcattttgaggactatcgacttgatttgacagtgtctgatacatggcttgtaagttatgaaaacttatatctaggaagaaatcccaaccagagatagggagctgtcgcttggttatgagctactggctcaaagttcgagtttgaccagttaattttagatcttcggagtctaatcaaattctgaagcagtattgtgcactaataagaggaacacatcaaggatgtagcactgccaggttatgttaccttatttactaatgattgagaaccagcttaaatgatgacaaatggtcttagatttgttttttacattgctcatgactttgggatatttctggatcaacattttccagttctttatgtacttatcaaaaaattatccctgctagagcctagatgttagtgttcaagcaaccatgctagcttttaaggaagctccttctttgattcatgccatctttccgtaatcgatgccttacgttactgtcatttttctaattttcatttcagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgaggtatgtttgaaagtattgataacgatggcatgcattgtactgtgttatggatgaaagaaatgaaatcagcaattattttctagcaggcaatgctcttgagatgcttgtgtcaaattggtcagacttaatcctgagtttccatttgtttcagctttctgtttgactgactacaataattgtcccaatacctagttgttccggttggctcattcttacttctatttacgtgtcactgtttctctgaatggtccctttgtggtgaaaagtgcttttgctatgaagaaaaactagcaaagatttcatttctgggacaatttatttttaccttacatcacgtctcataaaattgcttcctattgcatactttaattcttggagagatgctttaatgtgaagaaagttctttcactccacttttaattcttggagagatgctttaatgtgaagaaagttctttcactccactactgtaagcttgctgcatgaattttacttggccatattgggggcgtgttttgatttatcttcaaattcattttcttcatgtaattcttttgagtaattttttttctgttttctgtttgggaaaaaattcagacacaattggaggaaacatggaaagcactcttttcaagcctcacttatgctaagaactttagtggcccagtttgtttccgtcacgccgttctctcgcctttgggatatgaaactgccctgtttaagggactgacagaaactatagattgtaatggagcttctgcccatgatttgtggcaaaatcctgatgataagagaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcatctgtcattgttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcgacaagctcagcagcattttgaggagtcttgggtgctaa

SEQ ID NO: 6 - DNA, 1429 bp, Nicotiana tabacum
ggttatctcactctccaacttgccgcgcgggaggggggggggaggggagaggtattgaaattagttgcaaattgtgttacataacacctgttggttagtagtattttagttagtagtattagtttgttatttgtattgggccccgaagcccactttatatttattcagatgagtagtagtttaaaggtgagttgaggttaatagtttgggccatacgggtccgtatatatagagaagcccacatatattgtaaaccgaacgattatttacattctaacaagtaataagaaagccccaatttctctcagagttctcttaaccctaaccatggaatcttcaatctctatctcgattcttagtcttaacaacttttttcttttcttgcgctaatttgcaattttgattgttggtggaacaaaagtacgtaacttacgaatagaggcggagtcacgttttgaagtttatggctttgatattttaatattctaaagttactcaattataaatttatagtttttattcttatgacaaatatagagcttgaaccaaacttattaagttacgccgaactcgtaactcaaaggctggcgtaatactgatagtagtattgtctaatatatttgtactgcttgggatcaaagtgtactacattttcgtttagcaaagctaattcaacaaaactgagaaattaatttagattgaaattgatatttagaaaataaatgagatacaaattgcaactttccatctcaaagttcactaaaaatgaagtacaaatttgagtaatcaactattttatttgataatcgacgtaggaataagcatccttttgcatacaagatggaatttattaattagcggttttgtcgagaaattatcaaaaacaacatatcgtataaatagcaaacgtatagttatagaaatggtggagaattattattgaaaatctaaatatttgcggtaactacggattgataacttattggtacataatttaagttatatacaacaacattataaattttttaattgtatcgtgtaatagtttttaaagaatattatacctccttatttagaattaaattaatttatttttaaaattctcatttgattctaatttacagtcatatatttatgccttagtttagaaaaacacaaattttaaatttttttatttatttcttaaactaaatactcaatcaaataaattggtacgaaccgagtgtttgtataaaactcaatttaatcaccttagtaaaattattagtactgcatacaaataaggatataatataacggatcaaataccgtaaagtgtaattacaactcaaataatcacagtttactaacgtcgttaaaacctctgtactaagaaaactacatactcctagtacccacgaaaacaaccagtcagagagagaagaagatg

SEQ ID NO: 7 - DNA, 358 bp, Nicotiana tabacum
cggcgaagttttgagaagatcaactttgagagtaaacccattaccgaagtaaccctcgcacgatctcaaagaaacattaggattctgagaccaggggaggtaggagggcaaaattggccaaggcttggtatattgggaagtgacagaagagtggaaattatcatggtggttttccgacagggaatagtggttttgggtattaggaaagtggttttggggggatttgtgacggaagtgatcagagtgggaagagaagtagagatagagagtgattgagttgagagcgaagagagaaacaagaaatttcagctttttcttgttcatcttcttctctctctgactggttgttttcgtgggt

SEQ ID NO: 8 - DNA, 1572 bp, Nicotiana tabacum
atgaacaagaaaaagctgaaatttcttgtttctctcttcgctctcaactcaatcactctctatctctacttctcttcccactctgatcacttccgtcacaaatccccccaaaaccactttcctaatacccaaaaccactattccctgtcggaaaaccaccatgataatttccactcttctgtcacttcccaatataccaagccttggccaattttgccctcctacctcccctggtctcagaatcctaatgtttctttgagatcgtgcgagggttacttcggtaatgggtttactctcaaagttgatcttctcaaaacttcgccggagcttcaccagaaattcggcgaaaacaccgtatccggcgacggcggatggtttaggtgttttttcagtgagactttgcagagttcgatttgcgagggaggtgctatacgaatgaatccggacgagattttgatgtctcgtggaggcgagaaattggagtcggttattggtaggagtgaagatgatgagctgcccgtgttcaaaaatggagcttttcagattaaagttactgataaactgaaaattgggaaaaaattagtggatgaaaaaatcttgaataaatacttaccggaaggtgcaatttcaaggcacactatgcgtgaattaattgactctattcagttagttggcgccgatgaatttcactgttctgagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgagacacaattggaggaaacatggaaagcactcttttcaagcctcacttatgctaagaactttagtggcccagtttgtttccgtcacgccgttctctcgcctttgggatatgaaactgccctgtttaagggactgacagaaactatagattgtaatggagcttctgcccatgatttgtggcaaaatcctgatgataagagaactgcacggttgtccgagtttggggagatgatcagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcatctgtcattgttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcgacaagctcagcagcattttgaggagtcttgggtgctaa

SEQ ID NO: 9 - protein, 523 aa, Nicotiana tabacum
MNKKKLKFLVSLFALNSITLYLYFSSHSDHFRHKSPQNHFPNTQNHYSLSENHHDNFHSSVTSQYTKPWPILPSYLPWSQNPNVSLRSCEGYFGNGFTLKVDLLKTSPELHQKFGENTVSGDGGWFRCFFSETLQSSICEGGAIRMNPDEILMSRGGEKLESVIGRSEDDELPVFKNGAFQIKVTDKLKIGKKLVDEKILNKYLPEGAISRHTMRELIDSIQLVGADEFHCSEWIEEPSLLITRFEYANLFHTVTDWYSAYVASRVTGLPSRPHLVFVDGHCETQLEETWKALFSSLTYAKNFSGPVCFRHAVLSPLGYETALFKGLTETIDCNGASAHDLWQNPDDKRTARLSEFGEMIRAAFGFPVDRQNIPRTVTGPNVLFVRREDYLAHPRHGGKVQSRLSNEEQVFDSIKSWALNHSECKLNVINGLFAHMSMKEQVRAIQDASVIVGAHGAGLTHIVSAAPKAVILEIISSEYRRPHFALIAQWKGLEYHPIYLEGSYADPPVVIDKLSSILRSLGC

SEQ ID NO: 10 - DNA, 21 bp, artificial sequence
  /note=“Description of artificial sequence primer sequence Big3FN”
aagggctcttgttcaggatct

SEQ ID NO: 11 - DNA, 22 bp, artificial sequence
  /note=“Description of artificial sequence primer sequence Big3RN”
aaatctgagcggtaaagagcat

SEQ ID NO: 12 - DNA, 3504 bp, Nicotiana tabacum
ctgcttttagtcacagtaatatgaaatgtttgcctgtaataatgaaacccattgtacgtggcaaataaagatctgtcagtgtcaatgtgtctgttcatatcattgagttattaatattatgggctctaatcctagatatacccatgctacaagtatttgtacttatttatatagttgatattgttaatttatttgttacaggtaagggcataaaaaagttgatcggaaatgtacaggtgtacatacattctcatatcctcagtcatgctttcactatcaacatctgttgacttcatttctgtcaaatttgtgcatcacctaattactatatttactagatgccagtggctgctgtagttgttatggcttgcaatcgggctgactacctggaaaagactattaaatccatcttaaagtatgttttgtatcaaaacaattttgtctgcttcttattgcatattagatgcctcagctgataagcccggtacttccattgttgtcatcagataccaaatatctgttgcgccaaaatatcctcttttcatatcccaggtacccatttattttcgcacataactttctattgtatgcttgtcttctttttgttgttgaacctacttttcgatctacctccctttggcaggatggatcacatcctgatgttaggaagcttgctttgagctatgatcagctgacgtatatgcaggtaatcttctctaccgcgtgagaagggaaaacaggatgtttggcgtatctctatctttgaaatttaaatcaggtatatgtctttacttggaggggaagtatagacttaagaataagaactcattgttgccaggcttgtttttacttgcaatactcaatcatcatcattaccaataaccatattatgtacagggaaacaagttagtagaaatattgcccataaggagttttcatctgctaaaagattgaaagggaaaagatacattatttatatttaacctgtagatattttccttatcatttcgacccttttattacttcagctttgtatcattgtgtgacacaatttgtccttttccctataagacagcacaagtggaagaggcatgtattgtttgatttatgcttttatgttgcagcttttccccctctcttcatatatatgtgatttctctctctctctctctctctctctctcttatgagtagccacacttctgttccatatattcattcatctactgcaataggttcatagttttgtaacctatcgattgctttttctacctaatgtttttctctgataaaagctacgcattgcataggatatgaatctgtctgcttcattttatcatttggctgcagttactttagtctttatctttaaccttttgctgcctagctgataactgttctggcctggcaatgtgaaatgtagttaacaattgcttctgcttaagctcggtatcaaactcttcttggcgctttttcttgacagttcttaagaaaagactttttcgattctttatcaacagcacttggattttgaacctgtgcatactgaaagaccaggggagctgattgcatactacaaaattgcacgtaaggatgatttggtccttttttttcccatcttttttcgtaactcatttttattccaactagtgctagtcttgccttagccattgtcgatcactctttccgtaggtcattacaagtgggcattggatcagctgttttacaagcataattttagccgtgttatcatactagaaggtactgctgatctatcttaatcactatgttgcatgttctttgctctttttcttctcacaatatctgtgcctctgacatgcagatgatatggaaattgcccctgatttttttgacttttttgaggctggagctactcttcttgacagagacaagtaaggcactcttaaaggatccggatgttgcgttgttttactttcaaagaattattcaattcatcctagtctcaggaaaattactatttttttactcgtgtccaactcccccctcattttcttaaaagaaccaacataattgaatcagattcaacagcatccaagatctcctgctcttccaggcttgtgataggagaaaatctgatggcagcgagggggatagattgatttccattttggttatataatattcttagcaaaaggattaaaagcttttccctcgtagactgacgtccaaatatgctagatagtgaacgaactagaatgggattagcctaaaacatggggataaaaagcctgttctaaatgtcccaagtatgttataagaatttcttaaatacttatggtgaacatcccaggtcgattatggctatttcttcttggaatgacaatggacaaatgcagtttgtccaagatccttgtaagttttttctttcttccttcttttttgtcctttgtgattggtggttatgatttttcttttgaactcttctcctgtttcaattggaaattttactgaccgttattcaatgaagaaacccaaacgctgcttagtgcagatggtttctttttctgttctgttgaatggttatacttcattttctttttgattccttggaagaaattatatcctaaaacagcgtaaaggatttgcttttgagtactttacttttgatatacctctgcagttttttctttattccttttcgatgactggttcttggatttgtctgccacatgtctctctttctgtgactggttcctgaatttctctgccattgtctctctttctccttgctcaacccatatcctttttaatcatcaacttgaaattgaatcatattactcatgctaatacaagcatcagtaagaagactggtagtgttacaatatactagtggtttttctttcattcaatcatcacttgtttgacagcttaaactaggctccactttagagataggtttttggtcttaattaaaataggtcaagggcgcgtcggaacagtcggtagctgcttagtactgaattttaacgtctcctcttttcgttttggagaaaccaatgaaaaaggggaaaagttgaaaatttgctcgttggagttgtaacaggaagttttatgagaaattggaaaacaaaaacaagaaaagaaaatatatttttaaaatttttaggacagggaattaccttttcttgaactgataggagccaatcgttttcgcatgtgaatcaagcagtcgtaagtgacttgttcttttggtacaaacacaaatattttatggctaagattgtcgtaagagaaaattttggggcgctacggttctcttttcaaatccatagccctttctaggattggcttcaattgaatattttggactgtccaaaagaaaaaggagttgcatgtttttaccccattgatttcattgttgggctgagcaaaagtatatcctccatggaggttaatcccattgttttcttctcgatgttgcggaatttattgatattatttaggtgtcttttagcaaagtaca

SEQ ID NO: 13 - DNA, 2283 bp, Nicotiana tabacum
tctactgcaataggttcatagttttgtaacctatcgattgctttttctacctaatgtttttctctgataaaagctacgcattgcataggatatgaatctgtctgcttcattttatcatttggctgcagttactttagtctttatctttaaccttttgctgcctagctgataactgttctggcctggcaatgtgaaatgtagttaacaattgcttctgcttaagctcggtatcaaactcttcttggcgctttttcttgacagttcttaagaaaagactttttcgattctttatcaacagcacttggattttgaacctgtgcatactgaaagaccaggggagctgattgcatactacaaaattgcacgtaaggatgatttggtccttttttttcccatcttttttcgtaactcatttttattccaactagtgctagtcttgccttagccattgtcgatcactctttccgtaggtcattacaagtgggcattggatcagctgttttacaagcataattttagccgtgttatcatactagaaggtactgctgatctatcttaatcactatgttgcatgttctttgctctttttcttctcacaatatctgtgcctctgacatgcagatgatatggaaattgcccctgatttttttgacttttttgaggctggagctactcttcttgacagagacaagtaaggcactcttaaaggatccggatgttgcgttgttttactttcaaagaattattcaattcatcctagtctcaggaaaattactatttttttactcgtgtccaactcccccctcattttcttaaaagaaccaacataattgaatcagattcaacagcatccaagatctcctgctcttccaggcttgtgataggagaaaatctgatggcagcgagggggatagattgatttccattttggttatataatattcttagcaaaaggattaaaagcttttccctcgtagactgacgtccaaatatgctagatagtgaacgaactagaatgggattagcctaaaacatggggataaaaagcctgttctaaatgtcccaagtatgttataagaatttcttaaatacttatggtgaacatcccaggtcgattatggctatttcttcttggaatgacaatggacaaatgcagtttgtccaagatccttgtaagttttttctttcttccttcttttttgtcctttgtgattggtggttatgatttttcttttgaactcttctcctgtttcaattggaaattttactgaccgttattcaatgaagaaacccaaacgctgcttagtgcagatggtttctttttctgttctgttgaatggttatacttcattttctttttgattccttggaagaaattatatcctaaaacagcgtaaaggatttgcttttgagtactttacttttgatatacctctgcagttttttctttattccttttcgatgactggttcttggatttgtctgccacatgtctctctttctgtgactggttcctgaatttctctgccattgtctctctttctccttgctcaacccatatcctttttaatcatcaacttgaaattgaatcatattactcatgctaatacaagcatcagtaagaagactggtagtgttacaatatactagtggtttttctttcattcaatcatcacttgtttgacagcttaaactaggctccactttagagataggtttttggtcttaattaaaataggtcaagggcgcgtcggaacagtcggtagctgcttagtactgaattttaacgtctcctcttttcgttttggagaaaccaatgaaaaaggggaaaagttgaaaatttgctcgttggagttgtaacaggaagttttatgagaaattggaaaacaaaaacaagaaaagaaaatatatttttaaaatttttaggacagggaattaccttttcttgaactgataggagccaatcgttttcgcatgtgaatcaagcagtcgtaagtgacttgttcttttggtacaaacacaaatattttatggctaagattgtcgtaagagaaaattttggggcgctacggttctcttttcaaatccatagccctttctaggattggcttcaattgaatattttggactgtccaaaagaaaaaggagttgcatgtttttaccccattgatttcattgttgggctgagcaaaagtatatcctccatggaggttaatcccattgttttcttctcgatgttgcggaatttattgatattatttaggtgtcttttagcaaagtaca

SEQ ID NO: 14 - DNA, 3689 bp, Nicotiana benthamiana
aaagccactacttttagtcacagtaacatgaaatcataaactcctcttcttctttcagcttttagtccaaaagccactgcttttagtcaaagtaatatgaaatgtttgcctgtaataatgaaacccattgtacgtggcaaataaagatctgttagtgtcaatgtgtctgttcatatcattgaggtataaatattatgggctttaattctagatataaccatgctacatgtatttgtacttatttatatagttgatattgttaatttatttgttacaggtaagggcataaaaaagttgatcggaaatgtacaggtgtacatacattctcatatcctcagtcatgctttcactatcaacatctgttgacttcatttctgttaaatttgtgcatcacctaattactatatttactagatgccagtggctgctgtagttgttatggcttgcaatcgggctgactacctggaaaagactattaaatccatcttaaagtatgtttagtatcaaaacaattttatctgcttcttattgcatattagatgcctcagctgataagcccggtacttccattgttgtcatcagataccaaatatctgttgcgtcaaaatatcctcttttcatatcccaggtatccatttattttcgcacatagctttctattgtatgcttgtcttctttgtgttgttgaacctacttttcgatctacctccctttggcaggatggatcacatcctgaagttaggaagcttgctttgagctatgatcagctgacctatatgcaggtaatgttctctaccgtttgagaagggaaaacgggatgtttggggtatctctatctttgaaatttaaatcaggcaggtctttacttggagggaaagtacagacttaagtataagaactcattgttgccaggcttgtttttaattgcaatactcaatcatcatcattaccagtggtagagccacatcaactgaggggtgtcgatatcgacacccctttgccggaaattatactgtattgctagataaattttgttgttttatgtataattactatacattgacttcccttgattttacggtgtatataaattcttatattttgataccccttagtaaaaatcctgactctgccactgatcattaccaataataagattatgcactgggaaacaagttagtagaaatatgcccgtaaggagtttttatctgctaaaagatggaaagggaaaagatatattatttatatttaacctgtagatattttccttatcatttcaacccttttattacctcagctttgtatcattgtgtgacacagtttgttcttttccctgtaagacagcacaagtggaagaggcatgtattgtttgatttatgcttttatggcgcaaattttccctctctctccataagtccatatatatgtgatttttatgagtagacacacttctgttccatatattcatttatctactgcaatataaggttcatagttttgtaacctatcaattgctttttctacctaatgcttttctctgataaaagctacgcattgcataggatatgaatctatctgctttattttatcatttggctgcaattgctttagtctgtatctttaaccttttgctgcctagctgataacttgttctgacctggcaatgtgaaatgcagttaacaattgcttctgcttaagctcggtatcaaactcttgttagcgctttttcttgacagttcttaagaaacgactttttcgattctttatcaacagcacttggattttgaacctgtgcatactgaaagaccaggggagctgattgcatactacaaaattgcacgtaaggatgattggtccttttttccccatcatttttctagactcattttaattccaactagtgctagtcttgctttagccattgtcgatcactcttttcgtaggtcattacaagtgggcgttggatcagctgttttacaagcataattttagccgtgttatcatactagaaggtattgctgatctatcttaatcactatgttgcatgttctttgctcttcttcttctcacaataactgtgcctctgacatgcagatgatatggaaattgcccctgatttttttgacttttttgaggctggagctactcttcttgacagagacaagtaaggcactctttaaggatccggattttgcattgttttactctcaaataattattcaattcatcctagtttaaggaaaattactattttttactcgtgtccaactccccccgcattttctttaaagaaccaacataatttaatcagattcaacagcatccaagatctcctgctcttccaggcttgtgatagtagaaaatcagatggcagcgagggggatagattgatttccatttcggttatataacattcttagcaaaaggattaaaagcttttccctcatagacgtccaaatatgctaagtggtatagcgaacgaactagaatgggattagcttaaaacatggggataaaaagcctgttctaaatgtcccaagtatgttataagaatttcttaaatttttatggtgaacatcccaggtcgattatggctatttcatcttggaatgacaatggacaaatgcagttcgtccaagatccttgtaagttttttctttcttcctcctctttttgtcctttgcgattggtggttatgatttttcttttgaattcttctcctgtttcaattggaaactttattggccgttattcaatgaagaaacccaaactctgcttagtgcagatggtttccttttctgttctgttgaatggttatactttattttgtttttgattccttggaagaaatatatcctaaaacagcgtaaaggatttgcttttgagtactttacttttgatatacctctgcagttttttctttactccttttctatgactggttcttggatttgtcttccacatgtccctctttctgtgactggttccagaatttctctgccattgtctctctttctccttgctcaacccatatcctttttaatcaacttgaaatagaatcatatcactcatgctaatacaagcatcagtaagaagattggtagtgttacaatatactagtggtttttctttcatccaatcatcacttgtttgacagcttaaactaggctccactttagagataggtttttggtcttaatcaaaataggtcaagggcgcatcagaacggtcggtagctgcttagtactgaattttatcgtcctctcttttcattttggagaataaaaaaggggaaaatttgaaaattttctcattggagttgtaacaggaagttttatgagaaattggaaaacagaaacaagaaaagaaaatatattttaaaaaattttaggacagggaattaccttttcttgaacggatacgagccaaccgtttttgcctgtgaatcaagcattcgtaagtgacttgttcttttgatacaaacacaaatattatatggctaagattgtcacaagagtaaattttggggcgccacggttctcatttcaaatccatagtcctttctaggattatggggcttcgattgaatattttggactgcccaaaaaaatagaggtgcatgtttttaccccattgatttcattgtggggctgagcaaaagtatatcctccatggaggttaatcccatc

SEQ ID NO: 15 - DNA, 20 bp, artificial sequence
  /note=“Description of artificial sequence NGSG10046 forward primer”
tgattgcacaatggaaagga

SEQ ID NO: 16 - DNA, 20 bp, artificial sequence
  /note=“Description of artificial sequence NGSG10046 reverse primer”
attccatgctcccatttgat

SEQ ID NO: 17 - DNA, 7280 bp, Nicotiana tabacum
agcgcaatgcatctccttagatgacgaagtaaaaggaattctagaaaatattgatccaaattttcgatgaaaggaatatccgaatatggatgctatataaattggatttaagctatatatactcatagaataatttattttacaatatcagtataacattgatggtagaactagcaacttatacaaaagaattcaaaaaagatgtcaaaattaggatttaaacatgcgattaaagaaatttttgaatctagctaactagtgttactttgaccagttggtccttatttcgttgtttgtcctcaaagtttcatcctaaaatcttgaggtacaaccactttatttaactcttggcgactctattttttttaaaaaacttaatgtcaccattgcagtgagaggttgtattattgttattatcgtattataaacagaggcggatgcagtgtagaagcaatgggttcaattgaacccatagctttcgctcatactctttatttttttccaaaaaattattaaacgtgtacaaataataaatttagaacccattaaattagatgagatgtggtaattaagaatctgaacccataaaatttaaaccttgaatccgcctctgattataaatgaagcataacaggatagagttaaagtgaaattcaagcacaagtacataaagaagaaaaaaagaaatttgtaaaggaatgttgaatgtcctcattaacgtctcaaaaacctgaacactatttagcggcaatgtaagagaataacagagacagagcttaaaatatgacaaaatctttggtcaaagtctcatttcttttgtttggttacaagttacaaccatatattcactgaaacatcatactgacgttcttgttaatctgcttcttcgacatctgatgggatgaatgcaaaacagtaactgtgagtacaagtattacaactacaaaacaaactctgttgaaatcatctctacataaagaggaaagagggtggccagattgatgggaaaacaacagagaacatggatggttttctcatgttcttatcctatgatcattaataagaatgacgaaaaacaattgacaatgaattgcaagaaagtgtaacaacctaatctttcacatacacgtgttactcgtcaactgtaatcttcaacaccataaagaatatgctcttcaatgagctcaccattccacaatcaggattccatgctcccatttgatatgattgctctggtagtccataactgagtcagagtgggctaataagttagtagctgctggaaatttccaagcttatgaacaccagattgcagcaattgactatagaactgcgtgtgaaacatattttacacaattcccaggctccaggttccagaaccccgaattcctctatccttcccagtcttttagagaaaagacgaactaaactgtcgagcagatttagcacccaagactcctcaaaatgctgctgagcttgtcgatcacaactggaggatccgcataagacccctccaaatatatgggatggtactccaatcctttccattgtgcaatcagagcaaaatgggggcgcctatattcgctgcttataatttctagtattacagcttttggtgctgcagaaactatgtgagttagacctgctccatgagcaccaactatgacagaagcatcttggattgctcgaacttgctctttcatggacatgtgggcaaacaatccgttaattacatttaatttgcactccgagtggttcaaggcccagctctttatggaatcaaatacttgctcttcattgctaagcctagactgtacctttccaccatgacgtgggtgagctaaataatcctcacgtctaacaaagaggacattagggcctgtgactgtccttgggatgttctgtctatccacaggaaatccaaaggctgccctaatcatctccccaaactcggacaaccgtgcagttttcttatcatcaggattttgccacaaatcatgggcagaagctccattacaatctatagtttctgacagtcccttaaacagggcagtttcatatcccaaaggcgagagggcggcatgacggaaacaaactgggccactaaagttcttagcataagtgaggcttgaaaaaagtgctttccatgtttcctccaattgtgtctgaatttttttcaaacagaaaagaggaaaaaagaattactcgaaagaactatatgaagaaaatgaatttgaagataaatcaaagcacggccacaatatggccaagtaaaattcatacggcaagcttccagtagtggagtgaaagaactttcttcacatgaaagcatctctccaagaattaaagtatacagttagaagcaattttataagacatgatgtaaggtaaaaataaattgtttcagctatgaaatcattgctagtttttctacatagcaaaagctcttttcaccacaaagggaccattcagagaaacagtgacgtaaatagaagtaagaatgagcaaactgcaacaactaggtattgggactaggggtgtcaatggttcggttcggccggttattttataaaatttgtaccataccaatttttcggttattctattatgtataaccaaaattagacttttcgaaaccgtcccaatcatgtcggtttctcttcggtagcggtaaggttcggttaattttttaatatcatgtaaaattcaccagtagaagtagaatgcaataacatacgttcttttataggacttagaaaaattctttagacatttttactgtttaaaaggtgatgaattaaaaaaagaaaaagaaagatggctagagtatagatccatcgactattctacaacaacgtaaaagaaatcaaacaaaggcaaagaaaatataaatcacacgagttgaaagatataccaagctgggactcaagaatagagtctatagaagattaaatattcaaaaagataaatctaaaatatatgaaaggaaacatattcaatacattgtagtttgctcataatcgctagaatactttgtgtcttgctaataaagatacttgaaataacttagtttaagtagaagtaacataataggttttaggaattagtattttgagtctaattacttgttggcttgtaatagttttcataattccatggcccaaagaaaatttaatgcattattatttttaaacttactaaataaatatattttccacatgtaaaatttattcggtacggttcggtattttttcggcttatttttataaaataaaaaacctaccctaattatcggtgcggttatagatttatataaaaacctacggtttcttaaaaagaaacctaaaaatcggtacggtacggttcggtcggtttagtcggctttcgaatatccattgacacccctaattgggacaattattgtagtcagtcaaacagaaagctgaaacaaatggaaactcaggattaagtctgaccaatttgacacaagcatctcaagagcattgcctgctagaaaataattgctggtttcatttctttcatccgtaacacagtacaatgcatgccatcgttatcaatactttcaaacatacctcacaatggccatctacaaaaaccaaatgtggccgactgggcaagccagtaaccctggatgccacgtatgcactataccaatcggtaactgtgtggaaaaggtttgcatactcaaatcgtgtaatcaaaagtgacggctcctcaatccactgaaatgaaaattagaaaaatgacagtaacgtaaggcatcgatttcggaaagatggcatgaatcaaagaaggagcttccttaaaagctagcatggttgcttgaacactaacatctagcagggataattttttgataagtagttaagtacataaagaactaaaatatgttgattcggaaatatcccaagtcatgagcaatgtgaaaaagcaaatataagactatttgtcatcatttaagctggttgtcaatcattagtaaataaggtaacataacctagtagtgctacatccttcatgtgttcctcttattagtgcacaatactgcttcagaatttgattagactctaaatgttatcctggtgaagatctaaaattaactggtcaaacttgaactttgagccagtagctcataaccaagtgacagctccctatctctggttgggatttcttcctagataaaagttttcataacttacaagccatgtatcagacaatgtcaagtgtcaaatcaagtcgatagtcctcaaaatgcaacaaccagttgtagaactattcaactaaagcttcaaaaggcatttactcgaattagttatgcaaattttaaggaataaaattaaccacttcagacagatgctaccatattctgtatgtcaaacggaaataaagagatgaaaggagaaaagaatggccttcactttttgctattgctactccgtctaaggttcaatttgagtagcaatcaaacaaaggaattgatagaatttaaaaaccaacaccaataaagggaaaaggaaaaagcataaatcaactcactttctccatattagacatcaaaagttataggaataaccatcttagaacacatacatttaccgttttccaagttaaagtggttaatttctgactcagtattacttttcaacccttgaatttgatctattgctcctccccagatgactggggagtacaagttcagtaatccattcaatactagatccaaatccagaattccaaatacatacacctacatataaactctgaaatcttgagtaattctttagtgaaacatttaagaatgaacaatttatctgatcaaatgaaatgcaaccaataatccaaaagtcaactaactggtgttatctggcatctagtcgggatattaaggaattctctttaattaatgaagcaaccaaaccatatttataacattgaggttctagacaatcttcattaaacagatcaaacaaaatccattagtgtattccatatctgccaacattcacaaagtttaaacctttaatttaaagagcaaacaaaaattcaaaatctaacctcagaacagtgaaaatcatcggcgccaaccaactgaatagagtcaattaactcacgcatagtgtgccttgaaattgcaccttccggtaagtatttattcaagaatttttcatccactaattttttcccaattttcagtttatcagtaactttaatctgaaaagctccatttttgaacacgggcagctcatcatcttcactcctaccaataaccgactccaatttctcacctccacgagacatcaaaatctcgtccggattcattcgtattgcgcctccctcgcagatcgaactctgcaaagtctcactgaaaaaacacctaaaccatccgccgtcgccggagacggtgttttcgccgaatttccggtgaaactccggcgaagttttgagaaggtcaactttgagagtaaacccattaccgaagtaaccctcgcacgatctccaaacaacattagggttttgagaccaagggaggtaggagggcaaaataggccaaggcttggaatattgagaagtgattgaagagtggaaattatgatggcggttttccgacaaggaaaagtggttttggcgggatttgtggcggaagtgatcagggtgggaagagaagtagagatagagagtgattgagttgagagcgaagagagaaacaagaattttcagctttttcttgttcatcttcttcttccctctctctctcactgcctgttttcctgtgtactacttagtacagagggtttaacgacgttagtaaaatatgattatttgagttgtaattacactttacggtatttcaaaaaaaaaaatcattttctttcgaaatttggccataaaatttttaatttttacttgaagataaattttgaatttttctgaaaatttaaaaaaactttaaaatattattttttaaaattttcactcagaccatttacaaaaatacaataacaatccaaaattatattcatgtccaaacacaattctaattttcaaatactattttcatttgaaaaaaaattaaactttttttgtatttttacaattcttatgtccaaacgcccactaattatatccttatttaaatgcagtactaataattttactaaggtgattaaattgaattttatacaaacactcggttcgtttcaatttatttgattgagtattaagtttaagaaacaaatatattttttttaatttgtgtttttctaaactaaggcataaatatatgactgtgaattagaataaaatgagaatcttaaaattaaattaatttacttctaaataaggaggtataatattctttaaaaactactacataattaaaaaatttataatgtctatgtatataacttaaattatgtactaataagttatcaactcgtaattaccgaaaatacctaccagtatatagatttctataactatacgtttcctatttatacgatatgttgttttcgataatttccagacaaaatcgctgattaataaattccatcttgtatgcaaaaggatgcttatgcctacgtggattatcaaataaaataattgatggctcaaatttatacttcatttttagtgaattttgagatgagaagtttcaatttgtatctcctttattttctaaatatcaatttcaaatctaaattaattcctcaattttgttgaattagctttgctgaacgaaaatatagtacaatttgatcccaagcgcagtaaaaatatatactactatcagtattgttcttgaacatggacgacatcagccttttaagttacaaatttgacgtaacctaataagtttgattcaagctcagcattattcttaagaataaaaatttttaatttataattgaataactttaaaatattaaagtcaaagtttcaaattgtggctccgttgtgaattaccggaagaaaagagaaaagcacaagaaaaaaatatcaggaaaattaagataaattagtgggactagaattaaatttatatctaactttttctatacctttccttctcaccaaacaaagaaaggaggtttgttaatattttcttatctgttttatccgaacagccttattttccatttgtggtgtatttgtttgtcactttaatgtttgtcaaattatatataaatgcttttgggacaatacttattgttactcctttattttttatggagaatattccccattacatcaaacacact

SEQ ID NO: 18 - DNA, 1548 bp, Nicotiana tabacum
atgaacaagaaaaagctgaaaattcttgtttctctcttcgctctcaactcaatcactctctatctctacttctcttcccaccctgatcacttccgccacaaatcccgccaaaaccacttttccttgtcggaaaaccgccatcataatttccactcttcaatcacttctcaatattccaagccttggcctattttgccctcctacctcccttggtctcaaaaccctaatgttgtttggagatcgtgcgagggttacttcggtaatgggtttactctcaaagttgaccttctcaaaacttcgccggagtttcaccggaaattcggcgaaaacaccgtctccggcgacggcggatggtttaggtgttttttcagtgagactttgcagagttcgatctgcgagggaggcgcaatacgaatgaatccggacgagattttgatgtctcgtggaggtgagaaattggagtcggttattggtaggagtgaagatgatgagctgcccgtgttcaaaaatggagcttttcagattaaagttactgataaactgaaaattgggaaaaaattagtggatgaaaaattcttgaataaatacttaccggaaggtgcaatttcaaggcacactatgcgtgagttaattgactctattcagttggttggcgccgatgattttcactgttctgagtggattgaggagccgtcacttttgattacacgatttgagtatgcaaaccttttccacacagttaccgattggtatagtgcatacgtggcatccagggttactggcttgcccagtcggccacatttggtttttgtagatggccattgtgagacacaattggaggaaacatggaaagcacttttttcaagcctcacttatgctaagaactttagtggcccagtttgtttccgtcatgccgccctctcgcctttgggatatgaaactgccctgtttaagggactgtcagaaactatagattgtaatggagcttctgcccatgatttgtggcaaaatcctgatgataagaaaactgcacggttgtccgagtttggggagatgattagggcagcctttggatttcctgtggatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttgaaccactcggagtgcaaattaaatgtaattaacggattgtttgcccacatgtccatgaaagagcaagttcgagcaatccaagatgcttctgtcatagttggtgctcatggagcaggtctaactcacatagtttctgcagcaccaaaagctgtaatactagaaattataagcagcgaatataggcgcccccattttgctctgattgcacaatggaaaggattggagtaccatcccatatatttggaggggtcttatgcggatcctccagttgtgatcgacaagctcagcagcattttgaggagtcttgggtgc

SEQ ID NO: 19 - protein, 516 aa, Nicotiana tabacum
MNKKKLKILVSLFALNSITLYLYFSSHPDHFRHKSRQNHFSLSENRHHNFHSSITSQYSKPWPILPSYLPWSQNPNVVWRSCEGYFGNGFTLKVDLLKTSPEFHRKFGENTVSGDGGWFRCFFSETLQSSICEGGAIRMNPDEILMSRGGEKLESVIGRSEDDELPVFKNGAFQIKVTDKLKIGKKLVDEKFLNKYLPEGAISRHTMRELIDSIQLVGADDFHCSEWIEEPSLLITRFEYANLFHTVTDWYSAYVASRVTGLPSRPHLVFVDGHCETQLEETWKALFSSLTYAKNFSGPVCFRHAALSPLGYETALFKGLSETIDCNGASAHDLWQNPDDKKTARLSEFGEMIRAAFGFPVDRQNIPRTVTGPNVLFVRREDYLAHPRHGGKVQSRLSNEEQVFDSIKSWALNHSECKLNVINGLFAHMSMKEQVRAIQDASVIVGAHGAGLTHIVSAAPKAVILEIISSEYRRPHFALIAQWKGLEYHPIYLEGSYADPPVVIDKLSSILRSLGC

SEQ ID NO: 20 - DNA, 243 bp, Nicotiana tabacum
atgccagtggctgctgtagttgttatggcttgcaatcgggctgactacctggaaaagactattaaatccatcttaaaataccaaatatctgttgcgccaaaatatcctcttttcatatcccaggatggatcacatcctgatgttaggaagcttgctttgagctatgatcagctgacgtatatgcagggaaacaagttagtagaaatattgcccataaggagttttcatctgctaaaagattga

SEQ ID NO: 21 - DNA, 219 bp, Nicotiana tabacum
atggctaaggcaagactagcactagttggaataaaaatgagttacgaaaaaagatgggaaaaaaaaggaccaaatcatccttacgtgcaattttgtagtatgcaatcagctcccctggtctttcagtatgcacaggttcaaaatccaagtgctgttgataaagaatcgaaaaagtcttttcttaagaactgtcaagaaaaagcgccaagaagagtttga

SEQ ID NO: 22 - DNA, 444 bp, Nicotiana tabacum
atgccagtggctgctgtagttgttatggcttgcaatcgggctgactacctggaaaagactattaaatccatcttaaagtatccatttattttcgcacatagctttctattgtatgcttgtcttctttgtgttgttgaacctacttttcgatctacctccctttggcaggatggatcacatcctgaagttaggaagcttgctttgagctatgatcagctgacctatatgcagcacttggattttgaacctgtgcatactgaaagaccaggggagctgattgcatactacaaaattgcacgtaaggatgattggtccttttttccccatcatttttctagactcattttaattccaactagtgctagtcttgctttagccattgtcgatcactcttttcgtaggtcattacaagtgggcgttggatcagctgttttacaagcataa

SEQ ID NO: 23 - DNA, 23 bp, artificial sequence
  /note=“Description of artificial sequence primer sequence Big1FN”
tgctgtcgccttcatctacatac

SEQ ID NO: 24 - DNA, 22 bp, artificial sequence
  /note=“Description of artificial sequence primer sequence Big1RN”
gtcctgacgcttcatttgttct

SEQ ID NO: 25 - DNA, 20 bp, artificial sequence
  /note=“Description of artificial sequenceNGSG10041 forward primer”
ctgtcggcttttcaactcaa

SEQ ID NO: 26 - DNA, 20 bp, artificial sequence
  /note=“Description of artificial sequenceNGSG10041 reverse primer”
tggcagttgtcatcgtaacc

SEQ ID NO: 27 - DNA, 10000 bp, Nicotiana tabacum
aggagttaactgataggatttgaagatgaaggaagcaataatatcgttcgatataaccaagttggaagaaatttaattttcttgttgttggggtgtttttggagaattttgaggttctcttgaagaaaattgatctcttgatcatgtacatatttgcatctttttttatttttcatgttttgtaaaaataacttaacttcttgtactttgctgtaatcatatgtatttgttggacaatggaaaactcatcaatagcatctcactattatatgaaatatcttaattttacccttcattactcaaagtcctcattagaagtaagaacgaatcgaaccactggagagtcgtctgcagttcacacttgggcaaagacggctgactttggaagcggaacacgaacccatttccgctattccaggtttcattaaagtcaaggccaaatacaagataatgtactaacatgaattgcccaaaagtataacggaggataaattaagatatttttctgtagtagtagaatgaacacaagataatattgtagcatccgccaatgcagaaaaactactaaaactattgtctagaagcatcaaaacaaaacctttcaaaataaatggcaaataataaacaaaactagtataaaaaagaacctttaattagtttctgcacttgtcattacttgtaacacaatggacatgttctctgaactagattacaaattgatcagctagaatttcccgctagcaaaaccaagtccaagtctctgtatatcacccgcaatttcgcaactgcactactttgggaggttaggcatgtccattcaaggttcagagtttgttcctttttacttgaaaatgtaaaagatcagaactttccaggacaacattttgtactgcaaaaatcataaccagaatgatgttaggcagtggggcggatggtaacttggtaagaatgagcattacgctatggactacaacaagctgctgtcgagactcaggtttggacttgatctatacaaatatggcttcaaagtttgcgcatgggtggctttcgatgtgattcttaaaatcagcgtctcctttgaatctaaaggtgtacaatgcctgtcgtgtcatgccaagaggatatactctgtagagctttagttcatcccctccacgaagtatttgaggtctttcttctttccaaataggaacatgctttagagatttcaactttgacagtactgcagattcaaaagcctccagtgacaaattagatgacctgcaaaggctccagcaataacagtgattcaaagataacataagacttgccaatgagacatgaaacagtttgtatatgctcagaaagtaaaaggagaagtgcaggagagaattcacattatgacattggttgtccatatttctttttcgtggccggatccccccaactttaaccccccagccctatcttctagctttactagtgaaccaaacaccacttgccaaatattttccggaacttccctagttaagtctatcaaatcgatcaagaactactttagatagattgacattgattttgtggaatatccaaccacaaacatgtaaactactttataagcccatagaccatggctaccaagcatatacttctcaattctcatacatcgagagtaagacacagtgagtagttactcaaataatttgatcaatatgtctcacccctatttgcggtcagctctacgaatcctctatatacattccactcttttcggatccttttcattccaatattggtaaacaatttgatcatcatggtacatattaaacataagaacagcggcacaggaacataaacagaaaaggcacttgtatatgcatgatatttagtcatggcagatcgagaataccttaggaaaacagactccatgtcaaacctccctctttcacgtacatatacatgatagacagtttctgaacctctggtacatttgcagggacgtttcgtaaattttggactcttctcttctttctccctaatactagttgctaagaagatacacaaacgacaagaagagtgaactgctgccatgtcaaccagggctttgaaagagtcagatggaccctcaaatttccacctaaacaatgaaaagaaaccatttgttagcaagacccaggaagtttaagaatctttaataaattttgaaaggaaaaggtcaaatgcaagtccgcacatgcagaaggggacacacactcacacacacggaataatcttcccaatttaggagttttagttagaccaaaactatatatcagtgtgtcaaaataggataaggtaataaacttttgtaactatagagacaggccagtgctactttctcaataacatgataacagaaaagtacagaaagatgttggaaaaggggtttttttttagagagagtggataaatcttcaggacaaacaaaaaaaagttaatttaggacaggaaaattcaaataatcaagaagagcacgtaagaaaagcatgactaattgatgcataccttaatgactcattatatgcattaggattttctgcaaggtacttcatagtcttggcaactgatgcagcgtctttcagctctttaatgtgtaaaagtgaattaggagaaggagcaaaatctaggatgtttggagcaccaatcaccacggggactgatcctgcaagtttacatgaacttccttttatcagcacaatagcgttgactaccataaatcataattttgaagtagtaacttgtgaaaaatcataaaaacaggattctgtgcataactctagcctaaagtatatcgactaggagcacaaagaatatgattaacaatgataattaacgtaaaatcatttaaaattttcctaaagccttttacatgaatataaagcagagcatatttgtgtgccgtaccagaaaatgcaaattcgggtacaaaacgcagttgaagtaacataaagtgctcagaagtttgtgctttttgctttgttcttcatggcgctctatagctaatatctcctatgtcttttgtgtaattatgaattgcagaaaaagtaaaacaaaatgcatcattgcagaggggtgggggctaatagaaaataatagatacatcggaaactaagtgttcaaatcagaaccaaaattaacaggcaaaaattattaccagctactaaggactggaagaatttttcagtgacataatcctcctcattagaattctcaaaagcgaagctaaatttgtagcgcttgagagtttccactttgtccactgccatgaaattgagaacaagatgacaagtgagcattcacaggatataatgtacaattaaactagaggcatgaataaacatattctgcattctaatatgacatttattaagactgccttttctcttaatttccctttccaacaaaccctggtctagcttcttcacaataaatttacaaaaagatgtctgcttttcaactctattctaataaatttacatataatgtaaagtttaaccacatgcatgcatattctacatcagaaaaataaactaaaactaaattttctctaaatttttcttttataacaaattagggtctaatttcattgcaatgaatttgcaaaaactgtcggcttttcaactcaatttaatgggttgcaaaatactaagaaggtattcaaaaaataacaagcataacagcaaaataaccaatccacaaaatgaactcttcaaactagcagaaaatctgactatttagaaaacttacatgtgtatgtgtacatttaccaaggaaacaaatataaggtttccattaccagtaaacatggcttttcaaaaacacattatattgaaatattttagacaattagctccttaatggtaactacttcaagaaaagaaccatcaatatattatcatatataatggagatacactgaccatttccatcccggttacgatgacaactgccaaaagaatcaatcttgatatttgccctttcaaggacttcaagagcctgcaaccggaagttgcgagcaccacaattagaaataaaagcagctgctaatgcattctcagttttaggttgcactggagccattatatcgtactccgcccaagagaagtacccaacaggaacatccgaagagaggcttgttgtcattacaatatcatatccccttctgaaacatgagaaagaataataaagttggttagagatttccctagagaaagatataaagattgagagaagaactataggagatctaaaaattaaattataaccacaacatgatcctaaggttattgtgtctatgaggcctttatgattgctgttattatctctatgatctgttgatagtactgatatattgtctcttttcatcttcttgagccgagggtctatcggaaacaacctctctactccctcagggtaggggtaaggtttgggtacacactaccctccccagaccccacttgtgggattttactaggttgttgttgtagttattgttgttgagggctttatgattgcatgtttgtagatgccctttccaaaggctatttgagcaaatgcaactgcttttttttgtaacgatggaaaatatgcagtatggaaggctgttattaagtcaatttatggcatggagagatattaggatccaaaaccagtaagtaatccttatgaggttgctttggggagaccaatagctaatttctagggacagttcaaagacaacataaaacaaaaagttgaaaatggtaggaaagtcaagctttggactgatagatggtgtaatgaaggtttacttacagatttattcccaactgtgatcagttgtggtttcaaatcaggattgtctatgtgaatctgttagtcatctcccgatgcatcgtaatctttcttggagtatctggtctttattccttaatatttagagggtatcctgataaaaaatatattttgggtgtaatactgggtaatgccacaaaatttcaaaggtgcactcatcagctggcaagcacagtggagaaaagagggaagaatatctggaagcttatttctttgtgtattgttttgacggtttggcaagaaagaaatatgagatattttgagggggaaaatgaggagattatagttatgaagtatagatgtttacaatatctcttttgctggcataaaacggatcttgtagttagtacaaatgagttcttggagtcgctggattcaggttttgagtagactttggaagtatcaatttttgtactttcttggtactgcttactaataatatgctaccttacttatccaaagaaaaaaggtctagtcagaaactaattgcaagcaaaaacttccaagtgagatatgtttcattaaaaaatgatcacaagatgaaaattcaattgtcgacaaatgctgccaaatgtggtgagaatgttttaagtctttttctagatgtgcttacccaccgtcgtgcggtaatgatgttgttctcaggatagtattgagcagactccattgaccgaagcacgctagccgtgccagcctgttgtggtgtcccaaatgccgcatcaggcttcttatcagaatccacaccaaagttacatcctacggcacaagacttccaatcctagataacgatcgaaggaacatcagtttttgcatatggtaagggaaaaaaatcaagaagaaagatggtaaccgagtaactgttacctgctctcacctatataccagtggataacatgggaacaagaactagtcatgataaactcaagcaataaatggcagttttactgattgatttctgctcattccacttaagtgctctatgaagaattcaatgtaatgaaggaaaaaaaggtaagaaaaaaagaagatactcaaggctcctccccttattccctactagtatataaaattcatgagtatttttacccatatagaagaggttttggaatattgtcaatagtctgtactttataagatattccactgtaaacatgatataagaacaaaaaaaaaacgagaagaaaacccaagcatcatttgaaacaacaaatttttatcattccatctaccaaagccatatgtctggagctctggcagctaactgatgtaataaataacaagacataaattgcaacaaaatccatataatagtctaaaagagatgctcgattaccaattaatgtttccatggagaattagctgctactaccttttaagtgacctaatggtgtaaatctattttacatcgttagtgcatagaacttatactcatcatcaaaatactcatcatcaaaatggcaggtcgatgcgctaagctccggctatgtagggttccgagaagagccggaccacaagggtctattttacgccgccttaccctgcatgtctgcaaaaggttgtttccacggctcaaaaccgtgactcacatggcaacaactttaccggttacgtcaaggctgcccttcactcatcactaaaaaaaaaaactattacatccttaagtgcatagaacttcccaaactattcgtgttcaactatatgaatcctctatatccattctgctctattcaccccatttattagtaaaaagaataaacatatacaagaaacaatctcaccttttcgccgccatgaacaaaaattggatctttgtcaaaatctctagaatactccacagaatcctccctttccaaccactcctcacagctcccagtttccaaattccgatcaatctcaccactccttaacacagccaacccagcctcattaatttccactttggaggttgaccaagacgacgtcgtaaactggtaaaatgagtcagtccaagagttgactaggttggctttttcagccatgtctagtcgacccagaaatgcaatttcaactataaccacaagtgcaactactagaggtagccaattggaccatttcttttggggaacatttgtaggtgatgatgacccaacaccttcaaatcttggtaatctttgaattggcacaactgttgccattaataactcaaaattttgtatcaaaaatttgttctttttgtggaagtaatatagctaaagatgtgatctttttttcttcttcttcagagggtttggtaattcttcatttggtgggacagttgggggtgggtggagggggggaggtaggagtgaaagtcaagcaaagagatatatgaatgggaagggaagtttgttggtcaatctaaaaacactgtaggtctacgccaccattggtgtcttagttctttttttctttttctttattttttgtatttttttgttttttgtttttgccgtacgttgttgtctcagttgacattagtcgtttccgtttcgtttttccaagtgttatttatgcactgccgggtagtttgggtcacaaataaattacggaggaattatgacgtagctttgacaagtaaataaagaaaattatttaattacactaattatgaaaaaaaatattatacagcattaatcataaaaatgcaaaagtagtctctgtaaaggggccttaaagcaacgataaagttgtctgtatgtaacatacgactatgtttgcatagggtaggccacacgctcttaggttgcagcccttccatgtaccttgcgtaaatacgggatacttcatgcaccggactgcccccattttttatcttaataattttttaattaattatcctttatttatttttagaatcattagtaaatgattaaatactttataatttttttagataaaaattttttgattaaattatcctatatttattcatgattttctgaaatgttatttatttttaaccaattttttttagctaaaacgacatttagtacaatagttaagatacaaaagttttttaaaaaaatgtaacggtaatccagtaatactagtacaataataacaaatctagcataaagagccttatccctacgttgtgaaggtatagagactgttttccattaacctaactcaacggtaaactagtaataataagaaatatttcccctaactcttttcctcaatctcatttatgaaatatcactaagcagttttctcgttatgttgcgtctgccaatatctacttagattgaaatttatttaattttctttcaaattaatttaattaagatcttttttcaacccaatttaaagccaacaccccctaaggaagaagtaattcaaaagataagacagtgcatgaagcaaaagagtagaaatgatagactttgtgggcagaatttcctatacttgtgcgtgcaagttggctcgaacatcattgtcataaaaaaaggttggatagacaatcaatggtcaagatattgtattaatatccctgttgtctagctggatcactcgctatttcaccaaatctaactaaaccctaatccaacttaaatatgaactcaacagcaactcatgacatctgatctggaaaagttggttaagtaggagcaactgttggcaacttaaaatgaatctgttaatgatggatgcctattccctgcggactatacattagaaataaaaagggaaaaggaataaagaaagtcaaacaattagccatttccttgtgattatcaagtcatatggaagttggtactcctactaaagatatcctttgacaatattctataagactacaaccaccagcaaaagattagttttttttaagtaggtacagaaatggcagtgattccacccacccctttcccacatccctaaaagaaacaatgcaactacttaattgacgattctattgtcgaaaataaaagaactttgtataacgtaaaacacaacaacagtgatctcaaacttcatttcaccctctaaccacttctctaaacatatctcatttacaaatttaatggaatgaagaaactctatcgagatgagccagataattcttttactagtaactactagctaaaaaaatggcccctttttctacagcaaaacaaaactcatctttagtagttagtacctccaaacttgtcaaatgtcattcttcaaacacggaccagcttgatggcaagagcttgtgcttgtaaagcagtgttctcggatattactctgcaccttagctgccagctgtcatgcaaaaaggggacttcacatcaccttggggacagatgaagtcgtggttcttttactaacattcaagatttacttctttctttcgctggaacacggagttggctgttcctttggaacaatctaaaagtaaatgttcattgattagaaattagaccttaaaaaccactacttaagttatggacgtaataagaagaagttgatgcacattacttgctcatcatctgcagtatccatcatagctgtcttcagttttttgctccttgagttttcttcgaaatcttcatccacaaaattctgacctgaaccttctgcaaatttttttaaaatttcagtgtcaaagaaactaacagaagtataattcacatagctatgagacatatcattaccctggatttcctgaacccagggaatcaagcattaaacaagtttttagaactctactaccctcccccaagaaagaagcacattcaccaccaccaaccaccaatatatgtgtgtgtgtgtgtgatatacgagcaatgctgcacccagaaaagaaaaacttaagagaagtatttaaacaagtaatataactgagaccgacagctataaattccatatttgagccatttgctggttatttctccaaacatgccttatattacccaaagaaggaagataaaagaaaaggaaacaatacaaacaattaacactggctccaaaacaactgatattttccaagttctatcctaatcgaaatggtaatagaactggtgtctgcaaaagggctacagcagaatatggacagtaactgcagaacctgaacagatatacttttaaacatttattccatgcagttatgaggactcagctctgagttagtagcccttttcttttc

SEQ ID NO: 28 - DNA, 1527 bp, Nicotiana tabacum
atggcaacagttgtgccaattcaaagattaccaagatttgaaggtgttgggtcatcatcacctacaaatgttccccaaaagaaatggtccaattggctacctctagtagttgcacttgtggttatagttgaaattgcatttctgggtcgactagacatggctgaaaaagccaacctagtcaactcttggactgactcattttaccagtttacgacgtcgtcttggtcaacctccaaagtggaaattaatgaggctgggttggctgtgttaaggagtggtgagattgatcggaatttggaaactgggagctgtgaggagtggttggaaagggaggattctgtggagtattctagagattttgacaaagatccaatttttgttcatggcggcgaaaaggtgagagcaggatgtaactttggtgtggattctgataagaagcctgatgcggcatttgggacaccacaacaggctggcacggctagcgtgcttcggtcaatggagtctgctcaatactatcctgagaacaacatcattaccgcacgacggggatatgatattgtaatgacaacaagcctctcttcggatgttcctgttgggtacttctcttgggcggagtacgatataatggctccagtgcaacctaaaactgagaatgcattagcagctgcttttatttctaattgtggtgctcgcaacttccggttgcaggctcttgaagtccttgaaagggcaaatatcaagattgattcttttggcagttgtcatcgtaaccgggatggaaatgtggacaaagtggaaactctcaagcgctacaaatttagcttcgcttttgagaattctaatgaggaggattatgtcactgaaaaattcttccagtccttagtagctggatcagtccccgtggtgattggtgctccaaacatcctagattttgctccttctcctaattcacttttacacattaaagagctgaaagacgctgcatcagttgccaagactatgaagtaccttgcagaaaatcctaatgcatataatgagtcattaaggtggaaatttgagggtccatctgactctttcaaagccctggttgacatggcagcagttcactcttcttgtcgtttgtgtatcttcttagcaactagtattagggagaaagaagagaagagtccaaaatttacgaaacgtccctgcaaatgtaccagaggttcagaaactgtctatcatgtatatgtacgtgaaagagggaggtttgacatggagtctgttttcctaaggtcatctaatttgtcactggaggcttttgaatctgcagtactgtcaaagttgaaatctctaaagcatgttcctatttggaaagaagaaagacctcaaatacttcgtggaggggatgaactaaagctctacagagtatatcctcttggcatgacacgacaggcattgtacacctttagattcaaaggagacgctgattttaagaatcacatcgaaagccacccatgcgcaaactttgaagccatatttgtatag

SEQ ID NO: 29 - protein, 508 aa, Nicotiana tabacum
MATVVPIQRLPRFEGVGSSSPTNVPQKKWSNWLPLVVALVVIVEIAFLGRLDMAEKANLVNSWTDSFYQFTTSSWSTSKVEINEAGLAVLRSGEIDRNLETGSCEEWLEREDSVEYSRDFDKDPIFVHGGEKVRAGCNFGVDSDKKPDAAFGTPQQAGTASVLRSMESAQYYPENNIITARRGYDIVMTTSLSSDVPVGYFSWAEYDIMAPVQPKTENALAAAFISNCGARNFRLQALEVLERANIKIDSFGSCHRNRDGNVDKVETLKRYKFSFAFENSNEEDYVTEKFFQSLVAGSVPVVIGAPNILDFAPSPNSLLHIKELKDAASVAKTMKYLAENPNAYNESLRWKFEGPSDSFKALVDMAAVHSSCRLCIFLATSIREKEEKSPKFTKRPCKCTRGSETVYHVYVRERGRFDMESVFLRSSNLSLEAFESAVLSKLKSLKHVPIWKEERPQILRGGDELKLYRVYPLGMTRQALYTFRFKGDADFKNHIESHPCANFEAIFV

SEQ ID NO: 30 - DNA, 20 bp, artificial sequence
  /note=“Description of artificial sequenceNGSG10032 forward primer”
gtcgcaactcacaaggaaca

SEQ ID NO: 31 - DNA, 20 bp, artificial sequence
  /note=“Description of artificial sequenceNGSG10032 reverse primer”
accaagaggctgtggtgaac

SEQ ID NO: 32 - DNA, 6698 bp, Nicotiana tabacum
tgtacttcagatcgttccaacaaggtatctgggagtccaggagttgctagttccatgtcccagagaaataaccaattgaatcttggaagttcatcagaaaacaaccataggagctaattccatgttccagagaaatgatcaagtaaaatccgaaagttcatcaggagactgatggatttgagtgtcctcagacgtgctatgagccgttattgaaatgcaggggtgggtaaagctggagaacactgagtcacaaagacaagcagttggtatttgctggtggcttctctaaaagataactttagcgaatttgagaaggtgatgttggataagattagatttgttacatgaaagttagtagtgttggcgtatcaaatttggttagtaatatagagtaggaaaaagtgtggcgtacatttgcagaggtttgcagtattgcactacaaatgtaaatgatagtagtagtagataattatctatacaagcacaagtattttgcaagaaagagaagggaaaaagctaggaaatcattaattttgttaattctgagatctctgtgatctgtctatatatagagtaattgtctgtatgaactttagccggagaaccgataatgagttaggagttctggtatctaattcgtttgttttcattcttttttccccttgcgttgtgatcttgaaccccatacagattcatgtatacgtaatcaaaaggaactctttcgattatttgtacagtgtattcgaaactcttttcttcttttattcgacgctgtttgctcgctttggtctttgtttgcagaatgagttgctttgttacaaaatggagatggaacaaactagtaggagtaatcaattcagcatgatttctgacttgatatacaaaaggaatagagaacttaaatagtttgacaggttgtatattaggaactcgtggagatttcacttgaaattcccacatctgactaatcctatatcaattggataccttccttgaatttttcataattttgcttttgacccaatagtttaatgctgtcacttcaggttcaatccaaggattaaacttggactagttaggtggatcgctaaaaatggaattactttgctacttctagactctacttgtgcaatattaagtgctatttttaagcttttgagcatcattttgttcaaatgggtaaaaataatctcaacatgaacgaaattattaggaacctgaaattaaaccactatcattaaaaaagaacatagaagatgtcgcaactcacaaggaacatttttcgttttccctaaaggaaaaataattaactctgatggaaataaatttaaaatatagaaactacattggaaaaggtaacaatttatacaagtctttagttgacctgtattcgctaaaacccctttcccgtaataataatgttaggaatccacttctctatttttctctctaatttctttacttccttatcaaaatttagcaaattaaagccaatagctattccaattcaaaggtttcctcaatgagatcttcgtcaaattcaaacgcacccaataaacaatggcgcaattggttgcctctgttctttgccctagtggttatagcagagatttcttttctggttcgactcgacgtggctgaaaaagccaactcttgggccgactcgttttatcagttcaccacagcctcttggtccacctctaaactggctgttgaccacggcgacgttgaggaggtccagttgggtattttgagtggtgagtttgatcagggcttcgtacccgggagttgcgaggagtggttggaaagggaagattctgtggcttattcgagggattttgataatgaaccaatttttgttcatgggcctggacaggttataaccacttctatttatgactgaatattgaatattgatataattggacttattagttcgggattgagccatagtcgtatttctttttctttttcttttctttttcttttttcgtccgttgtttgtggcatgcctaaatctcttttaatgtgtttatttatttttgtggattttatcgtaattcctagtgttagataatccttaaatacagggtattgaaatattatggacccgaatagagcaattatgatattgaggattcatacagccgactccagctagtttgggatagaggcgtggtagtagtagtagttgttgttgttgttgcaaaaaaatgtattggcatctcagtatgcgtaaggtgcatggttgatttcagacttttgactattattgtagctgggtcatagcaagagaggtttgcttagttgatggattttagttttttagcttcgtttgcttggagcttttcataaggactagagtttctaatacttttatttaaaaaggggaaagagcagagaagcattgtgaattttcatattgattgccttttgaagcatatagtcgttcagtattcctttacttatttcataaaaataacatgctaatggaatgatcagaaatcaatttatcataatgcgaataccacttcttattgttcttggtctctcatgctattcgcttgtgagtttgaatgtttaactgttaggcattacttatctctggctttatgaatgtcctgccctatacgaaattctgatgtctattcaatgattcaatcactatataggaattgaaaacctgttccgtaggatgtaagtttggaacagattccgataagaagcctgatgcagcatttcggctaccacaacaagctggtacagctagtgtgctacggtcaatggagtcagctcaatactatgcagagaacaacattactttggcacgacggtgggtaagcacattgtgaaagaagtcttatttcattccctgcctttattggcaattttctttttaatatttgttgtcattctctttcatttttatcacattcttatttaagttatgtattgctattagttttcccttccaggggtgggggtaaggctgcgtacatcttacggtcttaccctctccagacctactgggtttgttgttgttgttgttgttgttgttgttgttgttgtattgctattagttttagttaagaacttttgcattataagtatattggcagctataggtccttgaccaaacttttgccatagacaagatatttaaaaactgatgacatgaattctttccctttaggcactaaatatatttcctgtagaaaatagttaagattcaccttgactgaatcatcgctgacactgctttttgttgttttaatctggctgggttcctttctctttttcttcattccgttagggcaaaaaaataggaactctgcttttcaattggggagtttttgggatggagtggatcaccaaccatacttattgaagctaatttagagctaaagatgctaaagtacgtttttgataagtcataatgtgaatgtactagctttggttatttaaccgcacaagtcaaactagaacttagttttgacatggtataagtcaaattcttcctattttacttatatagaagttcttctccttttgtttactttataaagggtataagatgataaatatattgtctactcttgggggtctttgggtatgctaaatgagctaagaggtgtttagaactctagcaaggattttcatgacgtattaaggacatgatcagaacccatgttcagtgtttgcacagggttatcaccaactaatggtcaatgagcacatctaatctagtttaatgtttgagttattggattgacttttcaatatcaataaaccattggccaaatttcatgatattttactgagccatctgtaatatgatgtccaaccatgcctattcaataaaatgaaaattttaaaacttacagaattagttgagcgccaccagatacttaaagctatgccaactgcgtctaacagaagttgaaagacgaagttgagtgagagcactgtttttgatgtgtggattaggtgcatgtcacaagttcgaaccctgtagcagacaaaatcctggtatttacgtggagaaggatagagagctgggcgtattatccattgagtttcgaaccgtgcgtcactagcccttagggatttcggtcaacaacaacaacaacaacaacaacaacaacaacaacatacctagtaatttcccataagtggggtctggcgagggtagaatgtacgcagccttacccctaccctggaagggcagagaggctgtttccgatttcggttatcataaaataaaaaaaaaggttgaaacacaaagctaatttttctatcacaaaaatctttgaatttcattgtagttgagattttggcatcaccttgcttaaaaaattagcttagcatatagacatagatatttaaagctatgccagttgccatgatagagtttaaaattatcttggttagttggttagtgctcttcgttacattgagtcacacgattaatttatgaagccaaagttcttaaggaccatggcgtggttgagatttaaactttgcataagcttgctaaccaattttatttttttcactcacataccagaaggggatatgatgttgtaatgacaacaagcttctcttcagatgttcctgttggatacttctcttgggctgagtatgatatcatggctccagtacaacctaaaacagagaatgtcttagcagccgctttcatttctaattgtggtgctcgcaatttccgcttgcaagctttagaagcccttgaaagggcaaatatcagaattgattcttatggcagttgtcatcataacagggatggaagaggttagtatatttcaattatccaaacttactgaaggattagaggatagaatacggatggtgcaattttaagcagtgtcactagggagctaattcttgtccatagagtagtattatgggtttgattgactcttcctcgggtatcacaccttcctccagaagacaggattttactaccagtgcaaaccttttttttctctcctggctaatgtgagcacgcatgtcgtcgtttttttagtgatttgaatttatgctagtccaatgattgcttgtcaatggattattttgctctttttcttgtttaaaatttgagtttcaattttgccacctgataagaataaagttggaatacaacattcatttaaatagttcgatttcattctgaggaagttaggctgagatttgttggaaagagacgtatagcgagaaaaaatgttgtggacaaatcatctttctggatgccgtgtattttatacatgcatttggtttagggttgcactaatatccaactgaattgcgttatttgtcaataaaaaatttccaatgaaatctaacttctggtctctgttctcaatctgatggcagttgacaaagtggaagcactgaagcggtacaagtttagcttggcttttgagaattctaatgaggaggactatgtaactgaaaaattctttcagtctctggtagctggtaattacatttgttttttcttattgggtttgtagacttggattttcagagttgagagcatctattattttagctcagtccctcccttaacatgatagatacatttgttcctagttgtatttgatgtggttttggttagatcttctgcgtttactagcagaccttggaattgtagtatctaaagcgtacaattatttatagaagtttcaggaaggacaaacttctgagttctgataaattcttgatacattcaacaatggtttggatctagacttgcatttctgtagaatgcacaatgtgctctacactgagatggctcaaatatttttggaattttgttgagatgattttaggggtatgtcttagttgagcattttctttatgctctaagactaaattctctttttcgaggtctatcccatgtttaagattttgacaattttattagttcagaattgagatttaaggtttcaacttgctgataaaagtaagtctataaaacttgtaggatcaatccctgtggtggttggtgctccaaacatccaagactttgctccttctcctaattcagttttacacattaaagagataaaagatgctgaatcaatcgccaataccatgaagtaccttgctcaaaaccctattgcatacaatgagtcattaaggtatgcgtcaataagaattgttgttgttgtttgtttgttgttttttttttgtttttttgtttttgttactccagttgtttacttgataatgggatggtactcttcttaattgttcgatttcctgttttttgcaattacacactgtccaaatctctctcttttttaagccatttggtaccttttgaaaatagtattacgaagaaaatattacagacccatttcactaaaatgttttcactactgtatttccagttttgaccaatttatatatagatattgccttttgatgttaggtggataactgaattgaacgagaacacaatggatctctctgtttttctgtaattacaagcaacttcttccctttcaagattttacttaatatttcttaaatttactggacatctaacaaatgatttactttaattgttcagatggaagtttgagggcccatctgatgccttcaaagccctggttgatatggcagcagttcattcatcttgtcgtttgtgcatcttcttggcaagtaggatcctc

SEQ ID NO: 33 - DNA, 1125 bp, Nicotiana tabacum
atgagatcttcgtcaaattcaaacgcacccaataaacaatggcgcaattggttgcctctgttctttgccctagtggttatagcagagatttcttttctggttcgactcgacgtggctgaaaaagccaactcttgggccgactcgttttatcagttcaccacagcctcttggtccacctctaaactggctgttgaccacggcgacgttgaggaggtccagttgggtattttgagtggtgagtttgatcagggcttcgtacccgggagttgcgaggagtggttggaaagggaagattctgtggcttattcgagggattttgataatgaaccaatttttgttcatgggcctggacaggaattgaaaacctgttccgtaggatgtaagtttggaacagattccgataagaagcctgatgcagcatttcggctaccacaacaagctggtacagctagtgtgctacggtcaatggagtcagctcaatactatgcagagaacaacattactttggcacgacggtggttttcccttccaggggtgggggtaaggctgcgtacatcttacggtcttaccctctccagacctactggaaggggatatgatgttgtaatgacaacaagcttctcttcagatgttcctgttggatacttctcttgggctgagtatgatatcatggctccagtacaacctaaaacagagaatgtcttagcagccgctttcatttctaattgtggtgctcgcaatttccgcttgcaagctttagaagcccttgaaagggcaaatatcagaattgattcttatggcagttgtcatcataacagggatggaagagttgacaaagtggaagcactgaagcggtacaagtttagcttggcttttgagaattctaatgaggaggactatgtaactgaaaaattctttcagtctctggtagctggatcaatccctgtggtggttggtgctccaaacatccaagactttgctccttctcctaattcagttttacacattaaagagataaaagatgctgaatcaatcgccaataccatgaagtaccttgctcaaaaccctattgcatacaatgagtcattaagttgtttacttgataatgggatgatggaagtttga

SEQ ID NO: 34 - protein, 374 aa, Nicotiana tabacum
MRSSSNSNAPNKQWRNWLPLFFALVVIAEISFLVRLDVAEKANSWADSFYQFTTASWSTSKLAVDHGDVEEVQLGILSGEFDQGFVPGSCEEWLEREDSVAYSRDFDNEPIFVHGPGQELKTCSVGCKFGTDSDKKPDAAFRLPQQAGTASVLRSMESAQYYAENNITLARRWFSLPGVGVRLRTSYGLTLSRPTGRGYDVVMTTSFSSDVPVGYFSWAEYDIMAPVQPKTENVLAAAFISNCGARNFRLQALEALERANIRIDSYGSCHHNRDGRVDKVEALKRYKFSLAFENSNEEDYVTEKFFQSLVAGSIPVVVGAPNIQDFAPSPNSVLHIKEIKDAESIANTMKYLAQNPIAYNESLSCLLDNGMMEV

SEQ ID NO: 35 - DNA, 20 bp, artificial sequence
  /note=“Description of artificial sequenceNGSG10034 forward primer”
agagacgtcaaggtgatcca

SEQ ID NO: 36 - DNA, 20 bp, artificial sequence
  /note=“Description of artificial sequenceNGSG10034 reverse primer”
ggcaagctggtttctttgag

SEQ ID NO: 37 - DNA, 4871 bp, Nicotiana tabacum
atatctcagagcatgttgtgtaccttgtattatatgactgtataactagttgagtttccatggaattgtagcaatgaaagcctttttcgtgtgctggtttctctttatctaagtttgggtttcggatacccgatgcttaaaggaaaaaagaaaagaattcagtggtctctggtgctgagtttcttaaaagatggggatagctgggatgacttgtttcaagatcaacttcccccccccccccccccccccacaaatccaacctaaaaacaaattaccaaagaaaaggaaaaccaaattgtaaaccagcagtagtagtttgggtttaatcttccattaaaacgtattcattagactcttgatgtgcatttccattacgaagaagaaaaaggatttaattgaactgcaagtctacaataaagactgtgtcatagtttcattctctaatttgacagataatgattagcgtaatttaccagcaaaagaagttgatactattgacatctagagacaagaagaattctaggatcatgaatgagccagagttcataatcaaggagaatattggtggaaattgacattatataatagataattttctttgaaccaaaatttttatgtatgattaatctgtatgggcacgggatctttacaagtcaccctacctagtctatatcctatcaacacaaacattgttaagaaacaacactaaaagacctaagtacacaataatgaaatttaaaacctagaaccctgtatttttgcagttgaataatcaagaagctcttgcatttctcccatttcttttactgatggcttcacataacctgcaaggtaaagtatccagacttgctgctcgggcctttctatagtttccaagcacaccacatcttctccatgcatgatactctgcataagctaccggatcattagcaacggccttaacgtaggaagccagttcctccaaagagctaaacttgcttccatcaattattgaatgtggaggtacaaaatcccagacattcgcggcaccaaaataaatggggaccgcgccagagtccagtgcataaaacaacttctctgttacataactctctgtcctggtgttctcaatcgcaaggacaaacttgtaatgtgacatagcgcaatgtaaatggtcccaccatttgggtgcttccttaaaatccttgatgcactcaggatagagggagagtgccttgtctagacctccaacattgttcaagcacttgccaaatgaatgggaaggtagcaagctgagtagacgtttggcaagctggtttctttgaggaagacaacgtgatgatgaccaataaacaagagtatcctacgaatttacaaattgctagagattataacaagcacttaaaagaatgagcaagtaaaatcattatgctcaacgattgaatatttacatgcaaatttagggaacaaagtggcctttcacttacattgttcttataaggcgaaaggtgataatttcgattattatggaaaagtgaaccagcataggttgactggacatcatcttctgcatgatagctaataaatatatcctcatacactgatttctttctaccagcttcaagatccatgtatacacgtaatggatcaccttgacgtctctgataaaggaaataacaattgtgaggagactgtagagaagaatatgatgcatgtggtgtgtgtgcaacaagaaagcaaaagttcgatgtaatttagattcgcagtggggattttccatcaacctttccatgtcaaacaataaaaatctgaatctcaatggttactagtcacacaattcctataacacacagccaaataatccattcattcgcaattcatcttctacaactcacacccttcttgggtaatgccaaacagtactaccatactcttaagcaactcaattcactctttgactcaagatcttttgcgtgggtaattctcccttcccttcgatgcagaggctcaaaaaagagggtgatggaatagcttagagacagatgcagaaataacagcactaagatatatttcgggtaactagagtcaacaatatgttttccaacagactaatcattcagcaaaatcagcatttcaagaaagaaacttaggaagaatatactacaataatggtcagaataaagcaagattcaatcgccaattctcaaatgatgaacccagaaaataaggaatggaaaatttactgattgactaaggaactttagaaccataacaaaaccaaagggtataattttagatcctgttacgtagctgcaattcgatccacctgtacatcaacatctatgagaacaaatagctatgttccatgtcctaacaagctgctagtaactattcttacaattaacatcgtaacaccctattatagcaaacaccttgaataagctatataactgaacataaaacaggaaagcaggatgccaaaacattatagtcaattagtcatacaccaaactctaattttgcaaagggtaggtctaagtggagcactgtacatgctaagttcacatcaatgtatatatagactttaactaattggttacttgaagcaagctagcaaaaacaccaagaaattgaatagttaaatatgtcatagagtacagcaggtacagcagacagtttttttggggtttacttgcaaaaaattgagtaaaaaacattgaccactatacacaaaaataagtcttaaactaagatagaatcataaattactagtaagataagaaatcatcgggtctgaagtattttttaccaacagtacttcactttatccataatgcaagtcggatagtcctgctgccattataagtaaaggatacttttttttttctattttctccatacatcaataacatatatctcgagaagaataagtgaaacaaccattgattggtcaacatcacagctttgaggctccattagaaaacaataatctgtgacatatggatgaaagtaatctgtgactgtgggttagtctatctgaaaaataaagggtaaaaggtttcgttttctggaaataaatgtttcctagctgcaagctccaccgattagcttcaaaaatatcagaagtttaatgtaaagtacttgcaaagagttagcaaacgaaaggaattgaggcttcaagattacatttctggagctgtgacagatcgactagtccagtttagttggagagacctcagaacgtttggaagctaaacatatacagggaatagcagcacgaaacagatgattcaaaactctcagtacattcacatgtatctatttcacataattaaaccggtaggatgatgtctgcagagcatctaatggaacatatattgttgatgttttagtatatcacctcaaacattctaaggaaaagagaattctaaatgtatttgaagtagaagcttgttaagaagcttaattgacggtctgagaattcggatcaaaacatgtgaatgcctgaattcatcatgcactaactttataagaaagggtaacgaggtagatggaatatacaagttgcaatcctcttgggaaatgattattcgctgtccatttcactcttctgtattgtgacatgtaagatgacataaaaaggtaattccatgggtctctccaacaaggcataaagaaatcatatgctgataccttgactgattttaagcgactgcaaacatatttatcaatcttagattattagacatttactcaactatatttggagaagtgattgcattcacactaaagcatcggaatactttcaaggctgaaggaagatctttcgagcccgcatcaaccccaaattagttgggattcggtatatgagtcctcaatttccattctgctctatttaagcccatgtcattccagtactaaataatttacttttttaagggaaatgaaggttctctaacattagagtttctctaaatgtattttacatggacaaaccagaataaaaagctcctaacaaacgccggaacccaacgtaagtgtaacaacaacaacttagtgttttttttttggagaaggtaacaacaacaacaacttagtctcatccagctagttgatcatttgcctccactttgtcatgttttgagccaagtccaacactgattcctaaagatagcagcttagacagtaatcaatcaggcactaccacgaactagaaaatataagttcatgcagatgcattattaatgttgatttccacagctaaaactgctcaatcacgcttaagctaaaagagctaatattggttaaaaataatacctcaaggggaggagttgctgtctcaaacaacaaagcatcaggcttatcagctagaacctcggatttagtccacaaacagctaagcccacatcgacaagagtacaaattatccaaattatcaggaatccaagtccagcctttaactaatacactcacatgatccatcttcagctcattacatttcaattcaccctcatcaactttttgcaaagaaccagaagaagaatttaacaacaaccctttgtgtttatccttgaattgagcacaacccacttgagaatcccatttcttgaaagcaccgaacaagttgctaaacgggtcgggcttggaagctgagatggtggtcaagatttgatttttggttgatgggattgaagaagcggtaagtgggaaatcaagaaatccagagaaaaataggatgataagtgcaaaaccaaacatgatagtgattgcaaatgtgttgacagatttcaattgcatttttcagtgcaagaatttacaaaaagaaatcagctattattctctctccccctttgtcgctcaaggagagagagagagagagagagagagtagta

SEQ ID NO: 38 - DNA, 1230 bp, Nicotiana tabacum
aaaatgatgcaattgaaatctgtcaacacatttgcaatcactatcatgtttggttttgcacttatcatcctatttttctctggatttcttgatttcccacttaccgcttcttcaatcccatcaaccaaaaatcaaatcttgaccaccatctcagcttccaagcccgacccgtttagcaacttgttcggtgctttcaagaaatgggattctcaagtgggttgtgctcaattcaaggataaacacaaagggttgttgttaaattcttcttctggttctttgcaaaaagttgatgagggtgaattgaaatgtaatgagctgaagatggatcatgtgagtgtattagttaaaggctggacttggattcctgataatttggataatttgtactcttgtcgatgtgggcttagctgtttgtggactaaatccgaggttctagctgataagcctgatgctttgttgtttgagacagcaactcctccccttgagagacgtcaaggtgatccattacgtgtatacatggatcttgaagctggtagaaagaaatcagtgtatgaggatatatttattagctatcatgcagaagatgatgtccagtcaacctatgctggttcacttttccataataatcgaaattatcacctttcgccttataagaacaatgatactcttgtttattggtcatcatcacgttgtcttcctcaaagaaaccagcttgccaaacgtctactcagcttgctaccttcccattcatttggcaagtgcttgaacaatgttggaggtctagacaaggcactctccctctatcctgagtgcatcaaggattttaaggaagcacccaaatggtgggaccatttacattgcgctatgtcacattacaagtttgtccttgcgattgagaacaccaggacagagagttatgtaacagagaagttgttttatgcactggactctggcgcggtccccatttattttggtgccgcgaatgtctgggattttgtacctccacattcaataattgatggaagcaagtttagctctttggaggaactggcttcctacgttaaggccgttgctaatgatccggtagcttatgcagagtatcatgcatggagaagatgtggtgtgcttggaaactatagaaaggcccgagcagcaagtctggatactttaccttgcaggttatgtgaagccatcagtaaaagaaatgggagaaatgcaagagcttcttga

SEQ ID NO: 39 - protein, 409 aa, Nicotiana tabacum
KMMQLKSVNTFAITIMFGFALIILFFSGFLDFPLTASSIPSTKNQILTTISASKPDPFSNLFGAFKKWDSQVGCAQFKDKHKGLLLNSSSGSLQKVDEGELKCNELKMDHVSVLVKGWTWIPDNLDNLYSCRCGLSCLWTKSEVLADKPDALLFETATPPLERRQGDPLRVYMDLEAGRKKSVYEDIFISYHAEDDVQSTYAGSLFHNNRNYHLSPYKNNDTLVYWSSSRCLPQRNQLAKRLLSLLPSHSFGKCLNNVGGLDKALSLYPECIKDFKEAPKWWDHLHCAMSHYKFVLAIENTRTESYVTEKLFYALDSGAVPIYFGAANVWDFVPPHSIIDGSKFSSLEELASYVKAVANDPVAYAEYHAWRRCGVLGNYRKARAASLDTLPCRLCEAISKRNGRNARAS

SEQ ID NO: 40 - DNA, 3152 bp, Nicotiana tabacum
ttatacatggcttatatctcagatctatctttcttgtacgattaagatcaccagcaatgaaataggttcattaggttaggtttcttttggaccttagccttctcttaaattaccactgtttcatatgaactctacatgaacataatttgcaatctttaatacagaaaattgatgactaagaaattagtggaactaattttgaattacgtagaatttagaacaagtttgttattaaatcttaggaaactagagaacaattttaacatcaacttgtgggcagtcaggatttatacctaggggattaaaaaaaaatgcaaacttgcagaatagcttaactatcaaggggattcaacaattttttttatatatataaaaaataatttttccctatttgtacagtgtaactttcctcgcaagagattaaagtgaacccccttcaatacatttattgatttagctgtgtcactagtggggtgtgccactttaagcagctggttccctcttttagtattttggtcgcaaattccccttggcaaagataaggtgaaccgctaggaaagaattgacattcacatgcccaaaagaacttctgtaggctatgcatttgaaattttcatggcttgtaggcgaagcaattgaaacttttttctgctattgcaaatttgcaatagattctgacgacactgtaccatctgaggtaaataacttttggtactgtactgtatggtttagttttggtatctctgttatctctttctaatgtattagacaaaagcaaatatcaagatttaacttctagccccaaggttctggcgtaacaaatgaacaatttgggcaacaatattctcatctgcctaagcttggtggatagagttacttgatatctgtgctagtaggaggtattaagtacccggtggattagtggagatgcatgcaaccgcaattgtaaaaagaaaagtttatattgcttagggaaagccaagcaatatatgaggttacttggttttgttgacatgggtattatgaaaagaatttaccttttttttttgatttctttctttttctttctggattagtgtttgcttaatggtgaattaggtatggttttaagtggttgcttttgctacattgctcagatgcggctttttgcgacacagtcagaatatgcagatcgccttgctgctgcagtatgtatctggactcatctagtcatcctccctacaggaaatctaaataccatagacatatttcttttgttctacagtttaagaatttgtattcatgtcatgtattgtgaatatgatgtttctaaaatcttcatatgctctacgtgaaggcatccttcaacaattcaaatgtcattccaaaaatcttctcttttcttctcagaaggatattgcataatctttctttgtgttgtcttaacagcatacaactgcgcccttcttcaatgatgcaggctaaagaaagaagtaaagaacttttaattgctcactatgtgtataaatcattgaatgacacagattgaagcagaaaatcactgtacaagtcagaccagattgcttattgaccagattagccagcagcaaggaagaatagttgctcttgaaggtgcaatgtgtttttcggtgtagtcctttctttcttcattgtcctcttgataaatggatttatttcctccattctacaaatggatctattggaaatagtctatcttgaaaattttatgtaagttttggtcctatcataagtgagtacactgaaaatatttgatcaagaagatgcaagagagtgtagaagatagtaatggttaactccaagtacaaaaatctagatcagagcatgagctaaccaataccaaaactttgcctgctaggccagagtaagagagctaatgaaatctaggaggggaataacgtcatttacaggggaaaggttactccaactaaaaagattcatcaaacatatagatttcagggagcaattaggagttgaaatgccatcaaaacatctgctatttctttctgtccaaatacaccaaaaaatacacgctgggatcatctgccaggtctttttgatggttccgtcaacttcccagaagctccaattttctactgcttcctttaggttctgaggtgttgtccagctaataccaaaaactgataggaacatttaccatatgtctgcagccactgaacaatgcaaaaaaagatgatttactgactctgaactctgatgacacatgtaacatctgttcaccatttgaaaaccccttctacaaatcttgagtcaggatagcttcttcaagggctgtccatgtaaagcacatgacttcagtggggagttttttttctagattagtttccaaggccaatgatcaatcacttcatttgatacgcacattttgttgtaccctgccttcactgaataaatgcccttgctggtgttgtcccacattaggatgtctgggttttgtgggttcatcgtgaggtcttcaagtattctgtatagatcaaagagttcgtccagttcccaatccagcatgttccttttgaattgaatgttcagttgttcccatccctgttatgtgctattgtgttgatgtagatctggtctcttttgagaaattttctgtttttgtgttgtaagtttcgagacatcttcatggatgagcagtgaggataggaccttttcagtttcttcgtgtcctctttcaatgctttgttccttatcctttctgtgataatacagatcatgttgaatatttgcttctgttactgctgatttatgatttactagaataataagtagtttagtagtaggaggggtctttgtttaaatgtaaatttagttggataagttagttgagatatttgaggtttttgaaatttgaatatttattctgcagattatgttttcaagttggctatttaaagccctctggttaataaaattaaaatgagagacaatttcaaccattcttttaatcttcttgctgctccatctctttaaaaaacctaacagatcccaattaataaaatctggtgtttgctgtcagaaactgaaatgctacttatctcttttgtatgaagggaacaggtagttgtattttttggggggaggggaagaaaggtaatgggtaattttacttt

SEQ ID NO: 41 - DNA, 3140 bp, Nicotiana tabacum
  misc_feature (2977)..(2978): n is a, c, g, or t
ttatatctcagatctatctttcttgtacgattaagatcaccagcaatgaaataggttcattaggttaggtttcttttggaccttagccttctcttaaattaccactgtttcatatgaactctacatgaacataatttgcaatctttaatacagaaaattgatgactaagaaattagtggaactaattttgaattacgtagaatttagaacaagtttgttattaaatcttaggaaactagagaacaattttaacatcaacttgtgggcagtcaggatttatacctaggggattaaaaaaaaatgcaaacttgcagaatagcttaactatcaaggggattcaacaattttttttatatatataaaaaataatttttccctatttgtacagtgtaactttcctcgcaagagattaaagtgaacccccttcaatacatttattgatttagctgtgtcactagtggggtgtgccactttaagcagctggttccctcttttagtattttggtcgcaaattccccttggcaaagataaggtgaaccgctaggaaagaattgacattcacatgcgcaaaagaacttctgtaggctatgcatttgaaattttcatggcttgtaggcgaagcaattgaaacttttttctgctattgcaaatttgcaatagattctgacgacactgtaccatctgaggtaaataacttttggtactgtactgtatggtttagttttggtatctctgttatctctttctaatgtattagacaaaagcaaatatcaagatttaacttctagccccaaggttctggcgtaacaaatgaacaatttgggcaacaatattctcatctgcctaagcttggtggatagagttacttgatatctgtgctagtaggaggtattaagtacccggtggattagtggagatgcatgcaaccgcaattgtaaaaagaaaagtttatattgcttagggaaagccaagcaatatatgaggttacttggttttgttgacatgggtattatgaaaagaatttaccttttttttttgatttctttctttttctttctggattagtgtttgcttaatggtgaattaggtatggttttaagtggttgcttttgctacattgctcagatgcggctttttgcgacacagtcagaatatgcagatcgccttgctgctgcagtatgtatctggactcatctagtcatcctccctacaggaaatctaaataccatagacatatttcttttgttctacagtttaagaatttgtattcatgtcatgtattgtgaatatgatgtttctaaaatcttcatatgctctacgtgaaggcatccttcaacaattcaaatgtcattccaaaaatcttctcttttcttctcagaaggatattgcataatctttctttgtgttgtcttaacagcatacaactgcgcccttcttcaatgatgcaggctaaagaaagaagtaaagaacttttaattgctcactatgtgtataaatcattgaatgacacagattgaagcagaaaatcactgtacaagtcagaccagattgcttattgaccagattagccagcagcaaggaagaatagttgctcttgaaggtgcaatgtgtttttcggtgtagtcctttctttcttcattgtcctcttgataaatggatttatttcctccattctacaaatggatctattggaaatagtctatcttgaaaattttatgtaagttttggtcctatcataagtgagtacactgaaaatatttgatcaagaagatgcaagagagtgtagaagatagtaatggttaactccaagtacaaaaatctagatcagagcatgagctaaccaataccaaaactttgcctgctaggccagagtaagagagctaatgaaatctaggaggggaataacgtcatttacaggggaaaggttactccaactaaaaagattcatcaaacatatagatttcagggagcaattaggagttgaaatgccatcaaaacatctgctatttctttctgtccaaatacaccaaaaaatacacgctgggatcatctgccaggtctttttgatggttccgtcaacttcccagaagctccaattttctactgcttcctttaggttctgaggtgttgtccagctaataccaaaaactgataggaacatttaccatatgtctgcagccactgaacaatgcaaaaaaagatgatttactgactctgaactctgatgacacatgtaacatctgttcaccatttgaaaaccccttctacaaatcttgagtcaggatagcttcttcaagggctgtccatgtaaagcacatgacttcagtggggagttttttttctagattagtttccaaggccaatgatcaatcacttcatttgatacgcacattttgttgtaccctgccttcactgaataaatgcccttgctggtgttgtcccacattaggatgtctgggttttgtgggttcatcgtgaggtcttcaagtattctgtatagatcaaagagttcgtccagttcccaatccagcatgttccttttgaattgaatgttcagttgttcccatccctgttatgtgctattgtgttgatgtagatctggtctcttttgagaaattttctgtttttgtgttgtaagtttcgagacatcttcatggatgagcagtgaggataggaccttttcagtttcttcgtgtcctctttcaatgctttgttccttatcctttctgtgataatacagatcatgttgaatatttgcttctgttactgctgatttatgatttactagaataataagtagtttagtcgtaggaggggtctttgtttaaatgtaaatttagttggataagttagttgagatatttgaggtttttgaaatttgaatatttattctgcagattatgttttcaagttggctatttaaagccctctggttaataaaattaaaatgagagacaatttcaaccattcttttaannttnntgctgntccatctctttaaaaaacnnancagnncccaattaataaaatntggtgttngntgncngaaactnaaatgnnanttatntnttttgnntnangggaacaggtagttgtattttttggggggaggggaagaaaggtaatgggtaattttactt

SEQ ID NO: 42 - DNA, 22 bp, artificial sequence
  /note=“Description of artificial sequence targeting sequence”
ttttcatttcagtggattgagg

SEQ ID NO: 43 - DNA, 22 bp, artificial sequence
  /note=“Description of artificial sequence first derivative target”
ttttcatttcatgaaatgaaaa

SEQ ID NO: 44 - DNA, 22 bp, artificial sequence
  /note=“Description of artificial sequence second derivative target”
cctcaatcctcgtggattgagg

SEQ ID NO: 45 - DNA, 20 bp, artificial sequence
  /note=“Description of artificial sequence NSGSG10035 forward primer”
ttgcaactccagacaagcag

SEQ ID NO: 46 - DNA, 20 bp, artificial sequence
  /note=“Description of artificial sequence NSGSG10035 reverse primer”
tgcatcagttgccgagacta

SEQ ID NO: 47 - DNA, 11000 bp, Nicotiana tabacum
attcgagctcggtacccgggatccatatccccgaatcttaaaatttatatctcgaagaatctgatatctagatccgcacccgtgtcggacacccgcacctgtatccaagcaacttagcccatagaccatgggctctcagccaagccatatacttctcatacattgatagtaagacacagtgaataattactcaagcaatttcatcactatgcctcaacccctatttgcgatcagctatatgaatcctctatatacattccactctattcggtcgagggtctatcggtaatagcctctctgccccatcggggtatgggtaaggtttgtgtacacacactttgtgggattttaacgggtcgttgttgttgttgttgtacattccactctgttcggacccttttcattccaatacttgtaaataatttgatcgccatggtacatattaaaaacaagaacagtggcacatgaacatatacagaaaatgcacttatatatgcatgaatagaaattagtcatgacagatagagaataccttaggaaaatggactccatgtcaaacctccctctttcacgtacatatatatgatagacagtttctgaacctctggtacatttgcagggacgttttgtaaattctggactcttctcttctttctccctaatactagttgctaagaagatacacaaacgacaagaagagtgaactgctgccatgtcaaccagggctttgaaagagtcagatggaccgtcaagcttccacctaaacaatgaaaaaaagcattagttagccagacacaggaaatttaagaaatccttaaaaaaatttgaaaggaaagagtaaattgcaactccagacaagcagagaggggaaacacacacacaaggagaacaatcttcccaattcaggagttttagttagaccaaaacaatatatcagtatgtaaataggataaggtaataaacttttgtaactatacagacagggctgttgttattttctcaataacatgataccgaaaaagtacagaaagatgctggaaaaaggtgttctttgagagtggataaatcttcaggacaaccaatgaaaagttatttaggacaggaaaattcaaataatcaagaagagcacgtaagaaaagcaccactaattgatgcataccttaatgactcattatatgcactaggattttctgcaaggtacttcatagtctcggcaactgatgcagcgtctttcagctctttaatgtgtaaaagtgaattaggagaaggagcaaagtctaggatgtttggagcaccaatcaccacagggactgatcctgcaagtttacatgaacttcagagttgaccaccataaatcttaattttgaagtagtaacttgtgaaacatcataaaaacaggattctgtgcataaatctagcctaaaatatattgactatgagcacaaagaatatgattaaacaatgataattcatgtaaaatcatttaaaattttcctaaagctttttaaatgaatatagagcagagcatatttgtgcgctgtaccagaaaatgcaaatccaggttcaaaatgtagttgaagtaccataaaatgctcataagtttgtgtttttgcaatatttgttcttcatggcacgctctatagctaatatcttcaaagtctttgtgaaagtatggcattgcagaaaaagtagaacaaaatgcatcaatgtagaggggtggggctgagagaaaataagagatcctacatcagaaactaagcgtacaaagcagaaccaaaattaacaggcaaaagttaataccagctaccagagactggaagaatttttcggtgacataatcctcctcattagaattctcaaaagcgaagctaaatttatagcgcttgagagtttccactttgtccactgccatgaaattgagaacaagatgacaagtgagcattcacaggatataatcatattatactaaaagtgggaacatcctatcctcaaagttaatttactaatataaccttaaaaaactgaatgacaattttatccttgagccaaaaaaatgaccttccagtaaatgaatacttttattacttttaaccgggtgaatgtaggcattgattgtttttttatacaagtttttttgagtttaattatcttaattattcattgattgtttgtttctttatcttccactgttataatatcgcactacacaaagagccacatcccttcattaatctctaaaaacaccttgatctctctctttccttagttttttactaaataattatttctgtgctttggttaaaactcaaaagccgcataatccttttctattcctcttctttctctccatcatttgttttatcgtatttttttaaaggcatcaatttaatcaatttttagcaaagtaaactaaagtaactcattatgttattttggaacttactttatgaatattaaatttcgggatgagttaaatataataaatctataatttggattcaacgcacgtgtccaagaactagtgtactgttaaactagaggcatgcataaaaatattctgcattcaaatatgagatttatcaagactgccttttctcttattttatttttttgttggataaaaaccttttctcttcatttctctttccaataaaccaaggtctagcttcttcacgatggatttactaaaaaaatgtctgcttctcaactctattctacgtgaatttatatctaacgtacagttaaaccacatgtatgcataaatatattctgcattccgggaaaaaaaattatctaggctaccttttctctaaatttttcttttctaactaatcgggaactagcttctttgcaatgaatttacaaaatgtgtctgctttcaactctattttaatgggttgcaaactaataggtattcaaaagataaataaaaaaagtgtttgctgtctttcttttgatataattatgtgaccttcttggtactcctattaataaaacttaaccttatccaaaagaaaaaaagataacaagaataacagcaaaataacctatccacaaaatgaaatctttagactatcagaacatctggctatttaaaaaacttgcattggtatgtgtccatttagcaaggaaacaaatataaggtttctattgccagtaaaattggcttttcaagggatataatacacttgaggaagagagaatcaaacccattatattgaaatatgctagacatttagctctttaatggtaactacttcaagaaatgaaccatcatattatatggagatacactgaccatttccatcccggttacgatgacaactgccaaaagaatcaatcttgatatttgccctttcaaggacttcaagagcctgtaaccggaagttgcgagcaccacaattagaaataaaagcagctgctaacgcattctcagttttaggttgcactggagccattatatcatactccgcccaagagaagtacccaacaggaacatccgaagagaggcttgttgtcattacaatatcatatccccttctgaaacatgagaaagaataataaagttggttagagatttccctagagaaagatataaagattgagagaagaactataggagatctaaaatttaaatttaaagatataaagattgagagacaattagaactataggagaactataggaagataatgaattgctacaaggagaatttgaagaacaagaagtgtttgagaccttgaagatatgtgcagctgacaaagcaccgggccctgatggtttctctatggggtttttccataattgctgggagattgtgaaagaagacatcatgcataccatcagaaacttccacaacaatgaatattttgagaagagtttcaatgctacttacatagcccttattccaaaaaagaatggtgctaaggaactcaaggacttcagaccaattagtcttacaggaagcatttacaagatcatttccaaactattgacagagaggctcaagaaagtggtggacaaactagtgaatgggcatcaaatggccttcataagaggcaggcaaatcatggatgcttccttgattgcaaatgaatgtgtggattcctgacttaaaagggcaggttcccggaattctctgtaagcttgatattgagaaggcctatgatcatgttaattggaactttttactcaaagttcttcaggatatgggttttggtaggaagtggatcaactggatatctttctgcatcagaacagtcagattttccattttggtgaatggttctccagagggattttttccttctgagagaggtttgagacaaggggatcccttatcaccttttcttttcctctttgctatggaaggattgaaccaaatgttaaaaaatgcaaacaacaatggttggctgaaaggttttaaagcttcaaacagggagggagatagtgtggagatcacccatcttctatatgctgatgactctttagttttttgtgaggcaaaggcagatcaactaaaatatctaagagtcattcttgttatatttgaagcagtctcagggctccatgtaaatcggaggaagagcatgttgttcccagttaaagaagtggatgatatccaggccctagcagctatactgggatatgaggtgggttctctaccaactatatatttggatttatctctaggctccaagaacaaagctcaagaaatatggaatggggttcttgaaagatgtgaaaaaaggctatcaacctggaagagccagtatttatccttgggtggaagggtggtattggttaatagtgtcttggatgcacttcctacatgtgtaatgtctttgtttcccttaccaagcaaggtgaggaaaagaattgatgcactaagaaggagctttatatggcaaggaaacagagagaaggaagctattcatttagttaactggaattcccttatcacaagcaaagacaaagggggtttgggcattaggaaccttaaagcccacaatcagagtttactcttgaagtggctttggaggtacaatctgaaggctaatgctttgtggagaaaggtcatctgtgacaagtatggacaaaatggactatggtgctctaactctgttaatagttcacatggggttggggtatggaagtcaatcagacttcactggaatactctggctgataatacaaccataaaggttggtaatggaaggaaaacacttttctggagtgataactggttaggacatggtcctctcaaagaattttttcaaaaatttttcagcattgcaacatcacctacatccactttagacactgcctggggtcagcaaggatggaatgttacttttagaaaggccttgaatgattgggaattagaaagggttgtaaatttctttaatatcttggaacaattccaaggtcttggagaaactgaagataaactatgctggaatcattgaaggagtgggaattttacggtcagatccgcgtatactctactcgctgcttcaaaccaacagttagaacaatggccctggaggaatatttggaaagtaaaggcacctttcaaggtggtatgcttttcatggttagttacaagaaaggcttgtctaacccaagagaacttgagaagaagaggttttcagctatgctctaaatgcctattatgtggtacagctatagaaactaatagccatttgtttcttcattgtccttttactgaccaactgtggcaactatttcttaatattgtgggccttaaatggagcatgccagctaacacttgtgatatgttgaagtgttggaattacaatgagggtatagtgagacagaagaaatggtggcgattggttcctgcgtgcatatggtggacagtctggaaggagagaaatttaagaacttttgaagacagaagcaattctttacagaacatcaagatgaagtgtttacttttgttttatttctggtgtaaagaaaactggatagaggaggcagaatctttagtagacctgataggtgcactgtaatcttaggttgtcatagctggtttctgttttcttgtaaatatggcttggcactgctctagtgctgttttatttattaatatatatgttaccattatcataaaaaaaacatgatcctaagattattgtctatgagggctttatgattgcatatttgtagatgccctttccaaaggctatttgagcaaatgcaactgcttttttttcgtaatgatagaaaatatgcagtatggaaggctgttataaagccgatttatggcatggagagatattagaatccaaaaccagtaagtaatccttatgggttgcttgtggagaccaatagctaatttttggaggcagttcaaagataaacataaaactaaaagctgaaaatgatgggaaagtcaagctttggagtgatgaatagtgtaatgaaggtttacttatagatttattctcagccgtgttcagtgtggttccaaatcaggattgtctgtgaatctgttagtcatctcctgatgcatcgtaatctttcttggagtagctggtctttattccttaatattttgagggtatactgataaaaaatatattttgggggtaatactgggtaatgccacaaatttcaaaggtggactcatcagctggcaagcacagtggagaaaagactgaagaagatctgcaagcttattcctttgtgtattatttggagattttgtcaagaaagaaacacgagatattttgaggggggaaaggagcataattcagttataaagaatagatgtttacagtatctcttttgctggcataaaaccgatcctctagttagtacaaatgagttcttcaactcactagattcgggttttgagttgtctttggaagtctaaatttttgtacttttttggtaccttcttggtactgcttactaataatatgctccttaaaagaaaggcctagtcagaaactaatagcaagcaaaaacttccaagtgagatatgtttgattaagatacgatcacaagatgaaaactcaaatgtggcaaatgctattatctttcaggtgccaaatgtaggtgagaatgttttaaatctttttcaagatgtgcttacccaccgtcgtgccataacgatgttgttctcaggatagtattgagctgactccattgaccgaagcacgctagccgtgccagcctgttgtggtgtcccaaatgccgcatcaggcttcttttcagaatccacaccaaagttacatcctacggcacaagacttccaatcctagataatgattgaaggaacatcagttttcgcatatggtaaggaaaaaaaatcaagaagaaagatggtaaccaggtaactgttacctgctctcactacatgccagtggataacatgggaacaagaactactcatgataaacttaagcaataaatggcagttttactgattaatttctgctcattccatttaagtgctctatgaagaattcaatgtaatgaaggaaaaaaaggtaagaaaaaagaagatattcaaggctcctccacttattccctactagtatataaaattcatgagtatttttacccatataatcggttttggaatatggtcaatagtccttataagatgtttgaccgtaaacatgataaaagaacaaaaaagctagaagaaaatgccaaagcatcatttgaaacaacaatttttttatcattccacctaccaaagccacaagtctggaacacacacacacacacacacacacacacgagctctggcagctaactgatgtaataaatagcaagacataaattgcaacaaaattcaaattatcgtctaaaagagatgctcgatcaccaattaatgtttctatggagaattagctgctactaccttttaagtgaccttatggcgtaaatcttttttgcatcgttagtgcctagaactaatactaaagggcagtagtccggtgcacgaaggggttataatgtaggcagcctaccctgatgcaagcatcgtggctgattccacggctcgaacccacggctcgaacctgtgacctacggagacaactttaccgttactccaagaatcccctgtatagaactaatacgcatcatcaaaaaaagagcaatcaactatgcctcctaaactgttcgggttcaactataagaatcctctatattcattctgctctaaaaaaaaataaacatttacaagaaaactatatcaccttttcgccgccatgaacaaaaattgggtctttgtcaaaatctctagaatactccacagaatcctccttttccaaccactcctcacagctcccagtttccaaattccgatcaacctcactactcctcaacacacccaacccagtctcactaatttccactttggaggttgaccaagacgacgtcgtaaactggtaaaatgagtcagtccaagagttgaccaggttggctttttcagccatgtccagtcgacccagaaatgtaatttcaactataaccacaagtgcaactactagaggtagccaattggaccatttcttaaggggaacgtttgtaggtgatgatgacccaacaccttcaaatcttggtaacctttgaattggaataactgttgccattaataactcgaaattttgttctttttgtggaagtaaaatagctaaagatgtgatctttttttcttcttcttcagagggtttgggaattcttcatttggtgggacagttgggggtggggggggggggggttaggagtgaaagtcaggcaaagacagatatatgaattgggaaggggagtttgttggtcaatctataaacactgtaaagtctacgccaccattggtgtcttagattttattttattttattttttggccgtacattggtgtctcagttgacatttgtcgtttccgtttcgtttttccaagtgttatttatgcactgccgggtcgtttggttcacaaataaattaaggagatattatgatgtagctttgacaagtaattaaagaaaattatttaattgcactaattatgagaaagttttttatatatcattaatcataataattttttgattaaattatcctttatttattttcagaatttgtaataaatgataaaatactttctagagtttttatagatcatttattgaagcaacagtagatttcaaatatatatatacatttattttggaccaaatttgtttagctaaaatgacatttaattgtaatacaataattaagataccaaagtttaaaaaaatgtaacggtaaacgagtaataataagaaaaatttgctaacttttttcctcagtcatttatgaaatatcactaagcatttttctcgtttatttctgaaaagttatcccctggacattgtttatgcgtctgtcaatatctacttagattgaaatttatttaattttctttcaaactaatttaattaaagattcttttcaacccaatctaacaccaacacccccaaaagaagaagtaattcaaaaggtaagacaagtgatgaagcaaaagagtagaaatgatagtttgtaggcagagttttaagttggctcgaacataactgtcataataaaaaaaaaagattgacaatcaaatggtcaagatattgtattaatatccctgttgtttagttggatcaatcataaattaccaaatctcactaaaaccgaatccaacttaaatatgagctcaacagcaactcataacatctgatctggaaaagttggttaagtaggagcaactgttggtaacttagaatgattccgttaatgatggagaaattcaaaaatagtcaaatttacaagtggtaattgaaaaataatcacagttttaaaagtaatcgaaatttagccacttttcatataaagataaatttgaacgaaaacactgttcaaaattcgggaaaaaatccagcataatatactgaagttctagtataatatactgaaactccaatatattatactggagttccagtataatatatcggtccaccagaatatgctcgaagttcatacacaggtgctccaatctccagtatattatgctggaactttgcgcgtgttggaaaatatgctggaagttcatacataggtgcatcaatctccagtatattatgttggaccagtccgtgttgcagcaaaatagtgactatttttcaatgactttacaaacgctggctattttttaattaccaatccgaaaactggctagcccgtgctatttttacgttaattatggatgcctaatccttgcggactatttattaaaataaaaaggaaaaagaataaagaaagtcaaacaattagccatttccttgtgattgattattaagtcatgtggaagttgttactcctgctaatgatgttcgacaatattctgcaagagaagtttttgaagtaggtacagaaatggctgtgattccacccaccctatcccacatccctaaatcaccacattatttttaacaactctttttcaatccttctcattttcttcttgtttcttttgtcaatttcatttcatgtcaaaggatttcattctgtaactacaacaacaaacccagcttaattccaaacgtgggttttgggaagggtagtgtgtacgcagccttacccctaccctatgaaggtagaaagattgtttccgataaaccctcactaacaagcaacaacagcagtaatatattaaggtaatgaaagcgagcggcataacaagtaataaagaactaagagatttcattctgtaaaggaagaacaaagacaggaaagaactaaagaaagttttctttccgtgtacagaaaattatctacaacggtaaaattatcttgtgtcacctataggtcatgggttcgagccgtgaaagcagccactaatgtttgcattaggctagactgtctacatcacaccccttggagtgtgtctcttccccagaccctacgtgaatgcaggatgctttgt

SEQ ID NO: 48 - DNA, 1032 bp, Nicotiana tabacum
aactcttggactgactcattttaccagtttacgacgtcgtcttggtcaacctccaaagggagctgtgaggagtggttggaaaaggaggattctgtggagtattctagagattttgacaaagacccaatttttgttcatggcaagtcttgtgccgtaggatgtaactttggtgtggattctgaaaagaagcctgatgcggcatttgggacaccacaacaggctggcacggctagcgtgcttcggtcaatggagtcagctcaatactatcctgagaacaacatcgttatggcacgaagaaggggatatgatattgtaatgacaacaagcctctcttcggatgttcctgttgggtacttctcttgggcggagtatgatataatggctccagtgcaacctaaaactgagaatgcgttagcagctgcttttatttctaattgtggtgctcgcaacttccggttacaggctcttgaagtccttgaaagggcaaatatcaagattgattcttttggcagttgtcatcgtaaccgggatggagtggacaaagtggaaactctcaagcgctataaatttagcttcgcttttgagaattctaatgaggaggattatgtcaccgaaaaattcttccagtctctggtagcaggatcagtccctgtggtgattggtgctccaaacatcctagactttgctccttctcctaattcacttttacacattaaagagctgaaagacgctgcatcagttgccgagactatgaagtaccttgcagaaaatcctagtgcatataatgagtcattaaggtggaagcttgacggtccatctgactctttcaaagccctggttgacatggcagcagttcactcttcttgtcgtttgtgtatcttcttagcaactagtattagggagaaagaagagaagagtccagaatttacaaaacgtccctgcaaatgtaccagaggttcagaaactgtctatcatatatatgtacgtgaaagagggaggtttgacatggagtccattttcctaaggtattct

SEQ ID NO: 49 - protein, 344 aa, Nicotiana tabacum
NSWTDSFYQFTTSSWSTSKGSCEEWLEKEDSVEYSRDFDKDPIFVHGKSCAVGCNFGVDSEKKPDAAFGTPQQAGTASVLRSMESAQYYPENNIVMARRRGYDIVMTTSLSSDVPVGYFSWAEYDIMAPVQPKTENALAAAFISNCGARNFRLQALEVLERANIKIDSFGSCHRNRDGVDKVETLKRYKFSFAFENSNEEDYVTEKFFQSLVAGSVPVVIGAPNILDFAPSPNSLLHIKELKDAASVAETMKYLAENPSAYNESLRWKLDGPSDSFKALVDMAAVHSSCRLCIFLATSIREKEEKSPEFTKRPCKCTRGSETVYHIYVRERGRFDMESIFLRYS

SEQ ID NO: 50 - DNA, 15 bp, artificial sequence
  /note=“Description of artificial sequence 15 basepair output nucleotide sequence”
accgtannnggcgac

SEQ ID NO: 51 - DNA, 15 bp, artificial sequence
  /note=“Description of artificial sequence 15 basepair output nucleotide sequence”
ccgtatnnngcgacg

SEQ ID NO: 52 - DNA, 15 bp, artificial sequence
  /note=“Description of artificial sequence 15 basepair output nucleotide sequence”
tatccgnnnacggcg

SEQ ID NO: 53 - DNA, 15 bp, artificial sequence
  /note=“Description of artificial sequence 15 basepair output nucleotide sequence”
gcgaggnnngtgcta

SEQ ID NO: 54 - DNA, 15 bp, artificial sequence
  /note=“Description of artificial sequence 15 basepair output nucleotide sequence”
tctcgtnnnggcgag

SEQ ID NO: 55 - DNA, 15 bp, artificial sequence
  /note=“Description of artificial sequence 15 basepair output nucleotide sequence”
cggttannngtagga

SEQ ID NO: 56 - DNA, 15 bp, artificial sequence
  /note=“Description of artificial sequence 15 basepair output nucleotide sequence”
agttagnnngcgccg

SEQ ID NO: 57 - DNA, 15 bp, artificial sequence
  /note=“Description of artificial sequence 15 basepair output nucleotide sequence”
cgtggcnnncagggt

SEQ ID NO: 58 - DNA, 15 bp, artificial sequence
  /note=“Description of artificial sequence 15 basepair output nucleotide sequence”
ccttacnnnacgtct

SEQ ID NO: 59 - DNA, 15 bp, artificial sequence
  /note=“Description of artificial sequence 15 basepair output nucleotide sequence”
ggccatnnngggggc

SEQ ID NO: 60 - DNA, 15 bp, artificial sequence
  /note=“Description of artificial sequence 15 basepair output nucleotide sequence”
gccatannnggggcg

SEQ ID NO: 61 - DNA, 15 bp, artificial sequence
  /note=“Description of artificial sequence 15 basepair output nucleotide sequence”
gcacggnnntccgag

SEQ ID NO: 62 - DNA, 15 bp, artificial sequence
  /note=“Description of artificial sequence 15 basepair output nucleotide sequence”
gcgaatnnnggcgcc

SEQ ID NO: 63 - DNA, 24 bp, artificial sequence
  /note=“Description of artificial sequence 24 basepair sequence”
ttttcatttcagtggattgaggag

SEQ ID NO: 64 - DNA, 24 bp, artificial sequence
  /note=“Description of artificial sequence 24 basepair sequence”
tttcatttcagtggattgaggagc

SEQ ID NO: 65 - DNA, 24 bp, artificial sequence
  /note=“Description of artificial sequence 24 basepair sequence”
ttcatttcagtggattgaggagcc

SEQ ID NO: 66 - DNA, 24 bp, artificial sequence
  /note=“Description of artificial sequence 24 basepair sequence”
tcatttcagtggattgaggagccg

SEQ ID NO: 67 - DNA, 24 bp, artificial sequence
  /note=“Description of artificial sequence 24 basepair sequence”
catttcagtggattgaggagccgt

SEQ ID NO: 68 - DNA, 24 bp, artificial sequence
  /note=“Description of artificial sequence 24 basepair sequence”
atttcagtggattgaggagccgtc

SEQ ID NO: 69 - DNA, 24 bp, artificial sequence
  /note=“Description of artificial sequence 24 basepair sequence”
tttcagtggattgaggagccgtca

SEQ ID NO: 70 - DNA, 24 bp, artificial sequence
  /note=“Description of artificial sequence 24 basepair sequence”
ttcagtggattgaggagccgtcac

SEQ ID NO: 71 - DNA, 24 bp, artificial sequence
  /note=“Description of artificial sequence 24 basepair sequence”
tcagtggattgaggagccgtcact

SEQ ID NO: 72 - DNA, 24 bp, artificial sequence
  /note=“Description of artificial sequence 24 basepair sequence”
cagtggattgaggagccgtcactt

SEQ ID NO: 73 - DNA, 24 bp, artificial sequence
  /note=“Description of artificial sequence 24 basepair sequence with 0 hit threshold run for SEQ ID NO 5 and the tobacco genome sequence assembly of Example 1”
agtggattgaggagccgtcacttt

SEQ ID NO: 74 - DNA, 24 bp, artificial sequence
  /note=“Description of artificial sequence 24 basepair sequence with 0 hit threshold run for SEQ ID NO 5 and the tobacco genome sequence assembly of Example 1”
gtggattgaggagccgtcactttt

SEQ ID NO: 75 - DNA, 24 bp, artificial sequence
  /note=“Description of artificial sequence 24 basepair sequence”
tggattgaggagccgtcacttttg

SEQ ID NO: 76 - DNA, 24 bp, artificial sequence
  /note=“Description of artificial sequence 24 basepair sequence”
ggattgaggagccgtcacttttga

SEQ ID NO: 77 - DNA, 24 bp, artificial sequence
  /note=“Description of artificial sequence 24 basepair sequence”
gattgaggagccgtcacttttgat

SEQ ID NO: 78 - DNA, 24 bp, artificial sequence
  /note=“Description of artificial sequence 24 basepair sequence”
attgaggagccgtcacttttgatt

SEQ ID NO: 79 - DNA, 24 bp, artificial sequence
  /note=“Description of artificial sequence 24 basepair sequence”
ttgaggagccgtcacttttgatta

SEQ ID NO: 80 - DNA, 24 bp, artificial sequence
  /note=“Description of artificial sequence 24 basepair sequence”
tgaggagccgtcacttttgattac

SEQ ID NO: 81 - DNA, 24 bp, artificial sequence
  /note=“Description of artificial sequence 24 basepair sequence”
gaggagccgtcacttttgattaca

SEQ ID NO: 82 - DNA, 24 bp, artificial sequence
  /note=“Description of artificial sequence 24 basepair sequence”
aggagccgtcacttttgattacac

SEQ ID NO: 83 - DNA, 24 bp, artificial sequence
  /note=“Description of artificial sequence 24 basepair sequence”
ggagccgtcacttttgattacacg

SEQ ID NO: 84 - DNA, 24 bp, artificial sequence
  /note=“Description of artificial sequence 24 basepair sequence”
gagccgtcacttttgattacacga

SEQ ID NO: 85 - DNA, 24 bp, artificial sequence
  /note=“Description of artificial sequence 24 basepair sequence”
agccgtcacttttgattacacgat

SEQ ID NO: 86 - DNA, 24 bp, artificial sequence
  /note=“Description of artificial sequence 24 basepair sequence”
gccgtcacttttgattacacgatt

SEQ ID NO: 87 - DNA, 24 bp, artificial sequence
  /note=“Description of artificial sequence 24 basepair sequence”
ccgtcacttttgattacacgattt

SEQ ID NO: 88 - DNA, 24 bp, artificial sequence
  /note=“Description of artificial sequence 24 basepair sequence”
cgtcacttttgattacacgatttg

SEQ ID NO: 89 - DNA, 24 bp, artificial sequence
  /note=“Description of artificial sequence 24 basepair sequence”
gtcacttttgattacacgatttga

SEQ ID NO: 90 - DNA, 24 bp, artificial sequence
  /note=“Description of artificial sequence 24 basepair sequence”
tcacttttgattacacgatttgag

SEQ ID NO: 91 - DNA, 24 bp, artificial sequence
  /note=“Description of artificial sequence 24 basepair sequence”
cacttttgattacacgatttgagt

SEQ ID NO: 92 - DNA, 24 bp, artificial sequence
  /note=“Description of artificial sequence 24 basepair sequence”
acttttgattacacgatttgagta

SEQ ID NO: 93 - DNA, 24 bp, artificial sequence
  /note=“Description of artificial sequence 24 basepair sequence”
cttttgattacacgatttgagtat

SEQ ID NO: 94 - DNA, 24 bp, artificial sequence
  /note=“Description of artificial sequence 24 basepair sequence”
ttttgattacacgatttgagtatg

SEQ ID NO: 95 - DNA, 24 bp, artificial sequence
  /note=“Description of artificial sequence 24 basepair sequence”
tttgattacacgatttgagtatgc

SEQ ID NO: 96 - DNA, 24 bp, artificial sequence
  /note=“Description of artificial sequence 24 basepair sequence”
ttgattacacgatttgagtatgca

SEQ ID NO: 97 - DNA, 24 bp, artificial sequence
  /note=“Description of artificial sequence 24 basepair sequence”
tgattacacgatttgagtatgcaa

SEQ ID NO: 98 - DNA, 24 bp, artificial sequence
  /note=“Description of artificial sequence 24 basepair sequence”
gattacacgatttgagtatgcaaa

SEQ ID NO: 99 - DNA, 24 bp, artificial sequence
  /note=“Description of artificial sequence 24 basepair sequence”
attacacgatttgagtatgcaaac

SEQ ID NO: 100 - DNA, 24 bp, artificial sequence
  /note=“Description of artificial sequence 24 basepair sequence”
ttacacgatttgagtatgcaaacc

SEQ ID NO: 101 - DNA, 24 bp, artificial sequence
  /note=“Description of artificial sequence 24 basepair sequence”
tacacgatttgagtatgcaaacct

SEQ ID NO: 102 - DNA, 24 bp, artificial sequence
  /note=“Description of artificial sequence 24 basepair sequence”
acacgatttgagtatgcaaacctt

SEQ ID NO: 103 - DNA, 24 bp, artificial sequence
  /note=“Description of artificial sequence 24 basepair sequence”
cacgatttgagtatgcaaaccttt

SEQ ID NO: 104 - DNA, 24 bp, artificial sequence
  /note=“Description of artificial sequence 24 basepair sequence”
acgatttgagtatgcaaacctttt

SEQ ID NO: 105 - DNA, 24 bp, artificial sequence
  /note=“Description of artificial sequence 24 basepair sequence”
cgatttgagtatgcaaaccttttc

SEQ ID NO: 106 - DNA, 24 bp, artificial sequence
  /note=“Description of artificial sequence 24 basepair sequence”
gatttgagtatgcaaaccttttcc

SEQ ID NO: 107 - DNA, 24 bp, artificial sequence
  /note=“Description of artificial sequence 24 basepair sequence”
atttgagtatgcaaaccttttcca

SEQ ID NO: 108 - DNA, 24 bp, artificial sequence
  /note=“Description of artificial sequence 24 basepair sequence”
tttgagtatgcaaaccttttccac

SEQ ID NO: 109 - DNA, 24 bp, artificial sequence
  /note=“Description of artificial sequence 24 basepair sequence”
ttgagtatgcaaaccttttccaca

SEQ ID NO: 110 - DNA, 24 bp, artificial sequence
  /note=“Description of artificial sequence 24 basepair sequence”
tgagtatgcaaaccttttccacac

SEQ ID NO: 111 - DNA, 24 bp, artificial sequence
  /note=“Description of artificial sequence 24 basepair sequence”
gagtatgcaaaccttttccacaca

SEQ ID NO: 112 - DNA, 24 bp, artificial sequence
  /note=“Description of artificial sequence 24 basepair sequence”
agtatgcaaaccttttccacacag

SEQ ID NO: 113 - DNA, 24 bp, artificial sequence
  /note=“Description of artificial sequence 24 basepair sequence”
gtatgcaaaccttttccacacagt

SEQ ID NO: 114 - DNA, 24 bp, artificial sequence
  /note=“Description of artificial sequence 24 basepair sequence”
tatgcaaaccttttccacacagtt

SEQ ID NO: 115 - DNA, 24 bp, artificial sequence
  /note=“Description of artificial sequence 24 basepair sequence”
atgcaaaccttttccacacagtta

SEQ ID NO: 116 - DNA, 24 bp, artificial sequence
  /note=“Description of artificial sequence 24 basepair sequence”
tgcaaaccttttccacacagttac

SEQ ID NO: 117 - DNA, 24 bp, artificial sequence
  /note=“Description of artificial sequence 24 basepair sequence”
gcaaaccttttccacacagttacc

SEQ ID NO: 118 - DNA, 24 bp, artificial sequence
  /note=“Description of artificial sequence 24 basepair sequence”
caaaccttttccacacagttaccg

SEQ ID NO: 119 - DNA, 24 bp, artificial sequence
  /note=“Description of artificial sequence 24 basepair sequence”
aaaccttttccacacagttaccga

SEQ ID NO: 120 - DNA, 24 bp, artificial sequence
  /note=“Description of artificial sequence 24 basepair sequence”
aaccttttccacacagttaccgat

SEQ ID NO: 121 - DNA, 24 bp, artificial sequence
  /note=“Description of artificial sequence 24 basepair sequence”
accttttccacacagttaccgatt

SEQ ID NO: 122 - DNA, 24 bp, artificial sequence
  /note=“Description of artificial sequence 24 basepair sequence”
ccttttccacacagttaccgattg

SEQ ID NO: 123 - DNA, 24 bp, artificial sequence
  /note=“Description of artificial sequence 24 basepair sequence”
cttttccacacagttaccgattgg

SEQ ID NO: 124 - DNA, 24 bp, artificial sequence
  /note=“Description of artificial sequence 24 basepair sequence”
ttttccacacagttaccgattggt

SEQ ID NO: 125 - DNA, 24 bp, artificial sequence
  /note=“Description of artificial sequence 24 basepair sequence”
tttccacacagttaccgattggta

SEQ ID NO: 126 - DNA, 24 bp, artificial sequence
  /note=“Description of artificial sequence 24 basepair sequence”
ttccacacagttaccgattggtat

SEQ ID NO: 127 - DNA, 24 bp, artificial sequence
  /note=“Description of artificial sequence 24 basepair sequence”
tccacacagttaccgattggtata

SEQ ID NO: 128 - DNA, 24 bp, artificial sequence
  /note=“Description of artificial sequence 24 basepair sequence”
ccacacagttaccgattggtatag

SEQ ID NO: 129 - DNA, 24 bp, artificial sequence
  /note=“Description of artificial sequence 24 basepair sequence”
cacacagttaccgattggtatagt

SEQ ID NO: 130 - DNA, 24 bp, artificial sequence
  /note=“Description of artificial sequence 24 basepair sequence”
acacagttaccgattggtatagtg

SEQ ID NO: 131 - DNA, 24 bp, artificial sequence
  /note=“Description of artificial sequence 24 basepair sequence”
cacagttaccgattggtatagtgc

SEQ ID NO: 132 - DNA, 24 bp, artificial sequence
  /note=“Description of artificial sequence 24 basepair sequence”
acagttaccgattggtatagtgca

SEQ ID NO: 133 - DNA, 24 bp, artificial sequence
  /note=“Description of artificial sequence 24 basepair sequence”
cagttaccgattggtatagtgcat

SEQ ID NO: 134 - DNA, 24 bp, artificial sequence
  /note=“Description of artificial sequence 24 basepair sequence”
agttaccgattggtatagtgcata

SEQ ID NO: 135 - DNA, 24 bp, artificial sequence
  /note=“Description of artificial sequence 24 basepair sequence”
gttaccgattggtatagtgcatac

SEQ ID NO: 136 - DNA, 24 bp, artificial sequence
  /note=“Description of artificial sequence 24 basepair sequence”
ttaccgattggtatagtgcatacg

SEQ ID NO: 137 - DNA, 24 bp, artificial sequence
  /note=“Description of artificial sequence 24 basepair sequence”
taccgattggtatagtgcatacgt

SEQ ID NO: 138 - DNA, 24 bp, artificial sequence
  /note=“Description of artificial sequence 24 basepair sequence”
accgattggtatagtgcatacgtg

SEQ ID NO: 139 - DNA, 24 bp, artificial sequence
  /note=“Description of artificial sequence 24 basepair sequence”
ccgattggtatagtgcatacgtgg

SEQ ID NO: 140 - DNA, 24 bp, artificial sequence
  /note=“Description of artificial sequence 24 basepair sequence”
cgattggtatagtgcatacgtggc

SEQ ID NO: 141 - DNA, 24 bp, artificial sequence
  /note=“Description of artificial sequence 24 basepair sequence”
gattggtatagtgcatacgtggca

SEQ ID NO: 142 - DNA, 24 bp, artificial sequence
  /note=“Description of artificial sequence 24 basepair sequence”
attggtatagtgcatacgtggcat

SEQ ID NO: 143 - DNA, 24 bp, artificial sequence
  /note=“Description of artificial sequence 24 basepair sequence”
ttggtatagtgcatacgtggcatc

SEQ ID NO: 144 - DNA, 24 bp, artificial sequence
  /note=“Description of artificial sequence 24 basepair sequence”
tggtatagtgcatacgtggcatcc

SEQ ID NO: 145 - DNA, 24 bp, artificial sequence
  /note=“Description of artificial sequence 24 basepair sequence”
ggtatagtgcatacgtggcatcca

SEQ ID NO: 146 - DNA, 24 bp, artificial sequence
  /note=“Description of artificial sequence 24 basepair sequence”
gtatagtgcatacgtggcatccag

SEQ ID NO: 147 - DNA, 24 bp, artificial sequence
  /note=“Description of artificial sequence 24 basepair sequence”
tatagtgcatacgtggcatccagg

SEQ ID NO: 148 - DNA, 24 bp, artificial sequence
  /note=“Description of artificial sequence 24 basepair sequence”
atagtgcatacgtggcatccaggg

SEQ ID NO: 149 - DNA, 24 bp, artificial sequence
  /note=“Description of artificial sequence 24 basepair sequence”
tagtgcatacgtggcatccagggt

SEQ ID NO: 150 - DNA, 24 bp, artificial sequence
  /note=“Description of artificial sequence 24 basepair sequence”
agtgcatacgtggcatccagggtt

SEQ ID NO: 151 - DNA, 24 bp, artificial sequence
  /note=“Description of artificial sequence 24 basepair sequence”
gtgcatacgtggcatccagggtta

SEQ ID NO: 152 - DNA, 24 bp, artificial sequence
  /note=“Description of artificial sequence 24 basepair sequence”
tgcatacgtggcatccagggttac

SEQ ID NO: 153 - DNA, 24 bp, artificial sequence
  /note=“Description of artificial sequence 24 basepair sequence”
gcatacgtggcatccagggttact

SEQ ID NO: 154 - DNA, 24 bp, artificial sequence
  /note=“Description of artificial sequence 24 basepair sequence”
catacgtggcatccagggttactg

SEQ ID NO: 155 - DNA, 24 bp, artificial sequence
  /note=“Description of artificial sequence 24 basepair sequence”
atacgtggcatccagggttactgg

SEQ ID NO: 156 - DNA, 24 bp, artificial sequence
  /note=“Description of artificial sequence 24 basepair sequence”
tacgtggcatccagggttactggc

SEQ ID NO: 157 - DNA, 24 bp, artificial sequence
  /note=“Description of artificial sequence 24 basepair sequence”
acgtggcatccagggttactggct

SEQ ID NO: 158 - DNA, 24 bp, artificial sequence
  /note=“Description of artificial sequence 24 basepair sequence”
cgtggcatccagggttactggctt

SEQ ID NO: 159 - DNA, 24 bp, artificial sequence
  /note=“Description of artificial sequence 24 basepair sequence”
gtggcatccagggttactggcttg

SEQ ID NO: 160 - DNA, 24 bp, artificial sequence
  /note=“Description of artificial sequence 24 basepair sequence”
tggcatccagggttactggcttgc

SEQ ID NO: 161 - DNA, 24 bp, artificial sequence
  /note=“Description of artificial sequence 24 basepair sequence”
ggcatccagggttactggcttgcc

SEQ ID NO: 162 - DNA, 24 bp, artificial sequence
  /note=“Description of artificial sequence 24 basepair sequence”
gcatccagggttactggcttgccc

SEQ ID NO: 163 - DNA, 24 bp, artificial sequence
  /note=“Description of artificial sequence 24 basepair sequence”
catccagggttactggcttgccca

SEQ ID NO: 164 - DNA, 24 bp, artificial sequence
  /note=“Description of artificial sequence 24 basepair sequence”
atccagggttactggcttgcccag

SEQ ID NO: 165 - DNA, 24 bp, artificial sequence
  /note=“Description of artificial sequence 24 basepair sequence”
tccagggttactggcttgcccagt

SEQ ID NO: 166 - DNA, 24 bp, artificial sequence
  /note=“Description of artificial sequence 24 basepair sequence”
ccagggttactggcttgcccagtc

SEQ ID NO: 167 - DNA, 24 bp, artificial sequence
  /note=“Description of artificial sequence 24 basepair sequence”
cagggttactggcttgcccagtcg

SEQ ID NO: 168 - DNA, 24 bp, artificial sequence
  /note=“Description of artificial sequence 24 basepair sequence”
agggttactggcttgcccagtcgg

SEQ ID NO: 169 - DNA, 24 bp, artificial sequence
  /note=“Description of artificial sequence 24 basepair sequence”
gggttactggcttgcccagtcggc

SEQ ID NO: 170 - DNA, 24 bp, artificial sequence
  /note=“Description of artificial sequence 24 basepair sequence”
ggttactggcttgcccagtcggcc

SEQ ID NO: 171 - DNA, 24 bp, artificial sequence
  /note=“Description of artificial sequence 24 basepair sequence”
gttactggcttgcccagtcggcca

SEQ ID NO: 172 - DNA, 24 bp, artificial sequence
  /note=“Description of artificial sequence 24 basepair sequence”
ttactggcttgcccagtcggccac

SEQ ID NO: 173 - DNA, 24 bp, artificial sequence
  /note=“Description of artificial sequence 24 basepair sequence”
tactggcttgcccagtcggccaca

SEQ ID NO: 174 - DNA, 24 bp, artificial sequence
  /note=“Description of artificial sequence 24 basepair sequence”
actggcttgcccagtcggccacat

SEQ ID NO: 175 - DNA, 24 bp, artificial sequence
  /note=“Description of artificial sequence 24 basepair sequence”
ctggcttgcccagtcggccacatt

SEQ ID NO: 176 - DNA, 24 bp, artificial sequence
  /note=“Description of artificial sequence 24 basepair sequence”
tggcttgcccagtcggccacattt

SEQ ID NO: 177 - DNA, 24 bp, artificial sequence
  /note=“Description of artificial sequence 24 basepair sequence”
ggcttgcccagtcggccacatttg

SEQ ID NO: 178 - DNA, 24 bp, artificial sequence
  /note=“Description of artificial sequence 24 basepair sequence”
gcttgcccagtcggccacatttgg

SEQ ID NO: 179 - DNA, 24 bp, artificial sequence
  /note=“Description of artificial sequence 24 basepair sequence”
cttgcccagtcggccacatttggt

SEQ ID NO: 180 - DNA, 24 bp, artificial sequence
  /note=“Description of artificial sequence 24 basepair sequence”
ttgcccagtcggccacatttggtt

SEQ ID NO: 181 - DNA, 24 bp, artificial sequence
  /note=“Description of artificial sequence 24 basepair sequence”
tgcccagtcggccacatttggttt

SEQ ID NO: 182 - DNA, 24 bp, artificial sequence
  /note=“Description of artificial sequence 24 basepair sequence”
gcccagtcggccacatttggtttt

SEQ ID NO: 183 - DNA, 24 bp, artificial sequence
  /note=“Description of artificial sequence 24 basepair sequence”
cccagtcggccacatttggttttt

SEQ ID NO: 184 - DNA, 24 bp, artificial sequence
  /note=“Description of artificial sequence 24 basepair sequence”
ccagtcggccacatttggtttttg

SEQ ID NO: 185 - DNA, 24 bp, artificial sequence
  /note=“Description of artificial sequence 24 basepair sequence”
cagtcggccacatttggtttttgt

SEQ ID NO: 186 - DNA, 24 bp, artificial sequence
  /note=“Description of artificial sequence 24 basepair sequence”
agtcggccacatttggtttttgta

SEQ ID NO: 187 - DNA, 24 bp, artificial sequence
  /note=“Description of artificial sequence 24 basepair sequence”
gtcggccacatttggtttttgtag

SEQ ID NO: 188 - DNA, 24 bp, artificial sequence
  /note=“Description of artificial sequence 24 basepair sequence”
tcggccacatttggtttttgtaga

SEQ ID NO: 189 - DNA, 24 bp, artificial sequence
  /note=“Description of artificial sequence 24 basepair sequence”
cggccacatttggtttttgtagat

SEQ ID NO: 190 - DNA, 24 bp, artificial sequence
  /note=“Description of artificial sequence 24 basepair sequence”
ggccacatttggtttttgtagatg

SEQ ID NO: 191 - DNA, 24 bp, artificial sequence
  /note=“Description of artificial sequence 24 basepair sequence”
gccacatttggtttttgtagatgg

SEQ ID NO: 192 - DNA, 24 bp, artificial sequence
  /note=“Description of artificial sequence 24 basepair sequence”
ccacatttggtttttgtagatggc

SEQ ID NO: 193 - DNA, 24 bp, artificial sequence
  /note=“Description of artificial sequence 24 basepair sequence”
cacatttggtttttgtagatggcc

SEQ ID NO: 194 - DNA, 24 bp, artificial sequence
  /note=“Description of artificial sequence 24 basepair sequence”
acatttggtttttgtagatggcca

SEQ ID NO: 195 - DNA, 24 bp, artificial sequence
  /note=“Description of artificial sequence 24 basepair sequence”
catttggtttttgtagatggccat

SEQ ID NO: 196 - DNA, 24 bp, artificial sequence
  /note=“Description of artificial sequence 24 basepair sequence”
atttggtttttgtagatggccatt

SEQ ID NO: 197 - DNA, 24 bp, artificial sequence
  /note=“Description of artificial sequence 24 basepair sequence”
tttggtttttgtagatggccattg

SEQ ID NO: 198 - DNA, 24 bp, artificial sequence
  /note=“Description of artificial sequence 24 basepair sequence”
ttggtttttgtagatggccattgt

SEQ ID NO: 199 - DNA, 24 bp, artificial sequence
  /note=“Description of artificial sequence 24 basepair sequence”
tggtttttgtagatggccattgtg

SEQ ID NO: 200 - DNA, 24 bp, artificial sequence
  /note=“Description of artificial sequence 24 basepair sequence”
ggtttttgtagatggccattgtga

SEQ ID NO: 201 - DNA, 24 bp, artificial sequence
  /note=“Description of artificial sequence 24 basepair sequence”
gtttttgtagatggccattgtgag

SEQ ID NO: 202 - DNA, 24 bp, artificial sequence
  /note=“Description of artificial sequence 24 basepair sequence”
tttttgtagatggccattgtgagg

SEQ ID NO: 203 - DNA, 24 bp, artificial sequence
  /note=“Description of artificial sequence 24 basepair sequence”
ttttgtagatggccattgtgaggt

SEQ ID NO: 204 - DNA, 24 bp, artificial sequence
  /note=“Description of artificial sequence 24 basepair sequence”
tttgtagatggccattgtgaggta

SEQ ID NO: 205 - DNA, 24 bp, artificial sequence
  /note=“Description of artificial sequence 24 basepair sequence”
ttgtagatggccattgtgaggtat

SEQ ID NO: 206 - DNA, 24 bp, artificial sequence
  /note=“Description of artificial sequence 24 basepair sequence”
tgtagatggccattgtgaggtatg

SEQ ID NO: 207 - DNA, 24 bp, artificial sequence
  /note=“Description of artificial sequence 24 basepair sequence”
gtagatggccattgtgaggtatgt

SEQ ID NO: 208 - DNA, 24 bp, artificial sequence
  /note=“Description of artificial sequence 24 basepair sequence”
tagatggccattgtgaggtatgtt

SEQ ID NO: 209 - DNA, 24 bp, artificial sequence
  /note=“Description of artificial sequence 24 basepair sequence”
agatggccattgtgaggtatgttt

SEQ ID NO: 210 - DNA, 24 bp, artificial sequence
  /note=“Description of artificial sequence 24 basepair sequence”
gatggccattgtgaggtatgtttg

SEQ ID NO: 211 - DNA, 24 bp, artificial sequence
  /note=“Description of artificial sequence 24 basepair sequence”
atggccattgtgaggtatgtttga

SEQ ID NO: 212 - DNA, 291 bp, Nicotiana tabacum
gaagcaattgaaacttttttctgctattgcaaatttgcaatagattctgacgacactgtaccatctgagtgtttgcttaatggtgaattaggtatggttttaagtggttgcttttgctacattgctcagatgcggctttttgcgacacagtcagaatatgcagatcgccttgctgctgcaattgaagcagaaaatcactgtacaagtcagaccagattgcttattgaccagattagccagcagcaaggaagaatagttgctcttgaaggtgcaatgtgtttttcggtgtag

SEQ ID NO: 213 - DNA, 291 bp, Nicotiana tabacum; identical to SEQ ID NO: 212

SEQ ID NO: 214 - protein, 101 aa, Nicotiana tabacum
LALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDP

SEQ ID NO: 215 - protein, 72 aa, Nicotiana tabacum
MAKARLALVGIKMSYEKRWEKKGPNHPYVQFCSMQSAPLVFQYAQVQNPSAVDKESKKSFLKNCQEKAPRRV

SEQ ID NO: 216 - protein, 147 aa, Nicotiana benthamiana
MPVAAVVVMACNRADYLEKTIKSILKYPFIFAHSFLLYACLLCVVEPTFRSTSLWQDGSHPEVRKLALSYDQLTYMQHLDFEPVHTERPGELIAYYKIARKDDWSFFPHHFSRLILIPTSASLALAIVDHSFRRSLQVGVGSAVLQA

SEQ ID NO: 217 - protein, 96 aa, Nicotiana tabacum
EAIETFFCYCKFAIDSDDTVPSECLLNGELGMVLSGCFCYIAQMRLFATQSEYADRLAAAIEAENHCTSQTRLLIDQISQQQGRIVALEGAMCFSV

SEQ ID NO: 218 - protein, 96 aa, Nicotiana tabacum; identical to SEQ ID NO: 217

SEQ ID NO: 219 - DNA, 219 bp, Nicotiana tabacum; identical to SEQ ID NO: 21

SEQ ID NO: 220 - DNA, 477 bp, Nicotiana tabacum
atgggattaacctccatggaggatatacttttgctcagcccaacaatgaaatcaatgggaacaagtcacttacgactgcttgattcacatgcgaaaacgattggctcctatcagttcaagaaaaggatatgggttgagcaaggagaaagagagacaatggcagagaaattcaggaaccagtcacagaaagagagacatgtggcagacaaatccaagaaccagtcatcgaaaaggaataaagaaaaaactgcagaggctaatcccattctagttcgttcactatctagcatatttggacgtcagtctacgagggaaaagcttttaatccttttgctaagaatattatataaccaaaatggaaatcaatctatccccctcgctgccatcagattttctcctatcacaagcctggaagagcaggagatcttggatgctgttgaatctgattcaattatgttggttcttttaagaaaatga

SEQ ID NO: 221 - protein, 72 aa, Nicotiana tabacum; identical to SEQ ID NO: 215

SEQ ID NO: 222 - protein, 158 aa, Nicotiana tabacum
MGLTSMEDILLLSPTMKSMGTSHLRLLDSHAKTIGSYQFKKRIWVEQGERETMAEKFRNQSQKERHVADKSKNQSSKRNKEKTAEANPILVRSLSSIFGRQSTREKLLILLLRILYNQNGNQSIPLAAIRFSPITSLEEQEILDAVESDSIMLVLLRK

SEQ ID NO: 223 - DNA, 477 bp, Nicotiana tabacum; identical to SEQ ID NO: 220

SEQ ID NO: 224 - protein, 158 aa, Nicotiana tabacum; identical to SEQ ID NO: 222

SEQ ID NO: 225 - DNA, 219 bp, Nicotiana benthamiana
atggctatttcatcttggaatgacaatggacaaatgcagttcgtccaagatccttttttttctttactccttttctatgactggttcttggatttgtcttccacatgtccctctttctgtgactggttccagaatttctctgccattgtctctctttctccttgctcaacccatatcctttttaatcaacttgaaatagaatcatatcactcatgctaa

SEQ ID NO: 226 - protein, 72 aa, Nicotiana benthamiana
MAISSWNDNGQMQFVQDPFFSLLLFYDWFLDLSSTCPSFCDWFQNFSAIVSLSPCSTHILFNQLEIESYHSC

SEQ ID NO: 227 - DNA, 318 bp, Nicotiana tabacum
gtaacagaagcaaatattcaacatgatctgtattatcacagaaaggataaggaacaaagcattgaaagaggacacgaagaaactgaaaagggatgggaacaactgaacattcaattcaaaaggaacatgctggattgggaactggacgaactctttgatctatacagaatacttgaagacctcacgatgaacccacaaaacccagacatcctaatgtgggacaacaccagcaagggcatttattcagtgaaggcagggtacaacaaaatgtgcgtatcaaatgaagtgattgatcattggccttggaaactaatctag

SEQ ID NO: 228 - protein, 105 aa, Nicotiana tabacum
VTEANIQHDLYYHRKDKEQSIERGHEETEKGWEQLNIQFKRNMLDWELDELFDLYRILEDLTMNPQNPDILMWDNTSKGIYSVKAGYNKMCVSNEVIDHWPWKLI

SEQ ID NO: 229 - DNA, 192 bp, Nicotiana tabacum
atgctggattgggaactggacgaactctttgatctatacagaatacttgaagacctcacgatgaacccacaaaacccagacatcctaatgtgggacaacaccagcaagggcatttattcagtgaaggcagggtacaacaaaatgtgcgtatcaaatgaagtgattgatcattggccttggaaactaatctag

SEQ ID NO: 230 - protein, 63 aa, Nicotiana tabacum
MLDWELDELFDLYRILEDLTMNPQNPDILMWDNTSKGIYSVKAGYNKMCVSNEVIDHWPWKLI

SEQ ID NO: 231 - DNA, 20 bp, artificial sequence
  /note=“Description of artificial sequence Primer sequence NGSG12045”
aacttgtgggcagtcaggat

SEQ ID NO: 232 - DNA, 20 bp, artificial sequence
  /note=“Description of artificial sequence primer sequence NGSG12045”
gcggttcaccttatctttgc

SEQ ID NO: 233 - DNA, 15001 bp, Nicotiana tabacum
ggcaagccaaggtctttcatttgttctgggtagggtagtagccatataaagtgaagttttagtcttttttctgaaggatatcacgagatatagacagttccctcaagtaaaagaaaaggaaattgtggagcacaccaaaatcaaaatggccaaccacccggagtaataaaaagttagtagaacatagctatgacaaaggcattagggattaaacaaagaaaaaataatccaaaaggatggatggacggtggcctgctttgacatatttgagatttattatgatatgagcagaatgagaatacttgagtatacaggaactttaggatataagtttaatagctagcttgtcattctaggattactccattatgcaacttgctcggttggacaaccactccactttccgcgcataaaacataaaagtaagatatccgttgttgtcattattaataccctccgccacagcgcacagggcttggattggaaattcggaaatctatgatgttatgacacatcttggtgcagcgcaaggattggaagataaaatgttgcagcatttatatttccctttggagctcaagcggcaaggagggtaggtcaattcttgttttactctgaggcatccatattatttccattgttcaaaaactatcagtttcatggatattaatagcataaactttcaacgcgaaattgagtatttatgtaagtattatcatgacaatttgctgggttataaatgtacgcagaaacactctttggatatacgcttaatctttattttaacgtgggctagtggtggcattcctttagtcctattgtatgatgaaacctactccttactttattatatctttgttcgttaataactaatataatgatcattttaacttgtcaatgaagcaacaaaaaaaaaaaacaatcatagacaatgatagtgtacatactgaggtaatattaatttataggagtaccatttaatgatcataacacatgatgtttgaacgaagacacaggagattatacagtaaatattgatcaaatgaagagacccagcacaacatagattagcaaagagtggagtggaagaccataacttagacgcattaggtttctcctgcaagaggaaaagggaaaatcaagaccaggattgcaacaagaaagagagaaaccactaagcttgattggtggatttgtcactacgtacacgatgacaagagaaaaatacttactggtcgtttagtttgtgggatagggataacaatttcagaataaaaatgcaagattcttttaattatgagattaattataccatagttatgatatcatttttatacattctcaatacggaataacaatccccgaattactaatctcaaaataacataccaaaatgactaagatacctttttccaaagctcttctctcaaagtcctttagaaaatcttaggtgaaaattagaaataaaaaattatctcaacttatctaagtataaaattaaatacatgttttatatcttgtatatattttatttttatctaattagccaaatatctactaataaaattatatcgactaaataatcccgccattatacttctggtattatttattcaccaaccaaacgaccctccttaattgttggttgcatgtacaagctattacaatatagtgtttggttgcctcttgaattttgtttaaaattcagcattatatataggatgtttggttgttgtttttattacctgcataaaaaatatataaataaattacgcaaaaattaataaatatattattttatagctgggatataaggtgtaataagaatatgaaaattagtaatatatgtattaaaacaactaaaaagattaaataattttcttctaaataagcaaaacacatattttaatccctgcattataattttatgcatattattcctgtattaaccgttatattattaatctacagaaaattcatcttatttaaaacacggtaatttttttatatttaatttgtgttttttccccttgtgaaatttaattgtcttgtcggagtttatttccaagagagaagagagtatgaaaaggaccaatattgacttgatcctaactgaacaggcaaagtaaatccacggatgaaacactcataactgaacagtgatagactattcgctttctcctaaagccttcaatcgaaatcgcacgatgagagggtacaagttttgctgtgatttccggtacctcctcatcttggctgctgtcgccttcatctacatacaggttctcttatacatggcttatatctcagatctatctttcttgtacgattaagatcaccagcaatgaaataggttcattaggttaggtttcttttggaccttagccttctcttaaattaccactgtttcatatgaactctacatgaacataatttgcaatctttaatacagaaaattgatgactaagaaattagtggaactaattttgaattacgtagaatttagaacaagtttgttattaaatcttaggaaactagagaacaattttaacatcaacttgtgggcagtcaggatttatacctaggggattaaaaaaaaatgcaaacttgcagaatagcttaactatcaaggggattcaacaatttttttatatatataaaaaataatttttccctatttgtacagtgtaactttcctcgcaagagattaaagtgaacccccttcaatacatttattgatttagctgtgtcactagtggggtgtgccactttaagcagctggttccctcttttagtattttggtcgcaaattccccttggcaaagataaggtgaaccgctaggaaagaattgacattcacatgcccaaaagaacttctgtaggctatgcatttgaaattttcatggcttgtaggcgaagcaattgaaacttttttctgctattgcaaatttgcaatagattctgacgacactgtaccatctgaggtaaataacttttggtactgtactgtatggtttagttttggtatctctgttatctctttctaatgtattagacaaaagcaaatatcaagatttaacttctagccccaaggttctggcgtaacaaatgaacaatttgggcaacaatattctcatctgcctaagcttggtggatagagttacttgatatctgtgctagtaggaggtattaagtacccggtggattagtggagatgcatgcaaccgcaattgtaaaaagaaaagtttatattgcttagggaaagccaagcaatatatgaggttacttggttttgttgacatgggtattatgaaaagaatttacctttttttttgatttctttctttttctttctggattagtgtttgcttaatggtgaattaggtatggttttaagtggttgcttttgctacattgctcagatgcggctttttgcgacacagtcagaatatgcagatcgccttgctgctgcagtatgtatctggactcatctagtcatcctccctacaggaaatctaaataccatagacatatttcttttgttctacagtttaagaatttgtattcatgtcatgtattgtgaatatgatgtttctaaaatcttcatatgctctacgtgaaggcatccttcaacaattcaaatgtcattccaaaaatcttctcttttcttctcagaaggatattgcataatctttctttgtgttgtcttaacagcatacaactgcgcccttcttcaatgatgcaggctaaagaaagaagtaaagaacttttaattgctcactatgtgtataaatcattgaatgacacagattgaagcagaaaatcactgtacaagtcagaccagattgcttattgaccagattagccagcagcaaggaagaatagttgctcttgaaggtgcaatgtgtttttcggtgtagtcctttctttcttcattgtcctcttgataaatggatttatttcctccattctacaaatggatctattggaaatagtctatcttgaaaattttatgtaagttttggtcctatcataagtgagtacactgaaaatatttgatcaagaagatgcaagagagtgtagaagatagtaatggttaactccaagtacaaaaatctagatcagagcatgagctaaccaataccaaaactttgcctgctaggccagagtaagagagctaatgaaatctaggaggggaataacgtcatttacaggggaaaggttactccaactaaaaagattcatcaaacatatagatttcagggagcaattaggagttgaaatgccatcaaaacatctgctatttctttctgtccaaatacaccaaaaaatacacgctgggatcatctgccaggtctttttgatggttccgtcaacttcccagaagctccaattttctactgcttcctttaggttctgaggtgttgtccagctaataccaaaaactgataggaacatttaccatatgtctgcagccactgaacaatgcaaaaaaagatgatttactgactctgaactctgatgacacatgtaacatctgttcaccatttgaaaaccccttctacaaatcttgagtcaggatagcttcttcaagggctgtccatgtaaagcacatgacttcagtggggagtttttttctagattagtttccaaggccaatgatcaatcacttcatttgatacgcacattttgttgtaccctgccttcactgaataaatgcccttgctggtgttgtcccacattaggatgtctgggttttgtgggttcatcgtgaggtcttcaagtattctgtatagatcaaagagttcgtccagttcccaatccagcatgttccttttgaattgaatgttcagttgttcccatccctgttatgtgctattgtgttgatgtagatctggtctcttttgagaaattttctgtttttgtgttgtaagtttcgagacatcttcatggatgagcagtgaggataggaccttttcagtttcttcgtgtcctctttcaatgctttgttccttatcctttctgtgataatacagatcatgttgaatatttgcttctgttactgctgatttatgatttactagaataataagtagtttagtcgtaggaggggtctttgtttaaatgtaaatttagttggataagttagttgagatatttgaggtttttgaaatttgaatatttattctgcagattatgttttcaagttggctatttaaagccctctggttaataaaattaaaatgagagacaatttcaaccattcttttaatcttcttgctgctccatctctttaaaaaacctaacagatcccaattaataaaatctggtgtttgctgtcagaaactgaaatgctacttatctcttttgtatgaagggaacaggtagttgtattttttggggggaggggaagaaaggtaatgggtaattttactttccttatcttcatcttgctacattttcagaacaaatgaagcgtcaggaccaggagtgccgacagttaagggctcttgttcaggatcttgaaagtaagttcataaactcctcttcttctttcagcttttagtccaaaagccactgcttttagtcacagtaatatgaaatgtttgcctgtaataatgaaacccattgtacgtggcaaataaagatctgtcagtgtcaatgtgtctgttcatatcattgagttattaatattatgggctctaatcctagatatacccatgctacaagtatttgtacttatttatatagttgatattgttaatttatttgttacaggtaagggcataaaaaagttgatcggaaatgtacaggtgtacatacattctcatatcctcagtcatgctttcactatcaacatctgttgacttcatttctgtcaaatttgtgcatcacctaattactatatttactagatgccagtggctgctgtagttgttatggcttgcaatcgggctgactacctggaaaagactattaaatccatcttaaagtatgttttgtatcaaaacaattttgtctgcttcttattgcatattagatgcctcagctgataagcccggtacttccattgttgtcatcagataccaaatatctgttgcgccaaaatatcctcttttcatatcccaggtacccatttattttcgcacataactttctattgtatgcttgtcttctttttgttgttgaacctacttttcgatctacctccctttggcaggatggatcacatcctgatgttaggaagcttgctttgagctatgatcagctgacgtatatgcaggtaatcttctctaccgcgtgagaagggaaaacaggatgtttggcgtatctctatctttgaaatttaaatcaggtatatgtctttacttggaggggaagtatagacttaagaataagaactcattgttgccaggcttgtttttacttgcaatactcaatcatcatcattaccaataaccatattatgtacagggaaacaagttagtagaaatattgcccataaggagttttcatctgctaaaagattgaaagggaaaagatacattatttatatttaacctgtagatattttccttatcatttcgacccttttattacttcagctttgtatcattgtgtgacacaatttgtccttttccctataagacagcacaagtggaagaggcatgtattgtttgatttatgcttttatgttgcagcttttccccctctcttcatatatatgtgatttctctctctctctctctctctctctctcttatgagtagccacacttctgttccatatattcattcatctactgcaataggttcatagttttgtaacctatcgattgctttttctacctaatgtttttctctgataaaagctacgcattgcataggatatgaatctgtctgcttcattttatcatttggctgcagttactttagtctttatctttaaccttttgctgcctagctgataactgttctggcctggcaatgtgaaatgtagttaacaattgcttctgcttaagctcggtatcaaactcttcttggcgctttttcttgacagttcttaagaaaagactttttcgattctttatcaacagcacttggattttgaacctgtgcatactgaaagaccaggggagctgattgcatactacaaaattgcacgtaaggatgatttggtcctttttttcccatcttttttcgtaactcatttttattccaactagtgctagtcttgccttagccattgtcgatcactctttccgtaggtcattacaagtgggcattggatcagctgttttacaagcataattttagccgtgttatcatactagaaggtactgctgatctatcttaatcactatgttgcatgttctttgctctttttcttctcacaatatctgtgcctctgacatgcagatgatatggaaattgcccctgatttttttgacttttttgaggctggagctactcttcttgacagagacaagtaaggcactcttaaaggatccggatgttgcgttgttttactttcaaagaattattcaattcatcctagtctcaggaaaattactatttttttactcgtgtccaactcccccctcattttcttaaaagaaccaacataattgaatcagattcaacagcatccaagatctcctgctcttccaggcttgtgataggagaaaatctgatggcagcgagggggatagattgatttccattttggttatataatattcttagcaaaaggattaaaagcttttccctcgtagactgacgtccaaatatgctagatagtgaacgaactagaatgggattagcctaaaacatggggataaaaagcctgttctaaatgtcccaagtatgttataagaatttcttaaatacttatggtgaacatcccaggtcgattatggctatttcttcttggaatgacaatggacaaatgcagtttgtccaagatccttgtaagttttttctttcttccttcttttttgtcctttgtgattggtggttatgatttttcttttgaactcttctcctgtttcaattggaaattttactgaccgttattcaatgaagaaacccaaacgctgcttagtgcagatggtttctttttctgttctgttgaatggttatacttcattttctttttgattccttggaagaaattatatcctaaaacagcgtaaaggatttgcttttgagtactttacttttgatatacctctgcagttttttctttattccttttcgatgactggttcttggatttgtctgccacatgtctctctttctgtgactggttcctgaatttctctgccattgtctctctttctccttgctcaacccatatcctttttaatcatcaacttgaaattgaatcatattactcatgctaatacaagcatcagtaagaagactggtagtgttacaatatactagtggtttttctttcattcaatcatcacttgtttgacagcttaaactaggctccactttagagataggtttttggtcttaattaaaataggtcaagggcgcgtcggaacagtcggtagctgcttagtactgaattttaacgtctcctcttttcgttttggagaaaccaatgaaaaaggggaaaagttgaaaatttgctcgttggagttgtaacaggaagttttatgagaaattggaaaacaaaaacaagaaaagaaaatatatttttaaaatttttaggacagggaattaccttttcttgaactgataggagccaatcgttttcgcatgtgaatcaagcagtcgtaagtgacttgttcttttggtacaaacacaaatattttatggctaagattgtcgtaagagaaaattttggggcgctacggttctcttttcaaatccatagccctttctaggattggcttcaattgaatattttggactgtccaaaagaaaaaggagttgcatgtttttaccccattgatttcattgttgggctgagcaaaagtatatcctccatggaggttaatcccattgttttcttctcgatgttgcggaatttattgatattatttaggtgtcttttagcaaagtacacagagctctgcttttctgatctcactgaaatgctttataatttactctgcagatgctctttaccgctctgatttttttcccggtcttggatggatgctttcaaaatctacttgggacgaactatctccaaagtggccaaaggcatatcctttcgaactgatgtgcttatttcttgcctaaattgactaccttggaaacttcaaagattttctttgaccttacttttacttactgggacgactggctaagactcaaagagaatcacagaggtcgacaatttattcgcccagaagtttgcagatcatataattttggtgagcatgtatgtgctccttgaaatcagtgctagatgactttggctcagtagacatagttgagcttgaattctgatcttcaatggtgtgatattcttaatgtttcttactgatcaagaaaaagttaatatgtatctcattgctcttcttactcatttacatgcttatcaagagaaaaaatgtttttgctgttcttaaagatggaaattttattaatttccaccatctaagtcaataacattaaatctttccccatatttaccatcatttacagaaacttctccttaagccttgtcaacaatcttacattatttgcagggttctagtttggggcagtttttcaagcagtatcttgagccaattaaactaaatgatgtccaggcatgttattttattttattgccatcaccccttttcttgcctactcattctttccatttgtatgacatgtattctaccttgaattttgttggaaggttgattggaagtcaatggaccttagttaccttttggaggtaatgacttgaagattatttttgtgctgaaagatttagagaacttgtgaatgctgacaaattattagatggttgattgagaaatttgtcatttaaaccatcttgcgtaggtaacttgtggtttcgtgttctcaggacaattacgtgaaacactttggtgacttggttaaaaaggctaagcccatccatggagctgatgctgttttgaaagcatttaacatagatggtgatgtgcgtattcagtacagagatcaactagactttgaagatatcgcacggcaatttggcatttttgaagaatggaaggtaatgcatatgtgacccttctcttcatattgaattgattatgacctgagatttgatcatatttgtttgagtgggttctttagatgcagtcattacgtatgtcgagtatggctacgtatagcatattagccgtctatctacttaactctgaaacagactgttgagcagttcaaaattcatgcctgattttatccttttaccacttggagatttattgtttcacagccatatgacattttctttcgatatatcatcgatgcaaacagttgctgatctgataacacaaacgctggtaatagtattgcgacgcaaaaatatgcaggtgctcttagtgttagagtaagcaatcagaatccaattgcacaactcattcccttctagaattcaggtgcaaatggaggtgaaatttgaaatacatgcctgccatcttctctttcatttatgcctatctatgggcttgggccccagtaactttccatgcaatatgtgcttgggctagaggttgcgtctgcacgaacgaaaaatgaggtttgccaatatgggcaaaacttgaaccgtgttaggctagcctgcttggtctcatatatttattacaattcatatttttcaaataattgatatagaaagcattcttttggataggttgatatgttatattttgatatgtattcattcttggttttgtaccacatgtatagaatgagtaaaaatgaaataggagattttttaggttcatatattaaaatttagactgatctatagccattttaaatagaattagtgaaaatgaaataggaggagatcttttaggtccataggttgcaatttagattgagctatagtcatttacttgttttatttgtggctttggttacttggttacttaattcttaaacaaactgtttctgcaaatttagttactttttggtaaatatagcctagattaatagtcaatattatagttttcaaatttaaagataaaattttcttaacgcctatttgttgctcaaggccagtactatgggaaagggtggggtggagttgaaattagacctatgatagcccgaccgtagtgatgttaattgtggttacattcataagtagcttggtccatctttattccatttcatatatgtctgaggatgttaatattgaccattgactggcccatatctgttctttgcctgaaccgtggacaggtctattcacactagctgtgactggatttgtcctctttcatggttctccttttgctttctgtaaaacttgcactaactgttcgtttcatcaggatggtgtaccacgggcagcatataaaggaatagtggttttccggtaccaaacgtccagacgtgtattccttgttggccctgattcgcttcaacaactcggaaatgaagatacttaacaaagatatgattggtaagtttctgtccataatgagcaaaactattgagtactctatacacaaagctttagtactttgtcttttaattttttgcatggaatttttttattcttcttcatgaaggaaaatactcaaatgagaataatgtaggaatatgtttggaaacattgtaaaaccacttactttaactccaggaggctaatgtaaactattttggaacaaaatattgaagaaatagcatcaaatattttgagacgtaaggtagaaagatcccaacttgctttgggattgaggcgtagtagctcatcttgttgtaaaatagaaagagggtcatataaattgagatggagggtctatgttacggtcccctgttatagatctagttatgggacgctgtaagacaaaatcagagtaagtttgggtagaggttttcttttttcgacctatagtgttggttcgttaagagagaaagagagaacctgcaatctcgtgagttgaagtactcaaagattggaataattttttgcataccttttactgaattcaaataatttttgatacaaacactgatggattaaccatccacctaaaaattgggaaataacttcctaacataactggaatgagaaagtggcctcctactgataactgctactactaataagtaataactgccacaggaaatatatgaacataactaacagatgcctaaagttgctgagctcatctacttccgatcttctgaaacttattatgtgtaatttgttggtaggctaaaggggtgctaacattactcccctttgtcaaatcgcgcttgtcctcaagcgggaagtatggaaagcgttgttggttggcaaatcagtgttaggatatgactcttgtgcatcagattcttctcctcttcctttatagattcaatccacatttatggcctgggtgaatggttcatcacaattgaaacaaagtcccttaaggcgtctttcctccatctcagatctggtcaatttctttacaaatctggtgtgttgttgcgatgagaaatctgttgtctttgatctacggacatctgataattccgaatgaaggggttgtcccttgcgttcataaagccgggacatactcattgctgtcgccaaatctggagggttatgcaactccacttcagttgctatatagtcagcaagaccactgatataaagttcaatttcttgcgactgtgtgagggtaccagcctgcgaaaccaattgctcaaactttttctggtaatctgccacagacccgatctggcacaacttagccaattctcccaacttttgacttcttattggtggcccagaacgaaggtttcattgacgtttgaattcatccaagatggttgaggcatatctgtctctagtttaaagaaccagagttgtgcattcccctctaaatgaaaagaagcacgtccaacattttcttcttcttcagtttgcttgtgtcgaaagaagtgttcgcacctattcaaccatcccaaagggtcgtctttcccactaaaatgtgggaaattcaactttgtatatttgggaatgctggaacttcctccagtttctgatcctgaccttccttcaactccagctttacccttccaacctcgattgtacctggtattttcgattgattcaaaatcggccttcgaatttgctaaatcctttgtggttgcgagcatatatggcttcctgtctggttagacacgttcttcttggaacaagttcaccaactgtgctagttgttgttgcatttggtctccaataactctcggctgtgataccaagttgtcacggtccctttttatagatgtagttatgggacgctgtaagataaaatcagagttagtttgggaagagattttcttttttcgacctatggtgctggttcgttaagagagaacctgtaatctcttgagttgaagtactcaaaggcaggaataattttatgcatacctttcactgaattcaaataaatttaaatataaacactgatggattaaccacccacctaaaaattgggaaataacccctaacacaactggaatgagaaagtgatctaccaatgtgtgactgccacaggaaatatatgaacataactaatagatgactggactcatctacttcctgatcttttgaaactttccatgtgtaaattgttggtagactaaaggggtgctaacagtatattattgtgaaaataacatttgacctgtttttttaccaataagtaccatatttgctgacactgatgtgtatttcactctctactactccattcaacaggagcccggacaaagatttagacttattgggtaggatgcatcgagctgacaccaaaccatgagtttaccagttacatacaacgttttaattgttatatggaggagctcactgttctagtgttgaagggatatcggcttcttaatattggatgaatcatcacaacctattttttttaagccaagtgttccgaacataaagaggaaatgtagccctgtaaagacaatacctgggacgatcataatcacaggtcaatagttttgcttctcagaaggaacattacaattgtgagcactccgcacgccctcttttggaagaatatgagaacttttctcatttactctagtctattttggaaatgcagattcctcagaatttatattactcttagtgttgtcaaattgacgaacacaactgtgagcacgtaattttttccctacaaaatactcctacaaaaattcacaaaaaatggatttttctacttgtttttgattttatagtttttaggaattcctttttaattgtttatttgcattgtagttgcatttcttgtgcatgttaaatatcttaaaatcatagaaaataccataaaaatgtccaattcttctttgcatagcattttagattttaattgcattttttaggatttattcacatattaattacataattgataaatgaaaatcacaaaaataccctagtcattttacattttttgtttttggttttcagattaataattttcttttattagttcatattgttaaagtaattaattagttaattaataaataaagtagtaaaagaattaattttgcaatttgagttctaggtgctatttgggtttaaagtggctaacattgcaaaaattaaagaagggaaaggaagaggttagtcttcgttgaaaactgggctaagagcacatttgaataggtggcccaaattgccaaattcgcctaagcccaatcttcctaaaacccggtccagctcccctttaaacccaaaacgccctcgtttcagatccttaatcctagtgtcccttgagtttaatccgatggtccggaattgacaa

SEQ ID NO: 234 - DNA, 1341 bp, Nicotiana tabacum
atgagagggtacaagttttgctgtgatttccggtacctcctcatcttggctgctgtcgccttcatctacatacagatgcggctttttgcgacacagtcagaatatgcagatcgccttgctgctgcaattgaagcagaaaatcactgtacaagtcagaccagattgcttattgaccagattagccagcagcaaggaagaatagttgctcttgaagaacaaatgaagcgtcaggaccaggagtgccgacagttaagggctcttgttcaggatcttgaaagtaagggcataaaaaagttgatcggaaatgtacagatgccagtggctgctgtagttgttatggcttgcaatcgggctgactacctggaaaagactattaaatccatcttaaaataccaaatatctgttgcgccaaaatatcctcttttcatatcccaggatggatcacatcctgatgttaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatcctgatgctctttaccgctctgatttttttcccggtcttggatggatgctttcaaaatctacttgggacgaactatctccaaagtggccaaaggcatattgggacgactggctaagactcaaagagaatcacagaggtcgacaatttattcgcccagaagtttgcagatcatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgttttgaaagcatttaacatagatggtgatgtgcgtattcagtacagagatcaactagactttgaagatatcgcacggcaatttggcatttttgaagaatggaaggatggtgtaccacgggcagcatataaaggaatagtggttttccggtaccaaacgtccagacgtgtattccttgttggccctgattcgcttcaacaactcggaaatgaagatacttaa

SEQ ID NO: 235 - protein, 480 aa, Nicotiana tabacum
MRGYKFCCDFRYLLILAAVAFIYIQMRLFATQSEYADRLAAAIEAENHCTSQTRLLIDQISQQQGRIVALEEQMKRQDQECRQLRALVQDLESKGIKKLIGNVQMPVAAVVVMACNRADYLEKTIKSILKYQISVAPKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPDALYRSDFFPGLGWMLSKSTWDELSPKWPKAYWDDWLRLKENHRGRQFIRPEVCRSYNFGEHGSSLGQFFKQYLEPIKLNDVQVDWKSMDLSYLLEDNYVKHFGDLVKKAKPIHGADAVLKAFNIDGDVRIQYRDQLDFEDIARQFGIFEEWKDGVPRAAYKGIVVFRYQTSRRVFLVGPDSLQQLGNEDTCNFIDENTIALFTRSEQCNFIDENTIALFTRSEQ

SEQ ID NO: 236 - DNA, 18 bp, artificial sequence
  primer sequence FABIJI-forward
atcgcacgatgagagggt

SEQ ID NO: 237 - DNA, 24 bp, artificial sequence
  primer sequence FABIJI-reverse
ttaagtatcttcatttccgagttg

SEQ ID NO: 238 - DNA, 23 bp, artificial sequence
  primer sequence CPO-forward
atgagagggtacaagttttgctg

SEQ ID NO: 239 - DNA, 21 bp, artificial sequence
  primer sequence CPO-reverse
gtttggtaccggaaaaccact

SEQ ID NO: 240 - DNA, 23 bp, artificial sequence
  primer sequence CAC80702.1-forward
cagggctacatttcctctttatg

SEQ ID NO: 241 - DNA, 18 bp, artificial sequence
  primer sequence CAC80702.1-reverse
atcgcacgatgagaggga

SEQ ID NO: 242 - DNA, 20 bp, artificial sequence
  primer sequence FABIJI-1 homolog-forward
aacttgtgggcagtcaggat

SEQ ID NO: 243 - DNA, 20 bp, artificial sequence
  primer sequence FABIJI-1 homolog-reverse
gcggttcaccttatctttgc

SEQ ID NO: 244 - DNA, 21 bp, artificial sequence
  primer sequence FABIJI-1 homolog-forward
taatcgacctgggatgttcac

SEQ ID NO: 245 - DNA, 20 bp, artificial sequence
  primer sequence FABIJI-1 homolog-reverse
gcatccaagatctcctgctc

SEQ ID NO: 246 - DNA, 21 bp, artificial sequence
  primer sequence PC181F
tcgctttctcctaaagccttc

SEQ ID NO: 247 - DNA, 25 bp, artificial sequence
  primer sequence PC190R
tgggatatgaaaagaggatattttg

SEQ ID NO: 248 - DNA, 20 bp, artificial sequence
  primer sequence PC191F
aaatgaagcgtcaggaccag

SEQ ID NO: 249 - DNA, 20 bp, artificial sequence
  primer sequence PC192R
gaaagcatccatccaagacc

SEQ ID NO: 250 - DNA, 21 bp, artificial sequence
  primer sequence PC193F
ggaatgacaatggacaaatgc

SEQ ID NO: 251 - DNA, 20 bp, artificial sequence
  primer sequence PC187R
aacatgcacaagaaatgcaa

SEQ ID NO: 252 - DNA, 21 bp, artificial sequence
  primer sequence PC193F
ggaatgacaatggacaaatgc

SEQ ID NO: 253 - DNA, 21 bp, artificial sequence
  primer sequence PC188R
gctcacagttgtgttcgtcaa

SEQ ID NO: 254 - DNA, 21 bp, artificial sequence
  primer sequence PC193F
ggaatgacaatggacaaatgc

SEQ ID NO: 255 - DNA, 23 bp, artificial sequence
  primer sequence PC189R
cagggctacatttcctctttatg

SEQ ID NO: 256 - DNA, 19600 bp, Nicotiana tabacum
atgcaatatccttggaccactccactaccttccttttctgaaacaaaagctctgaagcccactctccttgggactccaatccttaacggcctcccattgtctggaaatacccatccacgcggtctgattttagttttccctggccatataacctgatccaaccgttgagttgcacttgacctattagctggtttggcataaagagactccggaggcacaacggatagcccagagtagttacaccagtatcctatttgccttaaccatcctttgccaactacattgagaatatcaaacgagggacggaacatggatctatctggtttaaatgcaatgggaccacttacccctgtcatgttggtctttaatatgttactaagcaacttcttaccaccatcaaaaatgctaagtgcagcaaggttcatcgtctctccagcaaaactgtccaaattagaatcatttgagtaggagattttgcctccttgatctaaaaactctttaactgcgtaagcaatcatccaaacagtatcataggcgtatagaccgtaggcattcaaaccaacggagctattgctcaacttgttccaccttgatacaaaagccctcttcttttgggaatcaggtgtatggggccgaagggtgagagcaccttgtatagagctagccacctttgttgaaactgaagtcgaatcaaggacaccggaaagccaagaagtagcaatccaaacatattcactcgtcatcatgccaagctcctgggcaacctcaaaaaccttgagacctgttatggatagtgtatgtagaacaataactcgggattcgattgatttaaccttgagcaactcagccacgatcaggtcacgactagacatgagttcaggtggaagaattgccttgtaagaaatcttacaacgtctctcaacaagtttatcacctagagcggcaatactatttcgaccttgatcatcgtctgagaaaattgcaatgacttctctgtattgaaaataactgatcatatcggctacggcagtcattagaaaaagatcactgggggcagtctgaatgaaataggggtactgaagaggtgagagtgtggggtccaatgctgtgaaagaaaggagcgggacatggagttcattcgcaaggtgagagagtacatgggccattacagaactttgagggccaatcacagctactgtatcggtctccatgaattgtaatgctgggaagccagaaaagtagaaaagagttaacaagacgatctagtcaagtgatatctaagagcagtgagagataaattgaaaaagtgtagtatgaaaaggtgagaactatatatatatacctccaatgatcccaaggaatccgctgtagtttgaatcatggagggtgagagcaagttttcttccgtcaagaagagtggtatcagaattgacgtcttggacagcagcttccattgcgattctagcaaccttgccgttggtggtgccaaaagaaaagatggctccaatcttcacctcataagcttgtctctgctcctctgaagattgtccaataaagcagacgaacagaattagcagaaaacaatttaaattcatgatgacgcctccaattgcaattaatgcgttggtaactgtagaaggatcagattaccaacaaaagtaaaataaaacccaatgtgacgaacaactgttagaaatggaggagagagcagggctaaagggacgggcaggaagaacttttcaagtctgagaacttggaagttaattctgtcatgatagaaaataaaaggagacaaccgcagagacagagaggaagcgaccttcaaatcttaaagtttataaactccgagagaggaaacagagaggacaagaaatgtcctttcgaagaggaagtagtgatactagattactaaagtggcaagccaaggtctttcatttgttctgggtagggtagtagccatataaagtgaagttttagtcttttttctgaaggatatcacgagatatagacagttccctcaagtaaaagaaaaggaaattgtggagcacaccaaaatcaaaatggccaaccacccggagtaataaaaagttagtagaacatagctatgacaaaggcattagggattaaacaaagaaaaaataatccaaaaggatggatggacggtggcctgctttgacatatttgagatttattatgatatgagcagaatgagaatacttgagtatacaggaactttaggatataagtttaatagctagcttgtcattctaggattactccattatgcaacttgctcggttggacaaccactccactttccgcgcataaaacataaaagtaagatatccgttgttgtcattattaataccctccgccacagcgcacagggcttggattggaaattcggaaatctatgatgttatgacacatcttggtgcagcgcaaggattggaagataaaatgttgcagcatttatatttccctttggagctcaagcggcaaggagggtaggtcaattcttgttttactctgaggcatccatattatttccattgttcaaaaactatcagtttcatggatattaatagcataaactttcaacgcgaaattgagtatttatgtaagtattatcatgacaatttgctgggttataaatgtacgcagaaacactctttggatatacgcttaatctttattttaacgtgggctagtggtggcattcctttagtcctattgtatgatgaaacctactccttactttattatatctttgttcgttaataactaatataatgatcattttaacttgtcaatgaagcaacaaaaaaaaaaaacaaaatcatagacaatgatagtgtacatactgaggtaatattaatttataggagtaccatttaatgatcataacacatgatgtttgaacgaagacacaggagattatacagtaaatattgatcaaatgaagagacccagcacaacatagattagcaaagagtggagtggaagaccataacttagacgcattaggtttctcctgcaagaggaaaagggaaaatcaagaccaggattgcaacaagaaagagagaaaccactaagcttgattggtggatttgtcactacgtacacgatgacaagagaaaaatacttactggtcgtttagtttgtgggatagggataacaatttcagaataaaaatgcaagattcttttaattatgagattaattataccatagttatgatatcatttttatacattctcaatacggaataacaatccccgaattactaatctcaaaataacataccaaaatgactaagatacctttttccaaagctcttctctcaaagtcctttagaaaatcttaggtgaaaattagaaataaaaaattatctcaacttatctaagtataaaattaaatacatgttttatatcttgtatatattttatttttatctaattagccaaatatctactaataaaattatatcgactaaataatcccgccattatacttctggtattatttattcaccaaccaaacgaccctccttaattgttggttgcatgtacaagctattacaatatagtgtttggttgcctcttgaattttgtttaaaattcagcattatatataggatgtttggttgttgtttttattacctgcataaaaaatatataaataaattacgcaaaaattaataaatatattattttatagctgggatataaggtgtaataagaatatgaaaattagtaatatatgtattaaaacaactaaaaagattaaataattttcttctaaataagcaaaacacatattttaatccctgcattataattttatgcatattattcctgtattaaccgttatattattaatctacagaaaattcatcttatttaaaacacggtaatttttttatatttaatttgtgttttttccccttgtgaaatttaattgtcttgtcggagtttatttccaagagagaagagagtatgaaaaggaccaatattgacttgatcctaactgaacaggcaaagtaaatccacggatgaaacactcataactgaacagtgatagactattcgctttctcctaaagccttcaatcgaaatcgcacgatgagagggtacaagttttgctgtgatttccggtacctcctcatcttggctgctgtcgccttcatctacatacaggttctcttatacatggcttatatctcagatctatctttcttgtacgattaagatcaccagcaatgaaataggttcattaggttaggtttcttttggaccttagccttctcttaaattaccactgtttcatatgaactctacatgaacataatttgcaatctttaatacagaaaattgatgactaagaaattagtggaactaattttgaattacgtagaatttagaacaagtttgttattaaatcttaggaaactagagaacaattttaacatcaacttgtgggcagtcaggatttatacctaggggattaaaaaaaaatgcaaacttgcagaatagcttaactatcaaggggattcaacaattttttttatatatataaaaaataatttttccctatttgtacagtgtaactttcctcgcaagagattaaagtgaacccccttcaatacatttattgatttagctgtgtcactagtggggtgtgccactttaagcagctggttccctcttttagtattttggtcgcaaattccccttggcaaagataaggtgaaccgctaggaaagaattgacattcacatgcccaaaagaacttctgtaggctatgcatttgaaattttcatggcttgtaggcgaagcaattgaaacttttttctgctattgcaaatttgcaatagattctgacgacactgtaccatctgaggtaaataacttttggtactgtactgtatggtttagttttggtatctctgttatctctttctaatgtattagacaaaagcaaatatcaagatttaacttctagccccaaggttctggcgtaacaaatgaacaatttgggcaacaatattctcatctgcctaagcttggtggatagagttacttgatatctgtgctagtaggaggtattaagtacccggtggattagtggagatgcatgcaaccgcaattgtaaaaagaaaagtttatattgcttagggaaagccaagcaatatatgaggttacttggttttgttgacatgggtattatgaaaagaatttaccttttttttttgatttctttctttttctttctggattagtgtttgcttaatggtgaattaggtatggttttaagtggttgcttttgctacattgctcagatgcggctttttgcgacacagtcagaatatgcagatcgccttgctgctgcagtatgtatctggactcatctagtcatcctccctacaggaaatctaaataccatagacatatttcttttgttctacagtttaagaatttgtattcatgtcatgtattgtgaatatgatgtttctaaaatcttcatatgctctacgtgaaggcatccttcaacaattcaaatgtcattccaaaaatcttctcttttcttctcagaaggatattgcataatctttctttgtgttgtcttaacagcatacaactgcgcccttcttcaatgatgcaggctaaagaaagaagtaaagaacttttaattgctcactatgtgtataaatcattgaatgacacagattgaagcagaaaatcactgtacaagtcagaccagattgcttattgaccagattagccagcagcaaggaagaatagttgctcttgaaggtgcaatgtgtttttcggtgtagtcctttctttcttcattgtcctcttgataaatggatttatttcctccattctacaaatggatctattggaaatagtctatcttgaaaattttatgtaagttttggtcctatcataagtgagtacactgaaaatatttgatcaagaagatgcaagagagtgtagaagatagtaatggttaactccaagtacaaaaatctagatcagagcatgagctaaccaataccaaaactttgcctgctaggccagagtaagagagctaatgaaatctaggaggggaataacgtcatttacaggggaaaggttactccaactaaaaagattcatcaaacatatagatttcagggagcaattaggagttgaaatgccatcaaaacatctgctatttctttctgtccaaatacaccaaaaaatacacgctgggatcatctgccaggtctttttgatggttccgtcaacttcccagaagctccaattttctactgcttcctttaggttctgaggtgttgtccagctaataccaaaaactgataggaacatttaccatatgtctgcagccactgaacaatgcaaaaaaagatgatttactgactctgaactctgatgacacatgtaacatctgttcaccatttgaaaaccccttctacaaatcttgagtcaggatagcttcttcaagggctgtccatgtaaagcacatgacttcagtggggagttttttttctagattagtttccaaggccaatgatcaatcacttcatttgatacgcacattttgttgtaccctgccttcactgaataaatgcccttgctggtgttgtcccacattaggatgtctgggttttgtgggttcatcgtgaggtcttcaagtattctgtatagatcaaagagttcgtccagttcccaatccagcatgttccttttgaattgaatgttcagttgttcccatccctgttatgtgctattgtgttgatgtagatctggtctcttttgagaaattttctgtttttgtgttgtaagtttcgagacatcttcatggatgagcagtgaggataggaccttttcagtttcttcgtgtcctctttcaatgctttgttccttatcctttctgtgataatacagatcatgttgaatatttgcttctgttactgctgatttatgatttactagaataataagtagtttagtcgtaggaggggtctttgtttaaatgtaaatttagttggataagttagttgagatatttgaggtttttgaaatttgaatatttattctgcagattatgttttcaagttggctatttaaagccctctggttaataaaattaaaatgagagacaatttcaaccattcttttaatcttcttgctgctccatctctttaaaaaacctaacagatcccaattaataaaatctggtgtttgctgtcagaaactgaaatgctacttatctcttttgtatgaagggaacaggtagttgtattttttggggggaggggaagaaaggtaatgggtaattttactttccttatcttcatcttgctacattttcagaacaaatgaagcgtcaggaccaggagtgccgacagttaagggctcttgttcaggatcttgaaagtaagttcataaactcctcttcttctttcagcttttagtccaaaagccactgcttttagtcacagtaatatgaaatgtttgcctgtaataatgaaacccattgtacgtggcaaataaagatctgtcagtgtcaatgtgtctgttcatatcattgagttattaatattatgggctctaatcctagatatacccatgctacaagtatttgtacttatttatatagttgatattgttaatttatttgttacaggtaagggcataaaaaagttgatcggaaatgtacaggtgtacatacattctcatatcctcagtcatgctttcactatcaacatctgttgacttcatttctgtcaaatttgtgcatcacctaattactatatttactagatgccagtggctgctgtagttgttatggcttgcaatcgggctgactacctggaaaagactattaaatccatcttaaagtatgttttgtatcaaaacaattttgtctgcttcttattgcatattagatgcctcagctgataagcccggtacttccattgttgtcatcagataccaaatatctgttgcgccaaaatatcctcttttcatatcccaggtacccatttattttcgcacataactttctattgtatgcttgtcttctttttgttgttgaacctacttttcgatctacctccctttggcaggatggatcacatcctgatgttaggaagcttgctttgagctatgatcagctgacgtatatgcaggtaatcttctctaccgcgtgagaagggaaaacaggatgtttggcgtatctctatctttgaaatttaaatcaggtatatgtctttacttggaggggaagtatagacttaagaataagaactcattgttgccaggcttgtttttacttgcaatactcaatcatcatcattaccaataaccatattatgtacagggaaacaagttagtagaaatattgcccataaggagttttcatctgctaaaagattgaaagggaaaagatacattatttatatttaacctgtagatattttccttatcatttcgacccttttattacttcagctttgtatcattgtgtgacacaatttgtccttttccctataagacagcacaagtggaagaggcatgtattgtttgatttatgcttttatgttgcagcttttccccctctcttcatatatatgtgatttctctctctctctctctctctctctctcttatgagtagccacacttctgttccatatattcattcatctactgcaataggttcatagttttgtaacctatcgattgctttttctacctaatgtttttctctgataaaagctacgcattgcataggatatgaatctgtctgcttcattttatcatttggctgcagttactttagtctttatctttaaccttttgctgcctagctgataactgttctggcctggcaatgtgaaatgtagttaacaattgcttctgcttaagctcggtatcaaactcttcttggcgctttttcttgacagttcttaagaaaagactttttcgattctttatcaacagcacttggattttgaacctgtgcatactgaaagaccaggggagctgattgcatactacaaaattgcacgtaaggatgatttggtcctttttttcccatcttttttcgtaactcatttttattccaactagtgctagtcttgccttagccattgtcgatcactctttccgtaggtcattacaagtgggcattggatcagctgttttacaagcataattttagccgtgttatcatactagaaggtactgctgatctatcttaatcactatgttgcatgttctttgctctttttcttctcacaatatctgtgcctctgacatgcagatgatatggaaattgcccctgatttttttgacttttttgaggctggagctactcttcttgacagagacaagtaaggcactcttaaaggatccggatgttgcgttgttttactttcaaagaattattcaattcatcctagtctcaggaaaattactatttttttactcgtgtccaactcccccctcattttcttaaaagaaccaacataattgaatcagattcaacagcatccaagatctcctgctcttccaggcttgtgataggagaaaatctgatggcagcgagggggatagattgatttccattttggttatataatattcttagcaaaaggattaaaagcttttccctcgtagactgacgtccaaatatgctagatagtgaacgaactagaatgggattagcctaaaacatggggataaaaagcctgttctaaatgtcccaagtatgttataagaatttcttaaatacttatggtgaacatcccaggtcgattatggctatttcttcttggaatgacaatggacaaatgcagtttgtccaagatccttgtaagttttttctttcttccttcttttttgtcctttgtgattggtggttatgatttttcttttgaactcttctcctgtttcaattggaaattttactgaccgttattcaatgaagaaacccaaacgctgcttagtgcagatggtttctttttctgttctgttgaatggttatacttcattttctttttgattccttggaagaaattatatcctaaaacagcgtaaaggatttgcttttgagtactttacttttgatatacctctgcagttttttctttattccttttcgatgactggttcttggatttgtctgccacatgtctctctttctgtgactggttcctgaatttctctgccattgtctctctttctccttgctcaacccatatcctttttaatcatcaacttgaaattgaatcatattactcatgctaatacaagcatcagtaagaagactggtagtgttacaatatactagtggtttttctttcattcaatcatcacttgtttgacagcttaaactaggctccactttagagataggtttttggtcttaattaaaataggtcaagggcgcgtcggaacagtcggtagctgcttagtactgaattttaacgtctcctcttttcgttttggagaaaccaatgaaaaaggggaaaagttgaaaatttgctcgttggagttgtaacaggaagttttatgagaaattggaaaacaaaaacaagaaaagaaaatatatttttaaaatttttaggacagggaattaccttttcttgaactgataggagccaatcgttttcgcatgtgaatcaagcagtcgtaagtgacttgttcttttggtacaaacacaaatattttatggctaagattgtcgtaagagaaaattttggggcgctacggttctcttttcaaatccatagccctttctaggattggcttcaattgaatattttggactgtccaaaagaaaaaggagttgcatgtttttaccccattgatttcattgttgggctgagcaaaagtatatcctccatggaggttaatcccattgttttcttctcgatgttgcggaatttattgatattatttaggtgtcttttagcaaagtacacagagctctgcttttctgatctcactgaaatgctttataatttactctgcagatgctctttaccgctctgatttttttcccggtcttggatggatgctttcaaaatctacttgggacgaactatctccaaagtggccaaaggcatatcctttcgaactgatgtgcttatttcttgcctaaattgactaccttggaaacttcaaagattttctttgaccttacttttacttactgggacgactggctaagactcaaagagaatcacagaggtcgacaatttattcgcccagaagtttgcagatcatataattttggtgagcatgtatgtgctccttgaaatcagtgctagatgactttggctcagtagacatagttgagcttgaattctgatcttcaatggtgtgatattcttaatgtttcttactgatcaagaaaaagttaatatgtatctcattgctcttcttactcatttacatgcttatcaagagaaaaaatgtttttgctgttcttaaagatggaaattttattaatttccaccatctaagtcaataacattaaatctttccccatatttaccatcatttacagaaacttctccttaagccttgtcaacaatcttacattatttgcagggttctagtttggggcagtttttcaagcagtatcttgagccaattaaactaaatgatgtccaggcatgttattttattttattgccatcaccccttttcttgcctactcattctttccatttgtatgacatgtattctaccttgaattttgttggaaggttgattggaagtcaatggaccttagttaccttttggaggtaatgacttgaagattatttttgtgctgaaagatttagagaacttgtgaatgctgacaaattattagatggttgattgagaaatttgtcatttaaaccatcttgcgtaggtaacttgtggtttcgtgttctcaggacaattacgtgaaacactttggtgacttggttaaaaaggctaagcccatccatggagctgatgctgttttgaaagcatttaacatagatggtgatgtgcgtattcagtacagagatcaactagactttgaagatatcgcacggcaatttggcatttttgaagaatggaaggtaatgcatatgtgacccttctcttcatattgaattgattatgacctgagatttgatcatatttgtttgagtgggttctttagatgcagtcattacgtatgtcgagtatggctacgtatagcatattagccgtctatctacttaactctgaaacagactgttgagcagttcaaaattcatgcctgattttatccttttaccacttggagatttattgtttcacagccatatgacattttctttcgatatatcatcgatgcaaacagttgctgatctgataacacaaacgctggtaatagtattgcgacgcaaaaatatgcaggtgctcttagtgttagagtaagcaatcagaatccaattgcacaactcattcccttctagaattcaggtgcaaatggaggtgaaatttgaaatacatgcctgccatcttctctttcatttatgcctatctatgggcttgggccccagtaactttccatgcaatatgtgcttgggctagaggttgcgtctgcacgaacgaaaaatgaggtttgccaatatgggcaaaacttgaaccgtgttaggctagcctgcttggtctcatatatttattacaattcatatttttcaaataattgatatagaaagcattcttttggataggttgatatgttatattttgatatgtattcattcttggttttgtaccacatgtatagaatgagtaaaaatgaaataggagattttttaggttcatatattaaaatttagactgatctatagccattttaaatagaattagtgaaaatgaaataggaggagatcttttaggtccataggttgcaatttagattgagctatagtcatttacttgttttatttgtggctttggttacttggttacttaattcttaaacaaactgtttctgcaaatttagttactttttggtaaatatagcctagattaatagtcaatattatagttttcaaatttaaagataaaattttcttaacgcctatttgttgctcaaggccagtactatgggaaagggtggggtggagttgaaattagacctatgatagcccgaccgtagtgatgttaattgtggttacattcataagtagcttggtccatctttattccatttcatatatgtctgaggatgttaatattgaccattgactggcccatatctgttctttgcctgaaccgtggacaggtctattcacactagctgtgactggatttgtcctctttcatggttctccttttgctttctgtaaaacttgcactaactgttcgtttcatcaggatggtgtaccacgggcagcatataaaggaatagtggttttccggtaccaaacgtccagacgtgtattccttgttggccctgattcgcttcaacaactcggaaatgaagatacttaacaaagatatgattggtaagtttctgtccataatgagcaaaactattgagtactctatacacaaagctttagtactttgtcttttaattttttgcatggaattttttttattcttcttcatgaaggaaaatactcaaatgagaataatgtaggaatatgtttggaaacattgtaaaaccacttactttaactccaggaggctaatgtaaactattttggaacaaaatattgaagaaatagcatcaaatattttgagacgtaaggtagaaagatcccaacttgctttgggattgaggcgtagtagctcatcttgttgtaaaatagaaagagggtcatataaattgagatggagggtctatgttacggtcccctgttatagatctagttatgggacgctgtaagacaaaatcagagtaagtttgggtagaggttttcttttttcgacctatagtgttggttcgttaagagagaaagagagaacctgcaatctcgtgagttgaagtactcaaagattggaataattttttgcataccttttactgaattcaaataatttttgatacaaacactgatggattaaccatccacctaaaaattgggaaataacttcctaacataactggaatgagaaagtggcctcctactgataactgctactactaataagtaataactgccacaggaaatatatgaacataactaacagatgcctaaagttgctgagctcatctacttccgatcttctgaaacttattatgtgtaatttgttggtaggctaaaggggtgctaacattactcccctttgtcaaatcgcgcttgtcctcaagcgggaagtatggaaagcgttgttggttggcaaatcagtgttaggatatgactcttgtgcatcagattcttctcctcttcctttatagattcaatccacatttatggcctgggtgaatggttcatcacaattgaaacaaagtcccttaaggcgtctttcctccatctcagatctggtcaatttctttacaaatctggtgtgttgttgcgatgagaaatctgttgtctttgatctacggacatctgataattccgaatgaaggggttgtcccttgcgttcataaagccgggacatactcattgctgtcgccaaatctggagggttatgcaactccacttcagttgctatatagtcagcaagaccactgatataaagttcaatttcttgcgactgtgtgagggtaccagcctgcgaaaccaattgctcaaactttttctggtaatctgccacagacccgatctggcacaacttagccaattctcccaacttttgacttcttattggtggcccagaacgaaggtttcattgacgtttgaattcatccaagatggttgaggcatatctgtctctagtttaaagaaccagagttgtgcattcccctctaaatgaaaagaagcacgtccaacattttcttcttcttcagtttgcttgtgtcgaaagaagtgttcgcacctattcaaccatcccaaagggtcgtctttcccactaaaatgtgggaaattcaactttgtatatttgggaatgctggaacttcctccagtttctgatcctgaccttccttcaactccagctttacccttccaacctcgattgtacctggtattttcgattgattcaaaatcggccttcgaatttgctaaatcctttgtggttgcgagcatatatggcttcctgtctggttagacacgttcttcttggaacaagttcaccaactgtgctagttgttgttgcatttggtctccaataactctcggctgtgataccaagttgtcacggtccctttttatagatgtagttatgggacgctgtaagataaaatcagagttagtttgggaagagattttcttttttcgacctatggtgctggttcgttaagagagaacctgtaatctcttgagttgaagtactcaaaggcaggaataattttatgcatacctttcactgaattcaaataaatttaaatataaacactgatggattaaccacccacctaaaaattgggaaataacccctaacacaactggaatgagaaagtgatctaccaatgtgtgactgccacaggaaatatatgaacataactaatagatgactggactcatctacttcctgatcttttgaaactttccatgtgtaaattgttggtagactaaaggggtgctaacagtatattattgtgaaaataacatttgacctgtttttttaccaataagtaccatatttgctgacactgatgtgtatttcactctctactactccattcaacaggagcccggacaaagatttagacttattgggtaggatgcatcgagctgacaccaaaccatgagtttaccagttacatacaacgttttaattgttatatggaggagctcactgttctagtgttgaagggatatcggcttcttaatattggatgaatcatcacaacctattttttttaagccaagtgttccgaacataaagaggaaatgtagccctgtaaagacaatacctgggacgatcataatcacaggtcaatagttttgcttctcagaaggaacattacaattgtgagcactccgcacgccctcttttggaagaatatgagaacttttctcatttactctagtctattttggaaatgcagattcctcagaatttatattactcttagtgttgtcaaattgacgaacacaactgtgagcacgtaattttttccctacaaaatactcctacaaaaattcacaaaaaatggatttttctacttgtttttgattttatagtttttaggaattcctttttaattgtttatttgcattgtagttgcatttcttgtgcatgttaaatatcttaaaatcatagaaaataccataaaaatgtccaattcttctttgcatagcattttagattttaattgcattttttaggatttattcacatattaattacataattgataaatgaaaatcacaaaaataccctagtcattttacattttttgtttttggttttcagattaataattttcttttattagttcatattgttaaagtaattaattagttaattaataaataaagtagtaaaagaattaattttgcaatttgagttctaggtgctatttgggtttaaagtggctaacattgcaaaaattaaagaagggaaaggaagaggttagtcttcgttgaaaactgggctaagagcacatttgaataggtggcccaaattgccaaattcgcctaagcccaatcttcctaaaacccggtccagctcccctttaaacccaaaacgccctcgtttcagatccttaatcctagtgtcccttgagtttaatccgatggtccggaattgacaacccccatcccatataactgtctcacccccctcccccccaaacctagagaccaaacctcgtttccccatctcccctatctctcccattccccactcaaaactctagccgccccaactctttaccccaactctttaccccatgaccctcaaagcctcttattccttaactcatttttatattcccctaaagagccctagaactcatcccgtaacagatctcacaataggttaaccccaaatcttttctttcgatttctaccattcggaggatgaacgcagcgaatttcatttttctctccgacttcagtagtcattagcacgtattcactagccgaattctaaaagcacaaggtcagtgattactcgttgatgaccactgttggtcagagaaacccttgaccaagcgtttgtttgcattttcaaaaggtaacctcgaaatctttgctttgtttttcgttttcgtttaaacccatcttgtggtgtttttcaatttctgttaaaatcgtcaaaaaaataaataattgcatgttctcgtttaaagtttataatctgtccggtttcgcacctttaacttgcaaatagttatataaattatgttttgatgttttgtttaatagtttgtttctaattttctttgttattagattttttttttttttggtttggtttatttttgtttattgtttagacctcaatcttagttaaatgagtttagtttttttaaccagagttcaagttaggaataatttagaatcagttggttaaagaagttttgaaagggcatgggtaattataaggaataggaagggtaattttgtatttaaaaattatgaaatattttctgttataaataagagagagaagagaactgtctctgaaggacataaaataaaaacggtttgggaatctggggtataagtgaacaaaaataaagtttaaaaggtataactgaattagaaaaatcaggtttggttgccctaaaaatcttgttataaaaggtctcattctcacccattttggtgagaaaaaacttagaaaaaagggtcatacggttgctagcaatttaggttccaaatctgagttttaatagctgcaaaaacactccaaaaatagaaagaaaacaatcaagaaagagggattaaaagctgattctaacctctttggactcttgcatcatttctggattcaaaaagcttggtttgatttgaaagatgttggttttactgttgctgtcactctgttgtttagatgggatttggattgttctcatgatttctgctgttgatctgttttgagctcactcaatagctgttttccttggcctttatttggcaaaagttcagcttgatttacaagtttaggtacatacctctcactcgtattttgttctgattttgtaaattttacctcttaccatttaattgaaggaaatttacgattttaaaaatactaaaatgagtaattaagttaaacttttattgttggcttgcgtgacagtggtgttaggcgccatcacgacctttaatggatttttggtcgtgacaccctatgtctaaaataaaatcaattaaggggtgaagaccctatgttagaaaagtgactagggagtgaagaccctatgtcagaaaattaaatcaactagggagtgtagaccctatgtcagaaaataaaatcaactagggagtggagaccctatgttgaaaaagagactagggagtagagaccctatgtctaaaataaaatcaactagggagtgaagaccctatgttggaaaataagactagtgagtggagaccctatgtctaaaataaatatcaactagggagtgaagaccctatgttggaaaagagactagggagtggagaccctatgttggaaaagagactagggagtggagatcctatgttggaaaagagactaggaagtggagaccctatgtctaaaataaaatcaactagggagtggagaccctatgttgaaaaacgcagctagggattggaaaccctatactaccatgattttgaactttttttttttactaagagaatgagtaaaatgcgggaaagaatttggaaaagacttccctttcagagttgttgctgctgcagagctgtttctagcccccgcaatttctttttggttgcacctgcttcttgcaaggttgcttttggattgcacacgtttcctatttttcaaacaaagaacaattgttagtttgaaacaatggttgattttgtggcattgagtgtttcggtcacttgatctcggtccggcttctttgatgatgatttcaaatgcaactggttgtttcctggataccgattgcatttctgaccctggagaacctttggcttttttgaaactctaccatgacgattggtcatgtgggacttaaccttttccaactttattttgcctttgtaggcctttgacttctttctcccaattttaaattcagagcaacggggaatccttggcttttcaaaccttgccacgacggttagtcgcgtgggactcaaccttttcaacttcatttttgctcttgtaggcacttcaatttgatttccttctttcgagagttttcaatttcaaaacatcagctaccatgcccagtcggggtcaacttgatatccctggcgaggttgggtacctttttgcatattagcttgtatcaaataa

SEQ ID NO: 257 - DNA, 1341 bp, Nicotiana tabacum
atgagagggtacaagttttgctgtgatttccggtacctcctcatcttggctgctgtcgccttcatctacatacagatgcggctttttgcgacacagtcagaatatgcagatcgccttgctgctgcaattgaagcagaaaatcactgtacaagtcagaccagattgcttattgaccagattagccagcagcaaggaagaatagttgctcttgaagaacaaatgaagcgtcaggaccaggagtgccgacagttaagggctcttgttcaggatcttgaaagtaagggcataaaaaagttgatcggaaatgtacagatgccagtggctgctgtagttgttatggcttgcaatcgggctgactacctggaaaagactattaaatccatcttaaaataccaaatatctgttgcgccaaaatatcctcttttcatatcccaggatggatcacatcctgatgttaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctctgatttttttcccggtcttggatggatgctttcaaaatctacttgggacgaactatctccaaagtggccaaaggcttactgggacgactggctaagactcaaagagaatcacagaggtcgacaatttattcgcccagaagtttgcagatcatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgttttgaaagcatttaacatagatggtgatgtgcgtattcagtacagagatcaactagactttgaagatatcgcacggcaatttggcatttttgaagaatggaaggatggtgtaccacgggcagcatataaaggaatagtggttttccggtaccaaacgtccagacgtgtattccttgttggccctgattcgcttcaacaactcggaaatgaagatacttaa

SEQ ID NO: 258 - protein, 446 aa, Nicotiana tabacum
MRGYKFCCDFRYLLILAAVAFIYIQMRLFATQSEYADRLAAAIEAENHCTSQTRLLIDQISQQQGRIVALEEQMKRQDQECRQLRALVQDLESKGIKKLIGNVQMPVAAVVVMACNRADYLEKTIKSILKYQISVAPKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPYALYRSDFFPGLGWMLSKSTWDELSPKWPKAYWDDWLRLKENHRGRQFIRPEVCRSYNFGEHGSSLGQFFKQYLEPIKLNDVQVDWKSMDLSYLLEDNYVKHFGDLVKKAKPIHGADAVLKAFNIDGDVRIQYRDQLDFEDIARQFGIFEEWKDGVPRAAYKGIVVFRYQTSRRVFLVGPDSLQQLGNEDT

SEQ ID NO: 259 - DNA, 9210 bp, Nicotiana tabacum
agactattcgctttctcctaaagccttcaatcgaaatcgcacgatgagagggtacaagttttgctgtgatttccggtacctcctcatcttggctgctgtcgccttcatctacatacaggttctcttatacatggcttatatctcagatctatctttcttgtacgattaagatcaccagcaatgaaataggttcattaggttaggtttcttttggaccttagccttctcttaaattaccactgtttcatatgaactctacatgaacataatttgcaatctttaatacagaaaattgatgactaagaaattagtggaactaattttgaattacgtagaatttagaacaagtttgttattaaatcttaggaaactagagaacaattttaacatcaacttgtgggcagtcaggatttatacctaggggattaaaaaaaaatgcaaacttgcagaatagcttaactatcaaggggattcaacaattttttttatatatataaaaaataatttttccctatttgtacagtgtaactttcctcgcaagagattaaagtgaacccccttcaatacatttattgatttagctgtgtcactagtggggtgtgccactttaagcagctggttccctcttttagtattttggtcgcaaattccccttggcaaagataaggtgaaccgctaggaaagaattgacattcacatgcccaaaagaacttctgtaggctatgcatttgaaattttcatggcttgtaggcgaagcaattgaaacttttttctgctattgcaaatttgcaatagattctgacgacactgtaccatctgaggtaaataacttttggtactgtactgtatggtttagttttggtatctctgttatctctttctaatgtattagacaaaagcaaatatcaagatttaacttctagccccaaggttctggcgtaacaaatgaacaatttgggcaacaatattctcatctgcctaagcttggtggatagagttacttgatatctgtgctagtaggaggtattaagtacccggtggattagtggagatgcatgcaaccgcaattgtaaaaagaaaagtttatattgcttagggaaagccaagcaatatatgaggttacttggttttgttgacatgggtattatgaaaagaatttaccttttttttttgatttctttctttttctttctggattagtgtttgcttaatggtgaattaggtatggttttaagtggttgcttttgctacattgctcagatgcggctttttgcgacacagtcagaatatgcagatcgccttgctgctgcagtatgtatctggactcatctagtcatcctccctacaggaaatctaaataccatagacatatttcttttgttctacagtttaagaatttgtattcatgtcatgtattgtgaatatgatgtttctaaaatcttcatatgctctacgtgaaggcatccttcaacaattcaaatgtcattccaaaaatcttctcttttcttctcagaaggatattgcataatctttctttgtgttgtcttaacagcatacaactgcgcccttcttcaatgatgcaggctaaagaaagaagtaaagaacttttaattgctcactatgtgtataaatcattgaatgacacagattgaagcagaaaatcactgtacaagtcagaccagattgcttattgaccagattagccagcagcaaggaagaatagttgctcttgaaggtgcaatgtgtttttcggtgtagtcctttctttcttcattgtcctcttgataaatggatttatttcctccattctacaaatggatctattggaaatagtctatcttgaaaattttatgtaagttttggtcctatcataagtgagtacactgaaaatatttgatcaagaagatgcaagagagtgtagaagatagtaatggttaactccaagtacaaaaatctagatcagagcatgagctaaccaataccaaaactttgcctgctaggccagagtaagagagctaatgaaatctaggaggggaataacgtcatttacaggggaaaggttactccaactaaaaagattcatcaaacatatagatttcagggagcaattaggagttgaaatgccatcaaaacatctgctatttctttctgtccaaatacaccaaaaaatacacgctgggatcatctgccaggtctttttgatggttccgtcaacttcccagaagctccaattttctactgcttcctttaggttctgaggtgttgtccagctaataccaaaaactgataggaacatttaccatatgtctgcagccactgaacaatgcaaaaaaagatgatttactgactctgaactctgatgacacatgtaacatctgttcaccatttgaaaaccccttctacaaatcttgagtcaggatagcttcttcaagggctgtccatgtaaagcacatgacttcagtggggagttttttttctagattagtttccaaggccaatgatcaatcacttcatttgatacgcacattttgttgtaccctgccttcactgaataaatgcccttgctggtgttgtcccacattaggatgtctgggttttgtgggttcatcgtgaggtcttcaagtattctgtatagatcaaagagttcgtccagttcccaatccagcatgttccttttgaattgaatgttcagttgttcccatccctgttatgtgctattgtgttgatgtagatctggtctcttttgagaaattttctgtttttgtgttgtaagtttcgagacatcttcatggatgagcagtgaggataggaccttttcagtttcttcgtgtcctctttcaatgctttgttccttatcctttctgtgataatacagatcatgttgaatatttgcttctgttactgctgatttatgatttactagaataataagtagtttagtagtaggaggggtctttgtttaaatgtaaatttagttggataagttagttgagatatttgaggtttttgaaatttgaatatttattctgcagattatgttttcaagttggctatttaaagccctctggttaataaaattaaaatgagagacaatttcaaccattcttttaatcttcttgctgctccatctctttaaaaaacctaacagatcccaattaataaaatctggtgtttgctgtcagaaactgaaatgctacttatctcttttgtatgaagggaacaggtagttgtattttttggggggaggggaagaaaggtaatgggtaattttactttccttatcttcatcttgctacattttcagaacaaatgaagcgtcaggaccaggagtgccgacagttaagggctcttgttcaggatcttgaaagtaagttcataaactcctcttcttctttcagcttttagtccaaaagccactgcttttagtcacagtaatatgaaatgtttgcctgtaataatgaaacccattgtacgtggcaaataaagatctgtcagtgtcaatgtgtctgttcatatcattgagttattaatattatgggctctaatcctagatatacccatgctacaagtatttgtacttatttatatagttgatattgttaatttatttgttacaggtaagggcataaaaaagttgatcggaaatgtacaggtgtacatacattctcatatcctcagtcatgctttcactatcaacatctgttgacttcatttctgtcaaatttgtgcatcacctaattactatatttactagatgccagtggctgctgtagttgttatggcttgcaatcgggctgactacctggaaaagactattaaatccatcttaaagtatgttttgtatcaaaacaattttgtctgcttcttattgcatattagatgcctcagctgataagcccggtacttccattgttgtcatcagataccaaatatctgttgcgccaaaatatcctcttttcatatcccaggtacccatttattttcgcacataactttctattgtatgcttgtcttctttttgttgttgaacctacttttcgatctacctccctttggcaggatggatcacatcctgatgttaggaagcttgctttgagctatgatcagctgacgtatatgcaggtaatcttctctaccgcgtgagaagggaaaacaggatgtttggcgtatctctatctttgaaatttaaatcaggtatatgtctttacttggaggggaagtatagacttaagaataagaactcattgttgccaggcttgtttttacttgcaatactcaatcatcatcattaccaataaccatattatgtacagggaaacaagttagtagaaatattgcccataaggagttttcatctgctaaaagattgaaagggaaaagatacattatttatatttaacctgtagatattttccttatcatttcgacccttttattacttcagctttgtatcattgtgtgacacaatttgtccttttccctataagacagcacaagtggaagaggcatgtattgtttgatttatgcttttatgttgcagcttttccccctctcttcatatatatgtgatttctctctctctctctctctctctctctcttatgagtagccacacttctgttccatatattcattcatctactgcaataggttcatagttttgtaacctatcgattgctttttctacctaatgtttttctctgataaaagctacgcattgcataggatatgaatctgtctgcttcattttatcatttggctgcagttactttagtctttatctttaaccttttgctgcctagctgataactgttctggcctggcaatgtgaaatgtagttaacaattgcttctgcttaagctcggtatcaaactcttcttggcgctttttcttgacagttcttaagaaaagactttttcgattctttatcaacagcacttggattttgaacctgtgcatactgaaagaccaggggagctgattgcatactacaaaattgcacgtaaggatgatttggtccttttttttcccatcttttttcgtaactcatttttattccaactagtgctagtcttgccttagccattgtcgatcactctttccgtaggtcattacaagtgggcattggatcagctgttttacaagcataattttagccgtgttatcatactagaaggtactgctgatctatcttaatcactatgttgcatgttctttgctctttttcttctcacaatatctgtgcctctgacatgcagatgatatggaaattgcccctgatttttttgacttttttgaggctggagctactcttcttgacagagacaagtaaggcactcttaaaggatccggatgttgcgttgttttactttcaaagaattattcaattcatcctagtctcaggaaaattactatttttttactcgtgtccaactcccccctcattttcttaaaagaaccaacataattgaatcagattcaacagcatccaagatctcctgctcttccaggcttgtgataggagaaaatctgatggcagcgagggggatagattgatttccattttggttatataatattcttagcaaaaggattaaaagcttttccctcgtagactgacgtccaaatatgctagatagtgaacgaactagaatgggattagcctaaaacatggggataaaaagcctgttctaaatgtcccaagtatgttataagaatttcttaaatacttatggtgaacatcccaggtcgattatggctatttcttcttggaatgacaatggacaaatgcagtttgtccaagatccttgtaagttttttctttcttccttcttttttgtcctttgtgattggtggttatgatttttcttttgaactcttctcctgtttcaattggaaattttactgaccgttattcaatgaagaaacccaaacgctgcttagtgcagatggtttctttttctgttctgttgaatggttatacttcattttctttttgattccttggaagaaattatatcctaaaacagcgtaaaggatttgcttttgagtactttacttttgatatacctctgcagttttttctttattccttttcgatgactggttcttggatttgtctgccacatgtctctctttctgtgactggttcctgaatttctctgccattgtctctctttctccttgctcaacccatatcctttttaatcatcaacttgaaattgaatcatattactcatgctaatacaagcatcagtaagaagactggtagtgttacaatatactagtggtttttctttcattcaatcatcacttgtttgacagcttaaactaggctccactttagagataggtttttggtcttaattaaaataggtcaagggcgcgtcggaacagtcggtagctgcttagtactgaattttaacgtctcctcttttcgttttggagaaaccaatgaaaaaggggaaaagttgaaaatttgctcgttggagttgtaacaggaagttttatgagaaattggaaaacaaaaacaagaaaagaaaatatatttttaaaatttttaggacagggaattaccttttcttgaactgataggagccaatcgttttcgcatgtgaatcaagcagtcgtaagtgacttgttcttttggtacaaacacaaatattttatggctaagattgtcgtaagagaaaattttggggcgctacggttctcttttcaaatccatagccctttctaggattggcttcaattgaatattttggactgtccaaaagaaaaaggagttgcatgtttttaccccattgatttcattgttgggctgagcaaaagtatatcctccatggaggttaatcccattgttttcttctcgatgttgcggaatttattgatattatttaggtgtcttttagcaaagtacaaacagagttccgcttttctgatctcactgaaatgctttataatttacactgcagatgctctttaccgctcagatttttttcccggtcttggatggatgctttcaaaatctacttgggacgaattatctccaaagtggccaaaggcatatcctttcgaactgatgtgcttatttcttgcctaaattgactaccttggaaccttcaaagatgttctttgaccttacttttacttactgggacgactggctaagactcaaagagaatcacagaggtcgacaatttattcgcccagaagtttgctgaacatataattttggtgagcatgtatgtgctccttgaaatcagtgctagatgatattggctcagtagacatagttgagcttgaattttgatcttcaatggtgtgatattcttagtgtttcttactgatcaagaatttaatatgtatctcattgctcttcttactcatttagatgcttatcaagaggaaaaatgtttcttgttcttaaagatggaaattttatcaatttccaccatctaagtcaataaaattaaatctttccccatttttaccatcgtttacagaaacttctccttaaaccttgtcaacaatcttacgttaattgcagggttttagtttggggcagtttttcaagcagtatcttgagccaattaaactaaatgatgtccaggcatgttattttattttattgccatcaccccttttcttgcctactcattctttccacttgtatgacatgtattctaccttgaattttgtaaggttgattgggagtcaatggaccttagttaccttttggaggtaatgacttgaagattatttttgtgctgaaagatttagacaacttatgaatgctggcaaattattacatggttgattgagaaatttgtcatttagaccatcttgcgtaggtaacttgtggtttcgtgttctcaggacaattacgtgaaacactttggtgacttggttaaaaaggctaagcccatccatggagctgatgctgttttgaaagcatttaacatagatggtgatgtgcgtattcagtacagagatcaacttgactttgaagatacttaactctttcgatatatcatcgacgcaaacagttgttgatctgatatcacaaacgctggtaatagtattgcgacgcaaaagtatgcaggtgctcttagtgttagagtaagcaatcagaatccaattgcataactcattcccttctataattcaggtgcaaatggaggtgaaatttgaaatacatgcttgccatcttctctttcacttatgcctatctatgggcttgggccccagtaactttccatgcaatatgtgcttgggctagaggctgcgtctgcaggaacaaaaaatggggtttgccaatatgggcaagacttggaccgtgttaggccagcctgtttggcctcatatatttattataattcatttttcatataattgatatagaaagcattcttttggataggttgatgtagtatattttgatatgtattcattctgggttttataccacatgtatagaatgagtacaaatgaaataggagatttcttaggttcatatattaaaatttagactgatctatagccattttgaatagaattagtgaaaatgaaataggaggagatcttttagttccataggttacaatttagattgagcttcagtcatttacttgttttatttgtggctttggttacttggttaattgattacttaattcttaaacaaactgtttctgcaaatttagttactttttggtaaataaagcctagattaatattcaatattatagtttttaaatttaaagataaaattttcttaacgcctatttgttgctcaaggccagtcctatgggaaagggtggggtggagttgaaattagacctatgatagcccgaccgtagtgatgttaattgtggttacattcataagtagcttggtccatctttattccatttcatatatgtctgaggatgttaatattgaggatattcaaggcccatatctgttctttgcctgtactgtggacaggtctattcacactagctgtgactggatttgtcctctttcatggttctccttttgctttccgtaaaacttgcactaactgttcatttcatcaggatggtgtaccacgggcagcatataaaggaatagtggttttccggtaccaaacgtccagacgtgtattccttgttggccctgattcgcttcaacaactcggaaatgaagatacttaacaaagatatgatt

SEQ ID NO: 260 - DNA, 930 bp, Nicotiana tabacum
atgagagggtacaagttttgctgtgatttccggtacctcctcatcttggctgctgtcgccttcatctacatacagatgcggctttttgcgacacagtcagaatatgcagatcgccttgctgctgcaattgaagcagaaaatcactgtacaagtcagaccagattgcttattgaccagattagccagcagcaaggaagaatagttgctcttgaagaacaaatgaagcgtcaggaccaggagtgccgacagttaagggctcttgttcaggatcttgaaagtaagggcataaaaaagttgatcggaaatgtacagatgccagtggctgctgtagttgttatggcttgcaatcgggctgactacctggaaaagactattaaatccatcttaaaataccaaatatctgttgcgccaaaatatcctcttttcatatcccaggatggatcacatcctgatgttaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatcctgatgctctttaccgctcagatttttttcccggtcttggatggatgctttcaaaatctacttgggacgaattatctccaaagtggccaaaggcatattgggacgactggctaagactcaaagagaatcacagaggtcgacaatttattcgcccagaagtttgctga

SEQ ID NO: 261 - protein, 309 aa, Nicotiana tabacum
MRGYKFCCDFRYLLILAAVAFIYIQMRLFATQSEYADRLAAAIEAENHCTSQTRLLIDQISQQQGRIVALEEQMKRQDQECRQLRALVQDLESKGIKKLIGNVQMPVAAVVVMACNRADYLEKTIKSILKYQISVAPKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPDALYRSDFFPGLGWMLSKSTWDELSPKWPKAYWDDWLRLKENHRGRQFIRPEVC

SEQ ID NO: 262 - DNA, 1708 bp, Nicotiana tabacum
cattgacttgatcctaactgaacaggcaaagtaaatccagcgatgaaacactcataactgaacactgagagactattcgctttctcctaaagccttcaatcgaattcgcacgatgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaattgaagatacttaacaaagatatgattgcaggagcccgggcaaaatttttgacttattgggtaggatgcatcgagctgacactaaaccatgattttaccagttacatacaacgttttaatgttatacggaggagctcactgttctagtgttgaagggatatcggcttcttagtattggatgaatcatcaacacaacctattattttaagtgttcagaacataaagaggaaatgtagccctgtaaagactatacatgggaccatcataat

SEQ ID NO: 263 - DNA, 1341 bp, Nicotiana tabacum
atgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaattgaagatacttaa

SEQ ID NO: 264 - protein, 446 aa, Nicotiana tabacum
MRGNKFCCDFRYLLILAAVAFIYTQMRLFATQSEYADRLAAAIEAENHCTSQTRLLIDQISLQQGRIVALEEQMKRQDQECRQLRALVQDLESKGIKKLIGNVQMPVAAVVVMACNRADYLEKTIKSILKYQISVASKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPYALYRSDFFPGLGWMLSKSTWDELSPKWPKAYWDDWLRLKENHRGRQFIRPEVCRTYNFGEHGSSLGQFFKQYLEPIKLNDVQVDWKSMDLSYLLEDNYVKHFGDLVKKAKPIHGADAVLKAFNIDGDVRIQYRDQLDFENIARQFGIFEEWKDGVPRAAYKGIVVFRYQTSRRVFLVGHDSLQQLGIEDT

SEQ ID NO: 265 - DNA, 2111 bp, Nicotiana tabacum
tttagcggccgcgaattcgcccttcattgacttgatcctaactgaacaggcaaagtaaatccagcgatgaaacactcataactgaacactgagagactattcgctttctcctaaagccttcaatcgaattcgcacgatgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaattgaagatacttaacaaagatatgattgcaggagcccgggcaaaatttttgacttattgggtaggatgcgtcgagctgacactaaaccatgattttaccagttacatacaacgttttaatgttatacggaggagctcactgttctagtgttgaagggatatcggcttcttagtattggatgaatcatcaacacaacctattattttaagtgttcagaacataaagaggaaatgtagccctgaagggcgaattcgtttaaacctgcaggactagtccctttagtgagggttaattctgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgca

SEQ ID NO: 266 - DNA, 1341 bp, Nicotiana tabacum; identical to SEQ ID NO: 263

SEQ ID NO: 267 - protein, 446 aa, Nicotiana tabacum; identical to SEQ ID NO: 264

SEQ ID NO: 268 - DNA, 2161 bp, Nicotiana tabacum
cattgacttgatcctaactgaacaggcaaagtaaatccagcgatgaaacactcataactgaacactgagagactattgaatttagcggccgcgaattcgcccttatcgcacgatgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagttgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatgtaattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaattgaagatacttaacaaagatatgattgcaggagcccgggcaaaatttttgacttattgggtaggatgcatcgagctgacactaaaccatgattttaccagttacatacaacgttttaatgttatacggaggagctcactgttctagtgttgaagggatatcggcttaagggcgaattcgtttaaacctgcaggactagtccctttagtgagggttaattctgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactat

SEQ ID NO: 269 - DNA, 1341 bp, Nicotiana tabacum
atgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagttgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatgtaattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaattgaagatacttaa

SEQ ID NO: 270 - protein, 446 aa, Nicotiana tabacum
MRGNKFCCDFRYLLILAAVAFIYTQMRLFATQSEYADRLAAAIEAENHCTSQTRLLIDQISLQQGRIVALEEQMKRQDQECRQLRALVQDLESKGIKKLIGNVQMPVAAVVVMACNRADYLEKTIKSILKYQISVASKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKLIMAISSWNDNGQMQFVQDPYALYRSDFFPGLGWMLSKSTWDELSPKWPKAYWDDWLRLKENHRGRQFIRPEVCRTCNFGEHGSSLGQFFKQYLEPIKLNDVQVDWKSMDLSYLLEDNYVKHFGDLVKKAKPIHGADAVLKAFNIDGDVRIQYRDQLDFENIARQFGIFEEWKDGVPRAAYKGIVVFRYQTSRRVFLVGHDSLQQLGIEDT

SEQ ID NO: 271 - DNA, 2134 bp, Nicotiana tabacum
cattgacttgatcctaactgaacaggcaaagtaaatccagcgatgaaacactcataactgaacactgagagactattcgctttctcggccgcgaattcgcccttatcgcacgatgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgccattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcgggattgaagatacttaacaaagatatgattgcaggagcccgggcaaaatttttgacttattgggtaggatgcatcgagctgacactaaaccatgattttaccagttacatacaacgttttaatgttatacggaggagctcactgttctagtgttgaagggatatcggcttaagggcgaattcgtttaaacctgcaggactagtccctttagtgagggttaattctgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtc

SEQ ID NO: 272 - DNA, 1341 bp, Nicotiana tabacum
atgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgccattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcgggattgaagatacttaa

SEQ ID NO: 273 - protein, 446 aa, Nicotiana tabacum; identical to SEQ ID NO: 264

SEQ ID NO: 274 - DNA, 1708 bp, Nicotiana tabacum
cattgacttgatcctaactgaacaggcaaagtaaatccagcgatgaaacactcataactgaacactgagagactattcgctttctcctaaagccttcaatcgaattcgcacgatgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaaatgaagatacttaacaaagatatgattgcaggagcccgggcaaaatttttgacttattgggtaggatgcatcgagctgacactaaaccatgattttaccagttacatacaacgttttaatgttatacggaggagctcactgttctagtgttgaagggatatcggcttcttagtattggatgaatcatcaacacaacctattattttaagtgttcagaacataaagaggaaatgtagccctgtaaagactatacatgggaccatcataat

SEQ ID NO: 275 - DNA, 1341 bp, Nicotiana tabacum
atgagagggaacaagttttgctgtgatttccggtacctcctcatcttggctgctgtcgccttcatctacacacagatgcggctttttgcgacacagtcagaatatgcagatcgccttgctgctgcaattgaagcagaaaatcattgtacaagccagaccagattgcttattgaccagattagcctgcagcaaggaagaatagttgctcttgaagaacaaatgaagcgtcaggaccaggagtgccgacaattaagggctcttgttcaggatcttgaaagtaagggcataaaaaagttgatcggaaatgtacagatgccagtggctgctgtagttgttatggcttgcaatcgggctgattacctggaaaagactattaaatccatcttaaaataccaaatatctgttgcgtcaaaatatcctcttttcatatcccaggatggatcacatcctgatgtcaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctcagatttttttcccggtcttggatggatgctttcaaaatctacttgggacgaattatctccaaagtggccaaaggcttactgggacgactggctaagactcaaagagaatcacagaggtcgacaatttattcgcccagaagtttgcagaacatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgtcttgaaagcatttaacatagatggtgatgtgcgtattcagtacagagatcaactagactttgaaaatatcgcacggcaatttggcatttttgaagaatggaaggatggtgtaccacgtgcagcatataaaggaatagtagttttccggtaccaaacgtccagacgtgtattccttgttggccatgattcgcttcaacaactcggaaatgaagatacttaa

SEQ ID NO: 276 - protein, 446 aa, Nicotiana tabacum
MRGNKFCCDFRYLLILAAVAFIYTQMRLFATQSEYADRLAAAIEAENHCTSQTRLLIDQISLQQGRIVALEEQMKRQDQECRQLRALVQDLESKGIKKLIGNVQMPVAAVVVMACNRADYLEKTIKSILKYQISVASKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPYALYRSDFFPGLGWMLSKSTWDELSPKWPKAYWDDWLRLKENHRGRQFIRPEVCRTYNFGEHGSSLGQFFKQYLEPIKLNDVQVDWKSMDLSYLLEDNYVKHFGDLVKKAKPIHGADAVLKAFNIDGDVRIQYRDQLDFENIARQFGIFEEWKDGVPRAAYKGIVVFRYQTSRRVFLVGHDSLQQLGNEDT

SEQ ID NO: 277 - DNA, 2100 bp, Nicotiana tabacum
gatttagcggccgcgaattcgcccttcattgacttgatcctaactgaacaggcaaagtaaatccacggatgaaacactcataactgaacagtgatagactattcgctttctcctaaagccttcaatcgaaatcgcacgatgagagggtacaagttttgctgtgatttccggtacctcctcatcttggctgctgtcgccttcatctacatacagatgcggctttttgcgacacagtcagaatatgcagaccgccttgctgctgcaattgaagcagaaaatcactgtacaagtcagaccagattgcttattgaccagattagccagcagcaaggaagaatagttgctcttgaagaacaaatgaagcgtcaggaccaggagtgccgacagttaagggctcttgttcaggatcttgaaagtaagggcataaaaaagttgatcggaaatgtacagatgccagtggctgctgtagttgttatggcttgcaatcgggctgactacctggaaaagactattaaatccatcttaaaataccaaatatctgttgcgccaaaatatcctcttttcatatcccaggatggatcacatcctgatgttaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgacttttttgacttttttgaggctggagctactcttcttgacagagacaagtcgattatggctatttcttcttggaatgacaatggacaaatgcagtttgtccaagatccttatgctctttaccgctctgatttttttcccggtcttggatggatgctttcaaaatctacttgggacgaactatctccaaagtggccaaaggcttactgggacgactggctaagactcaaagagaatcacagaggtcgacaatttattcgcccagaagtttgcagatcatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgttttgaaagcatttaacatagatggtgatgtgcgtattcagtacagagatcaactagactttgaagatatcgcacggcaatttggcatttttgaagaatggaaggatggtgtaccacgggcagcatataaaggaatagtggttttccggtaccaaacgtccagacgtgtattccttgttggccctgattcgcttcaacaactcggaaatgaagatacttaacaaagatatgattggagcccggacaaagatttagacttattgggtaggatgcatcgagctgacaccaaaccatgagtttaccagttacatacaacgttttaattgttatatggaggagctcactgttctagtgttgaagggatatcggcttcttaatattggatgaatcatcacaacctattttttttaagccaagtgttccgaacataaagaggaaatgtagcccaagggcgaattcgtttaaacctgcaggactagtccctttagtgagggttaattctgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatca

SEQ ID NO: 278 - DNA, 1341 bp, Nicotiana tabacum
atgagagggtacaagttttgctgtgatttccggtacctcctcatcttggctgctgtcgccttcatctacatacagatgcggctttttgcgacacagtcagaatatgcagaccgccttgctgctgcaattgaagcagaaaatcactgtacaagtcagaccagattgcttattgaccagattagccagcagcaaggaagaatagttgctcttgaagaacaaatgaagcgtcaggaccaggagtgccgacagttaagggctcttgttcaggatcttgaaagtaagggcataaaaaagttgatcggaaatgtacagatgccagtggctgctgtagttgttatggcttgcaatcgggctgactacctggaaaagactattaaatccatcttaaaataccaaatatctgttgcgccaaaatatcctcttttcatatcccaggatggatcacatcctgatgttaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgacttttttgacttttttgaggctggagctactcttcttgacagagacaagtcgattatggctatttcttcttggaatgacaatggacaaatgcagtttgtccaagatccttatgctctttaccgctctgatttttttcccggtcttggatggatgctttcaaaatctacttgggacgaactatctccaaagtggccaaaggcttactgggacgactggctaagactcaaagagaatcacagaggtcgacaatttattcgcccagaagtttgcagatcatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgttttgaaagcatttaacatagatggtgatgtgcgtattcagtacagagatcaactagactttgaagatatcgcacggcaatttggcatttttgaagaatggaaggatggtgtaccacgggcagcatataaaggaatagtggttttccggtaccaaacgtccagacgtgtattccttgttggccctgattcgcttcaacaactcggaaatgaagatacttaa

SEQ ID NO: 279 - protein, 446 aa, Nicotiana tabacum; identical to SEQ ID NO: 258

SEQ ID NO: 280 - DNA, 2302 bp, Nicotiana tabacum
taaagggactagtcctgcaggtttaaacgaattcgcccttcattgacttgatcctaactgaacaggcaaagtaaatccacggatgaaacactcataactgaacagtgatagactattcgctttctcctaaagccttcaatcgaaatcgcacgatgagagggtacaagttttgctgtgatttccggtacctcctcatcttggctgctgtcgccttcatctacatacagatgcggctttttgcgacacagtcagaatatgcagatcgccttgctgctgcaattgaagcagaaaatcactgtacaagtcagaccagattgcttattgaccagattagccagcagcaaggaagaatagttgctcttgaagaacaaatgaagcgtcaggaccaggagtgccgacagttaagggctcttgttcaggatcttgaaagtaagggcataaaaaagttgatcggaaatgtacagatgccagtggctgctgtagttgttatggcttgcaatcgggctgactacctggaaaagactattaaatccatcttaaaataccaaatatctgttgcgccaaaatatcctcttttcatatcccaggatggatcacatcctgatgttaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctctgatttttttcccggtcttggatggatgctttcaaaatctacttgggacgaactatctccaaagtggccaaaggcttactgggacgactggctaagactcaaagagaatcacagaggtcgacaatttattcgcccagaagtttgcagatcatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgttttgaaagcatttaacatagatggtgatgtgcgtattcagtacagagatcaactaaactttgaagatatcgcacggcaatttggcatttttgaagaatggaaggatggtgtaccacgggcagcatataaaggaatagtggttttccggtaccaaacgtccagacgtgtattccttgttggccctgattcgcctcaacaactcggaaatgaagatacttaacaaagatatgattggagcccggacaaagatttagacttattgggtaggatgcatcgagctgacaccaaaccatgagtttaccagttacatacaacgttttaattgttatatggaggagctcactgttctagcgttgaagggatatcggcttcttaatattggatgaatcatcacaacctattttttttaagccaagtgttccgaacataaagaggaaatgtagccctgaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtcc

SEQ ID NO: 281 - DNA, 1341 bp, Nicotiana tabacum
atgagagggtacaagttttgctgtgatttccggtacctcctcatcttggctgctgtcgccttcatctacatacagatgcggctttttgcgacacagtcagaatatgcagatcgccttgctgctgcaattgaagcagaaaatcactgtacaagtcagaccagattgcttattgaccagattagccagcagcaaggaagaatagttgctcttgaagaacaaatgaagcgtcaggaccaggagtgccgacagttaagggctcttgttcaggatcttgaaagtaagggcataaaaaagttgatcggaaatgtacagatgccagtggctgctgtagttgttatggcttgcaatcgggctgactacctggaaaagactattaaatccatcttaaaataccaaatatctgttgcgccaaaatatcctcttttcatatcccaggatggatcacatcctgatgttaggaagcttgctttgagctatgatcagctgacgtatatgcagcacttggattttgaacctgtgcatactgaaagaccaggggagctgattgcatactacaaaattgcacgtcattacaagtgggcattggatcagctgttttacaagcataattttagccgtgttatcatactagaagatgatatggaaattgcccctgatttttttgacttttttgaggctggagctactcttcttgacagagacaagtcgattatggctatttcttcttggaatgacaatggacaaatgcagtttgtccaagatccttatgctctttaccgctctgatttttttcccggtcttggatggatgctttcaaaatctacttgggacgaactatctccaaagtggccaaaggcttactgggacgactggctaagactcaaagagaatcacagaggtcgacaatttattcgcccagaagtttgcagatcatataattttggtgagcatggttctagtttggggcagtttttcaagcagtatcttgagccaattaaactaaatgatgtccaggttgattggaagtcaatggaccttagttaccttttggaggacaattacgtgaaacactttggtgacttggttaaaaaggctaagcccatccatggagctgatgctgttttgaaagcatttaacatagatggtgatgtgcgtattcagtacagagatcaactaaactttgaagatatcgcacggcaatttggcatttttgaagaatggaaggatggtgtaccacgggcagcatataaaggaatagtggttttccggtaccaaacgtccagacgtgtattccttgttggccctgattcgcctcaacaactcggaaatgaagatacttaa

SEQ ID NO: 282 - protein, 446 aa, Nicotiana tabacum
MRGYKFCCDFRYLLILAAVAFIYIQMRLFATQSEYADRLAAAIEAENHCTSQTRLLIDQISQQQGRIVALEEQMKRQDQECRQLRALVQDLESKGIKKLIGNVQMPVAAVVVMACNRADYLEKTIKSILKYQISVAPKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPYALYRSDFFPGLGWMLSKSTWDELSPKWPKAYWDDWLRLKENHRGRQFIRPEVCRSYNFGEHGSSLGQFFKQYLEPIKLNDVQVDWKSMDLSYLLEDNYVKHFGDLVKKAKPIHGADAVLKAFNIDGDVRIQYRDQLNFEDIARQFGIFEEWKDGVPRAAYKGIVVFRYQTSRRVFLVGPDSPQQLGNEDT